(19) United States
(12) Patent Application Publication
(10) Pub. No.: US 2008/0119517 A1
(43) Pub. Date: May 22, 2008

(54) COMPOSITIONS AND METHODS FOR PROTECTING AGAINST MITOCHONDRIA COMPONENT-MEDIATED PATHOLOGY

(75) Inventors: Bruce S. Kristal, White Plains, NY (US); Robert Friedlander, Brookline, MA (US); M. Flint Beal, New York, NY (US)

Correspondence Address: BURNS & LEVINSON, LLP, 125 SUMMER STREET, BOSTON, MA, 02110, US

(73) Assignees: Cornell Research Foundation, Inc., Ithaca, NY (US); Brigham and Women's Hospital, Inc., Boston, MA (US)

(21) Appl. No.: 11/950,022

(22) Filed: Dec. 4, 2007

Related U.S. Application Data
(62) Division of application No. 10/820,184, filed on Apr. 7, 2004, now abandoned.
(60) Provisional application No. 60/460,989, filed on Apr. 7, 2003.
(60) Provisional application No. 60/519,078, filed on Nov. 10, 2003.

(51) Int. Cl. A61K 31/4709; A61P 25/28; A61P 25/16

(52) U.S. Cl. 514/314

(57) ABSTRACT

Compositions and methods used for preventing mitochondrial component-based diseases are disclosed herein. In particular, heterocyclic compositions and methods that are directed toward protecting against changes in mitochondrial permeability transition that could result in cell death.

### RELATED APPLICATIONS

This application is a divisional of 10/820,184, filed Apr. 7, 2004 and claims priority to and the benefit of U.S. Provisional application 60/460,989, filed Apr. 7, 2003, and U.S. Provisional application 60/519,078, filed Nov. 10, 2003.

### GOVERNMENT SUPPORT

This invention was partially funded from a grant, NIH/NINDS R01 NS039324, the United States government has certain rights to this invention.

### FIELD OF THE INVENTION

The present invention relates to compositions and methods used for preventing cell death in pathological states in which mitochondria contribute to cell death cascades. In particular, heterocyclic, tricyclics and phenothiazine compositions and analogues of the same are directed toward protecting against induction in mitochondrial permeability transition or similar events that can trigger downstream changes (e.g., caspase activation, apoptosis—inducing factor release, energy deprivation) resulting in cell death.

### BACKGROUND OF THE INVENTION

Therapeutic interventions aimed at preventing cell death have potential clinical utility under many circumstances, including neurodegenerative conditions and stroke. A large, compelling body of data indicates that mitochondria function as a major checkpoint on several pathways leading to dysfunction and premature death of cells, including cells of the nervous system. More specifically, mitochondria appear to link inducers and effectors of cell death pathways by releasing a series of protein factors involved in activating both caspase dependent (cytochrome C, SMAC/DIABLO) and caspase independent pathways of cell death (AIF, endonuclease G). The mechanism(s) by which these factors exit the mitochondria, particularly mitochondria from the CNS, remains controversial, but data suggests both PT-dependent and PT-independent (“PT” referring to mitochondrial permeability transition) events may be initiated by different pathogenic insults.

Mitochondrial transport of Ca²⁺ is essential for cellular signaling cascades, as well as for the regulation of mitochondrial energy production. Excess uptake of C²⁺ by the mitochondria can, however, also contribute to cell death following induction of the permeability transition (PT). Permeability transition induction uncouples oxidative phosphorylation, leading to a potential collapse in ATP formation. Cell death may follow from energy deprivation (loss of ATP) or from PT-associated release of cytochrome C or other factors. An example of a disease whose etiology is associated with abnormal cerebral energy metabolism is Huntington's disease, this disease is also associated with abnormal sensitivity to the permeability transition.

Induction of PT has been linked to cytotoxicity following pathological insults such as cardiac ischemia-reperfusion injury, stroke and excitotoxicity, and hypoglycemia. The PT, which has been defined primarily based on studies in purified liver and heart mitochondria, is the opening of pores in the inner mitochondria membrane that allow free diffusion of all solutes<1.5 kD. Therefore, PT induction lads to loss of the proton gradient, to the inability to conduct oxidative phosphorylation, and to a potentially lethal efflux of any mitochondrially sequestered calcium into the cytosol. PT induction also appears associated with release of cytochrome C, AIF, and SMAC/DIABLO. These observations suggest direct linkages may exist between PT and the induction of the direct mediators of downstream caspase dependent and independent cell death pathways.

Data from studies at the level of isolated mitochondria, cells, and intact animal models support the existence of “PT-like” events in the nervous system; although it is worth noting that equally compelling data, including data from our group and others, suggest that “PT-like” events in the nervous system must be significantly different than those in the brain, and that cell death in the CNS may often be “PT-independent”. Most importantly, a comparison of observations in isolated liver and brain mitochondria suggest that “PT-like” events do occur in brain mitochondria, even if the full PT as described in liver and heart mitochondria does not.

Clearly, there exists a need to have compositions and methods for protecting against permeability transition and/or other triggers that cause mitochondrial release of sequestered factors that can promote the downstream stages of cell death.

### SUMMARY

The present invention relates to compositions and methods used for preventing cell death in pathological states in which mitochondria contribute to cell death cascades. In particular, heterocyclic, tricyclics and phenothiazine compositions and analogues of the same are directed toward protecting against induction of the mitochondrial permeability transition or similar events that can trigger the downstream changes (e.g., caspase activation, apoptosis-inducing factor release, energy deprivation) that could result in cell death. Other mitochondrial events that lead to factor release culminating in cell death are also considered to be with the scope of this invention.

The invention pertains to chemical compounds that can be used to protect against mitochondrial permeability transition or similar events that can trigger the downstream changes (e.g., caspase activation, apoptosis-inducing factor release, energy deprivation) that could result in cell death. These compounds am heterocylic in their chemistry and comprise two or more rings of which one or more is either a six or seven member ring. It should be noted that some active analogues of the heterocyclics have only carbon atoms in the rings and are within the scope of this invention. The compounds of the present invention are compounds that when subjected to the methods of the instant invention protect against mitochondrial permeability transition.

In one embodiment, methods for protecting a subject against mitochondrial permeability transition, and hence, a mitochondrial component-mediated disease, are disclosed. In one aspect of this embodiment, a subject is administered a therapeutically effective amount of a compound of the present invention, i.e., a compound that protects against mitochondrial permeability transition. These compounds can be administered as a prophylactic.

In another embodiment, methods for treating a subject having a disease or undergoing an event in which cell death mitochondrial triggers are activated or at risk of being activated are disclosed. In one aspect of this embodiment, a subject is administered a therapeutically effective amount of a compound of the present invention, i.e., a compound that protects against mitochondrial permeability transition and/or mitochondrial release of sequestered factors that contribute to cell death cascades.

### BRIEF DESCRIPTION OF THE DRAWINGS

FIG. 1 represents a library of compounds that was used for screening;

FIG. 2 is a schematic drawing of a screening plate and the data obtained therefrom;

FIG. 3 (a) is the drug structure, name, and therapeutic class of each heterocyclic-related structure in the initial compound library, and (b) represents the relative areas under the curve for the 32 heterocyclic compounds examined;

FIG. 4 depicts data showing that the inhibition of (a) phospholipase A and (b) calmodulin is not related to inhibition of PT induction;

FIG. 5 illustrates heterocyclic-mediated delay PT induction in isolated liver mitochondria without impairing mitochondrial physiology, where (a) is oxygen consumption measured by Clark electrode, (b) is Ca²⁺ transport measured by Ca²⁺ selective electrode, (c) is Δψ measured using a TPP⁺ electrode, and (d) is swelling (absorbance) monitored by a light emitting diode (LED) at A₆₆₀;

FIG. 6 illustrates protection provided by promethazine for pr cerebrocortical neurons from OGD, indicated by LDH activity (a) and active caspase 3 (b) in the supernatant and lysates respectively of control and OGD challenged pry cerebrocortical neurons in the presence or absence of promethazine, *P=0.05; **P<0.001;

FIG. 7 illustrates that promethazine protects mice from MCA occlusion/reperfusion where (a) total stroke volume in C57/B16 mice untreated and treated, P<0.005; means shown with standard deviation to emphasize reduced variability of stroke volume in drug treatment, and with standard error to show accuracy of population estimate, (b) neurological examination assessed at 24 hrs post reperfusion of right MCA, mean ± sem, P<0.05, (c) neurological examination assessed at 30 minutes post occlusion of right MCA, mean ± sem, P=1.0 and (d) Stroke Volume vs Neurologic examination, diagonal added for emphasis (left of line, 13 treated, 2 control, right of line, 11 control). Inverted triangles=promethazine treated; dots=controls; N=13 in each group;

FIG. 8 is a bar graph showing the effect of promethazine on striatal lesion volume;

FIG. 9 is a bar graph showing the effect of promethazine on dopamine depletion in the presence of MPTP;

FIG. 10 is a bar graph showing the effect of promethazine on dopaminergic neuron loss induced by MPTP;

FIG. 11 is a bar graph showing the effect of promethazine in the depletion of striatal dopamine levels;

FIG. 12 is a bar graph showing the reduction in neuronal cell death by nortriptyline;

FIG. 13 shows the effect of nortriptyline on OGD-mediated caspase-3 activation where (a) is a Western blot for caspase-3. The first lane is control, second following OGD, and the third is OGD with drug, and (b) is a bar graph that shows at caspase-3 activation is attenuated with nortriptyline; the bars represent the bands from (a).

FIG. 14 shows the effect of nortriptyline on OGD-mediated cytochrome C release, where (a) is Western blot for released cytochrome c. The first lane is control, second following OGD, and the third is OGD with drug, and (b) is a bar graph showing that cytochrome C release is minimized with nortriptyline;

FIG. 15 is a bar graph showing the effect of nortriptyline on infarct volume;

FIG. 16 is a graph showing the effect of nortriptyline on infarct volume; and

FIG. 17 is a bar graph showing the effect of nortriptyline on a neurological examination.

### DETAILED DESCRIPTION

The present invention relates to compositions and methods used for preventing and treating cell death in pathological states in which mitochondria contribute to cell death cascades. In particular, heterocyclic, tricyclics and phenothiazine compositions and analogues of the same are directed toward protecting against induction in mitochondrial permeability transition or similar events that can trigger the downstream changes (e.g., caspase activation, apoptosis-inducing factor release, energy deprivation) that may result in cell death. Other mitochondrial events that lead to factor release culminating in cell death are also considered to be within the scope of is invention.

In one particular aspect, a library comprising numerous compounds, many of which are FDA-approved drugs, some of which am heterocyclic compounds or analogues thereof were screened using the method of the present embodiment. This library was then screened blindly with the aim of ascertaining which compound was an effective neuroprotective compound, i.e., protective against mitochondrial permeability transition.

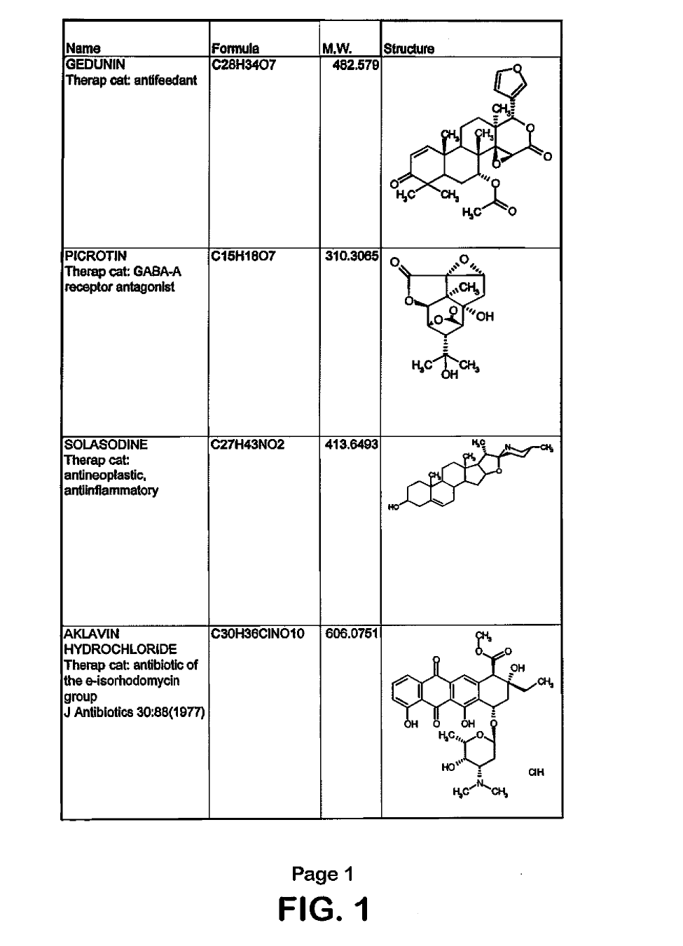

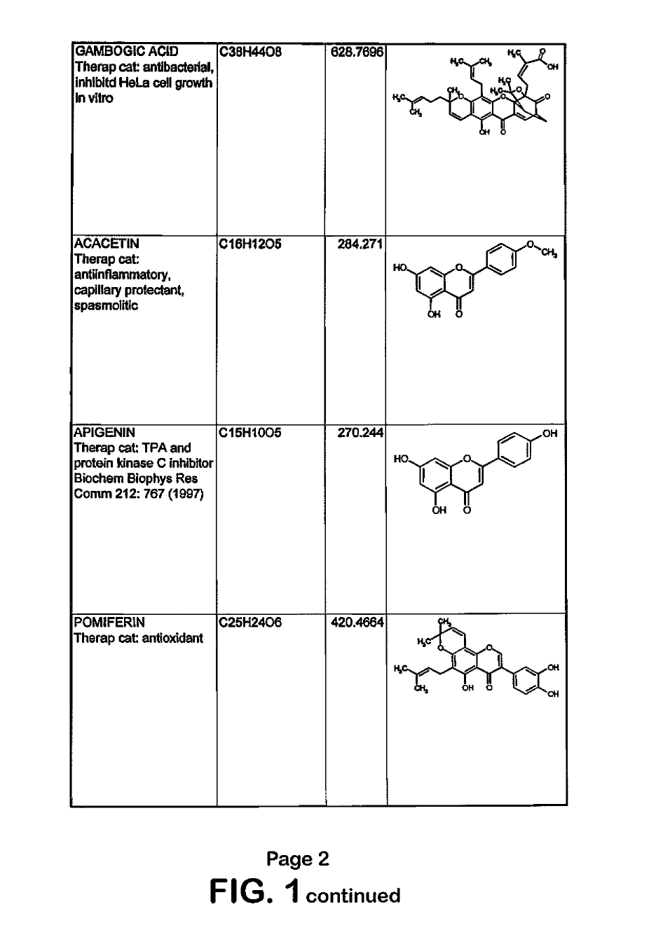

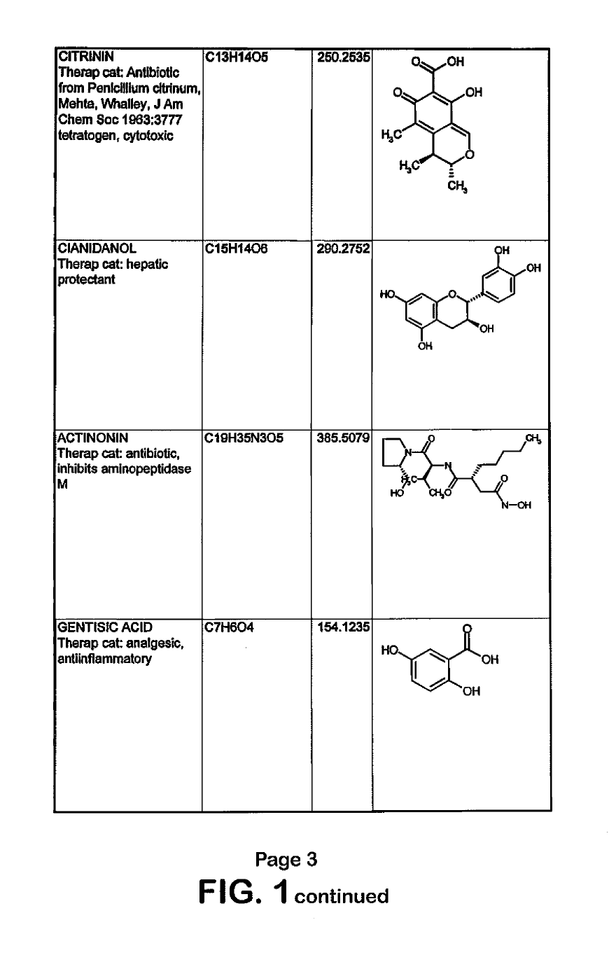

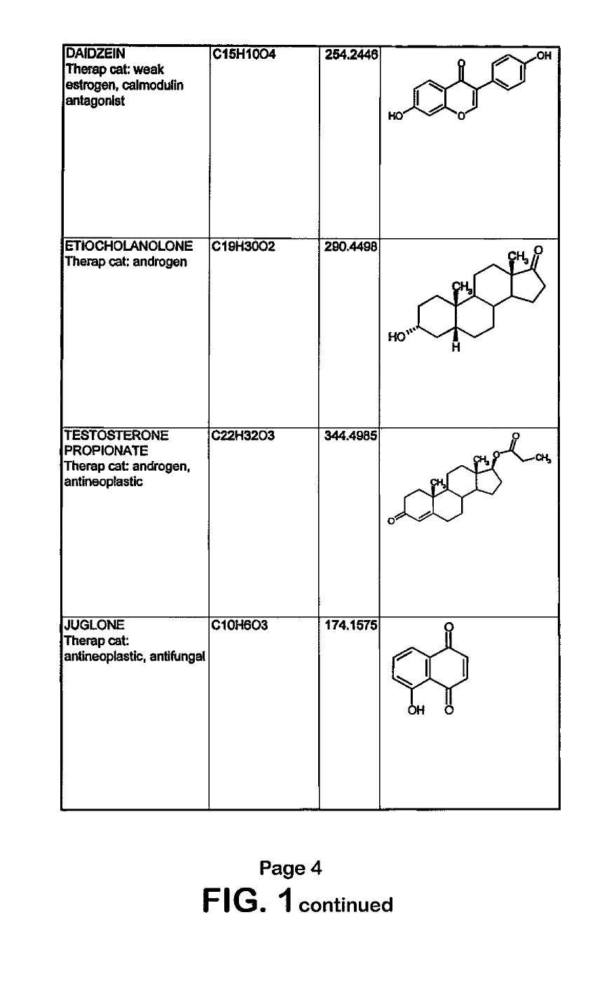

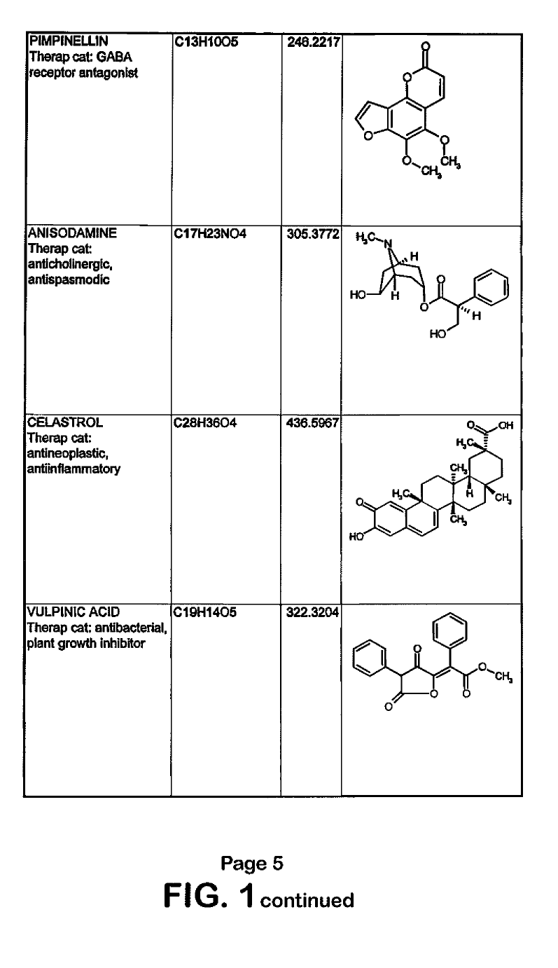

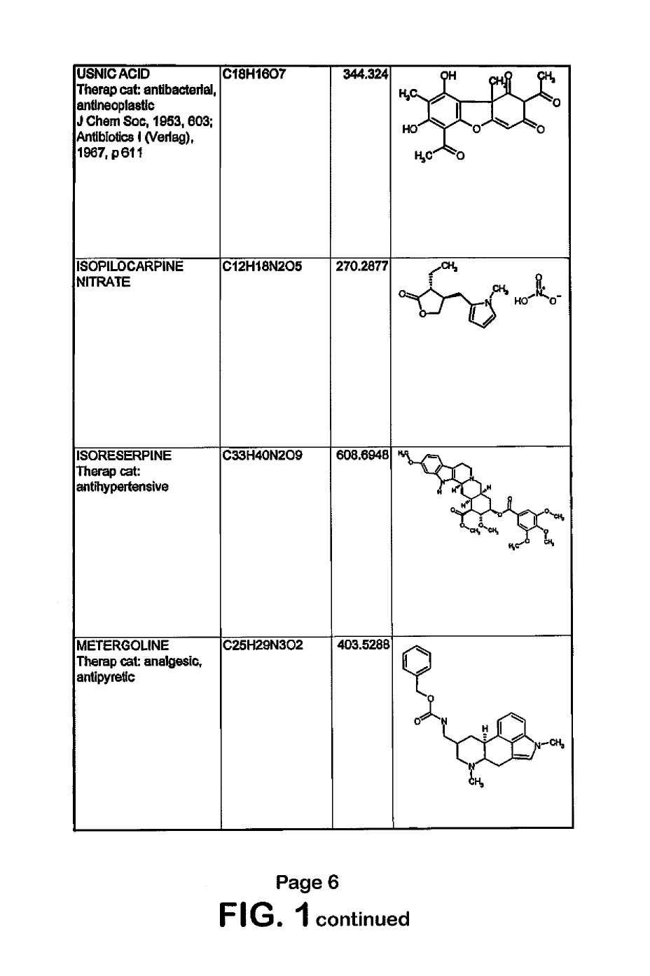

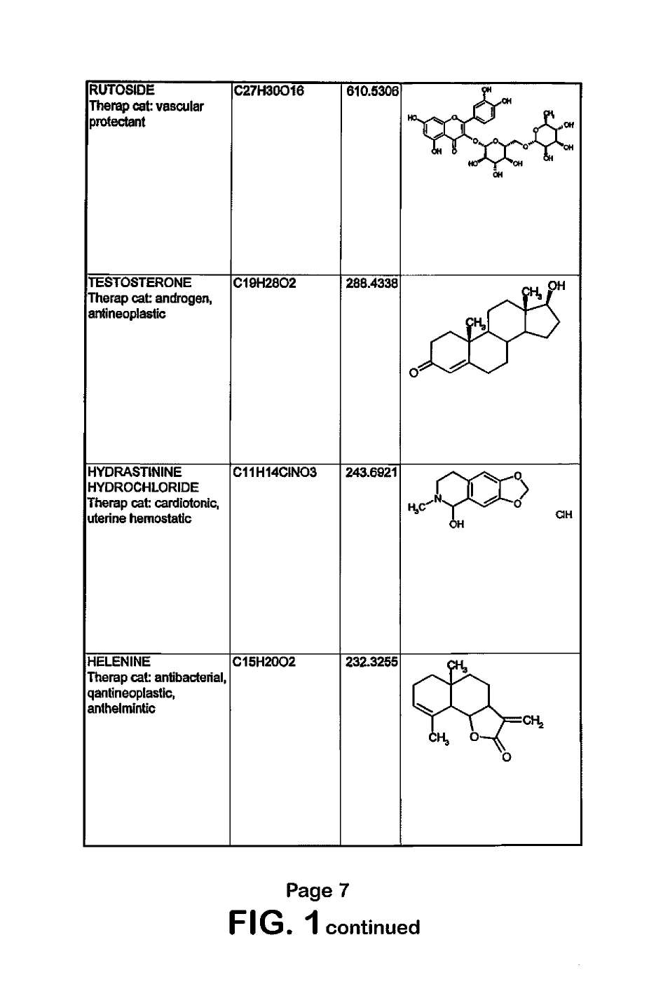

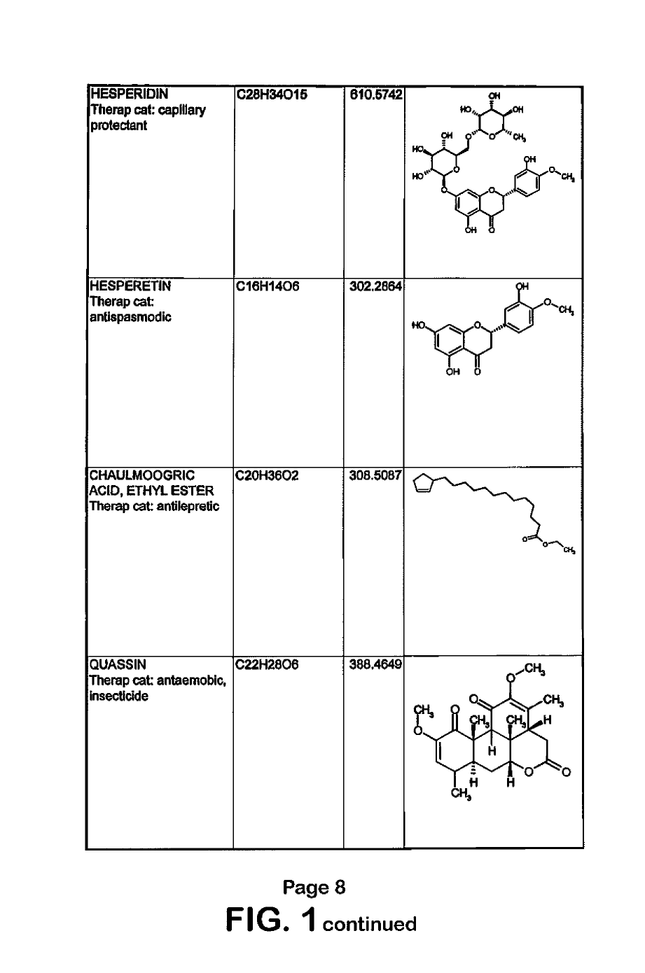

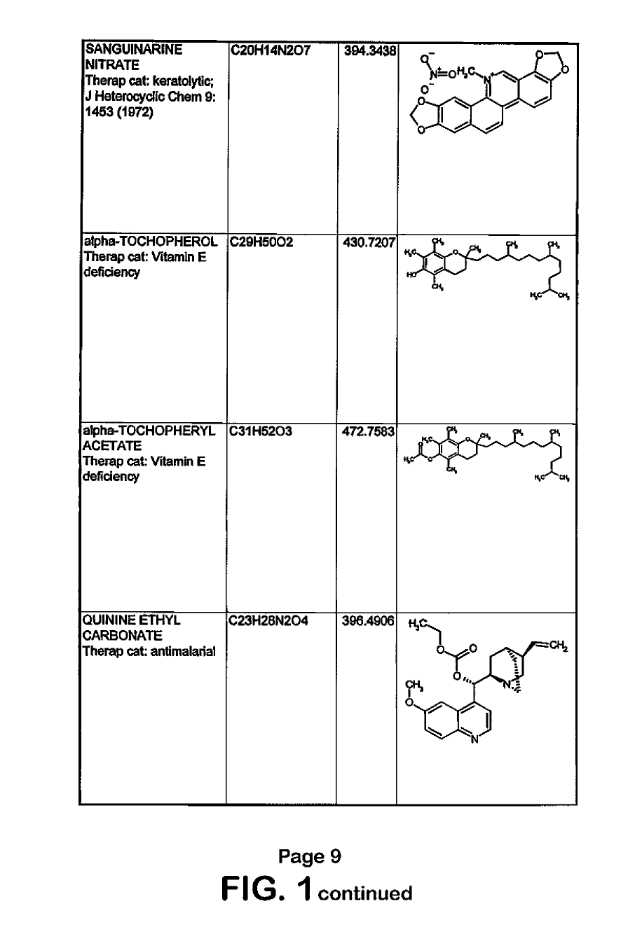

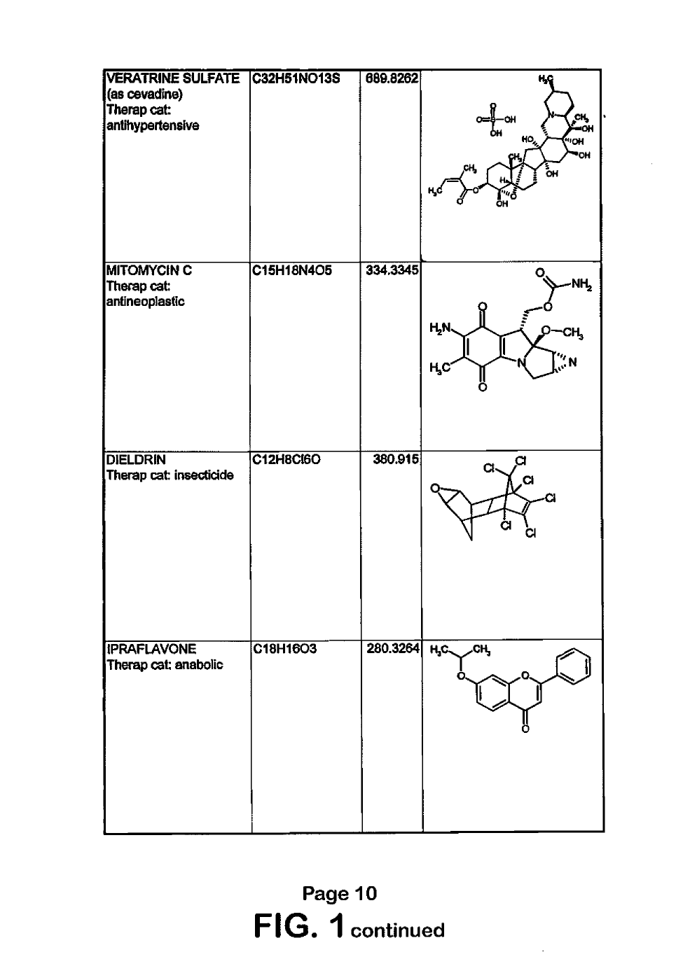

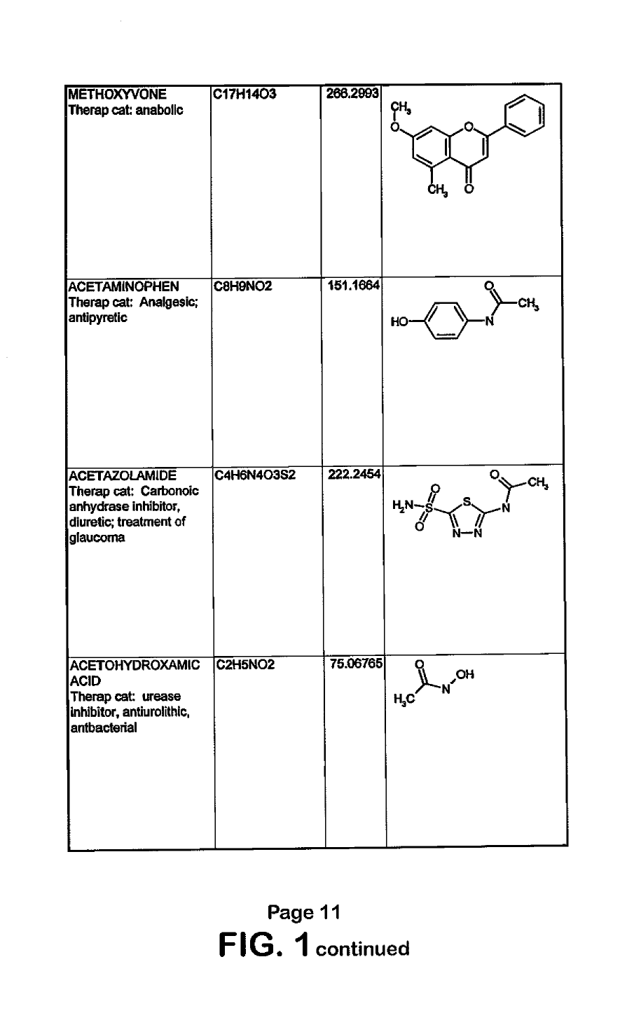

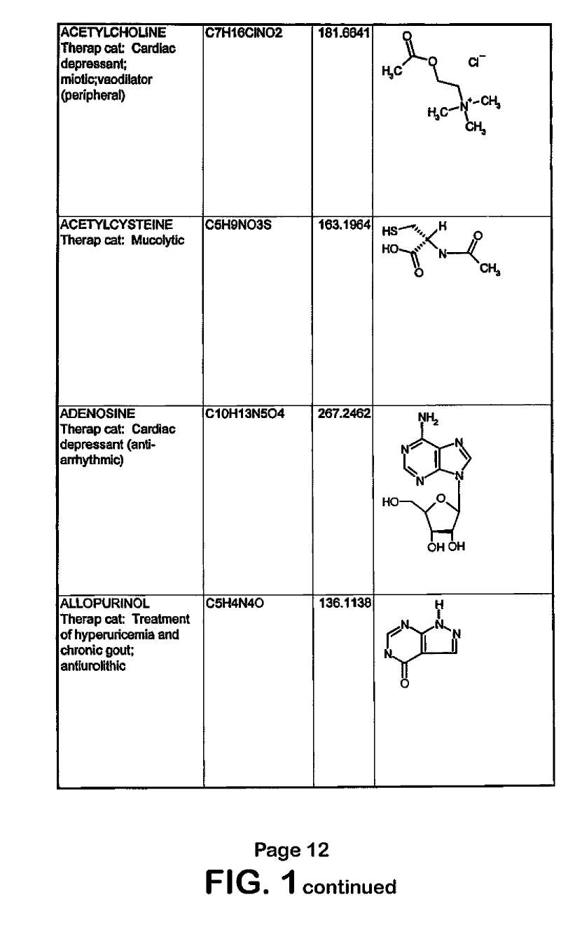

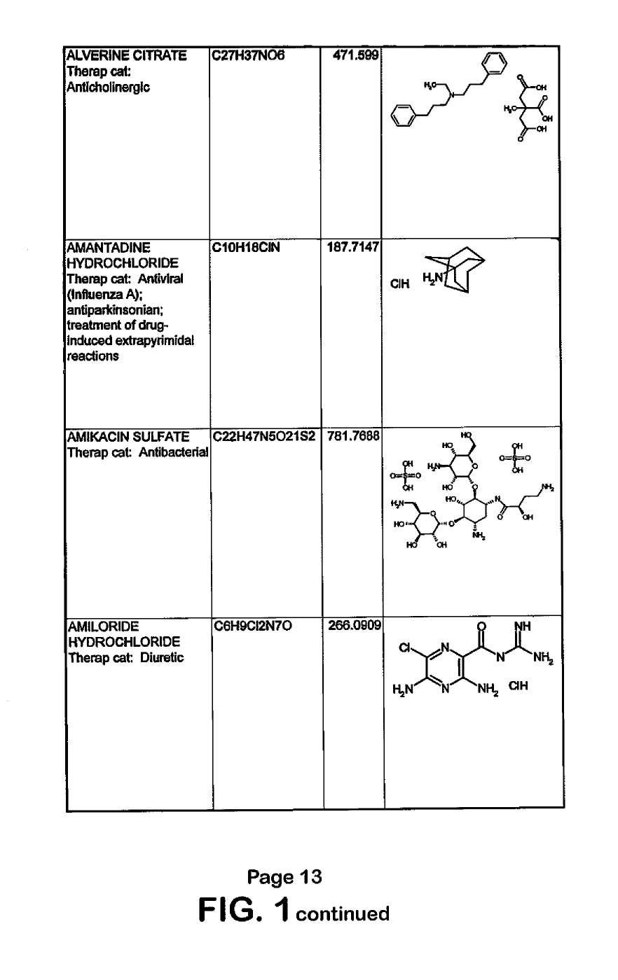

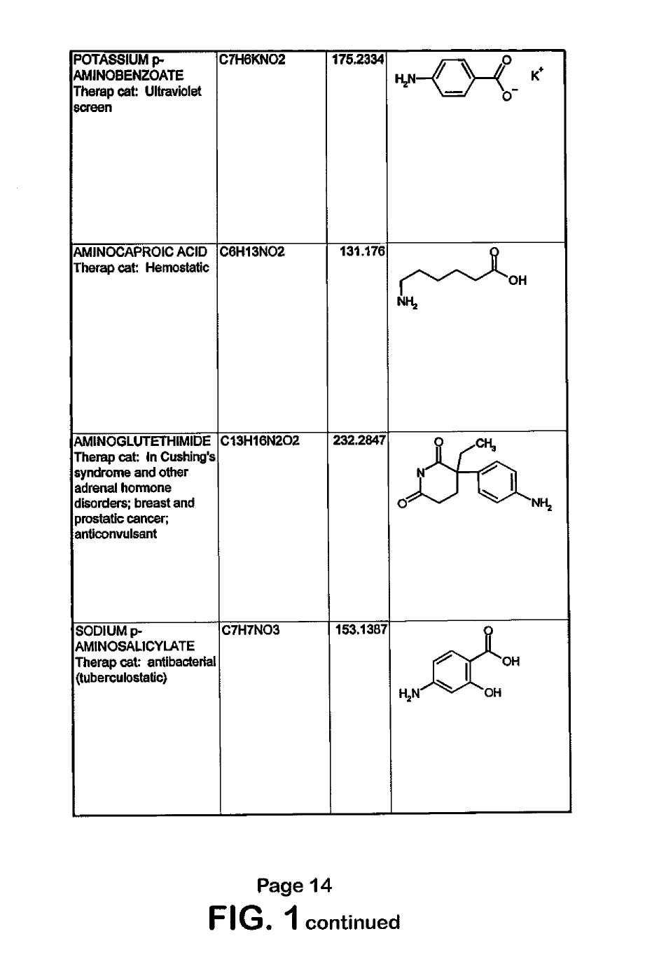

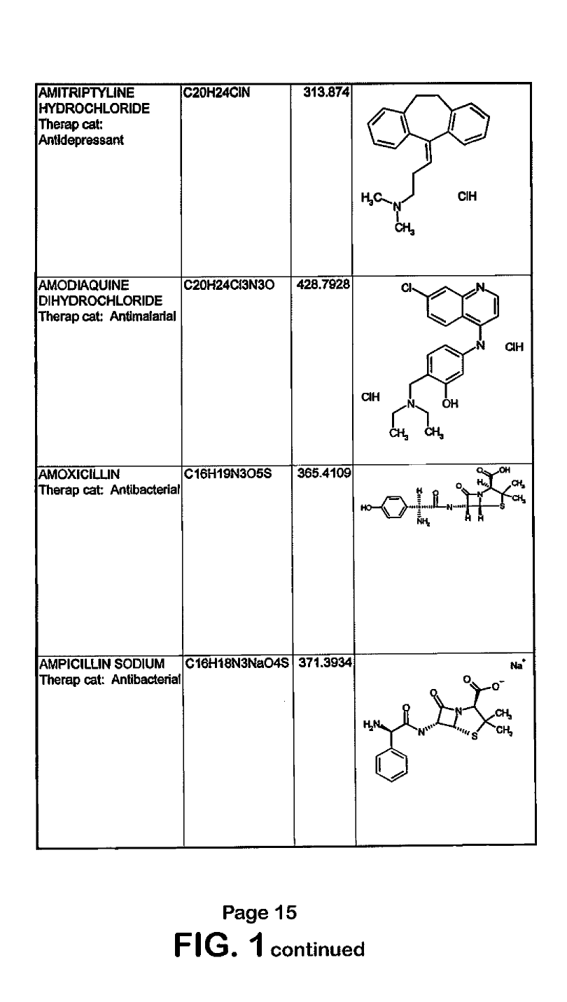

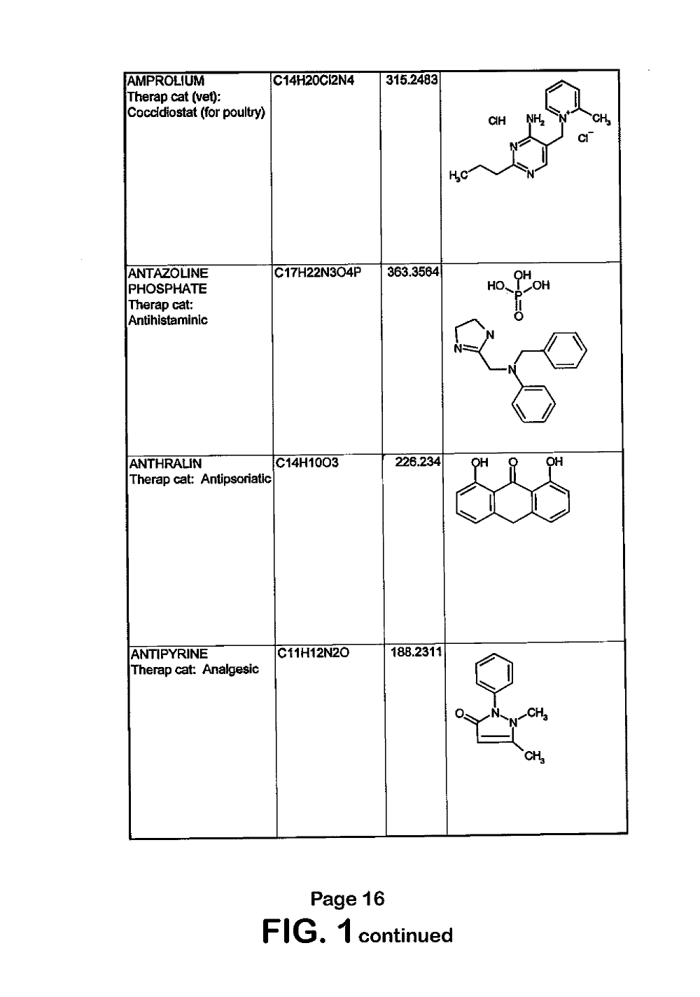

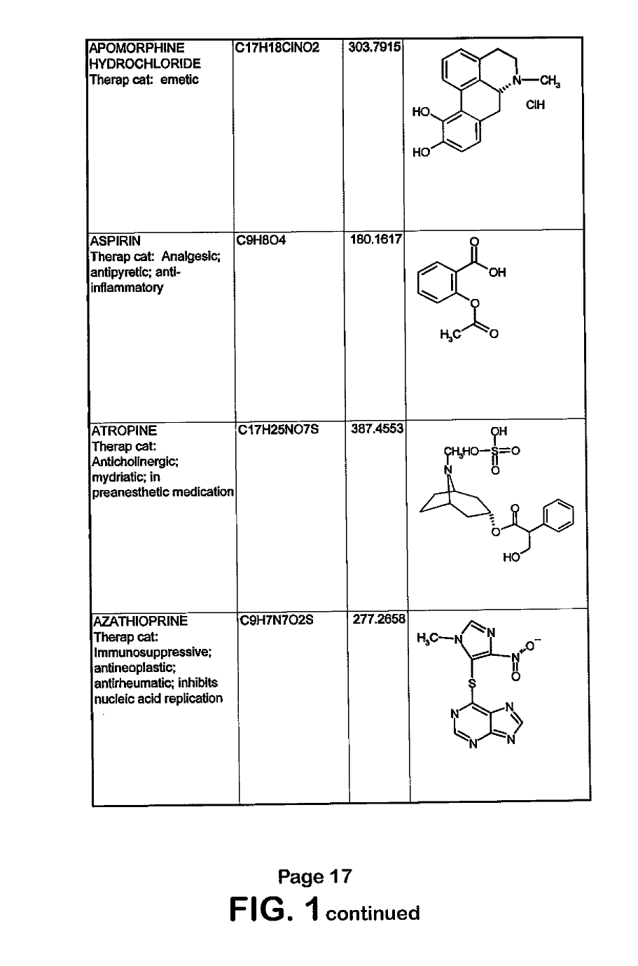

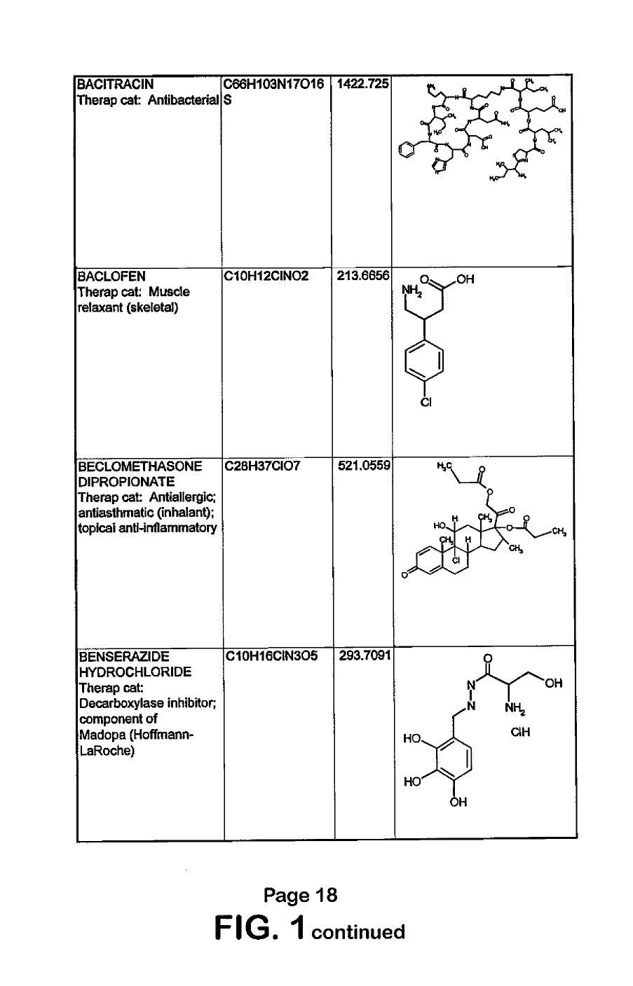

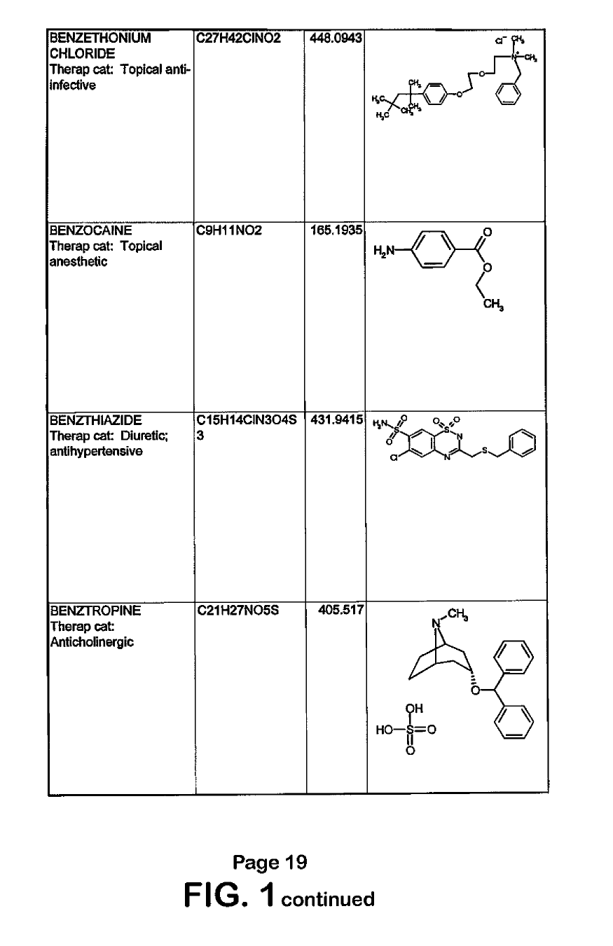

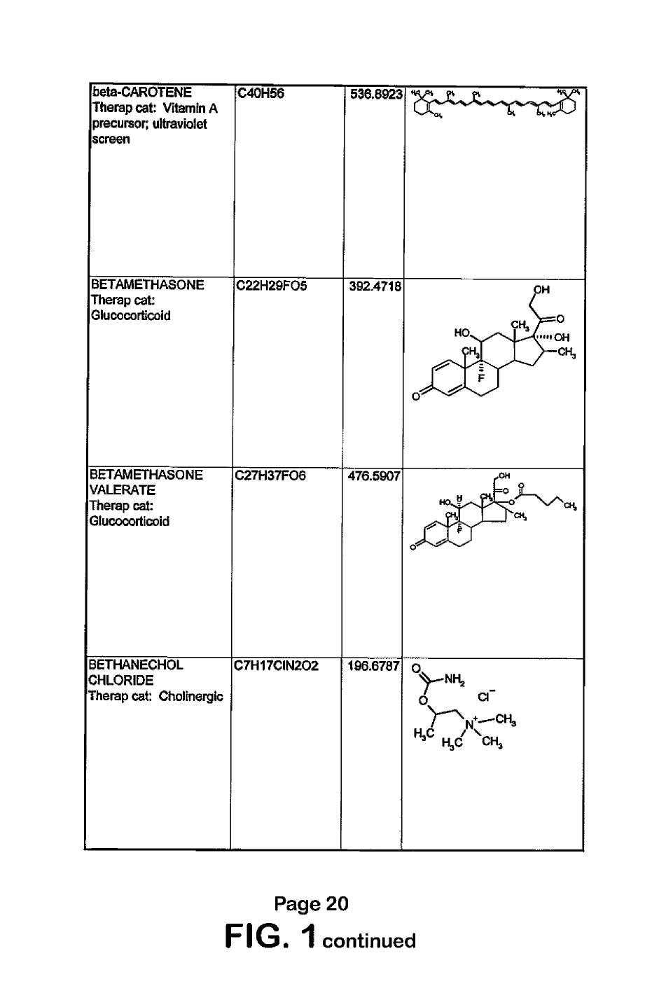

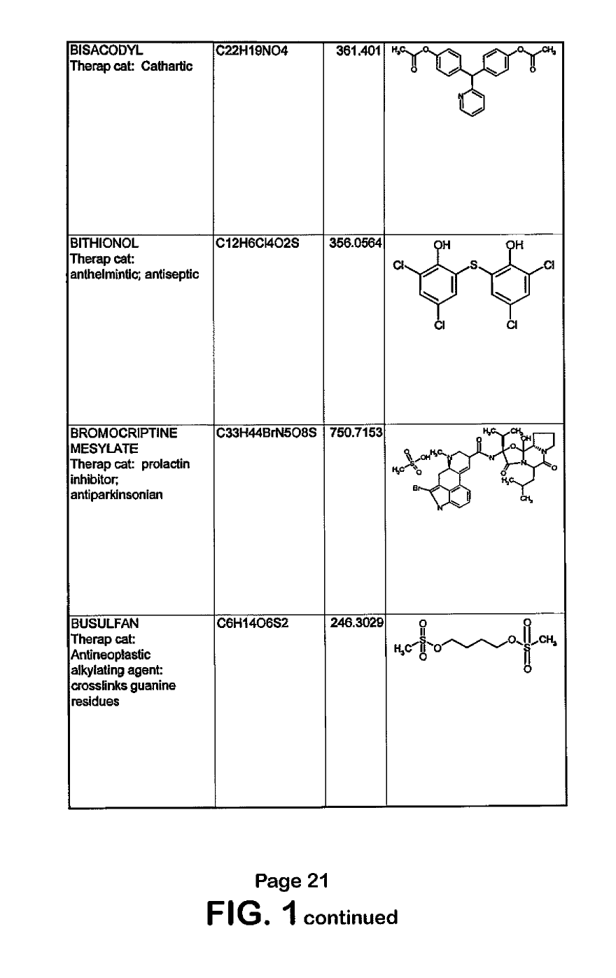

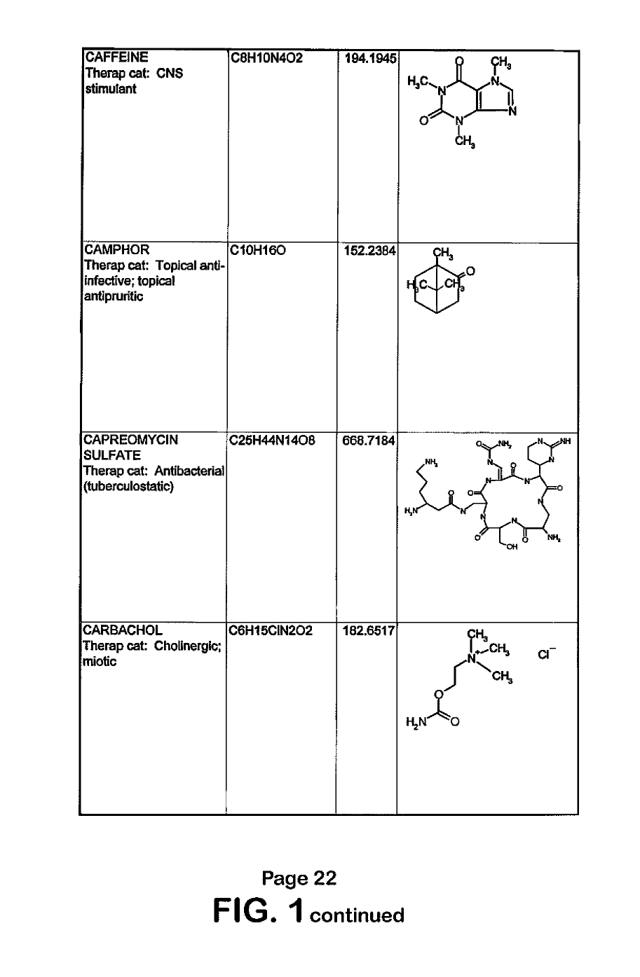

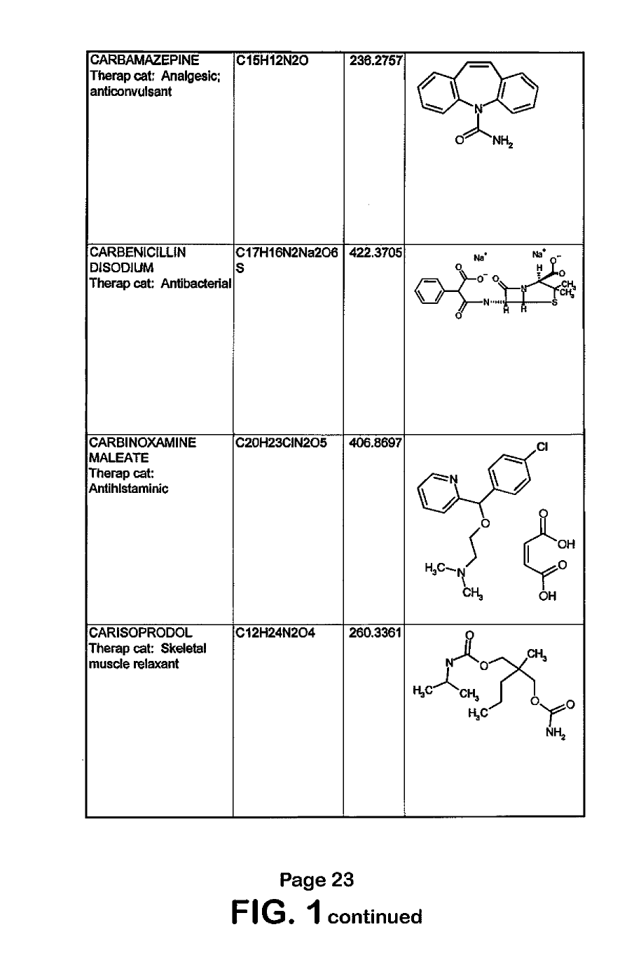

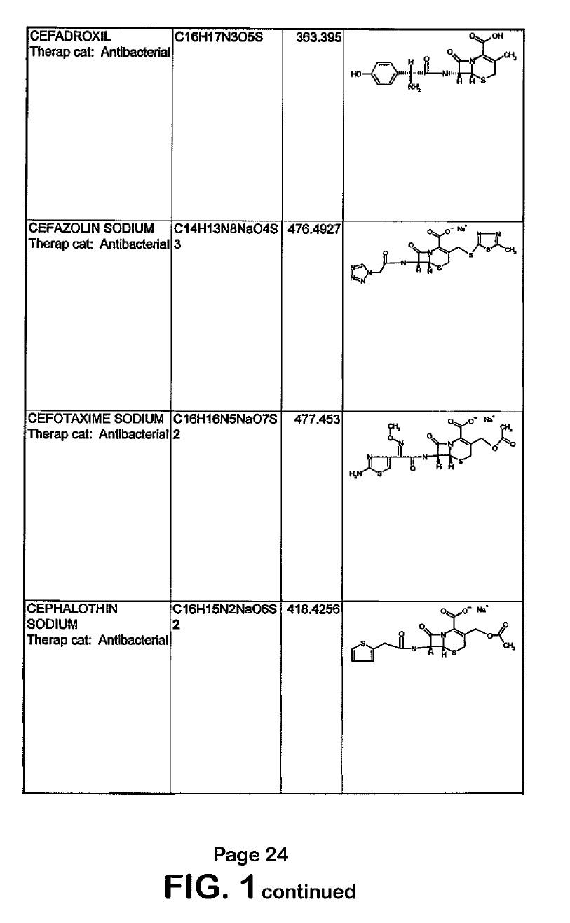

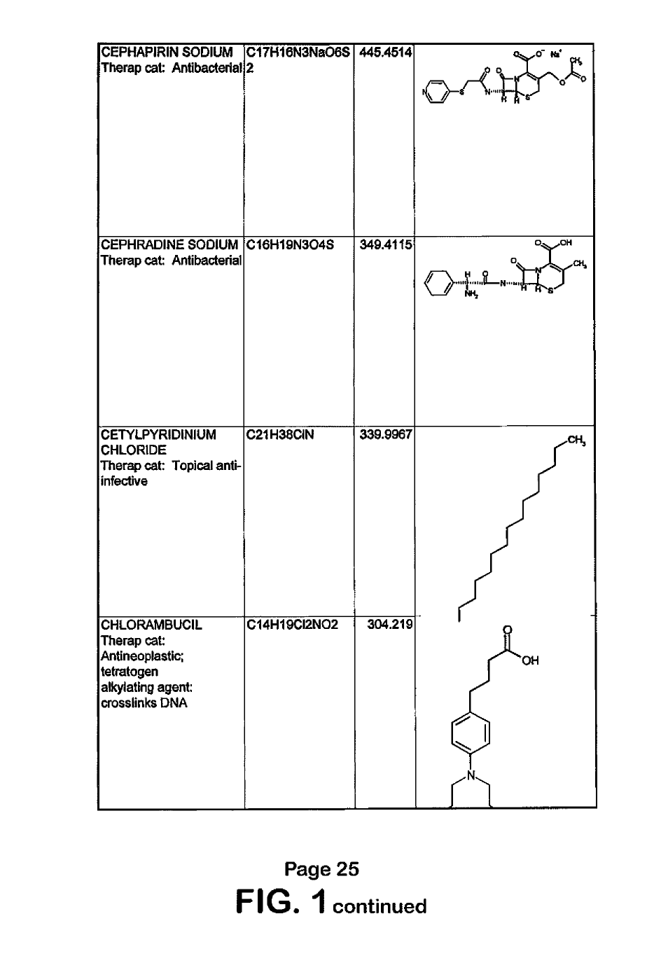

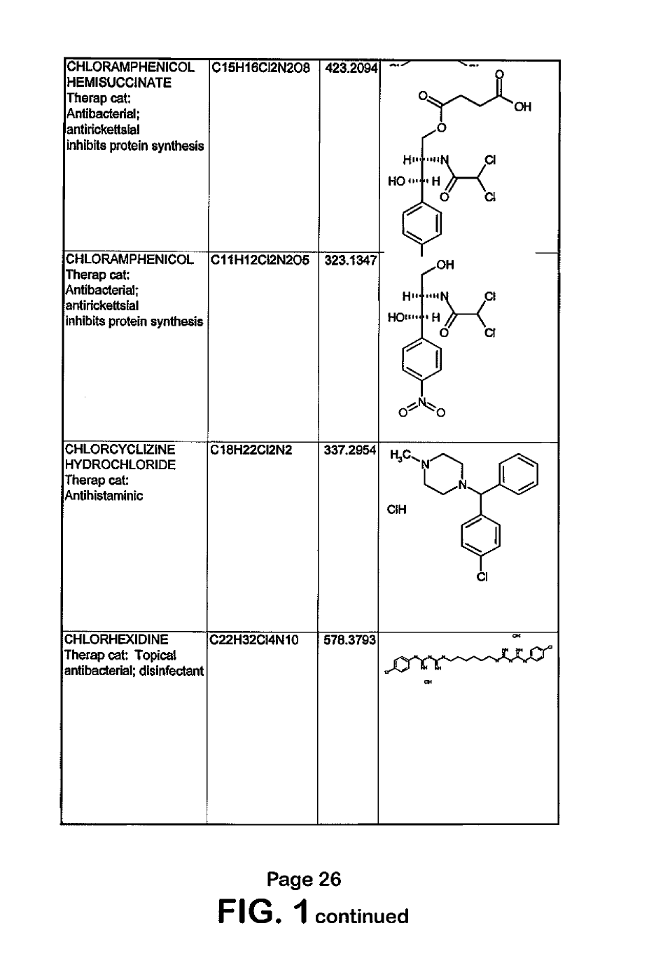

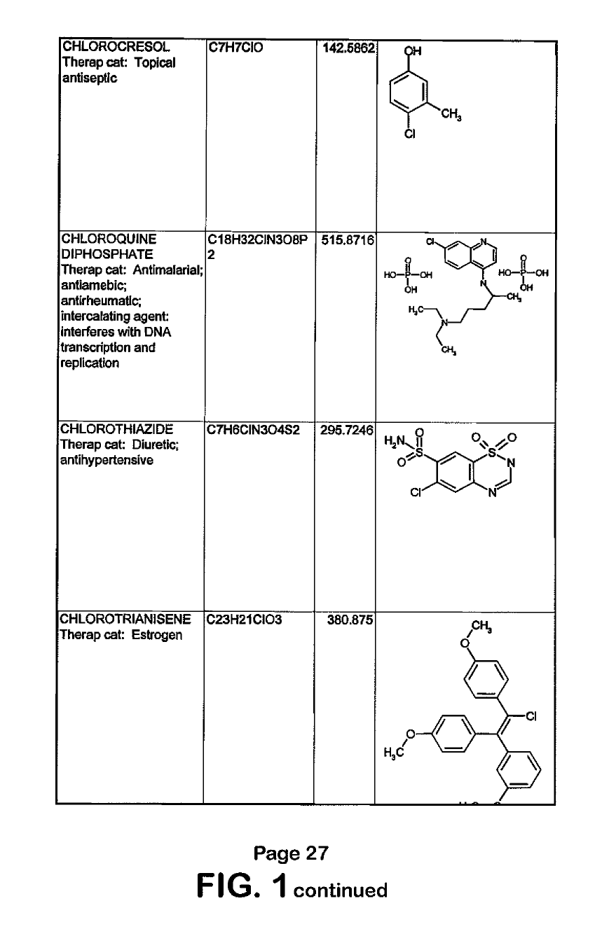

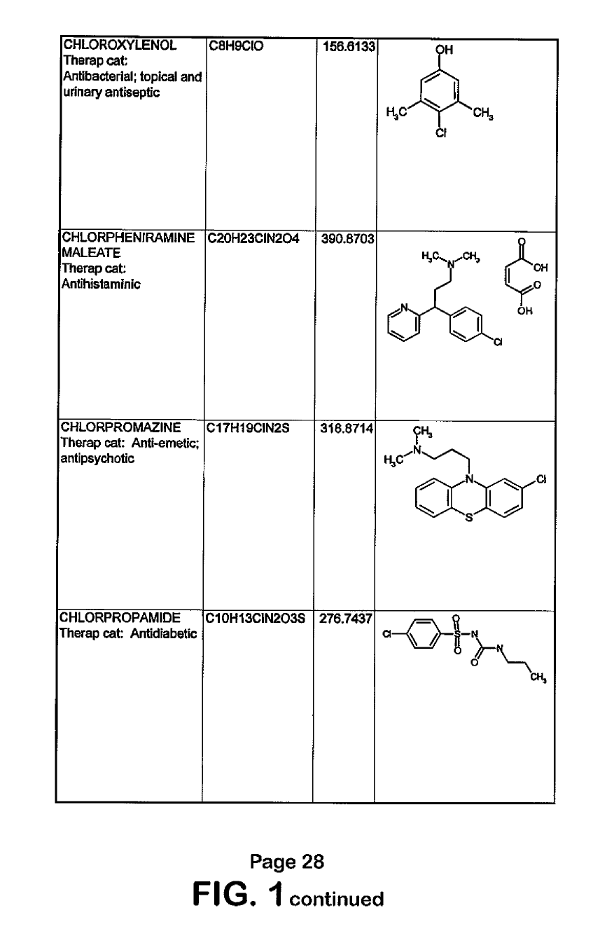

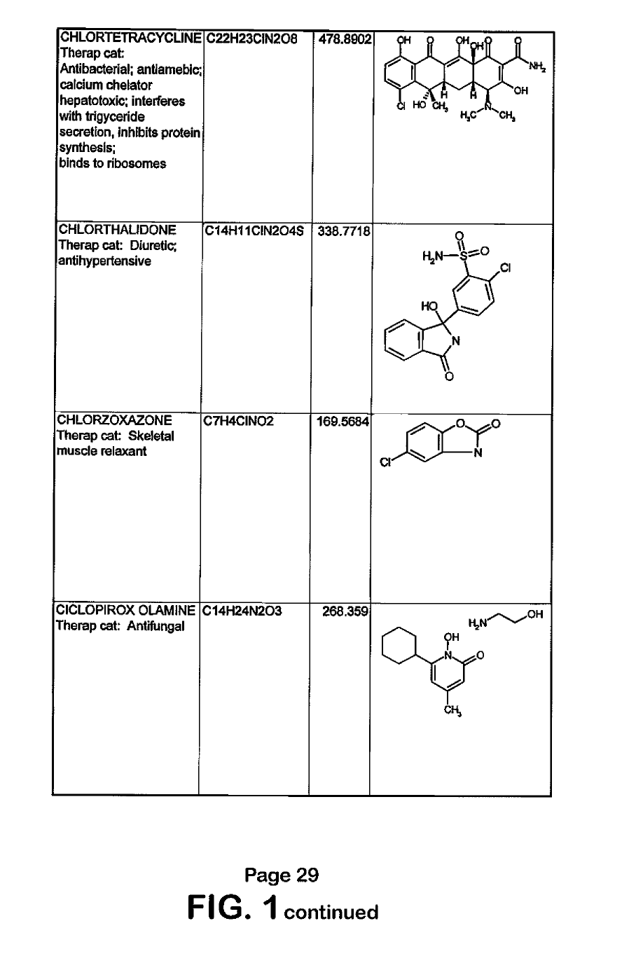

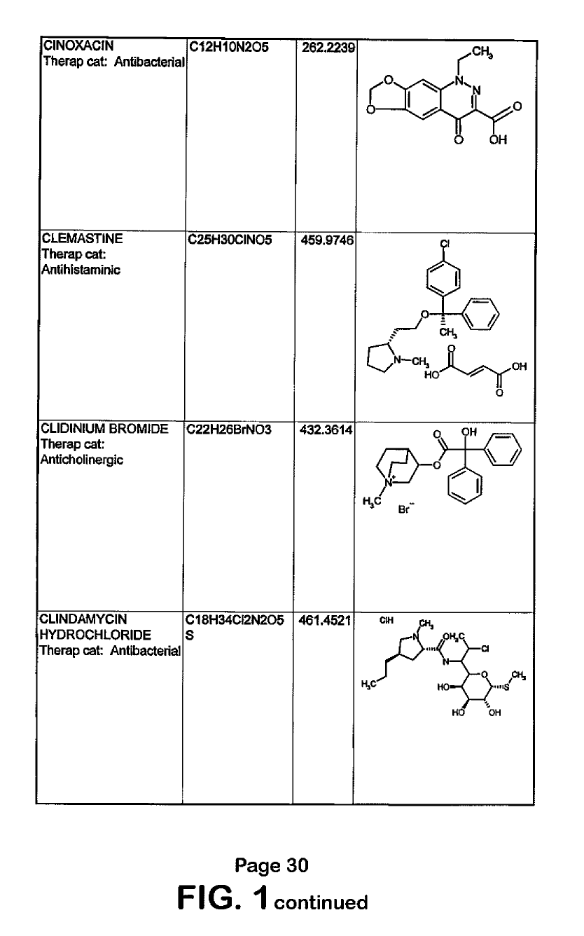

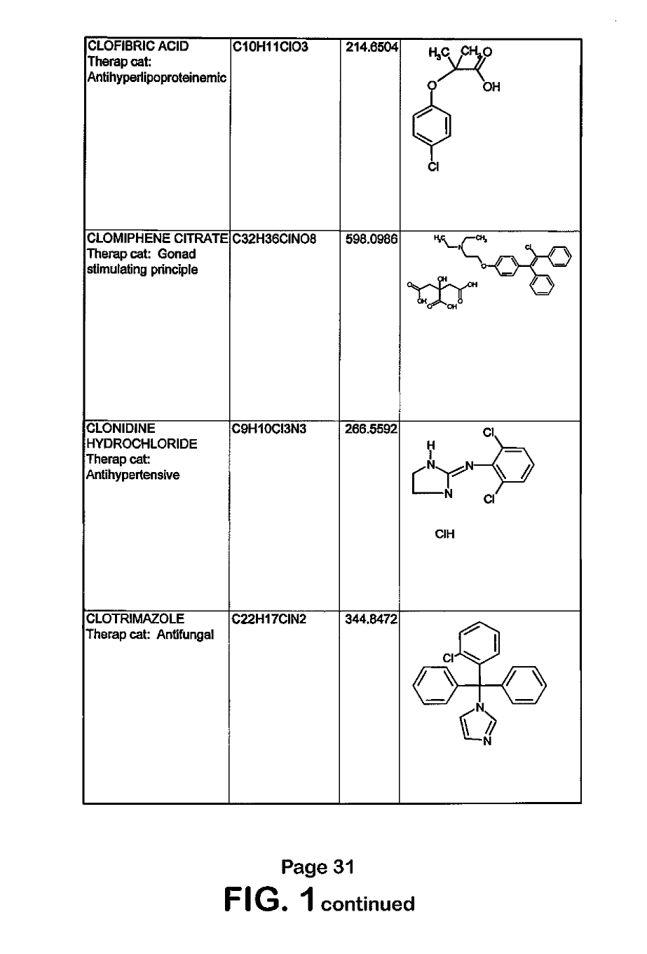

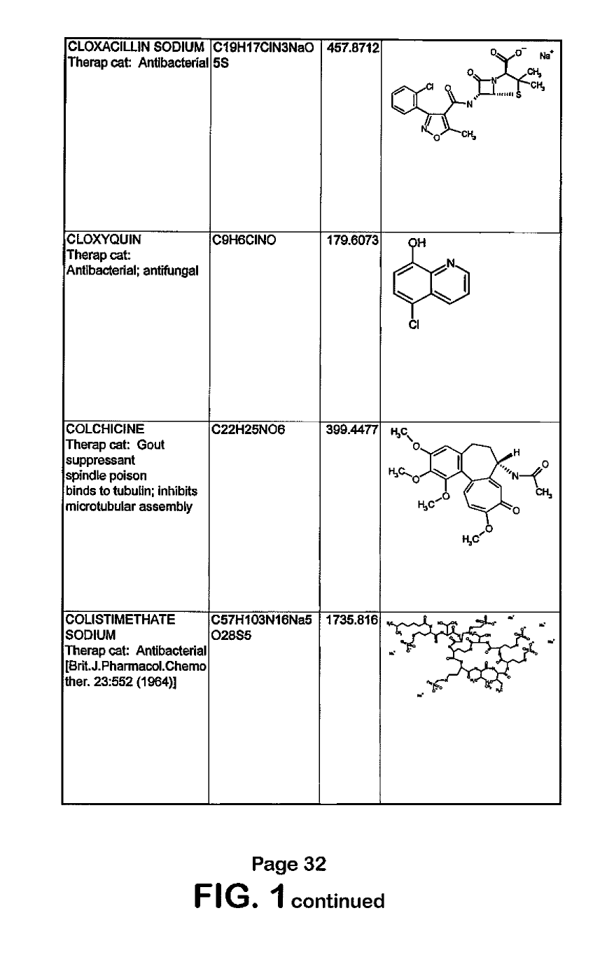

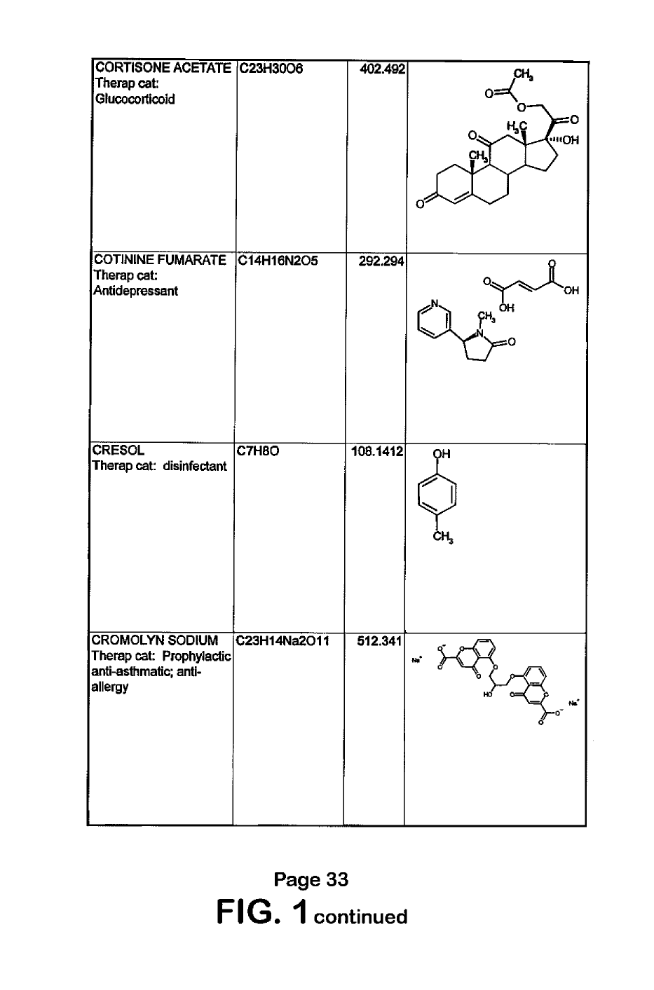

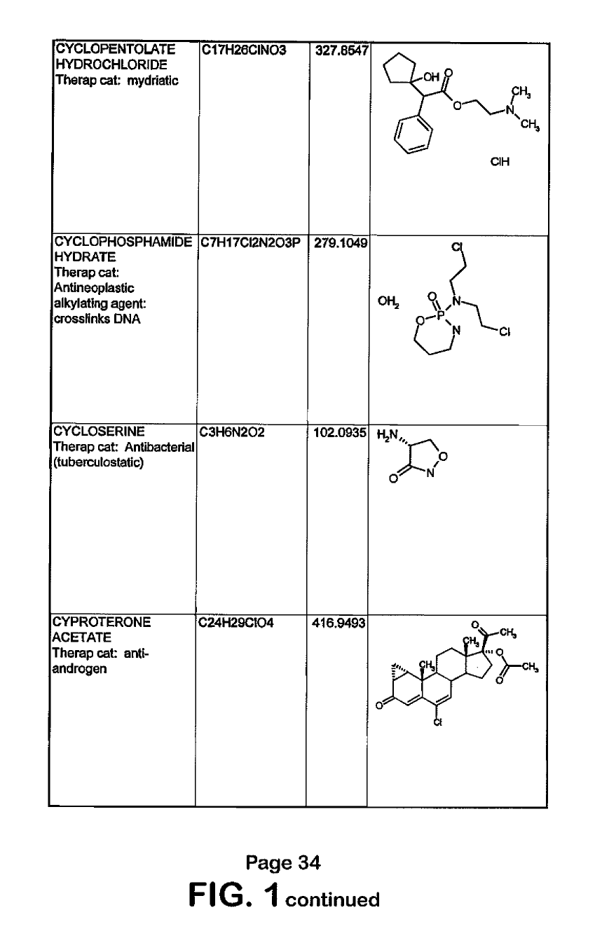

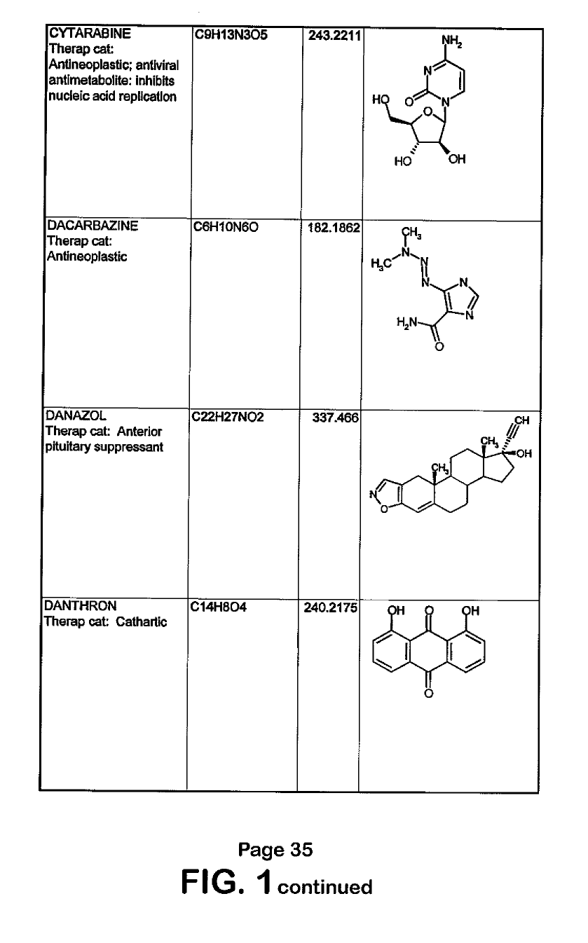

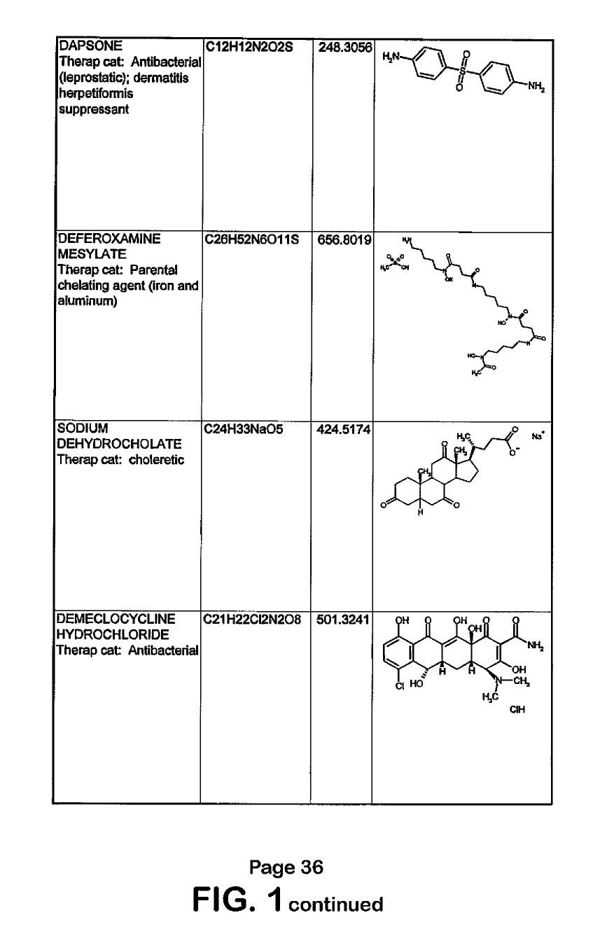

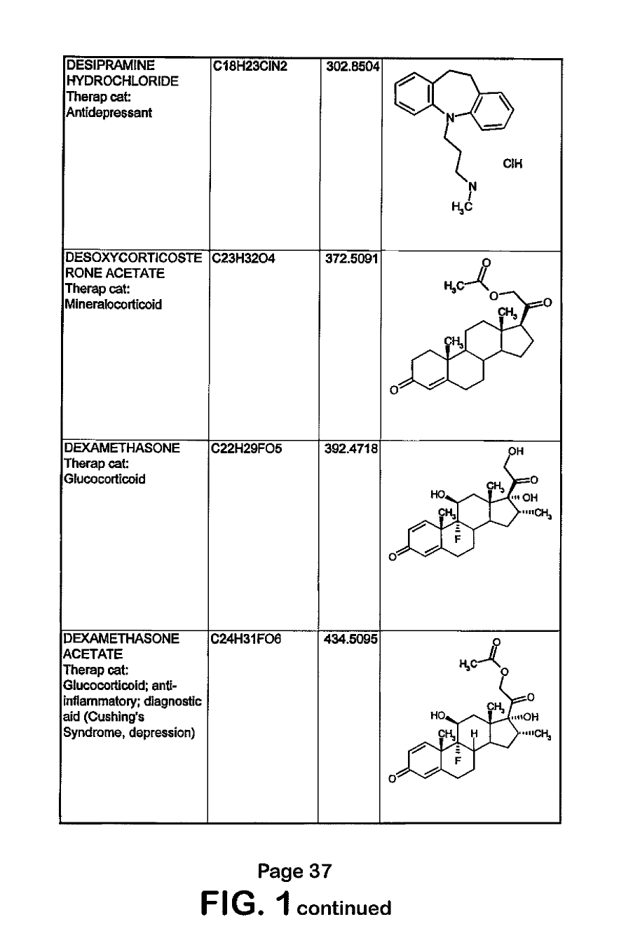

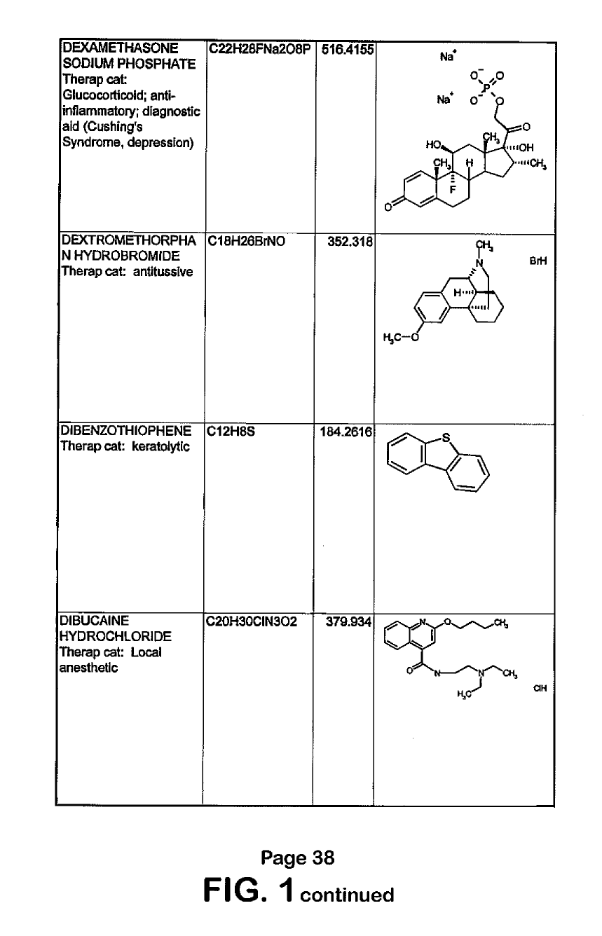

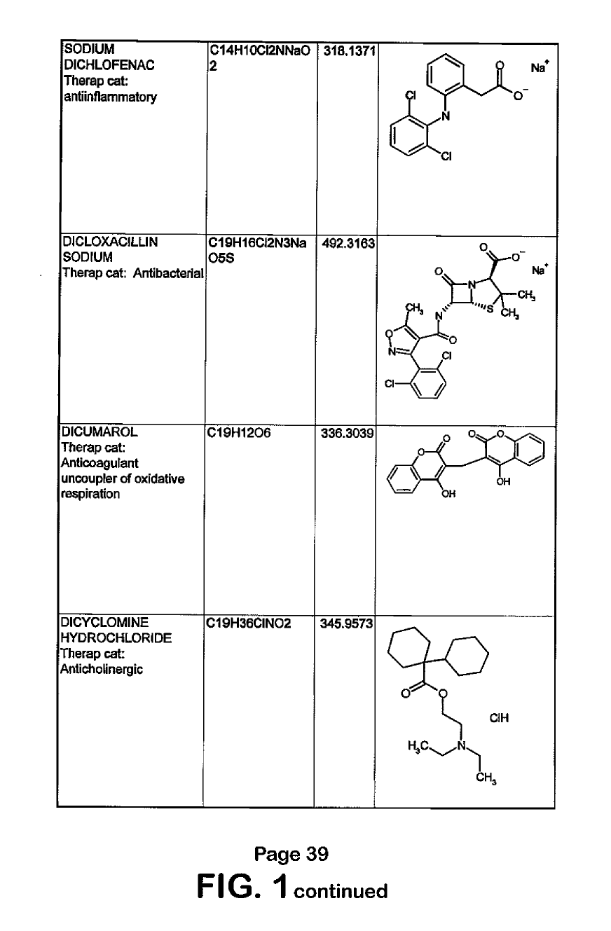

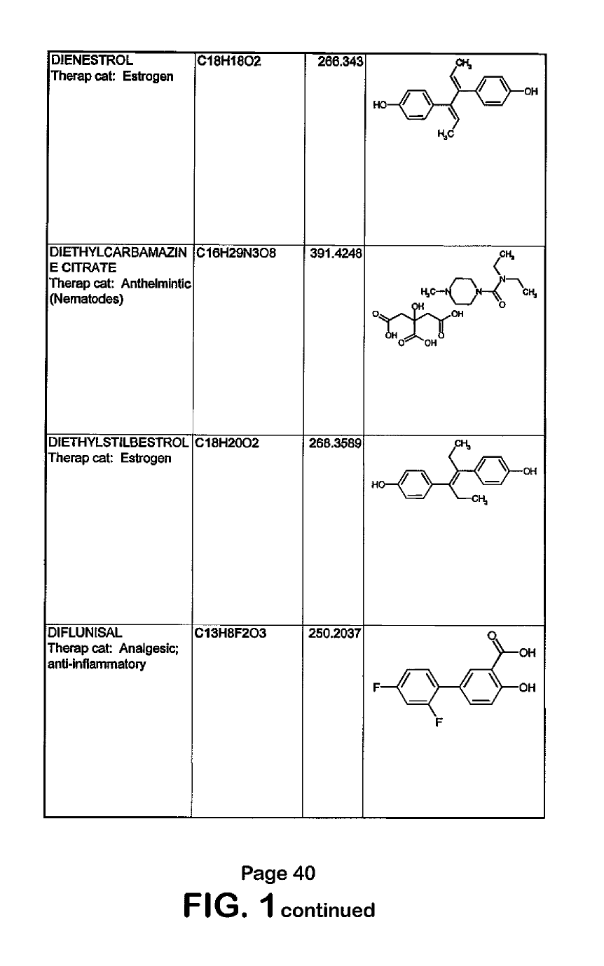

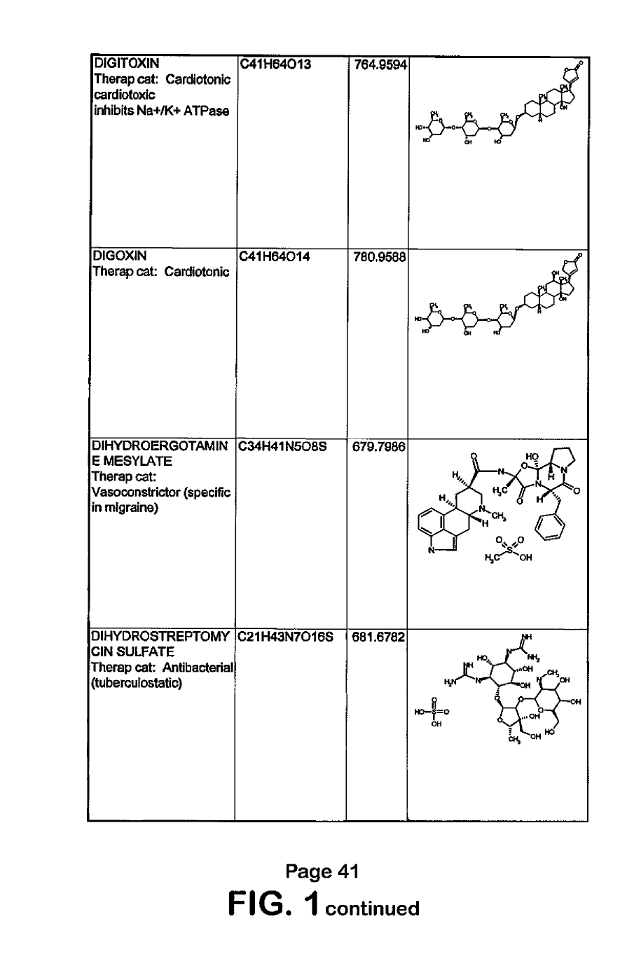

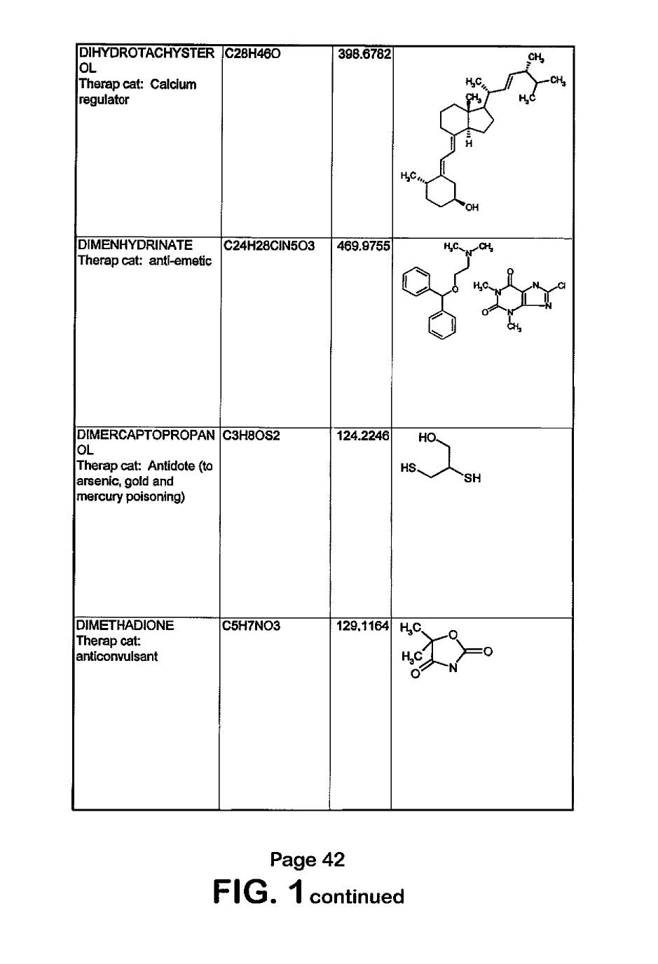

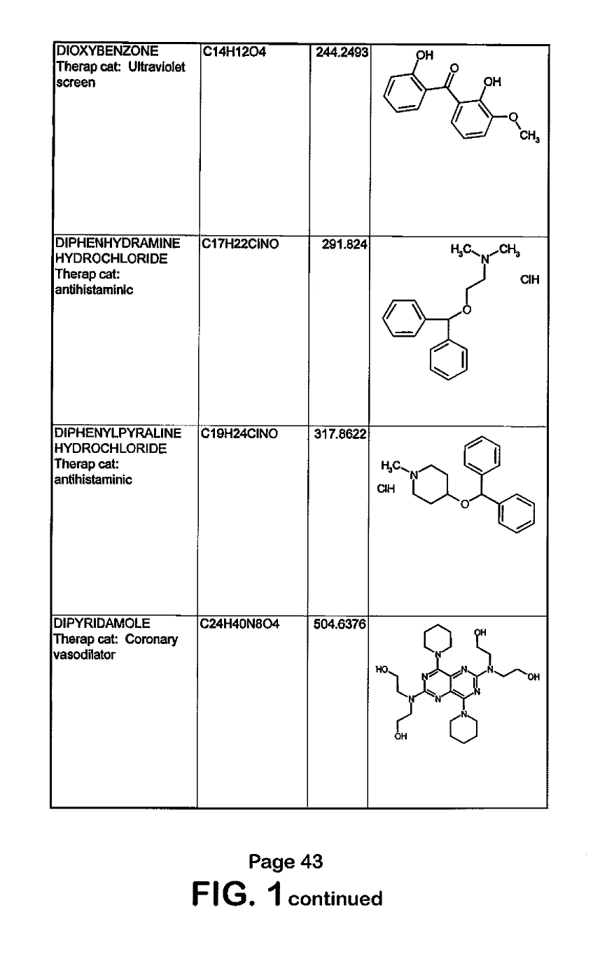

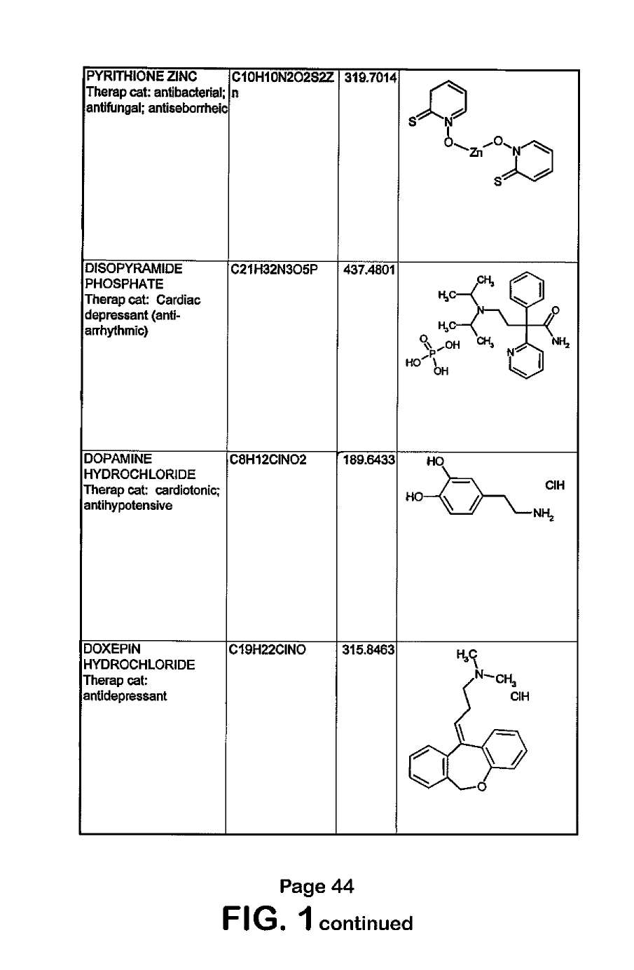

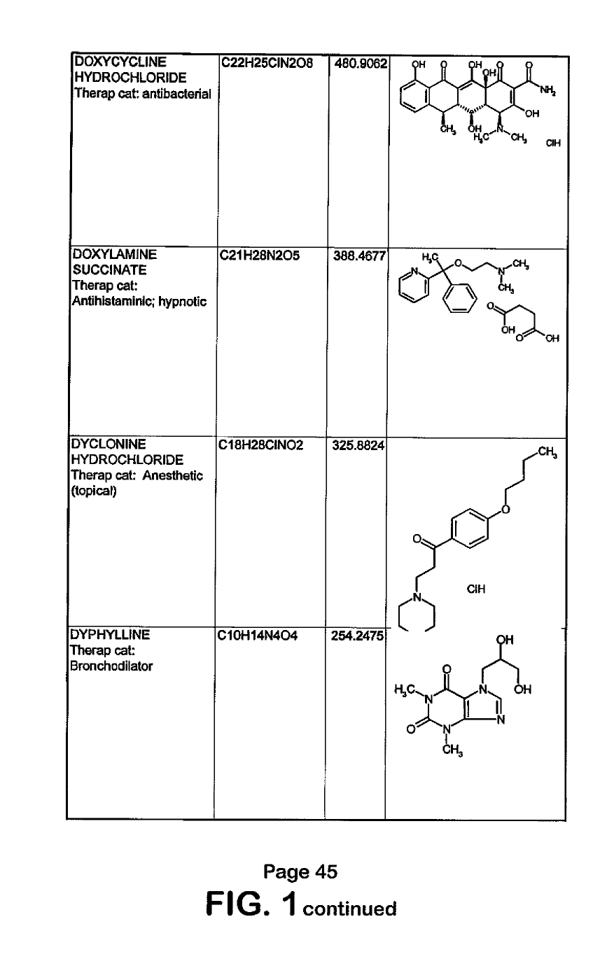

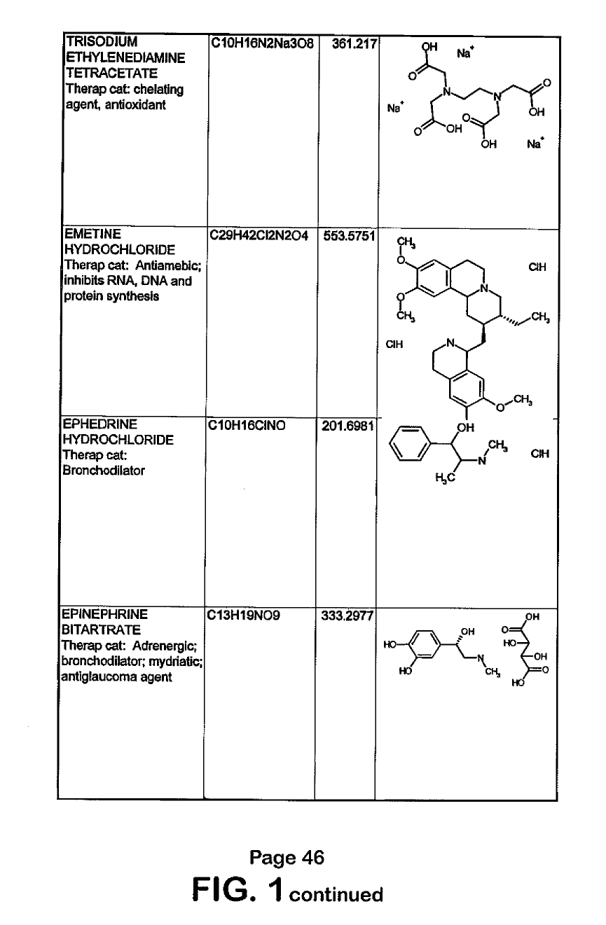

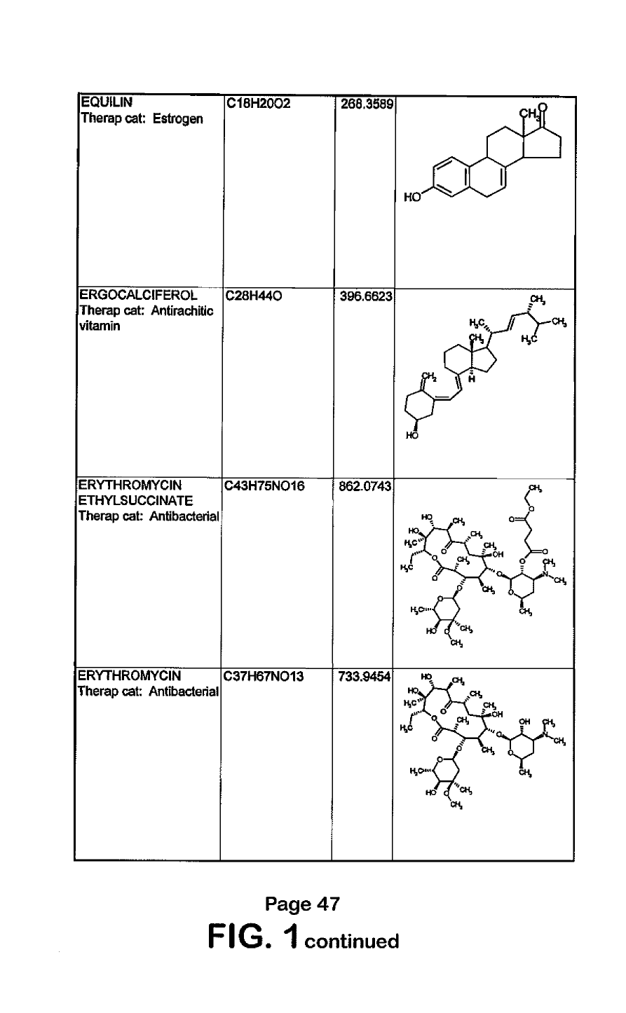

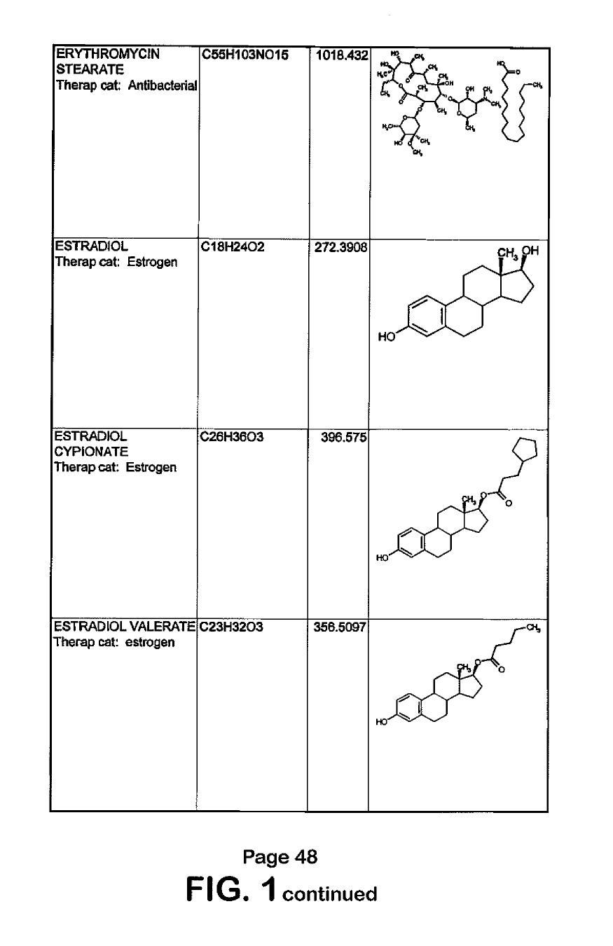

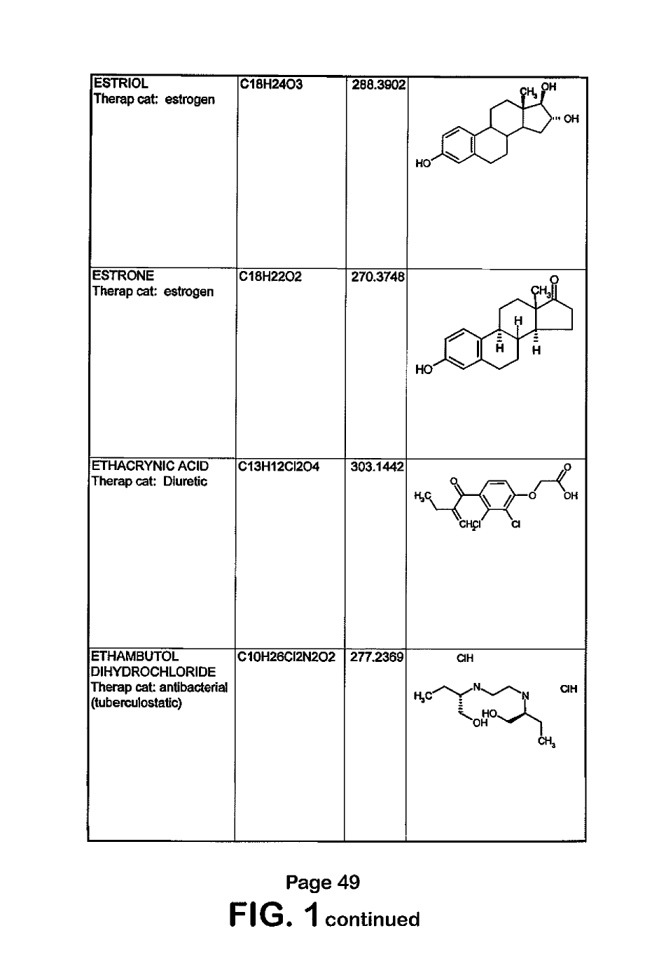

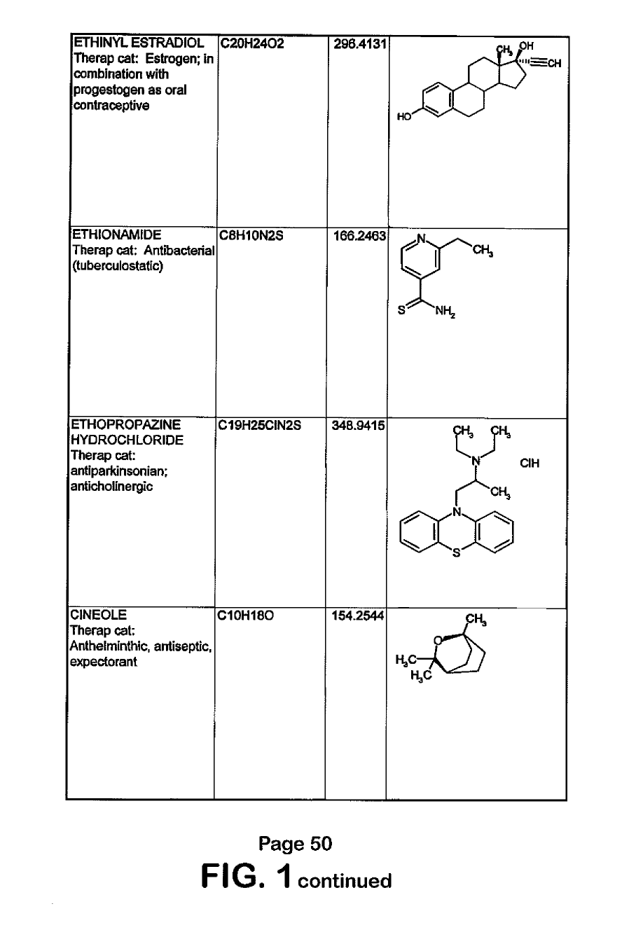

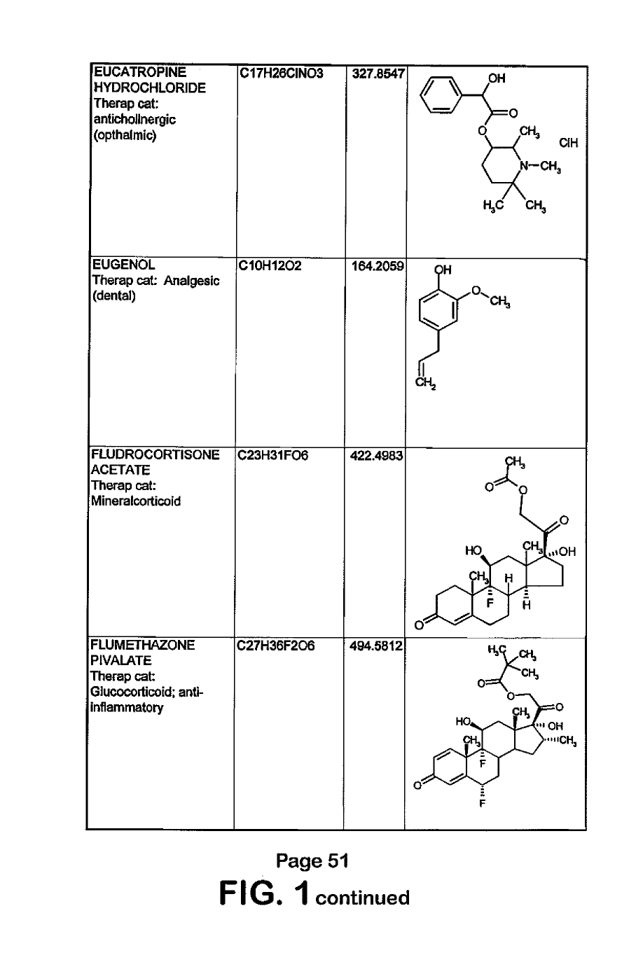

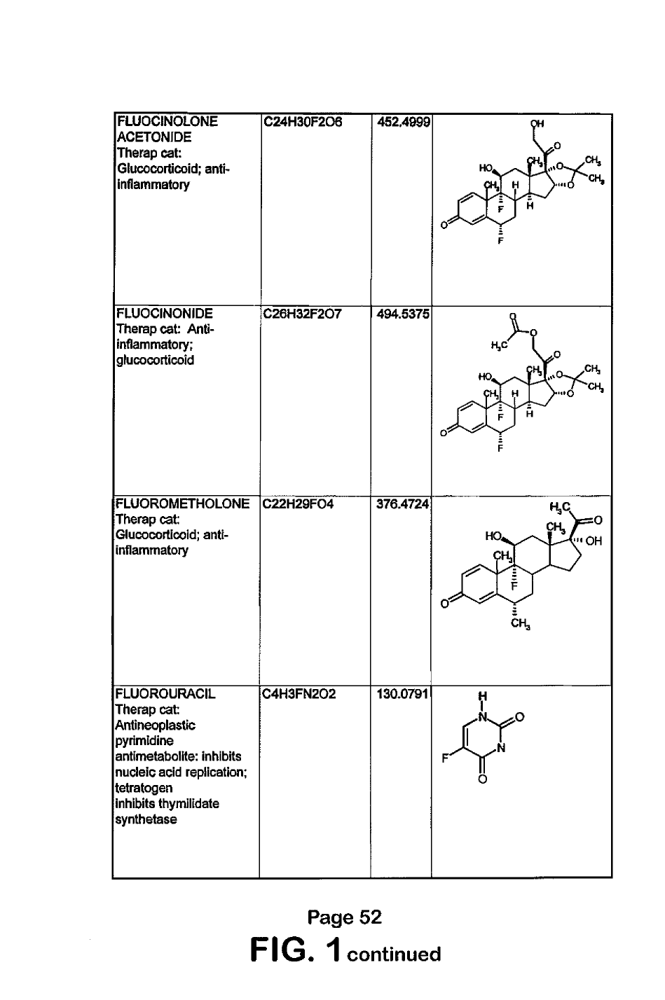

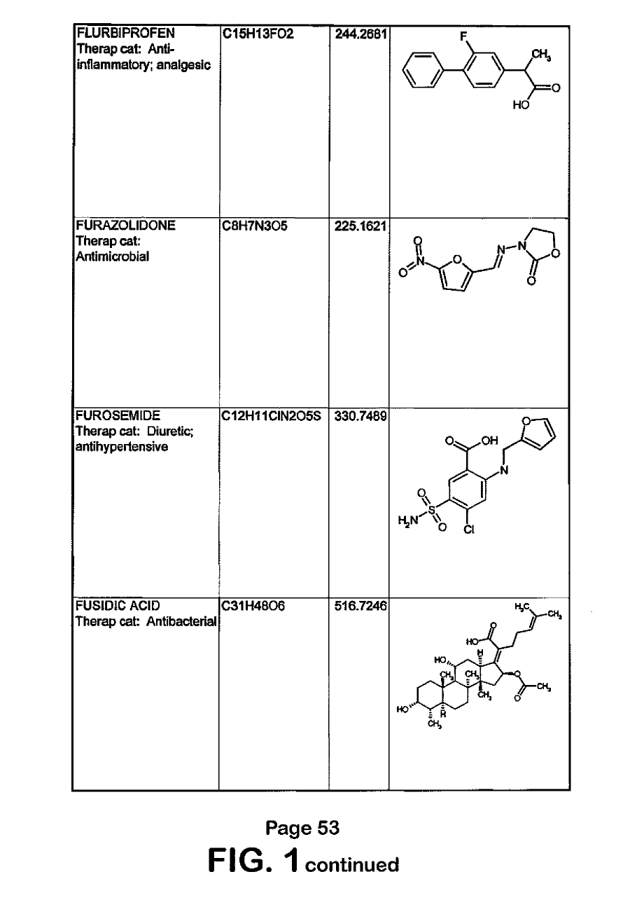

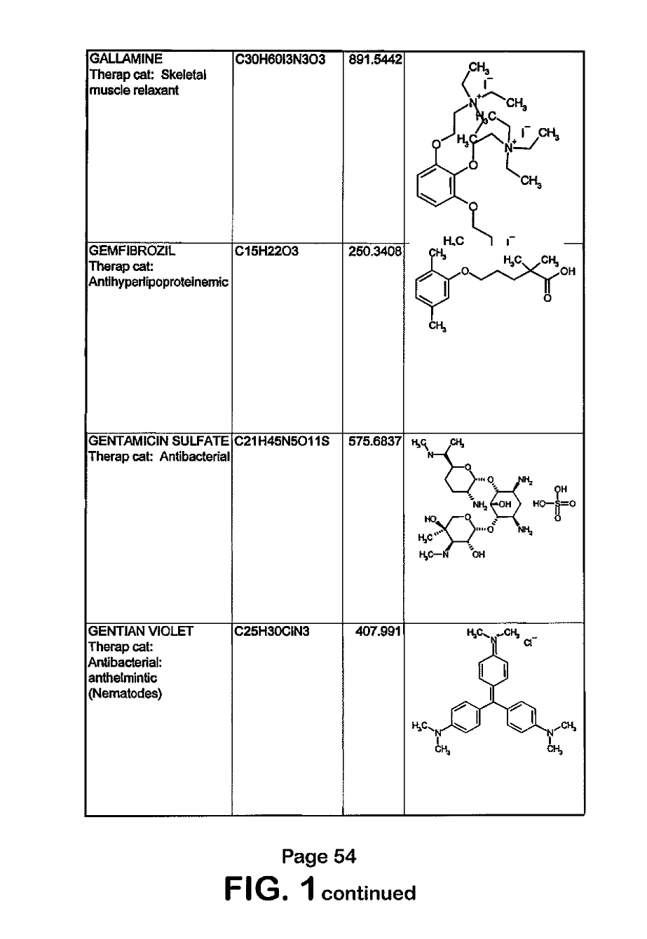

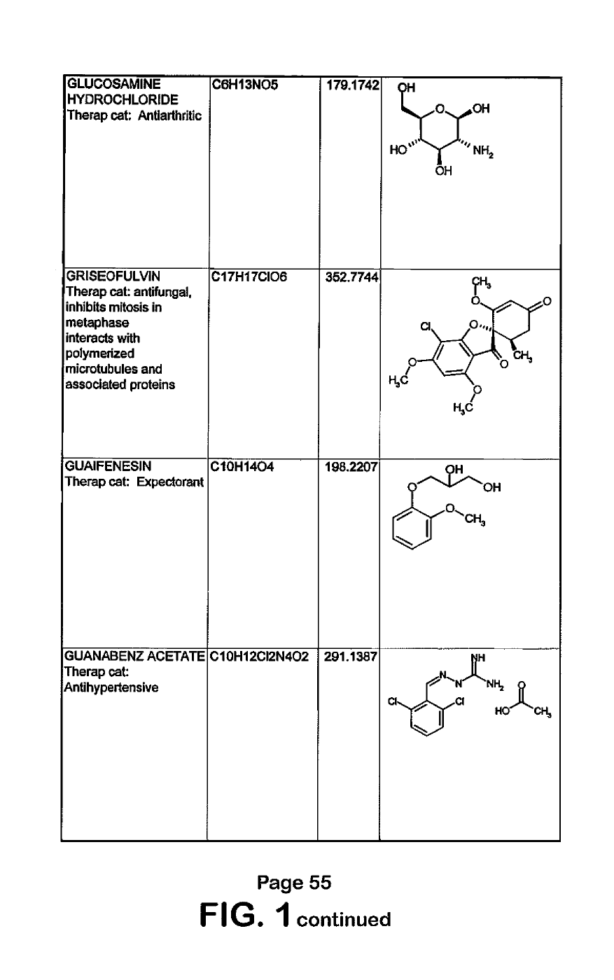

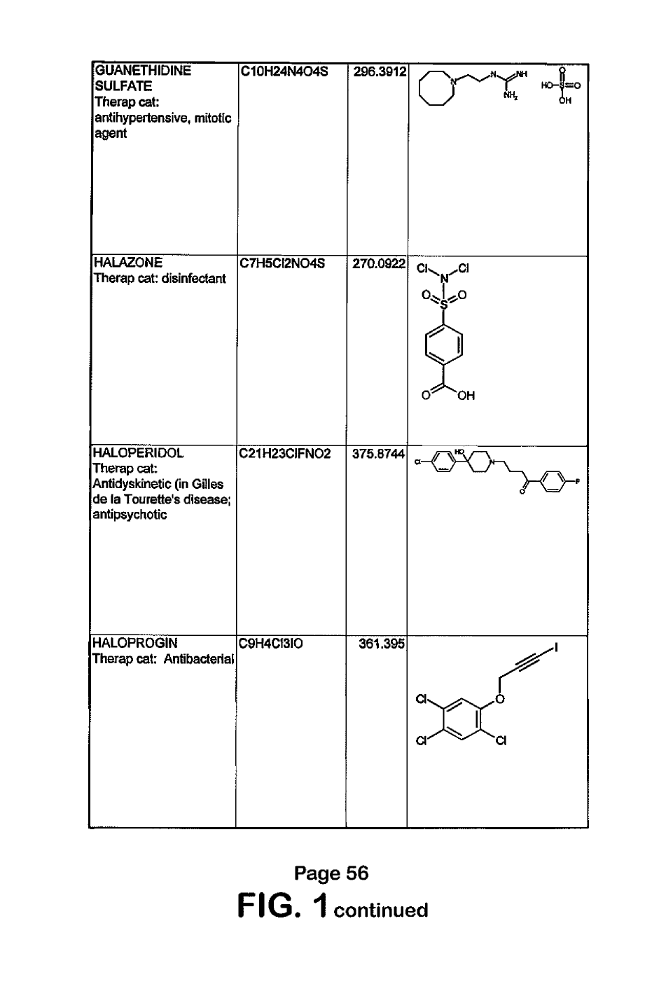

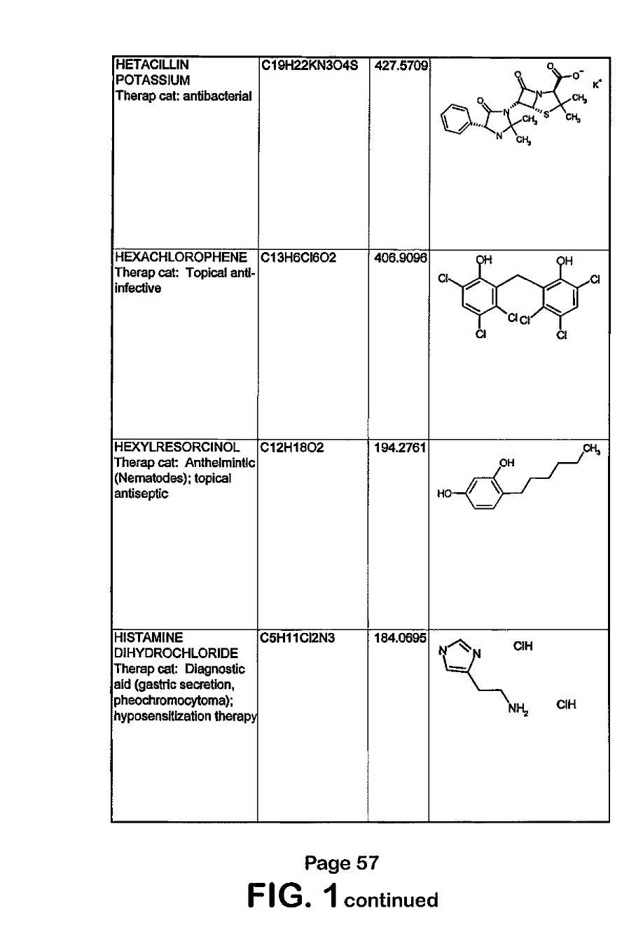

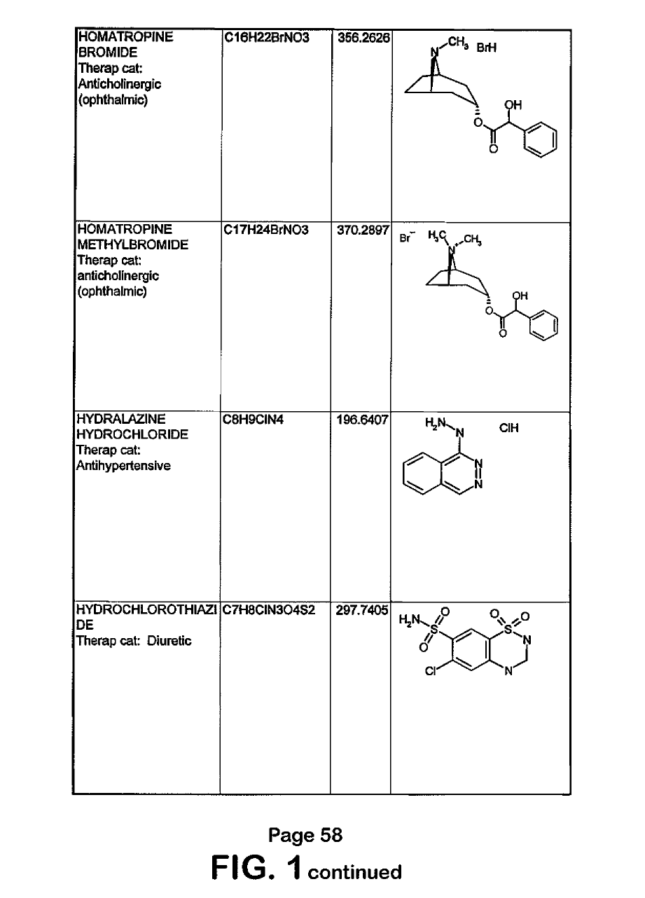

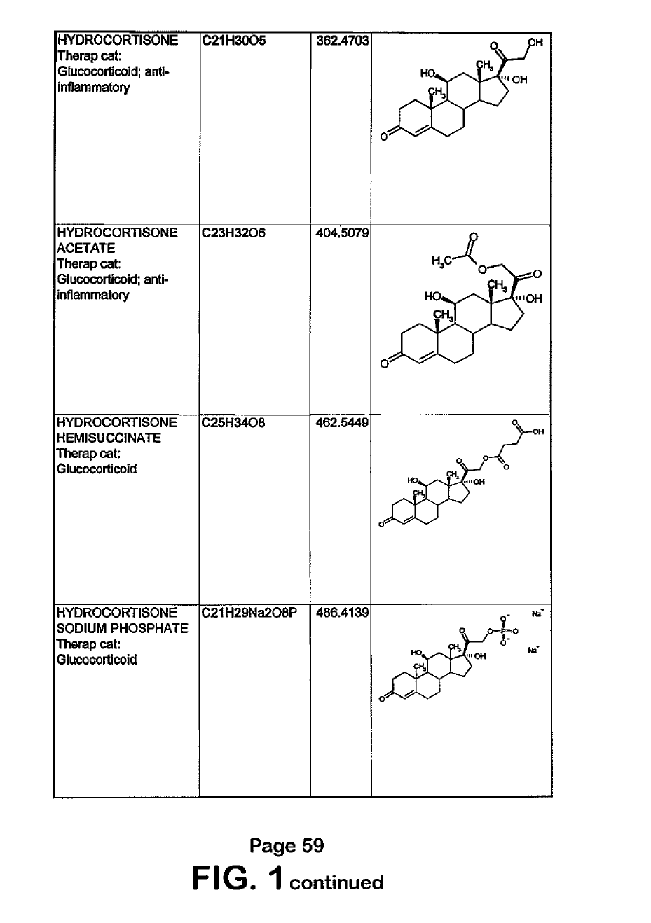

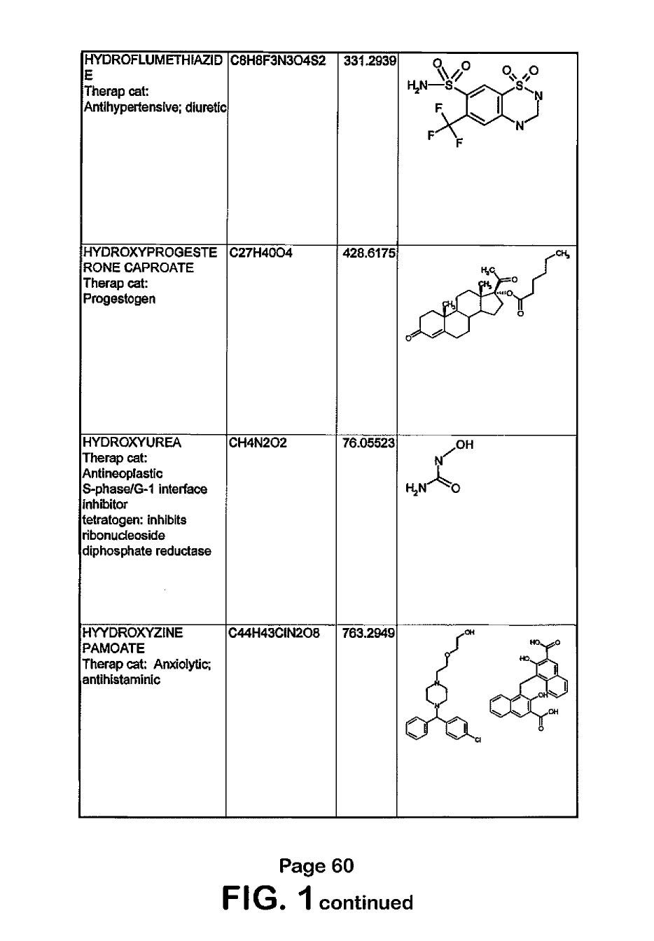

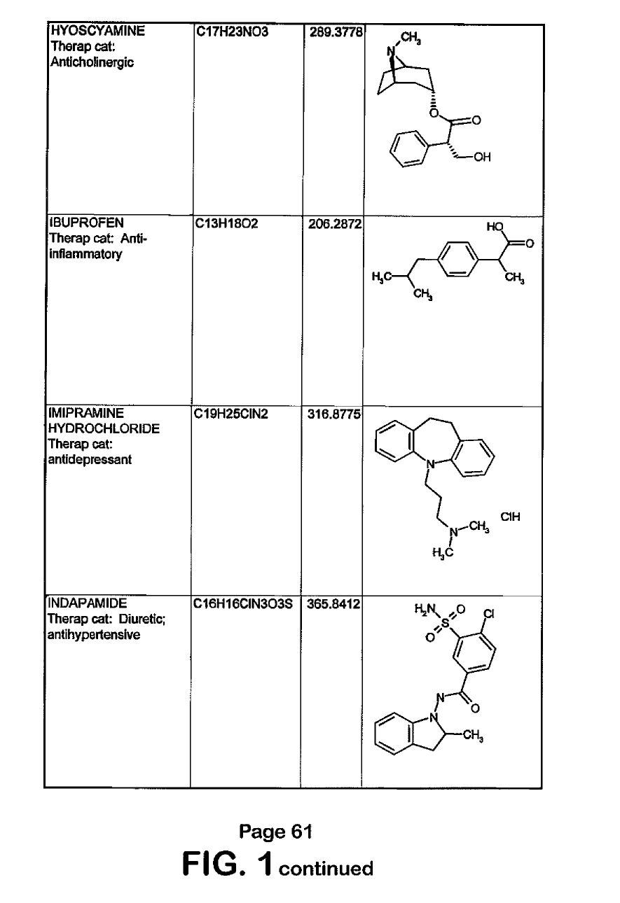

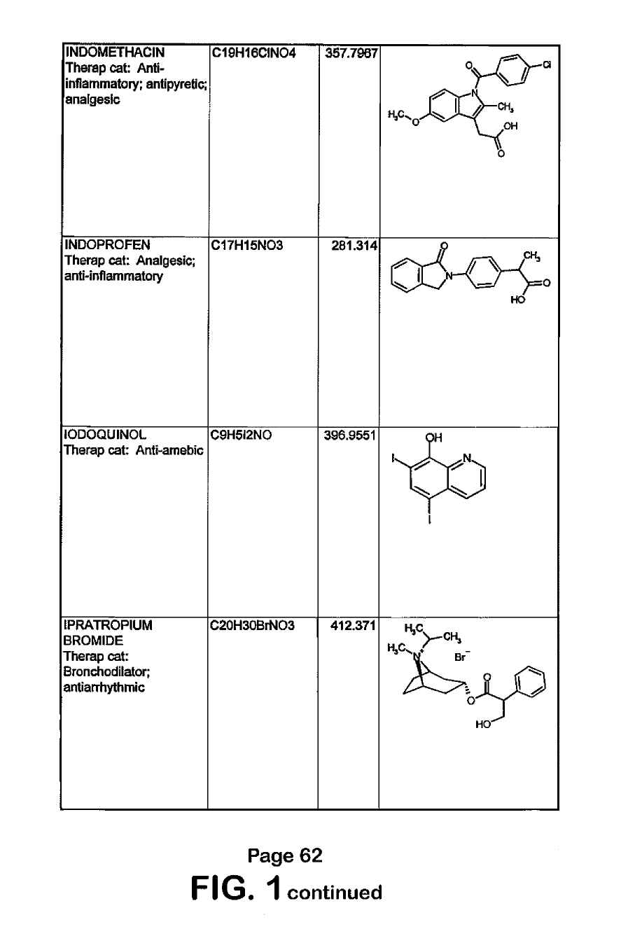

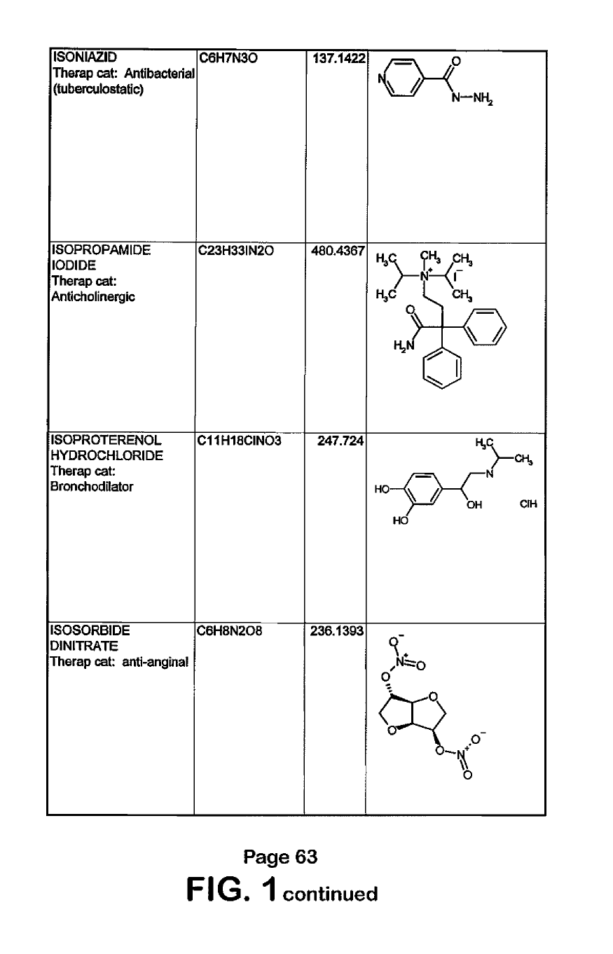

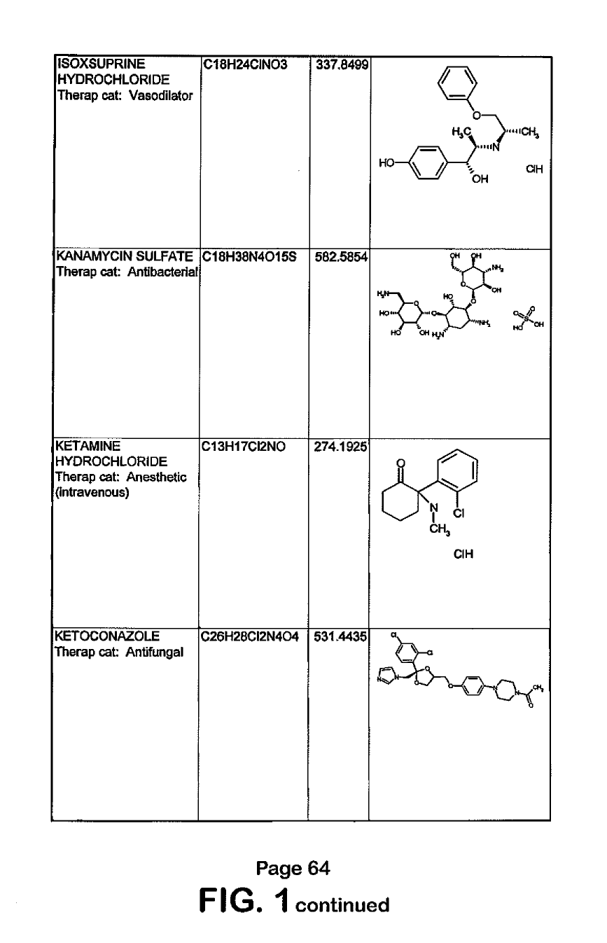

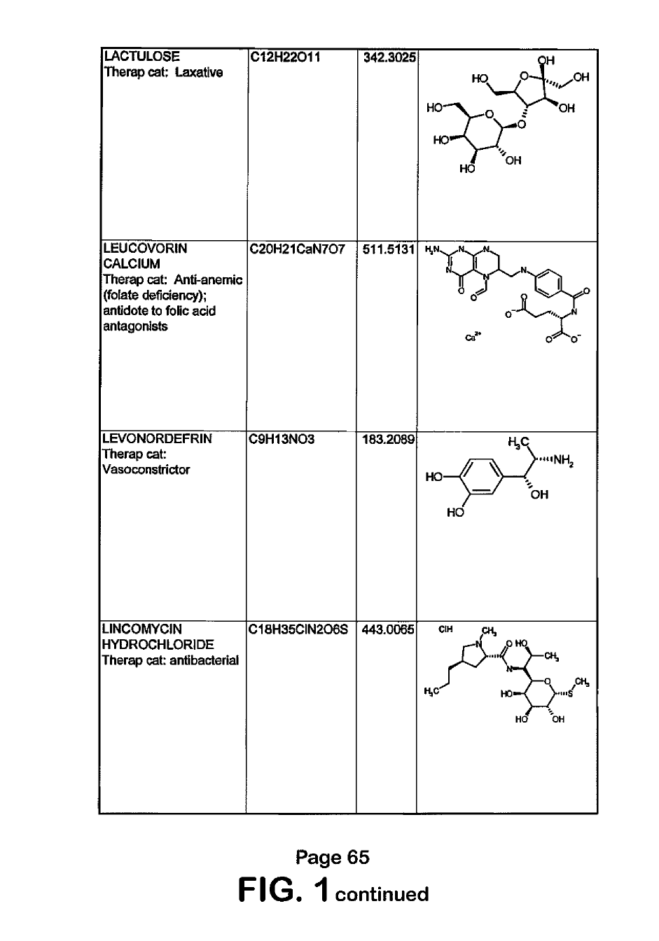

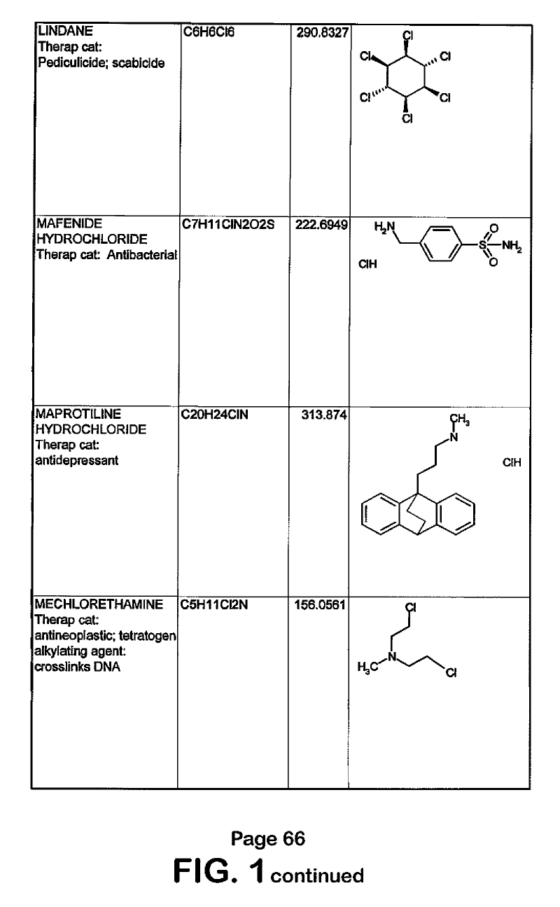

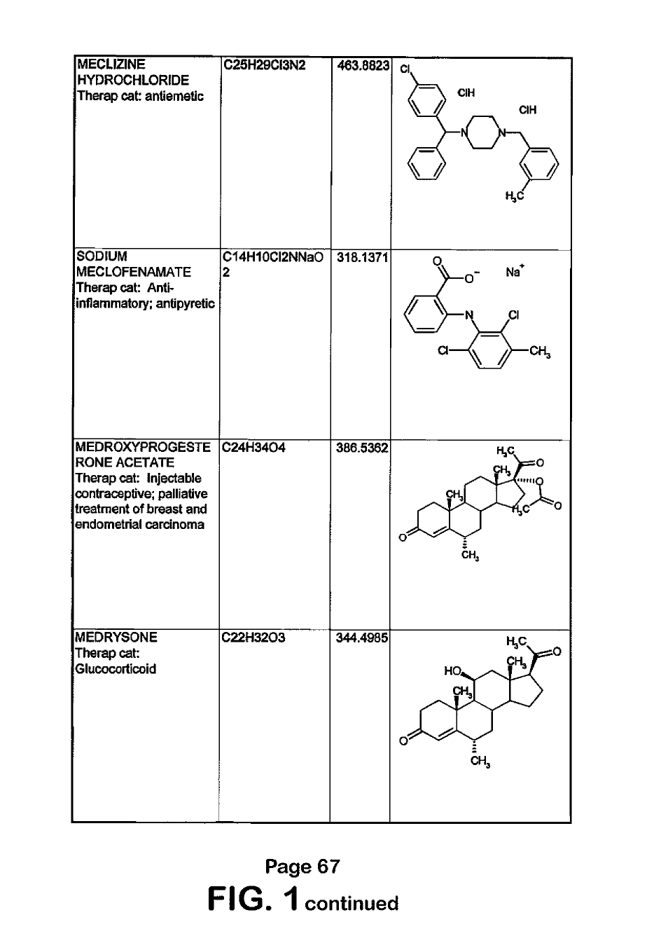

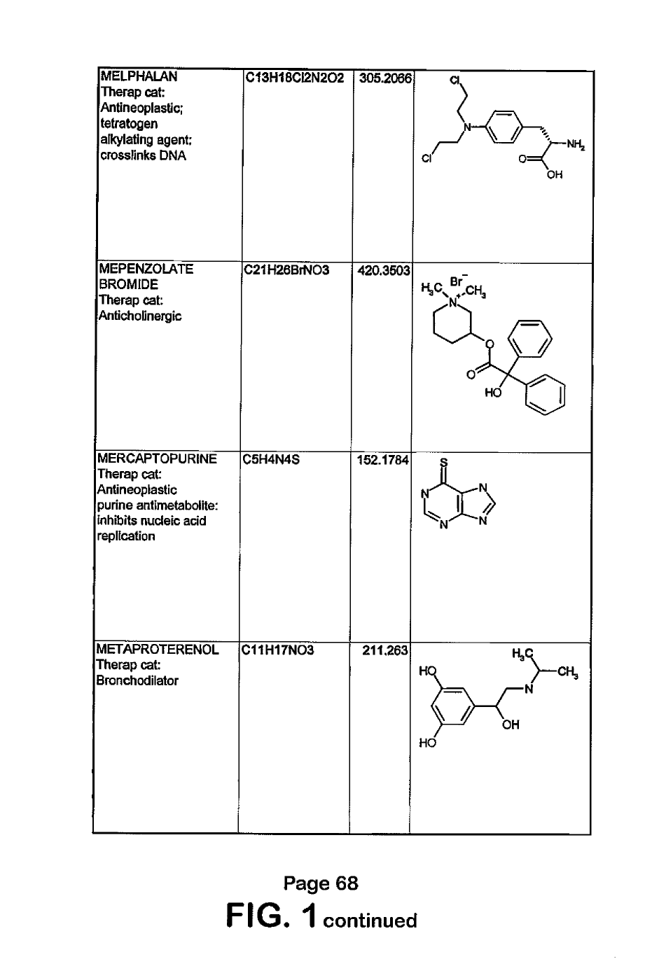

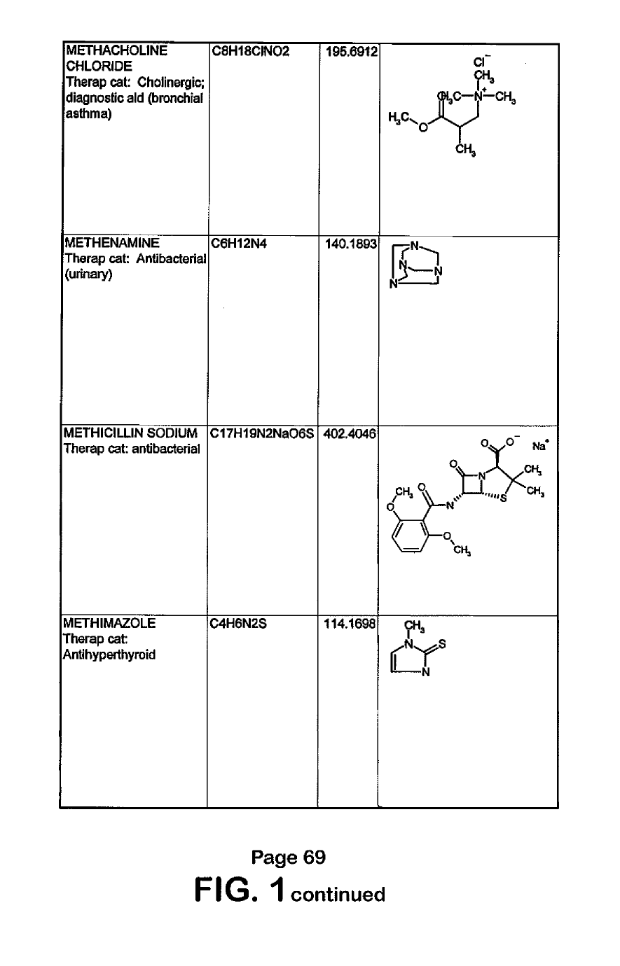

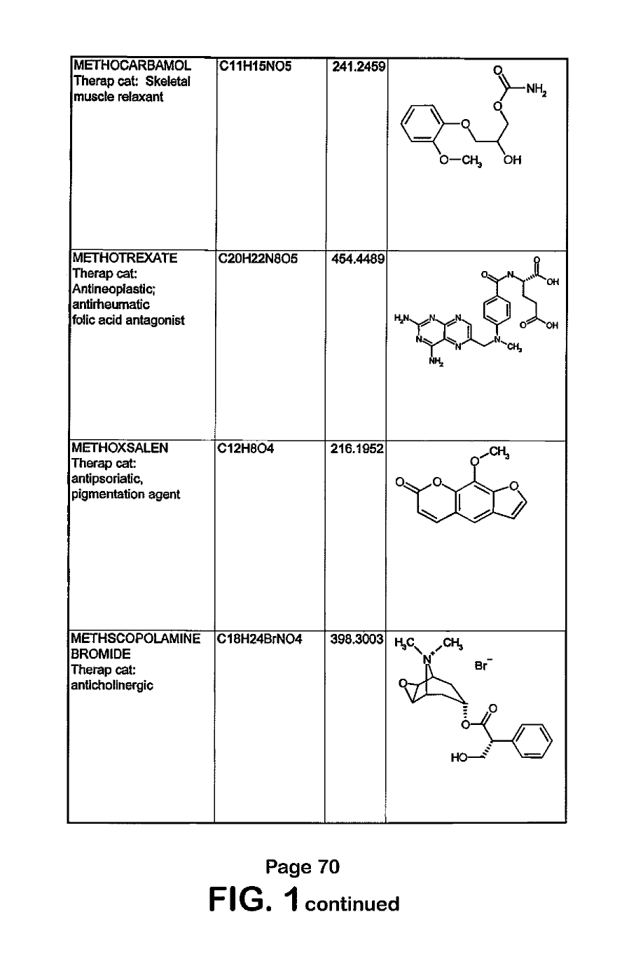

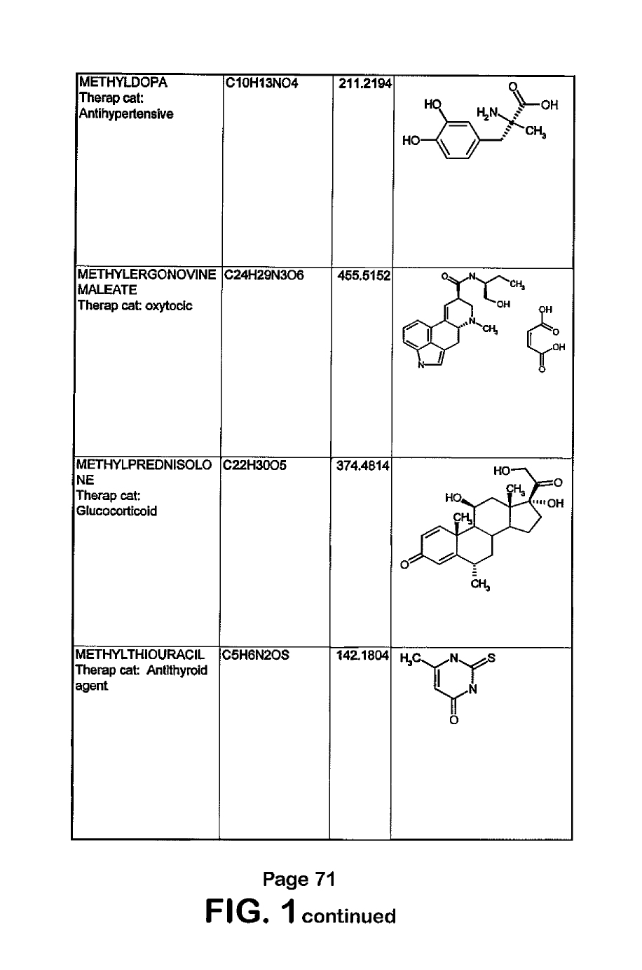

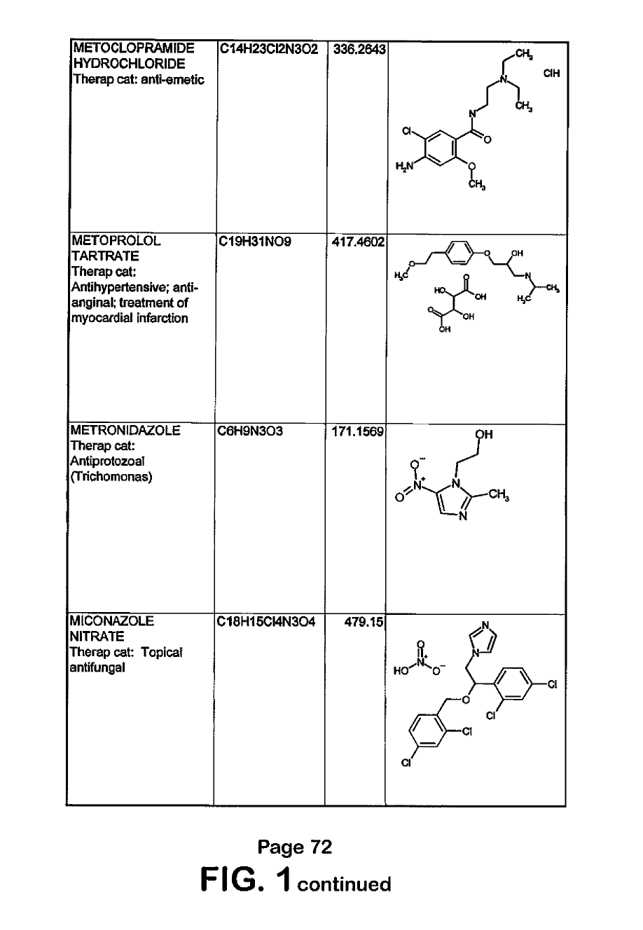

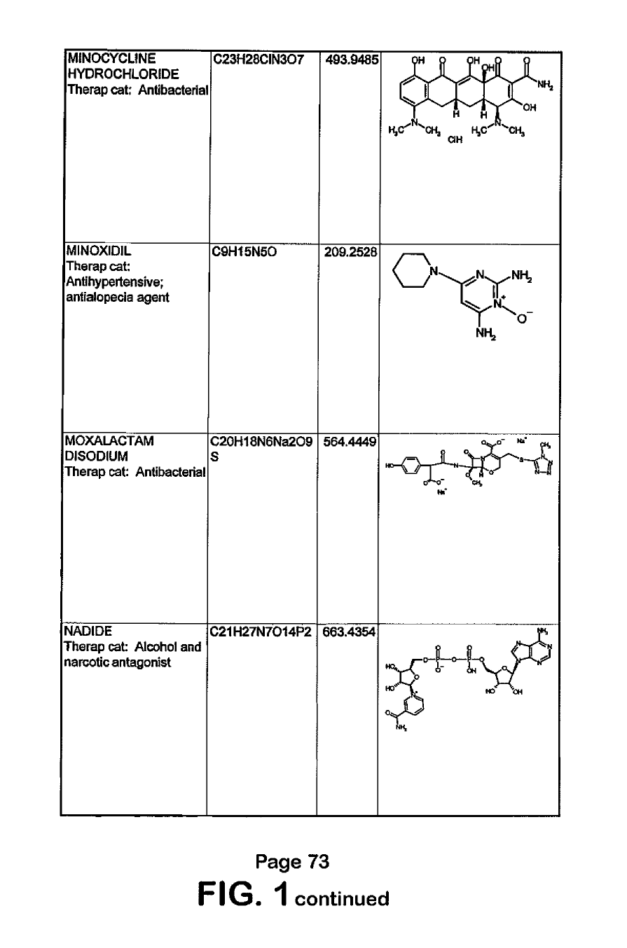

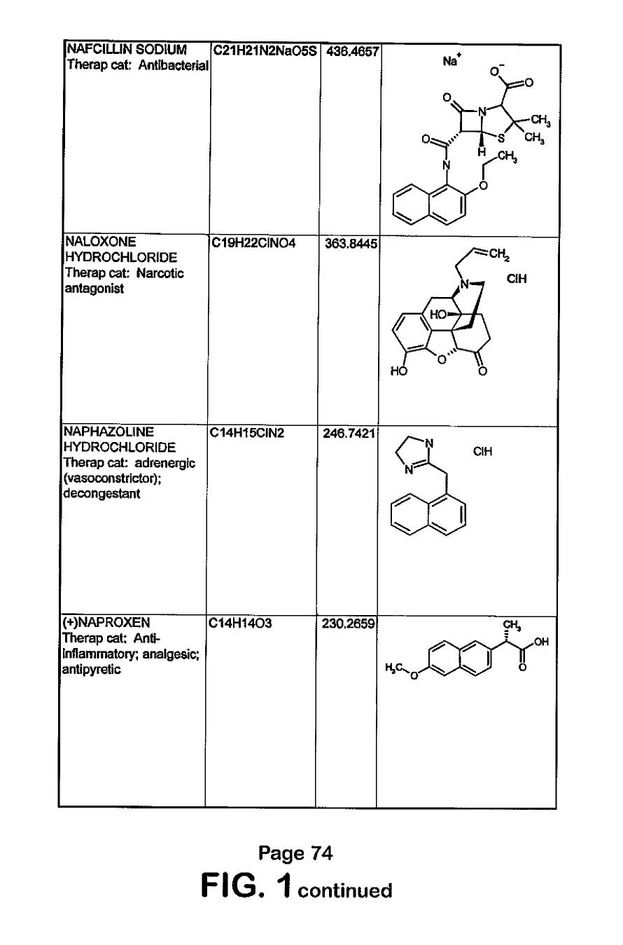

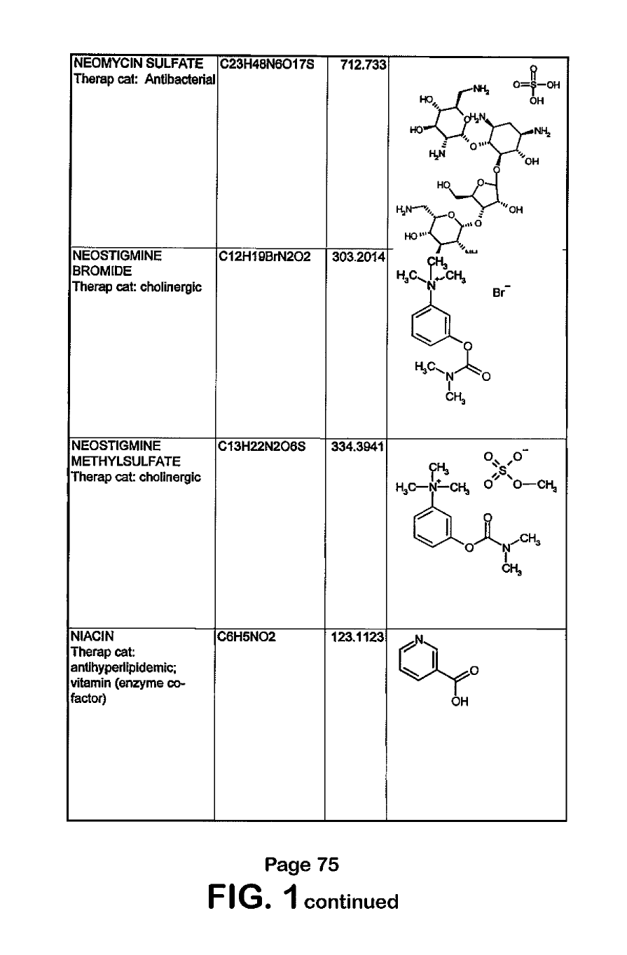

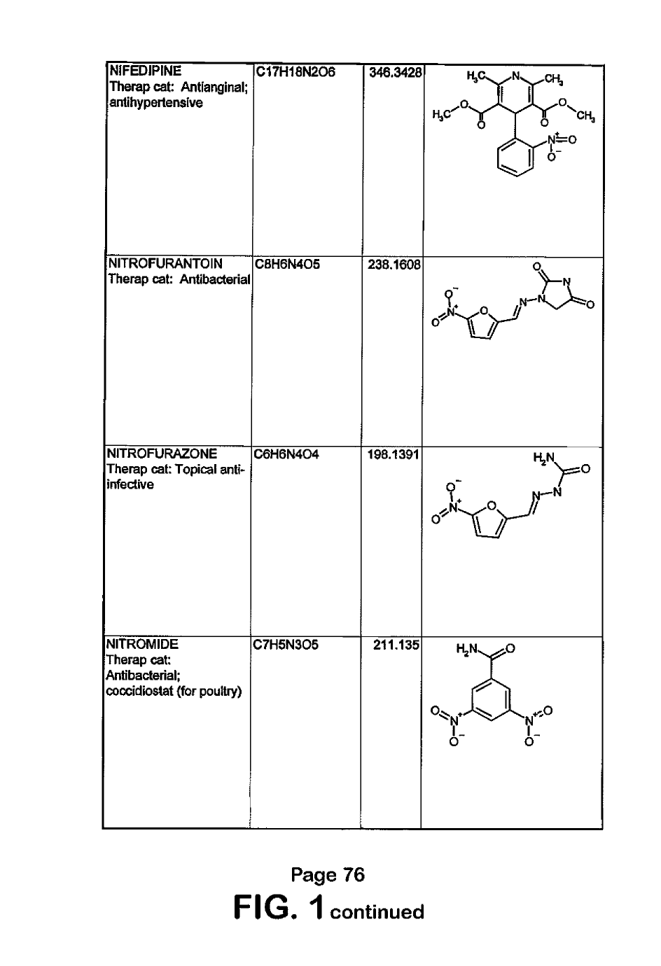

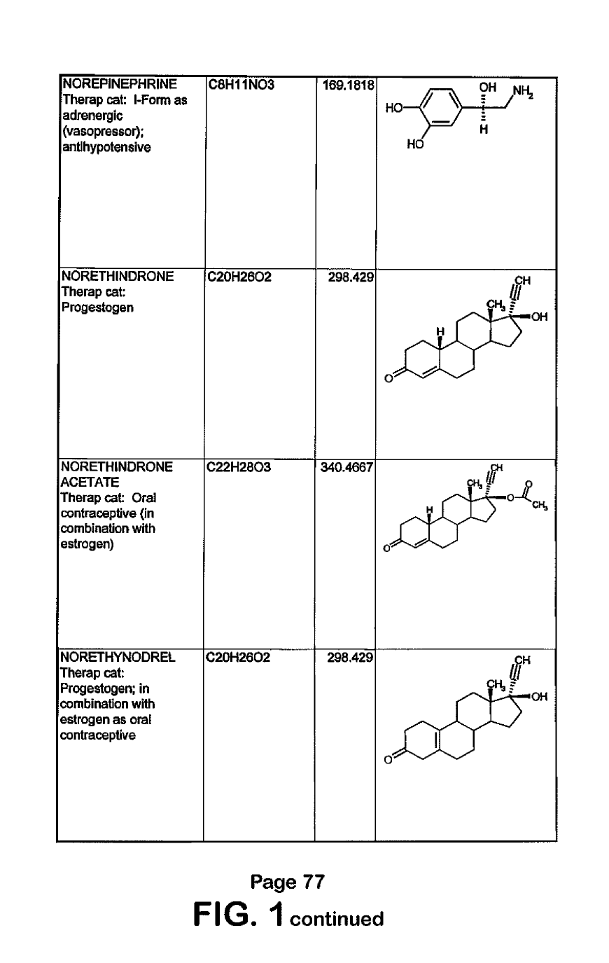

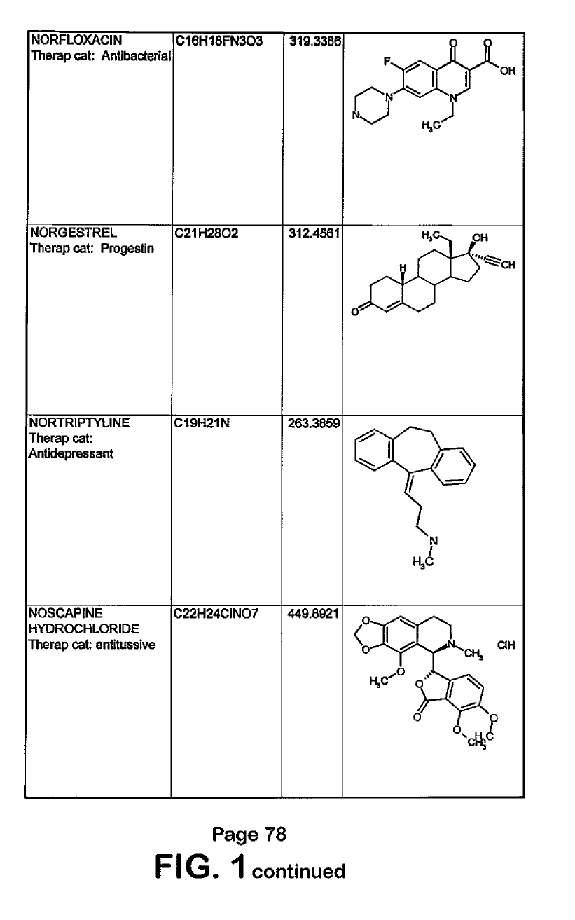

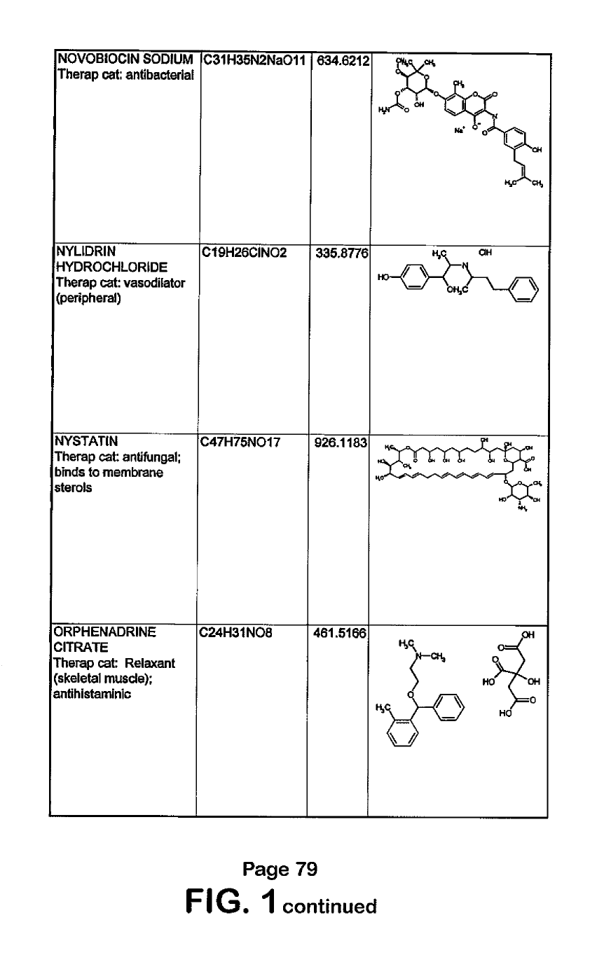

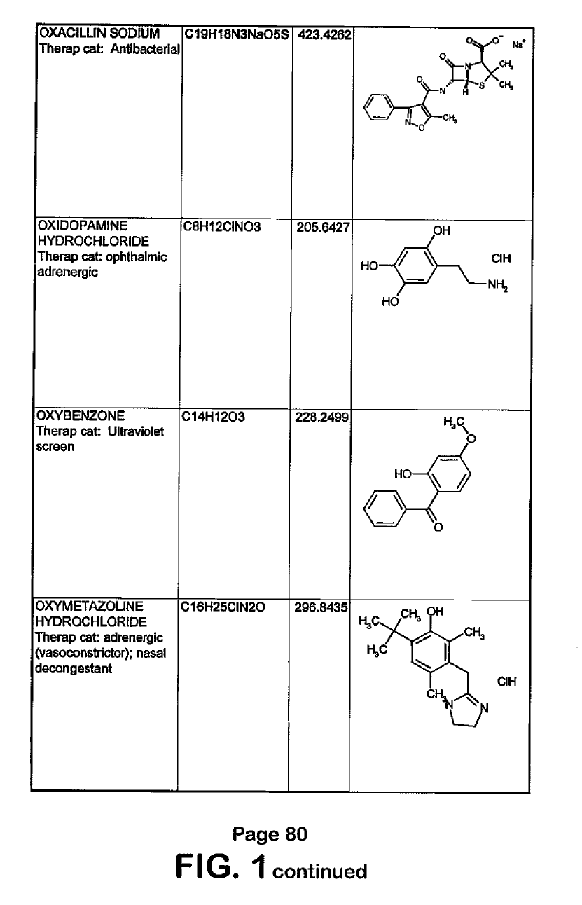

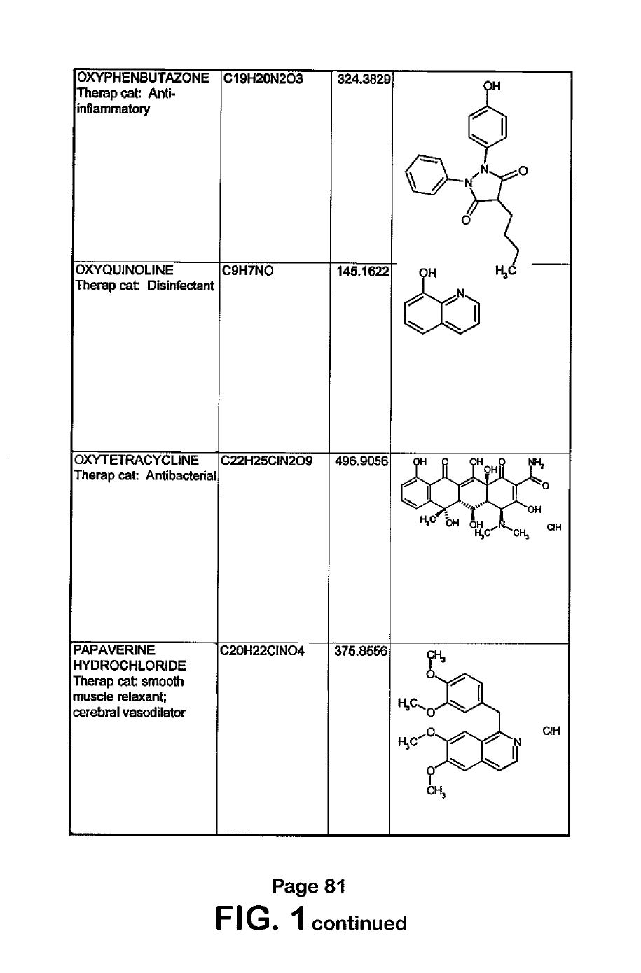

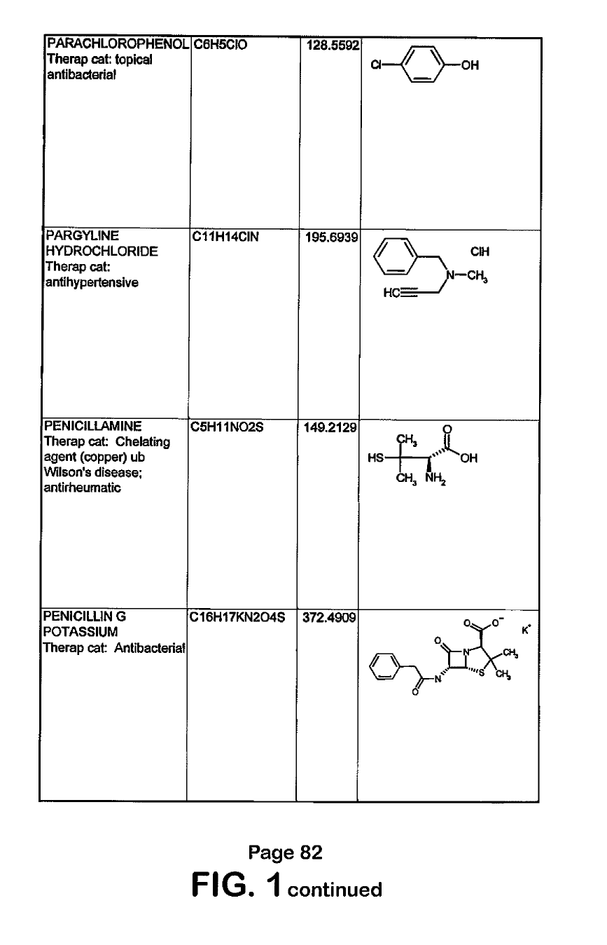

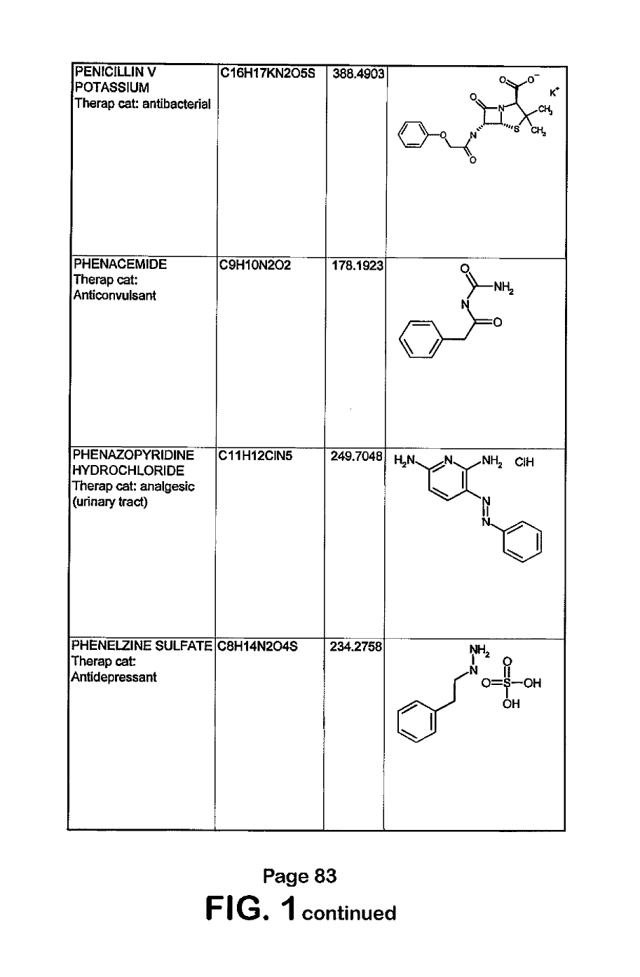

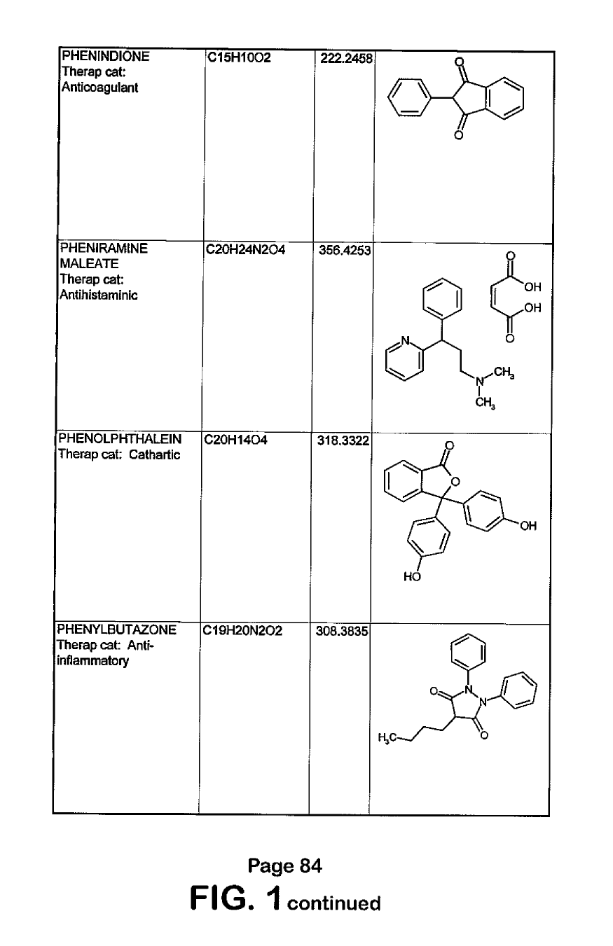

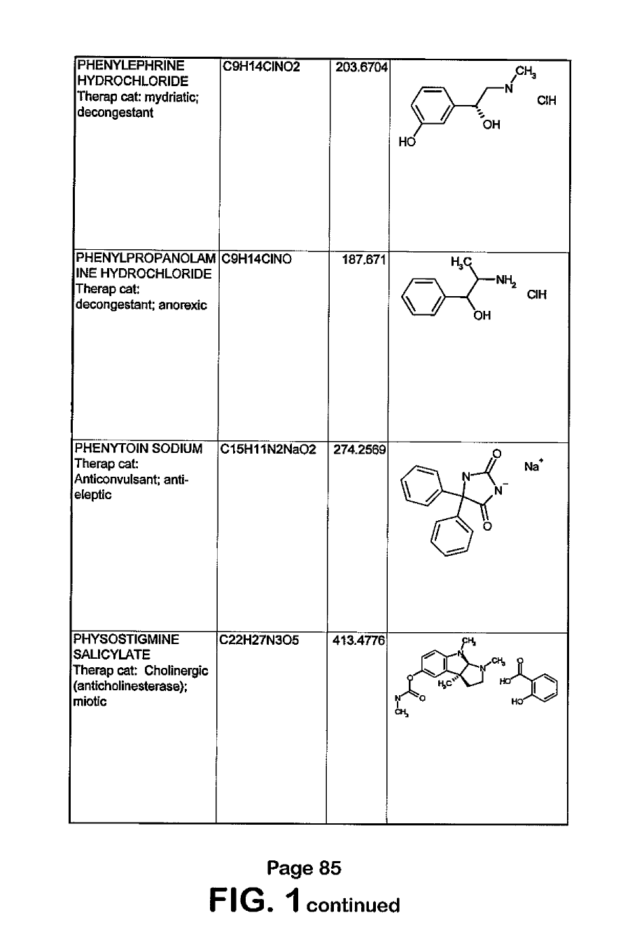

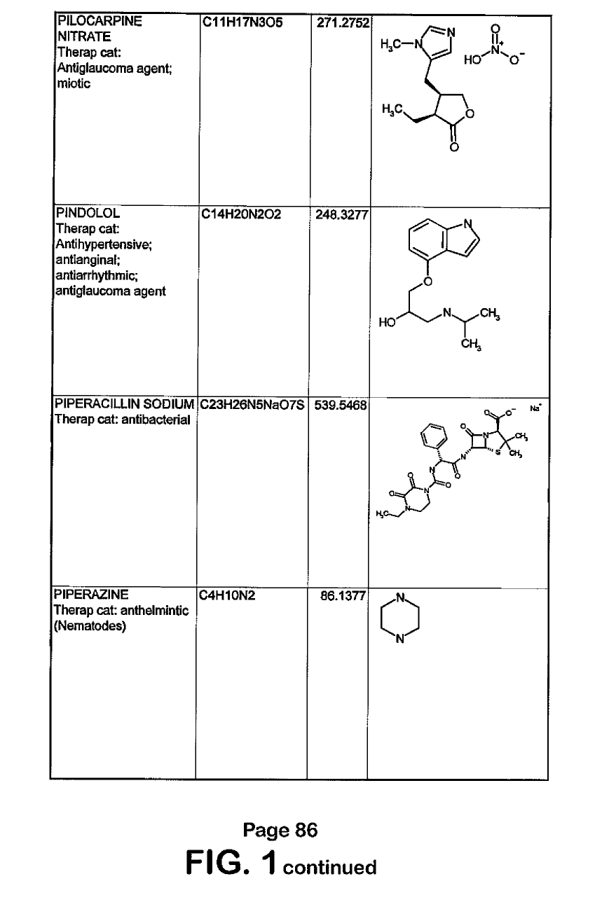

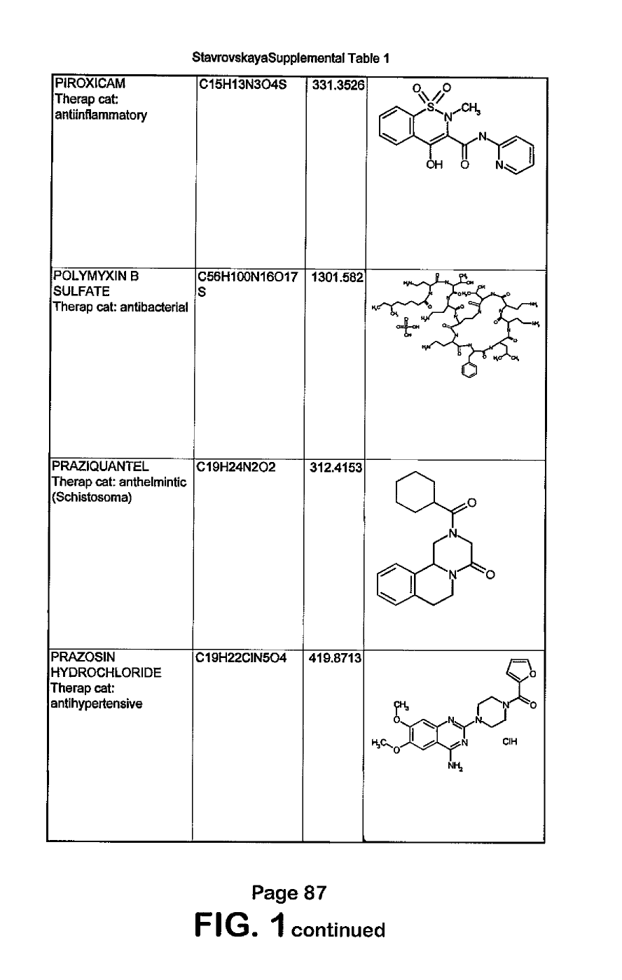

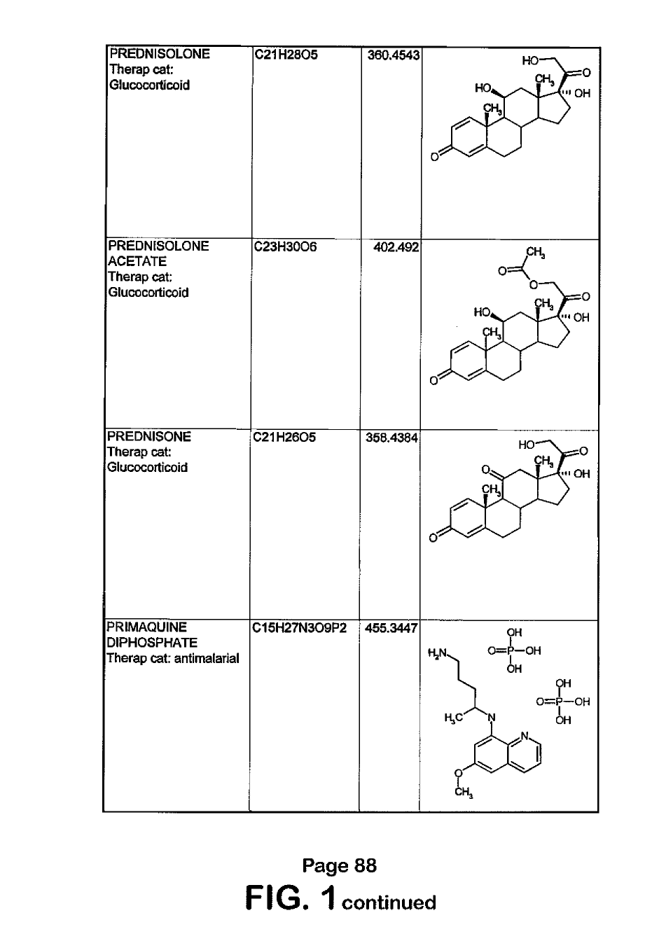

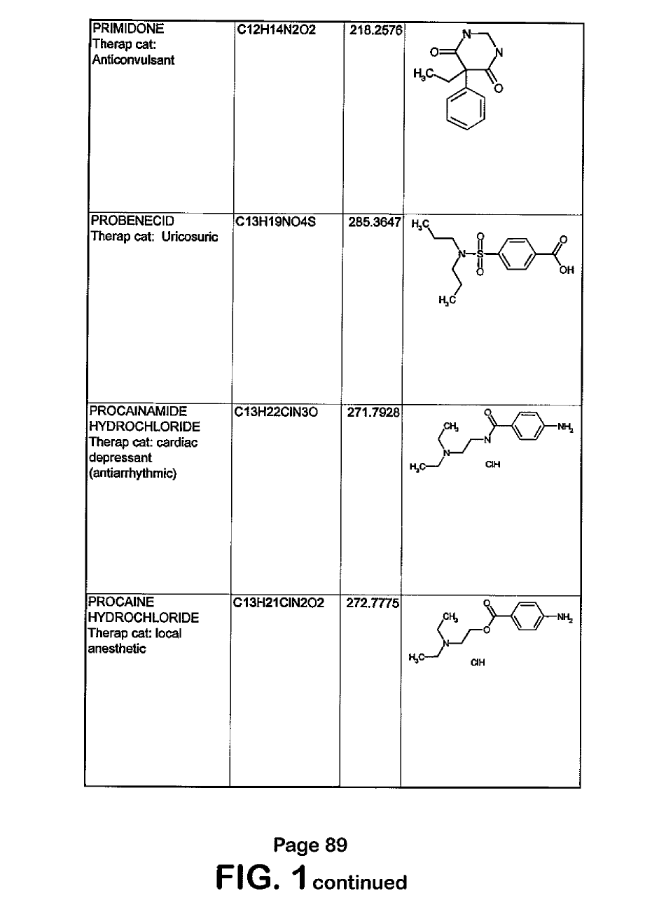

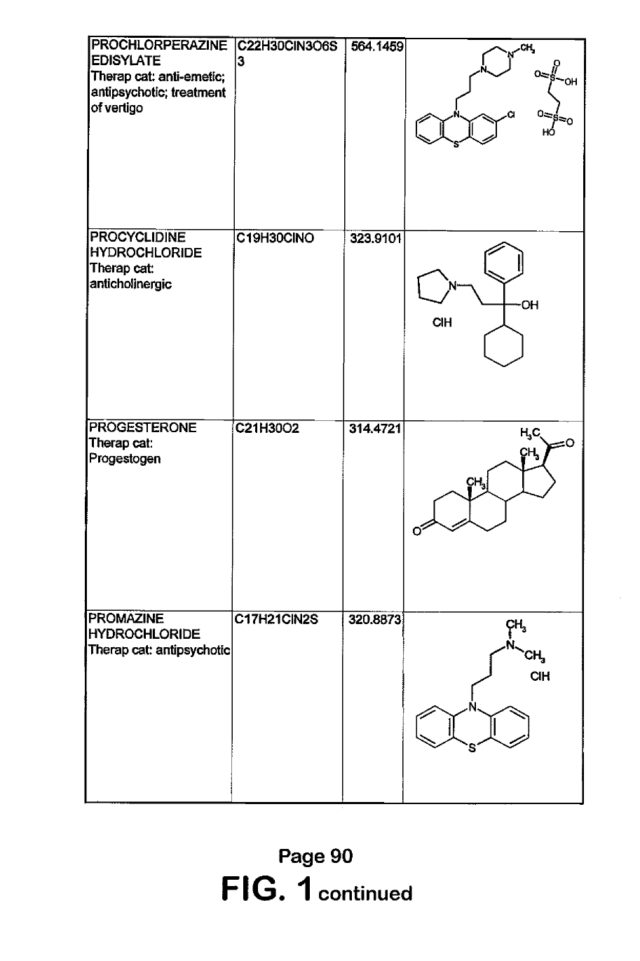

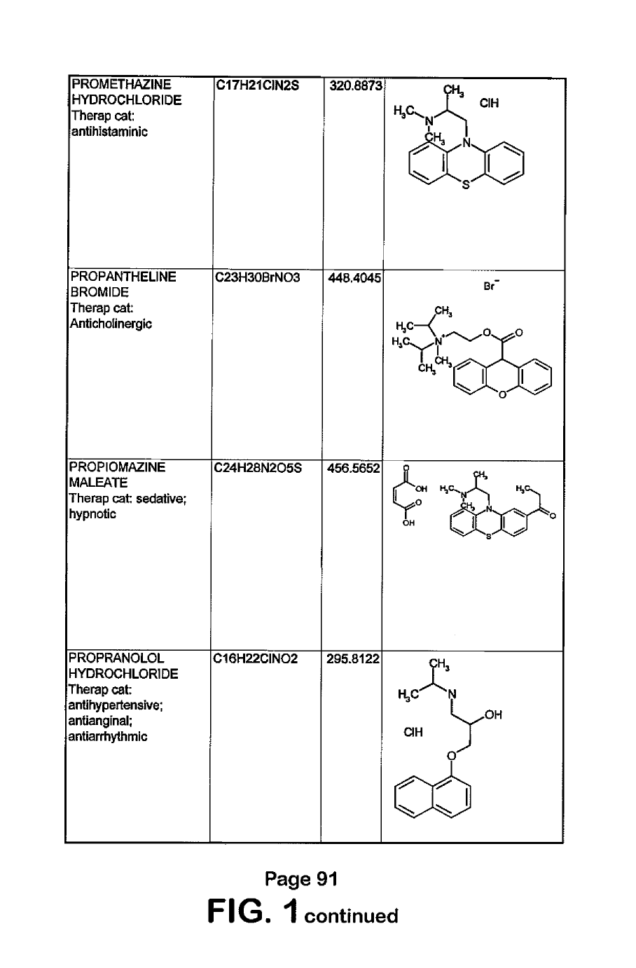

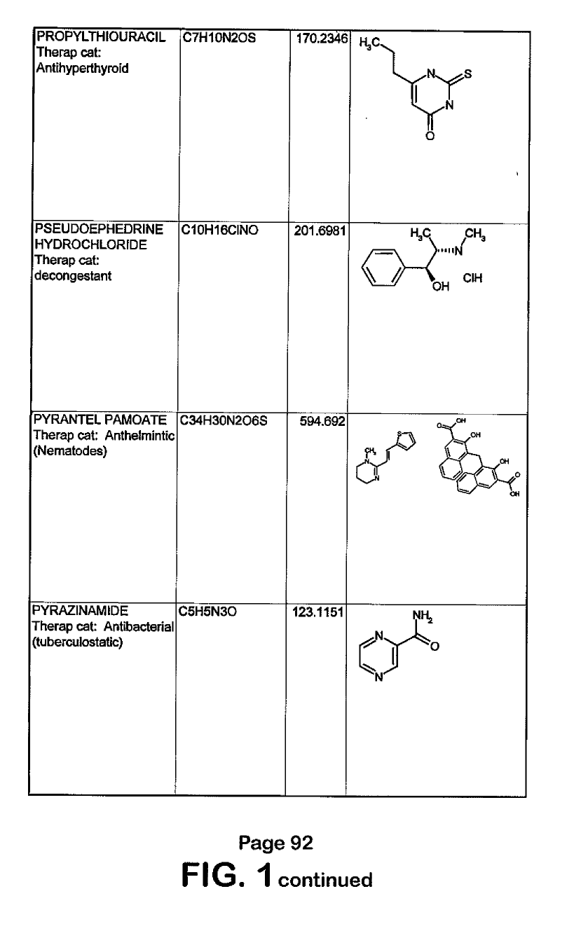

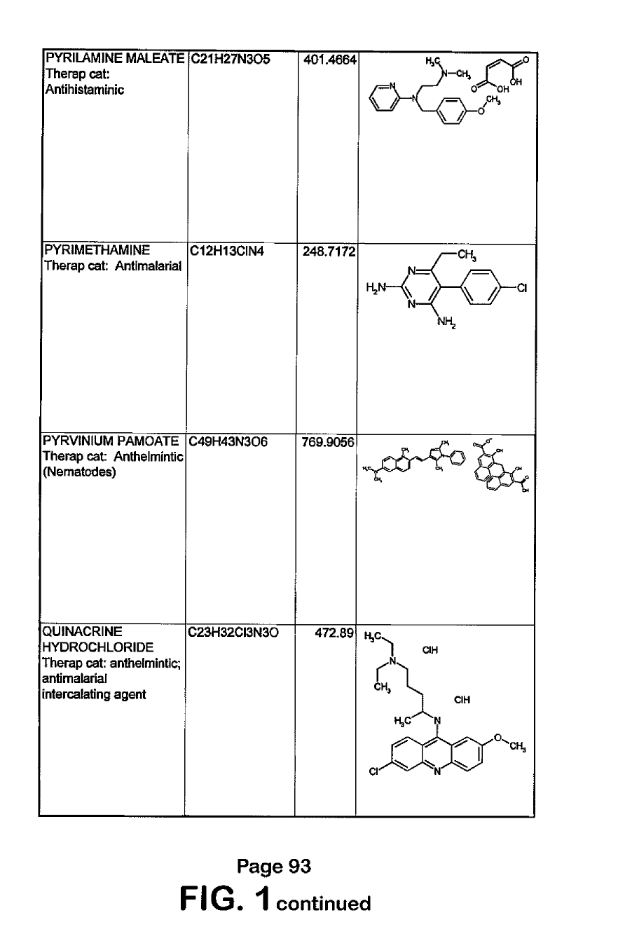

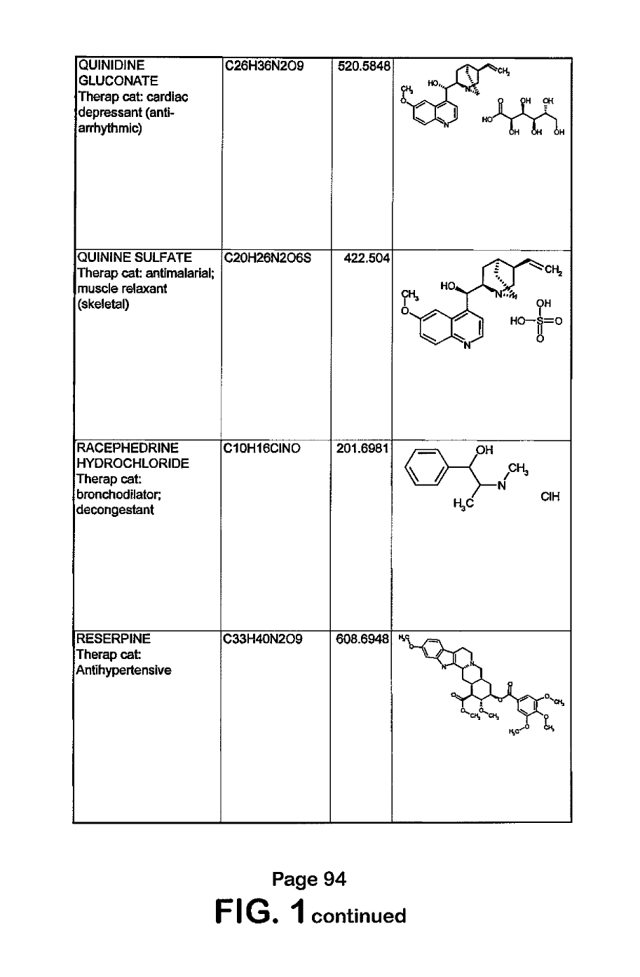

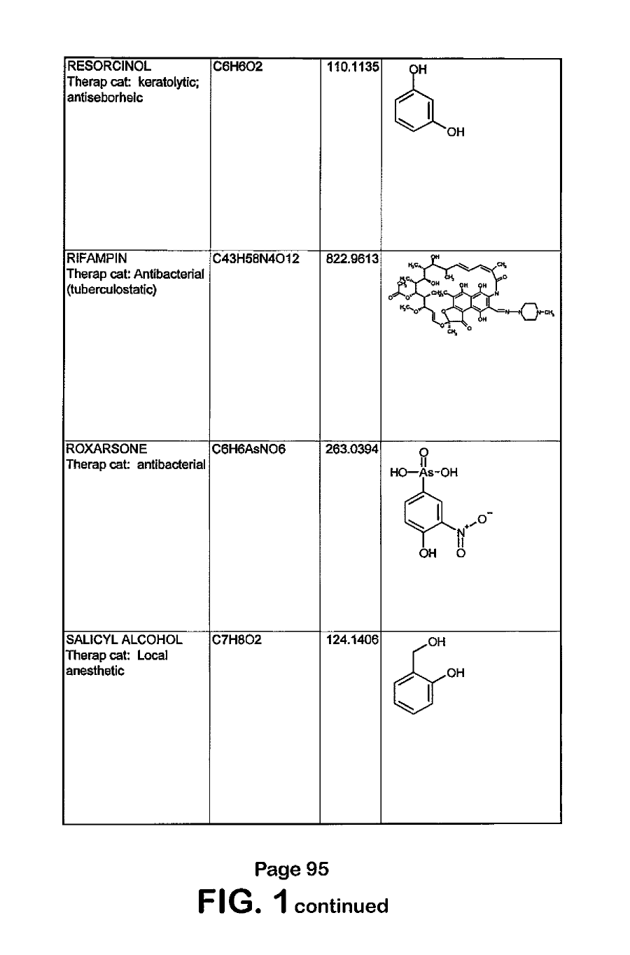

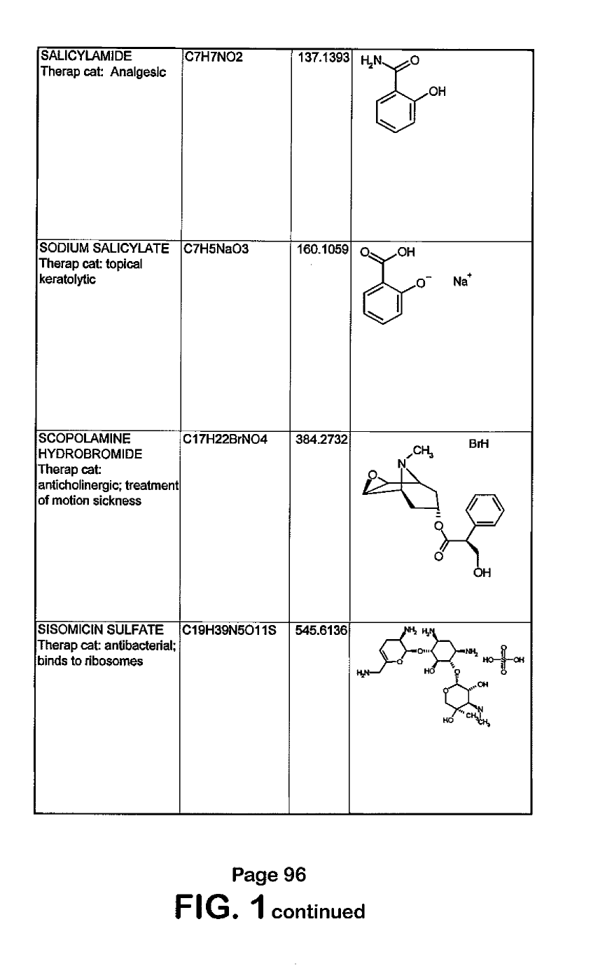

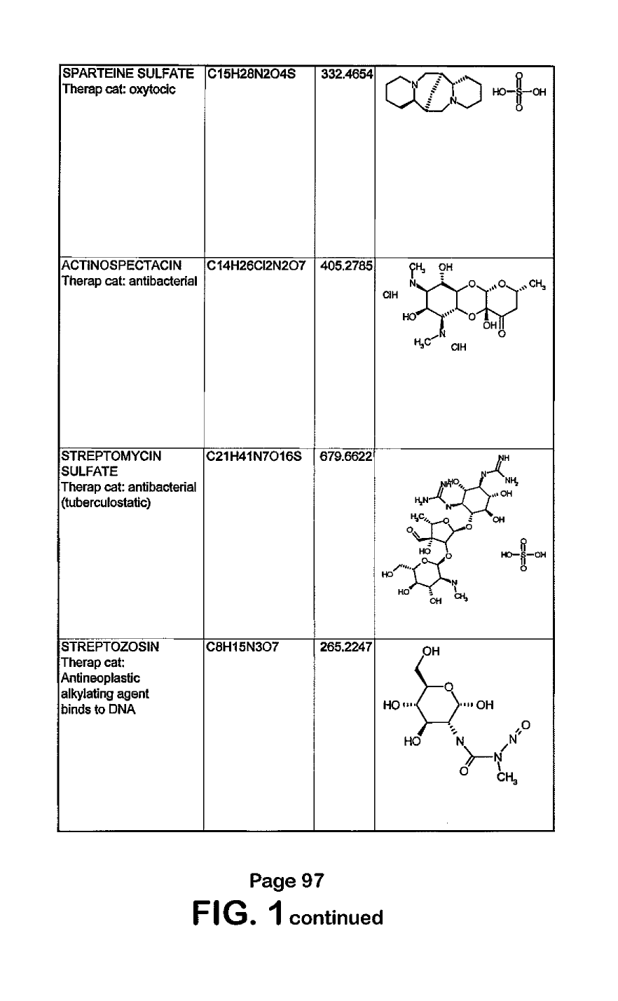

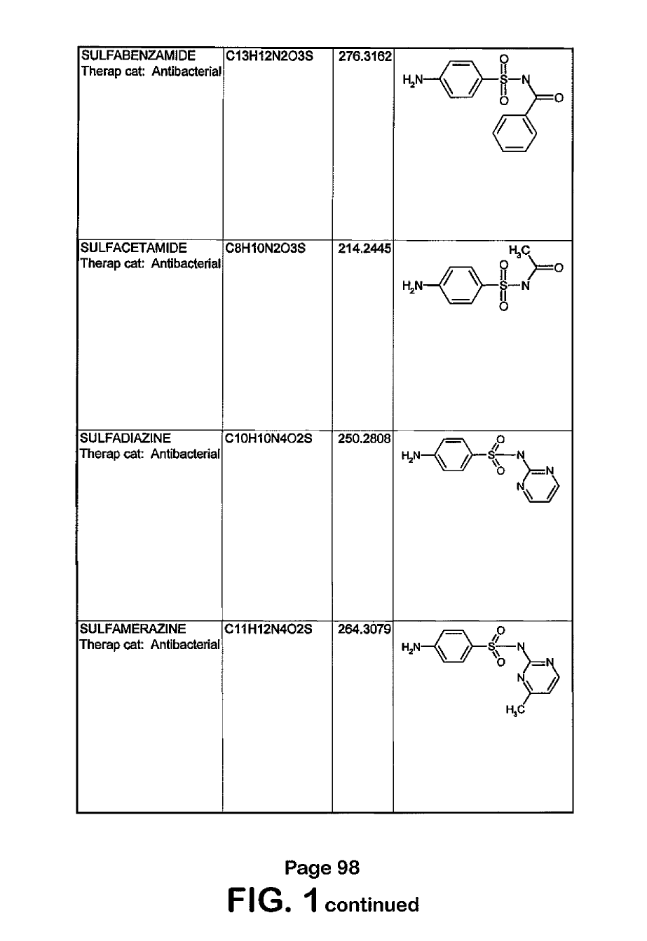

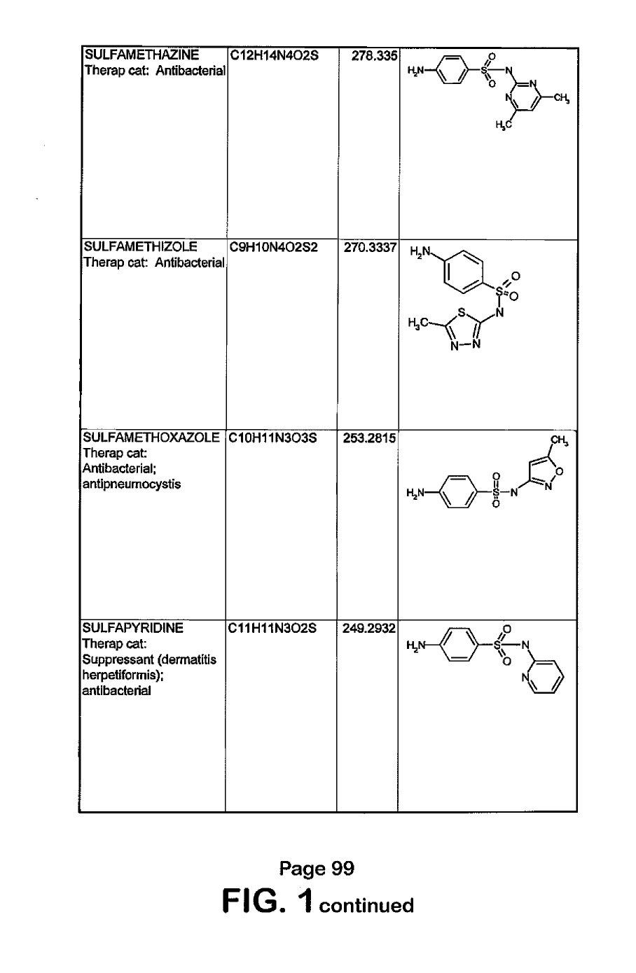

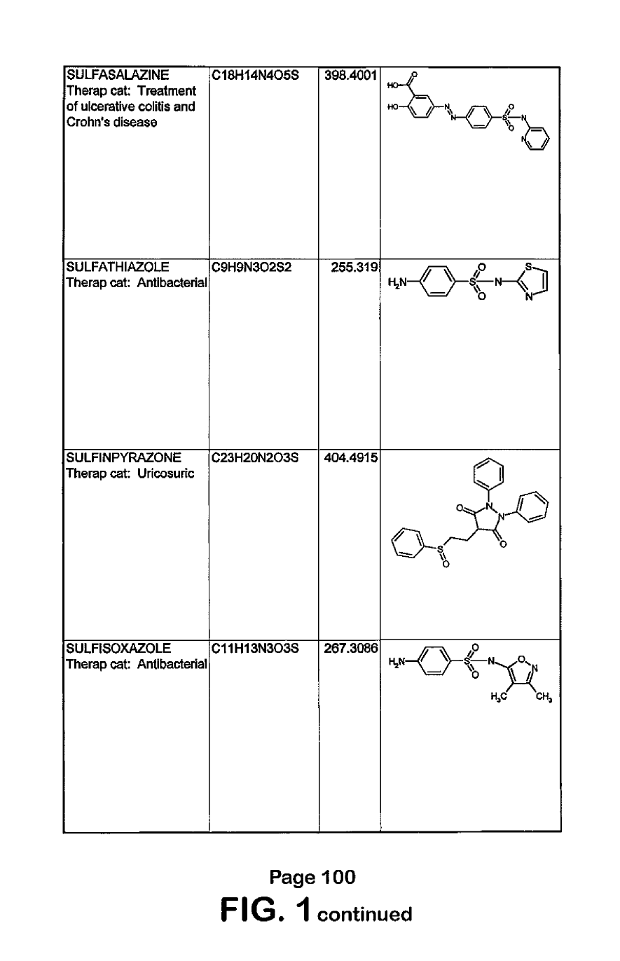

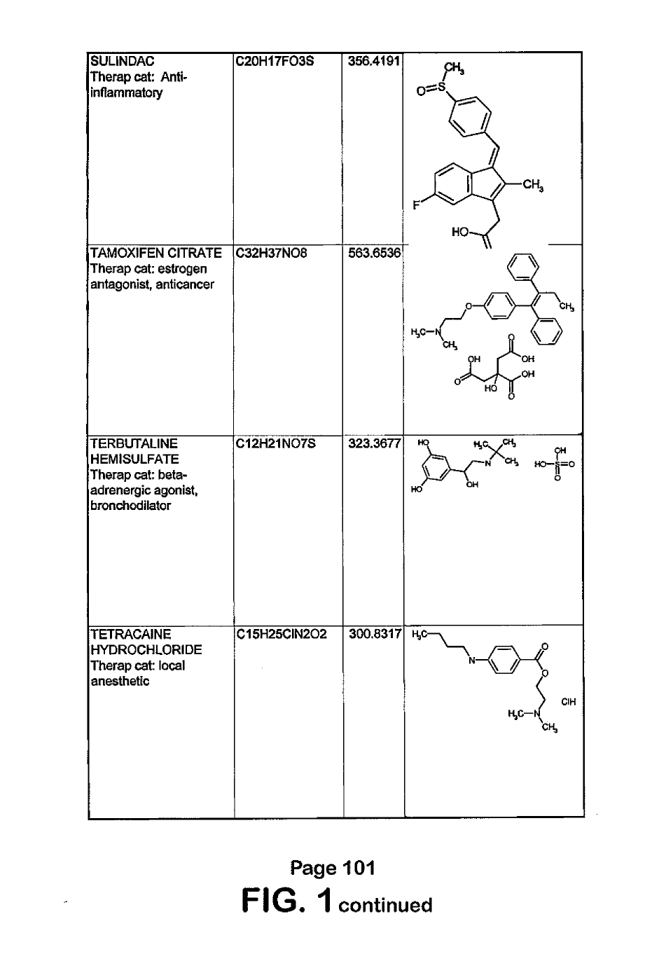

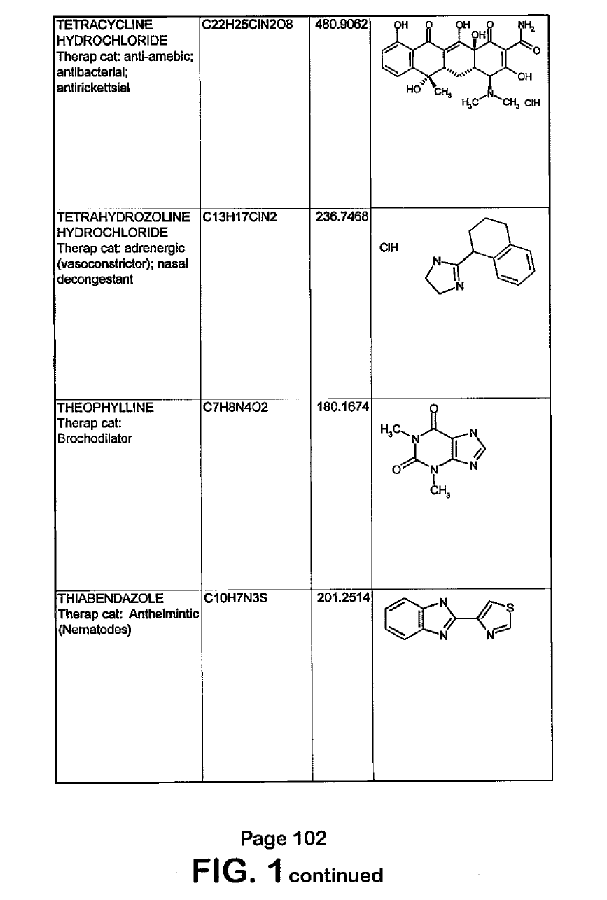

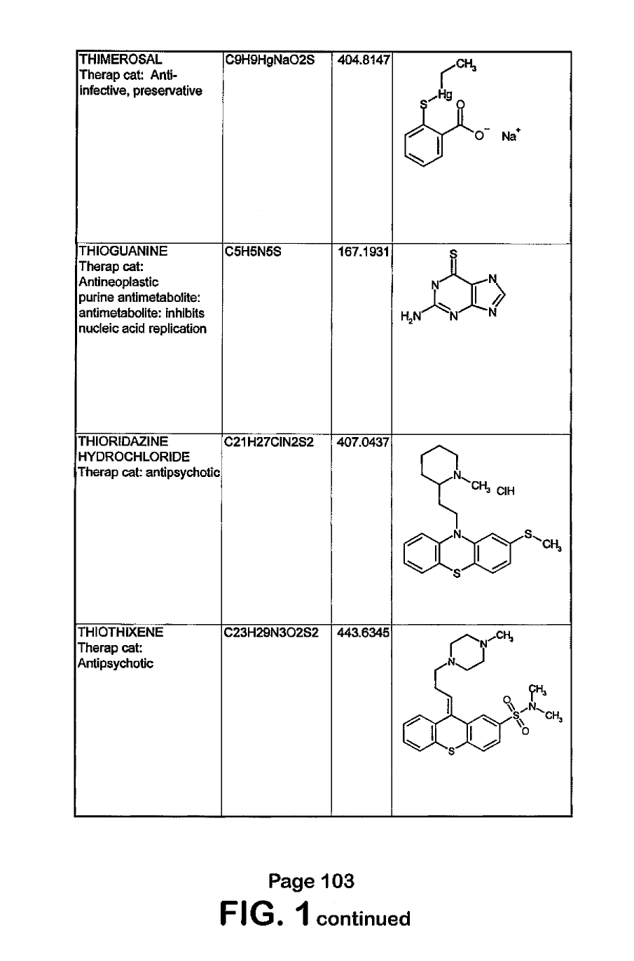

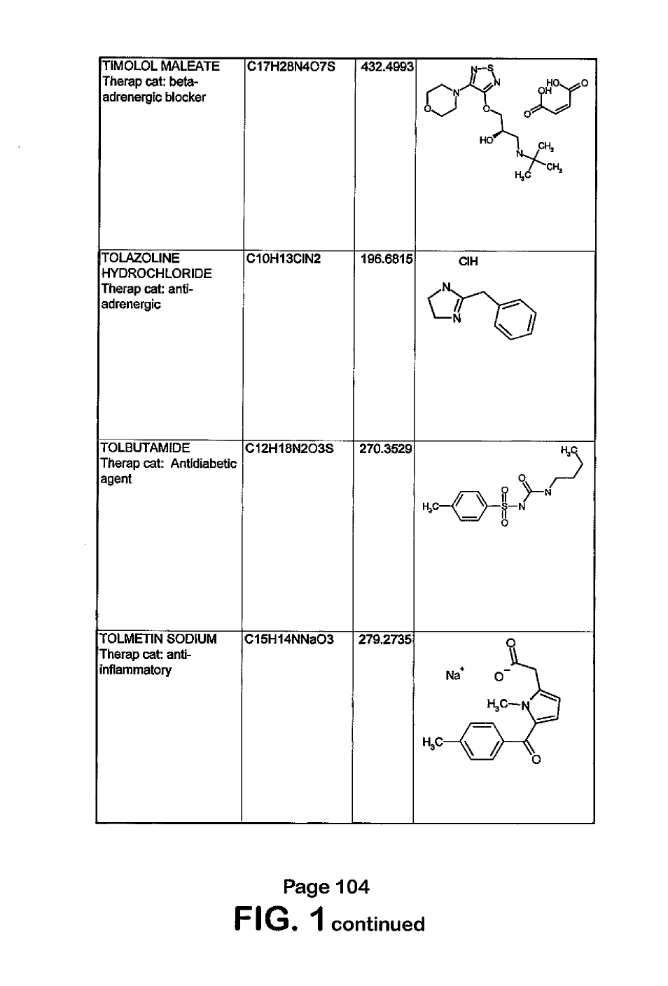

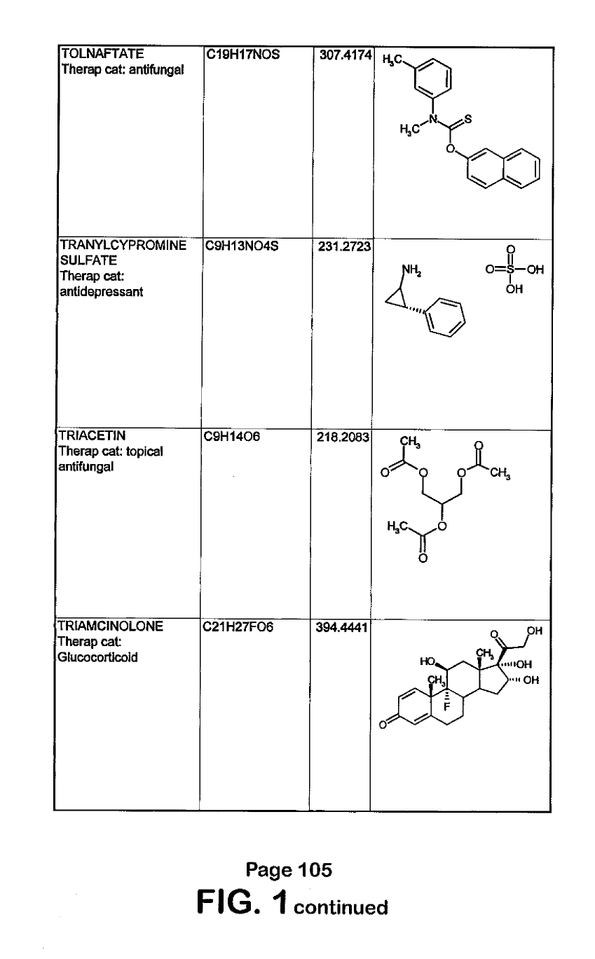

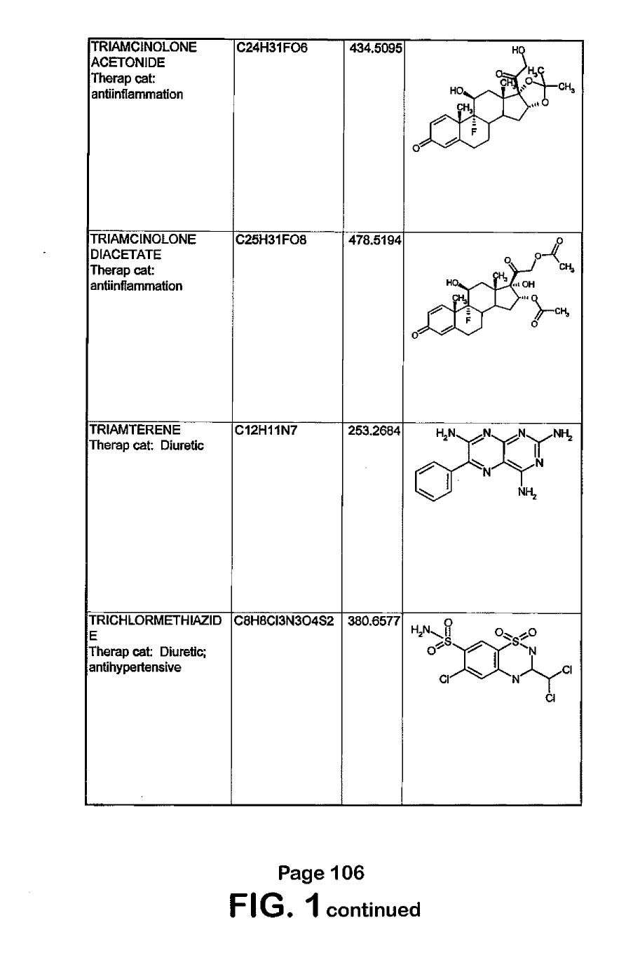

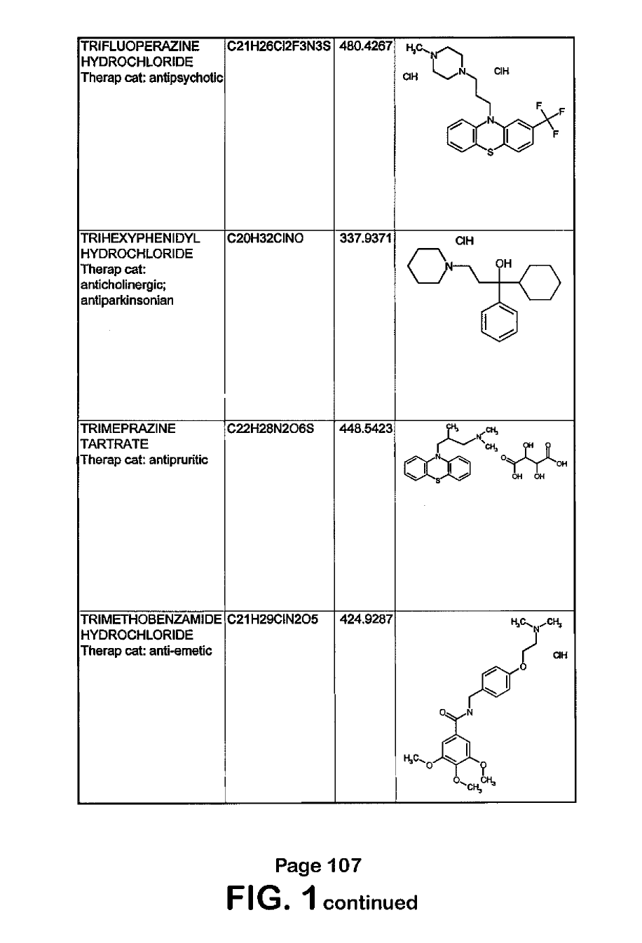

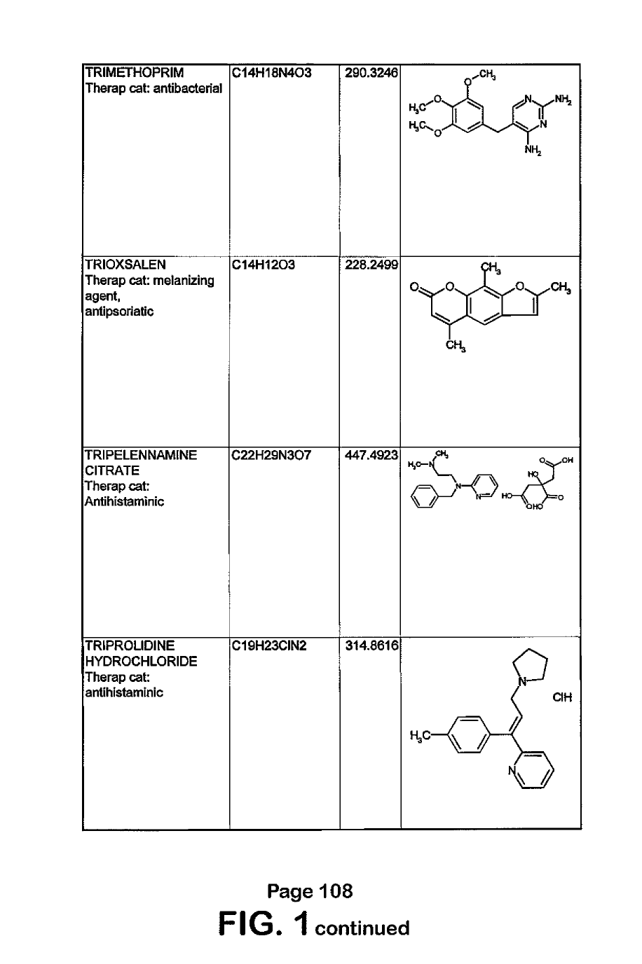

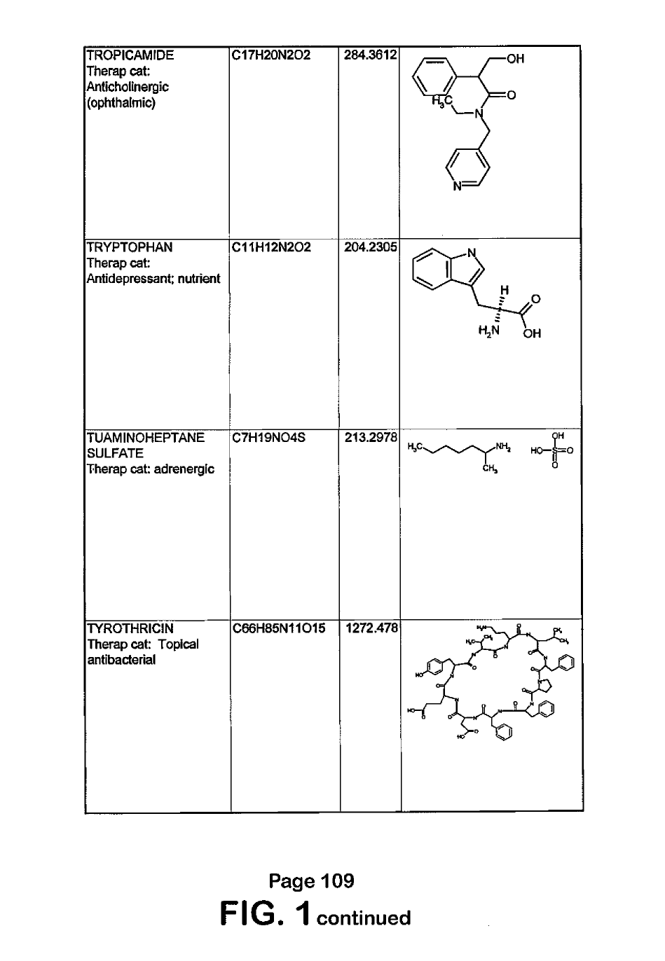

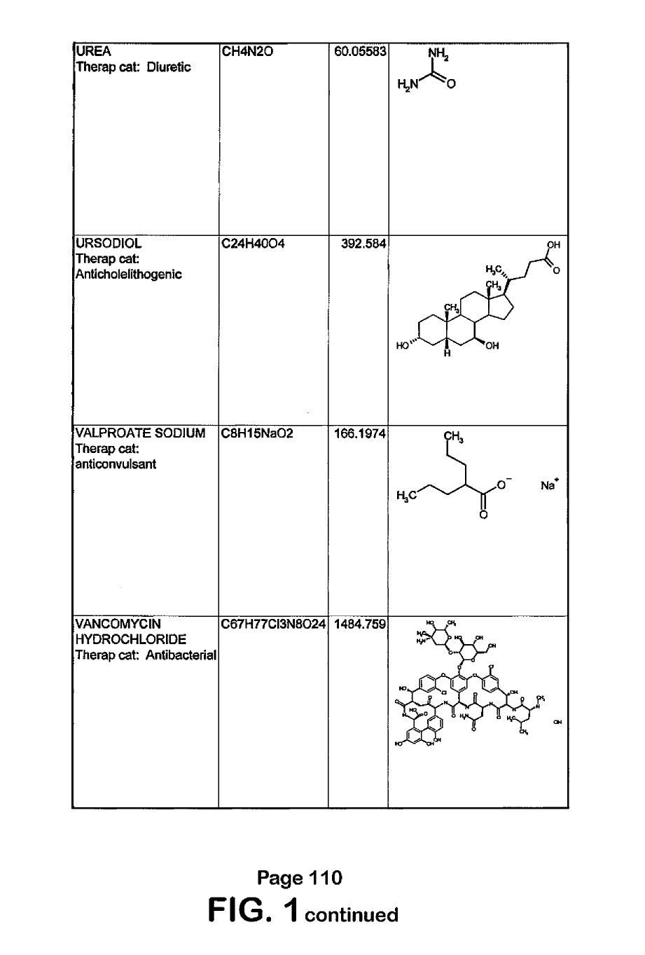

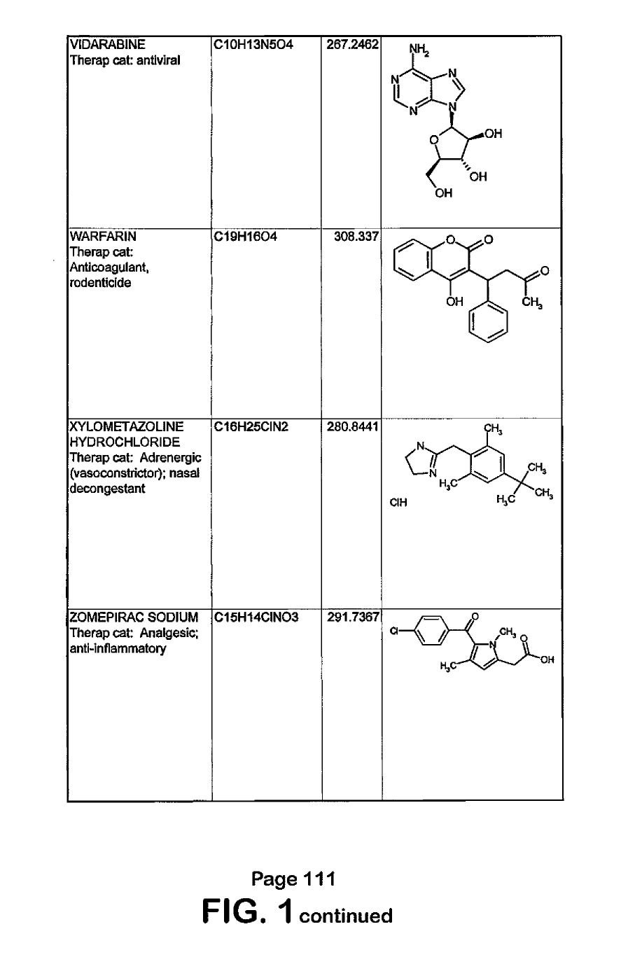

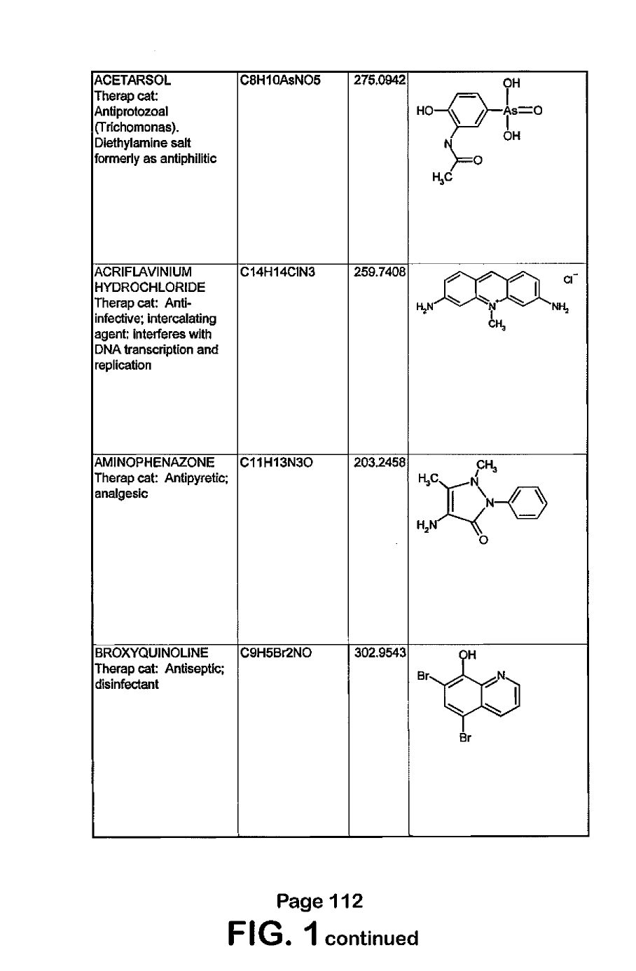

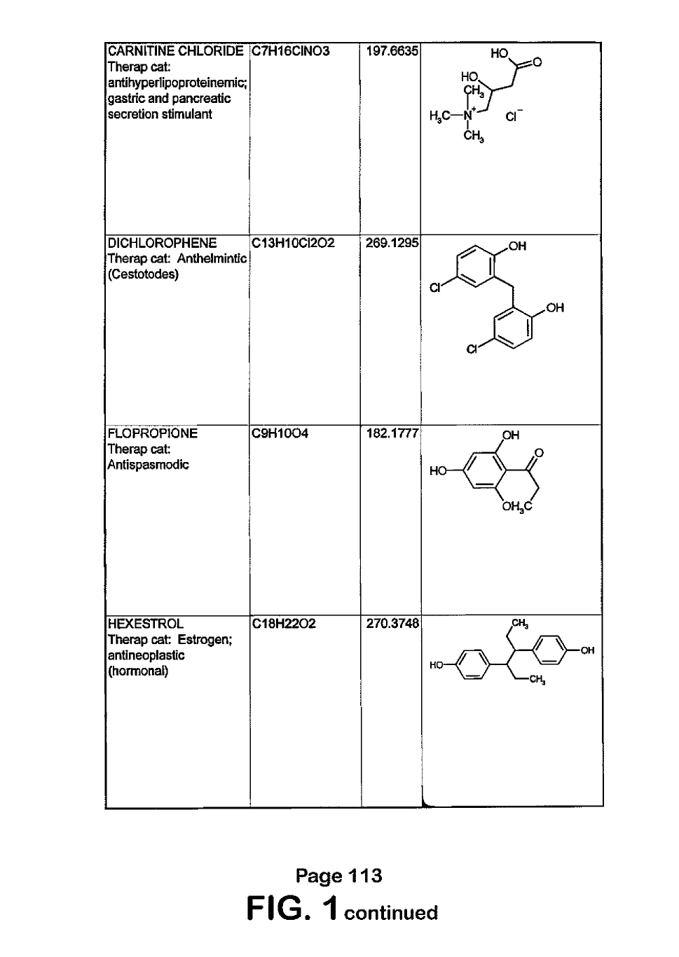

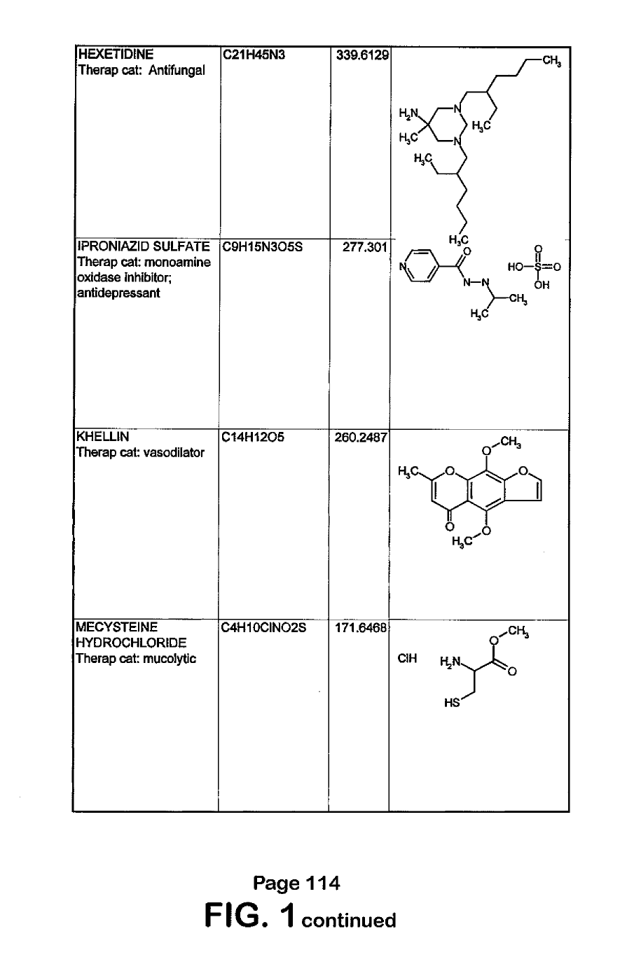

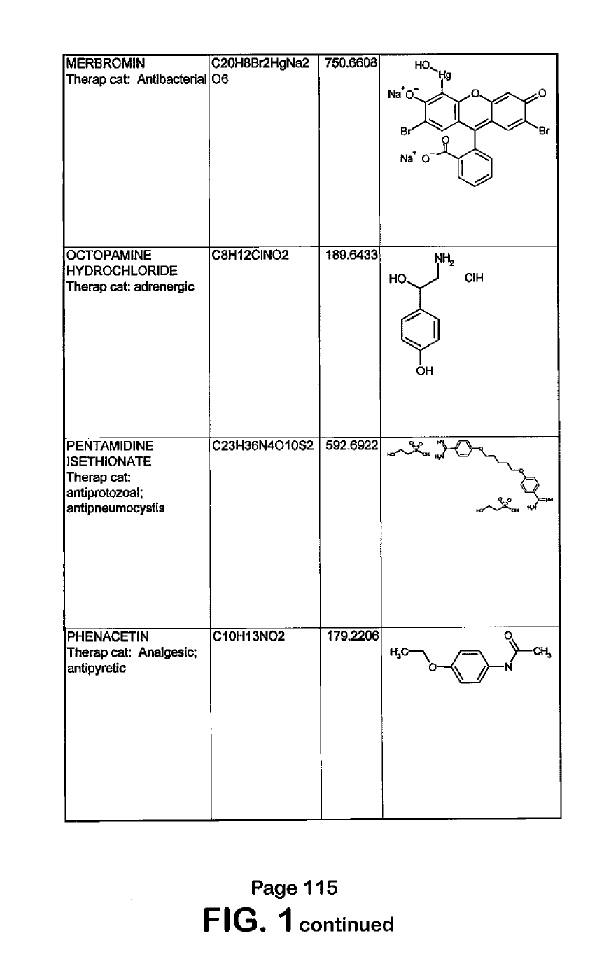

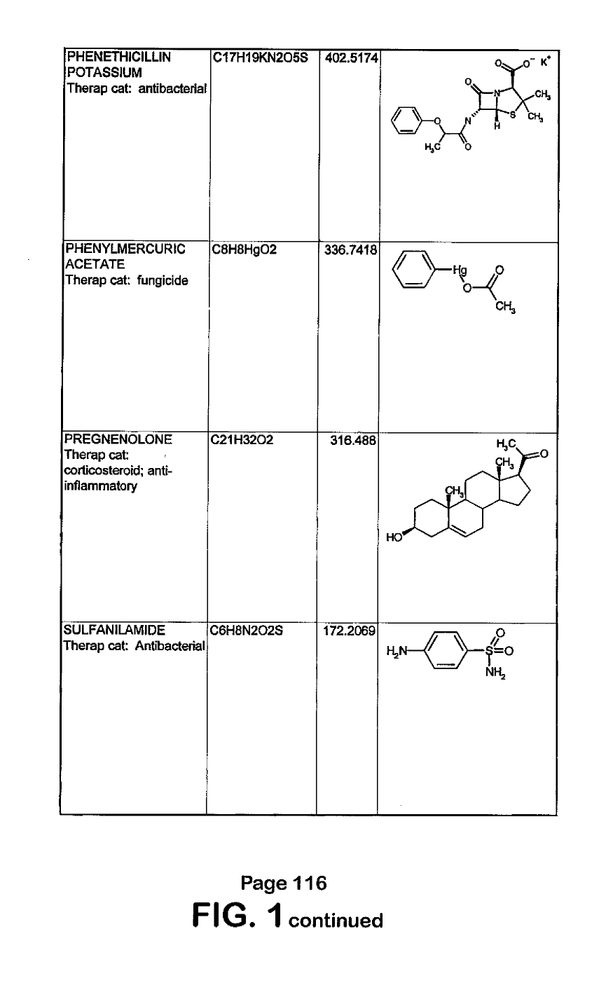

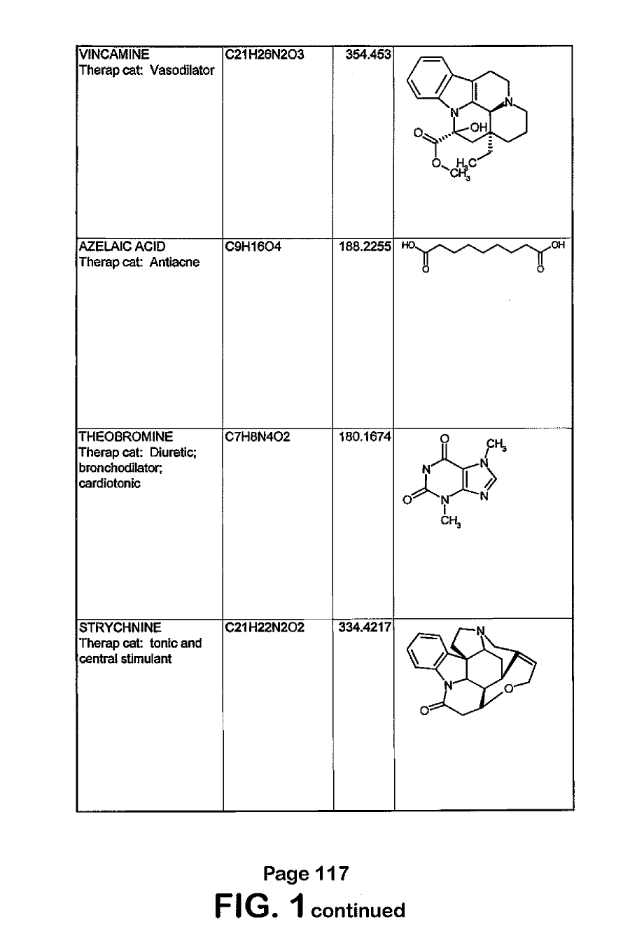

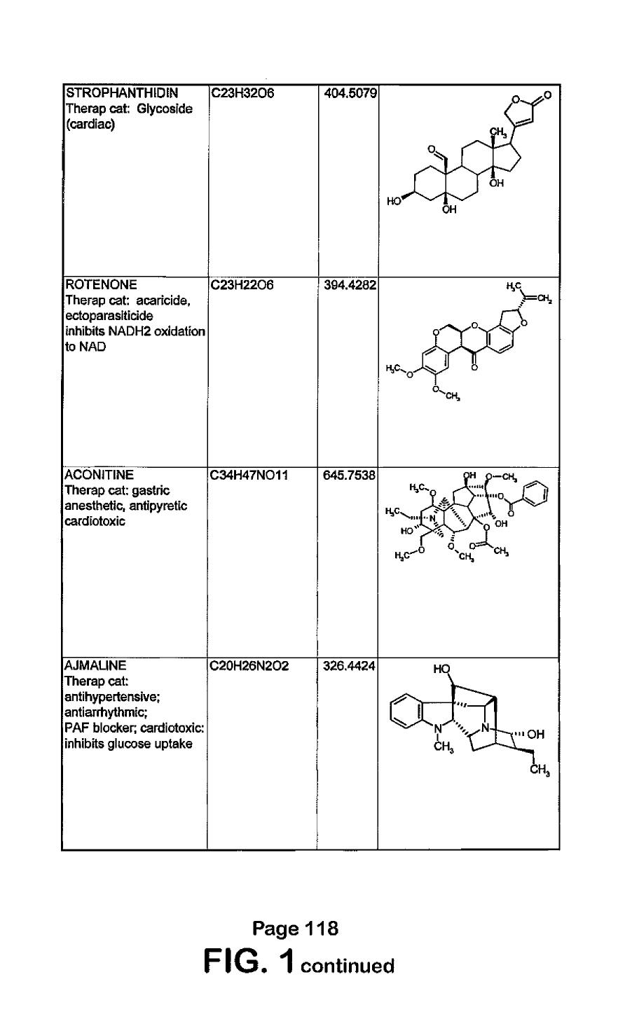

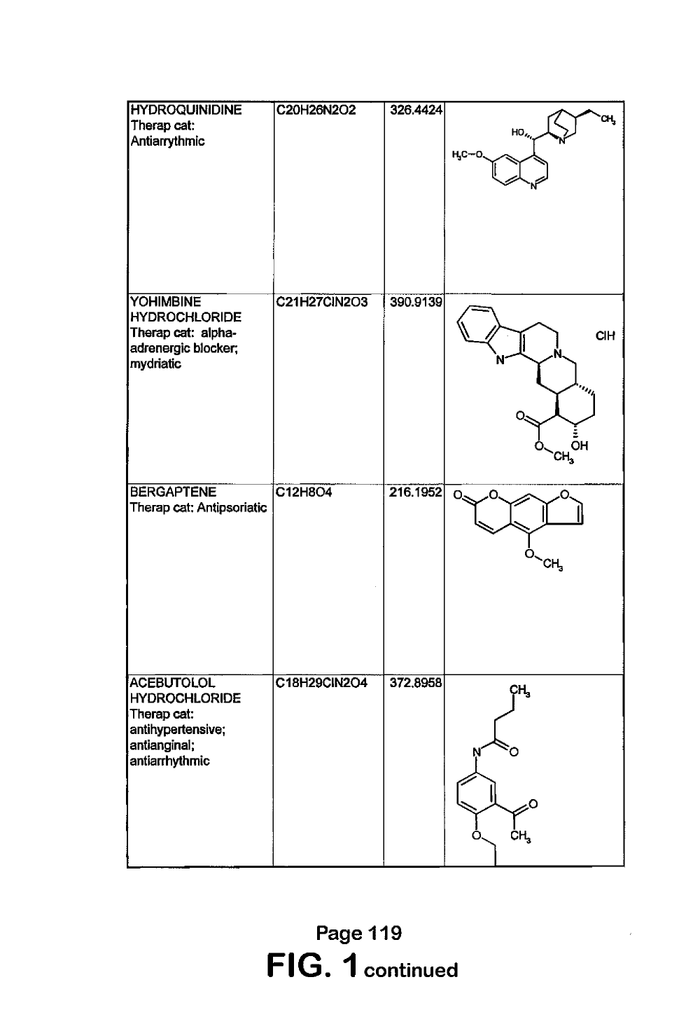

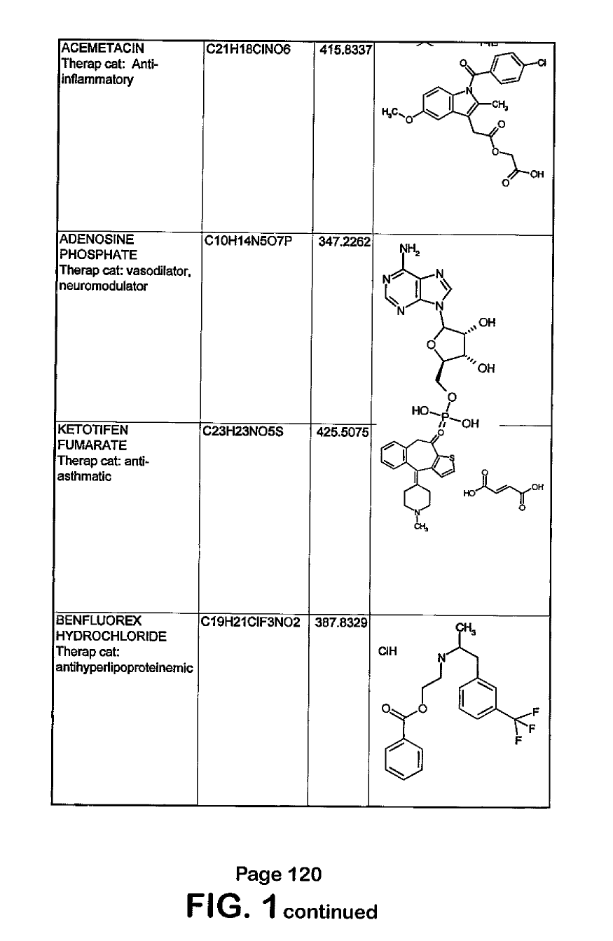

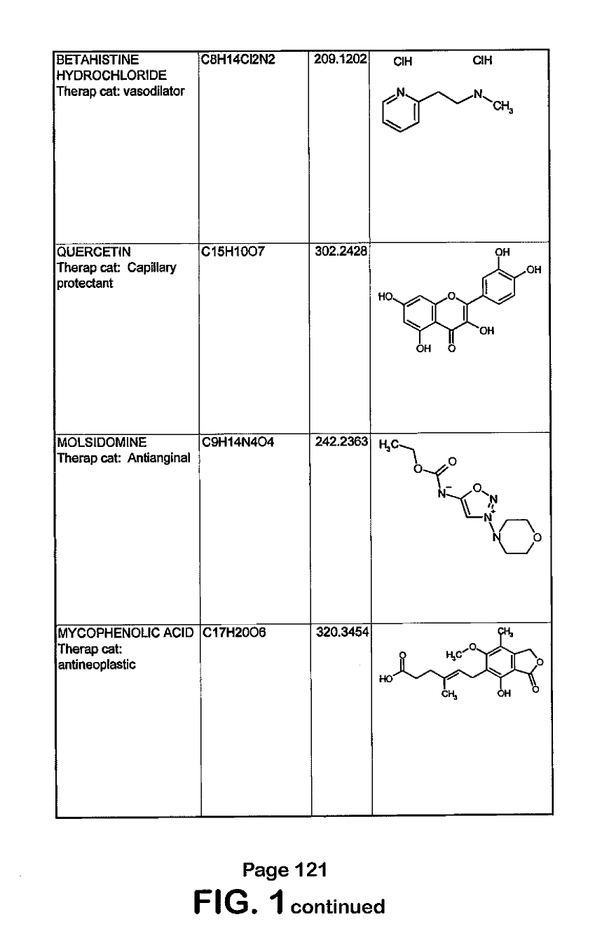

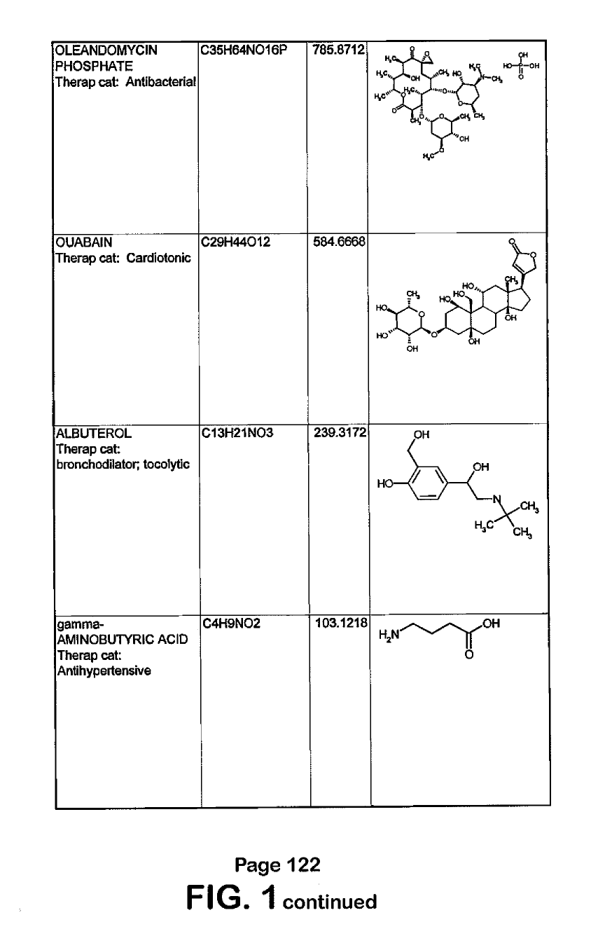

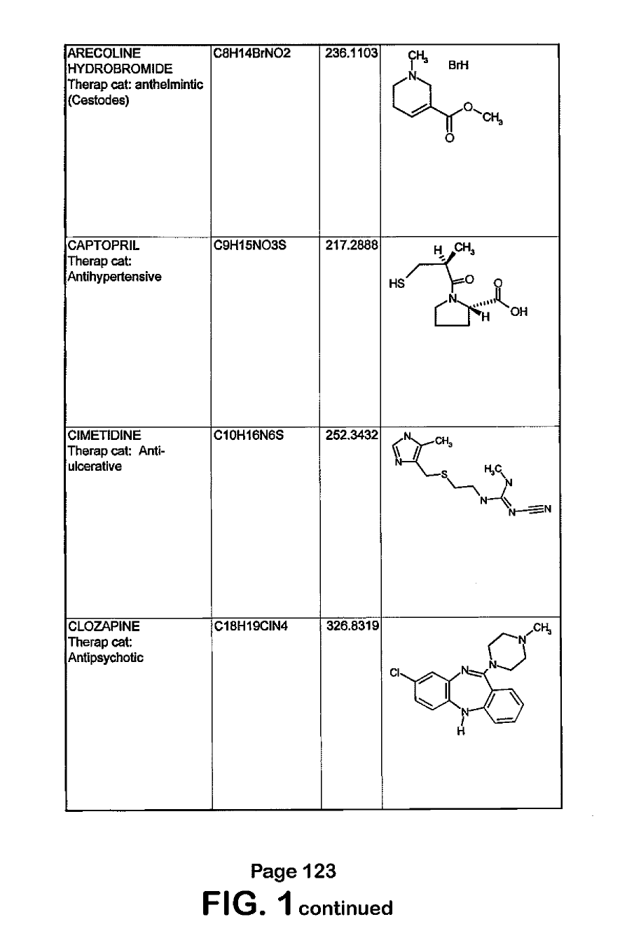

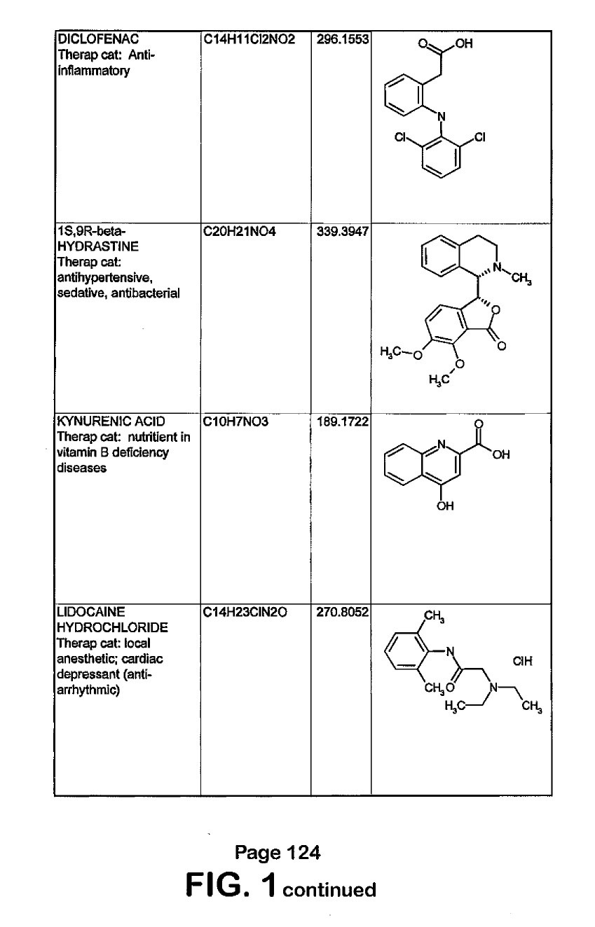

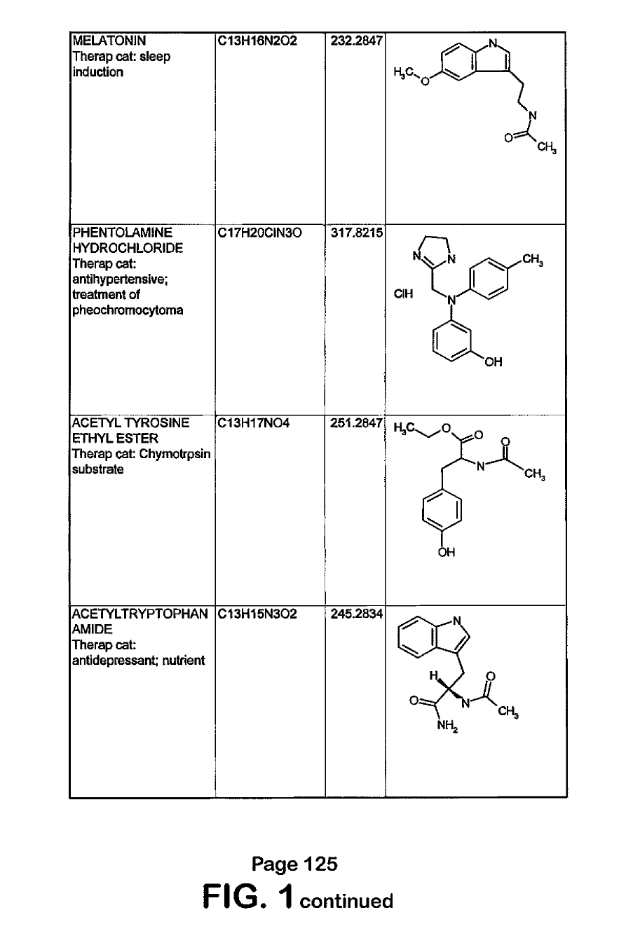

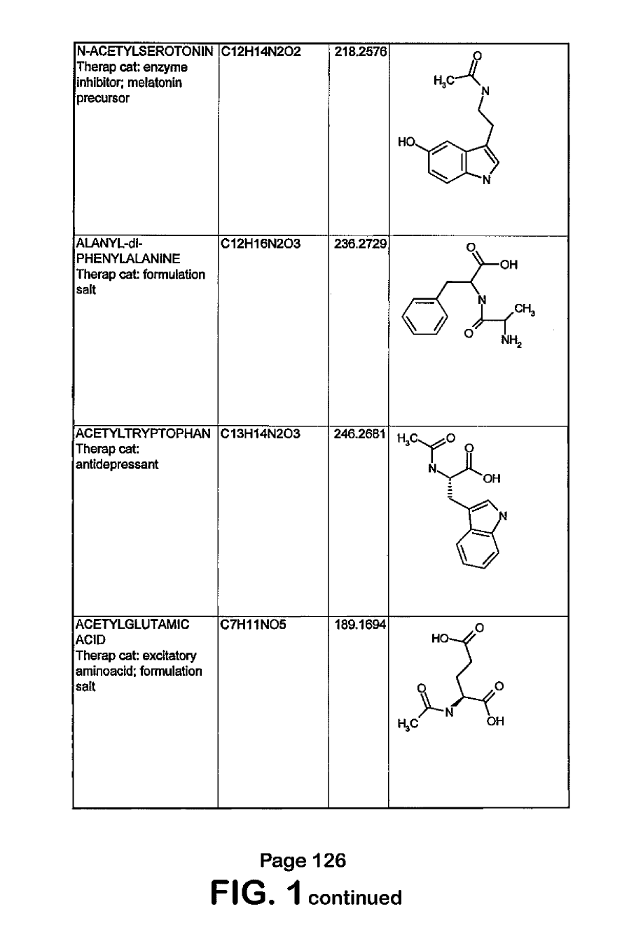

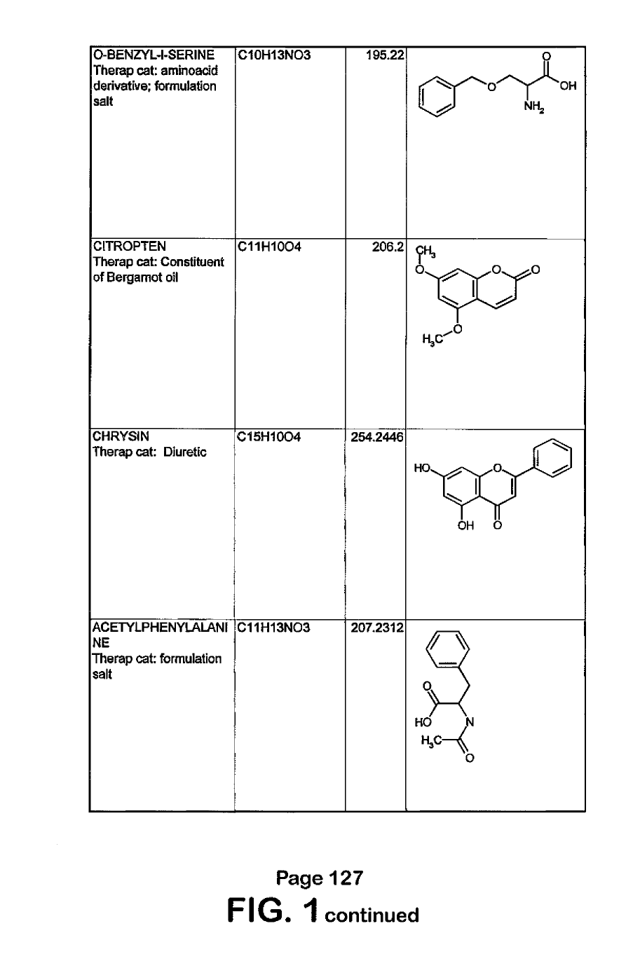

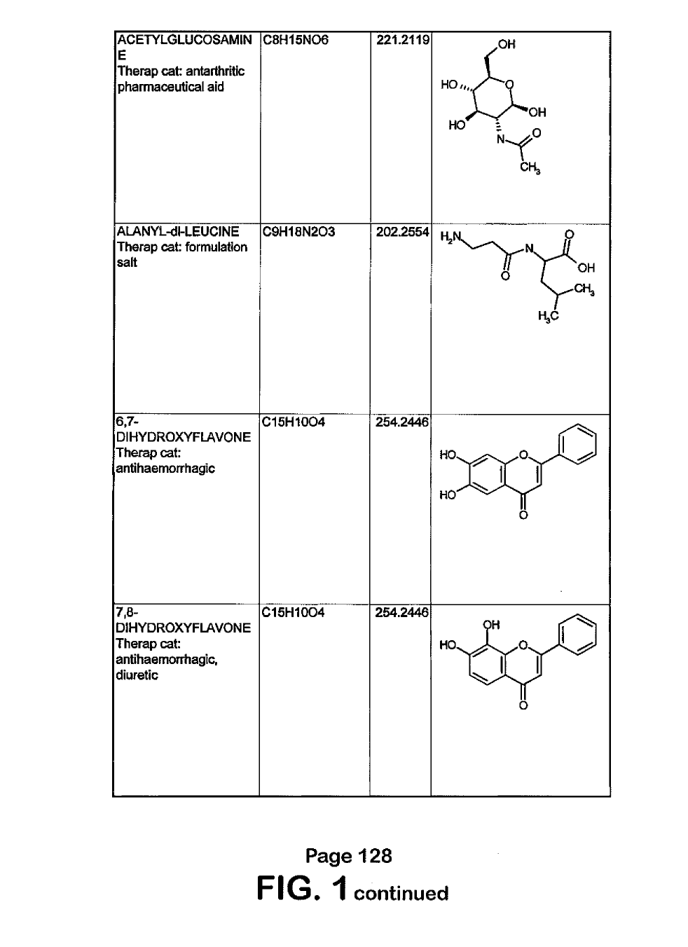

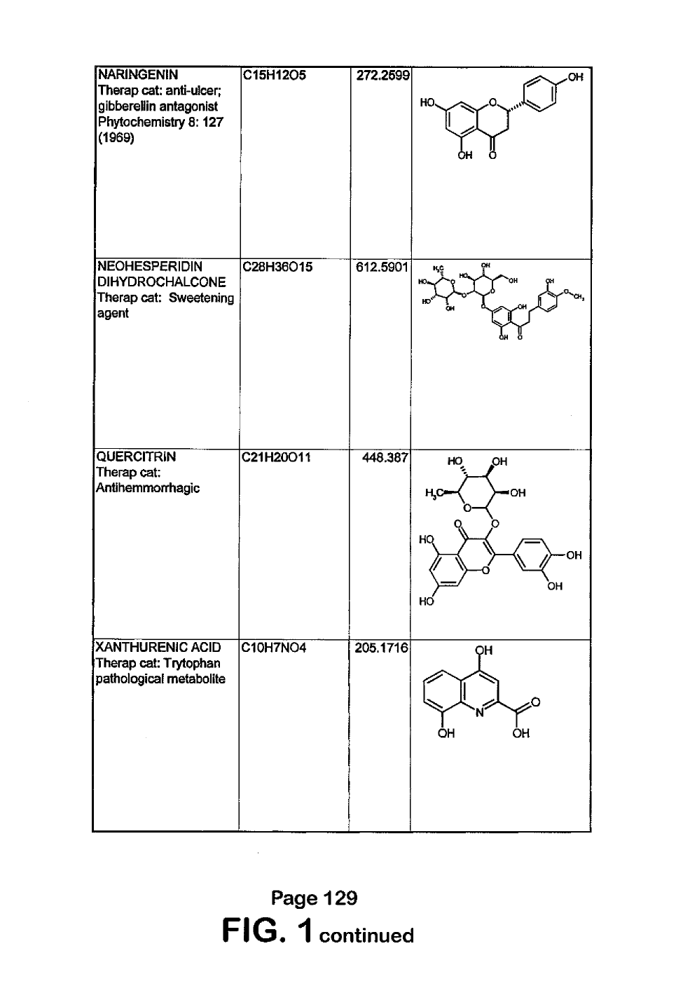

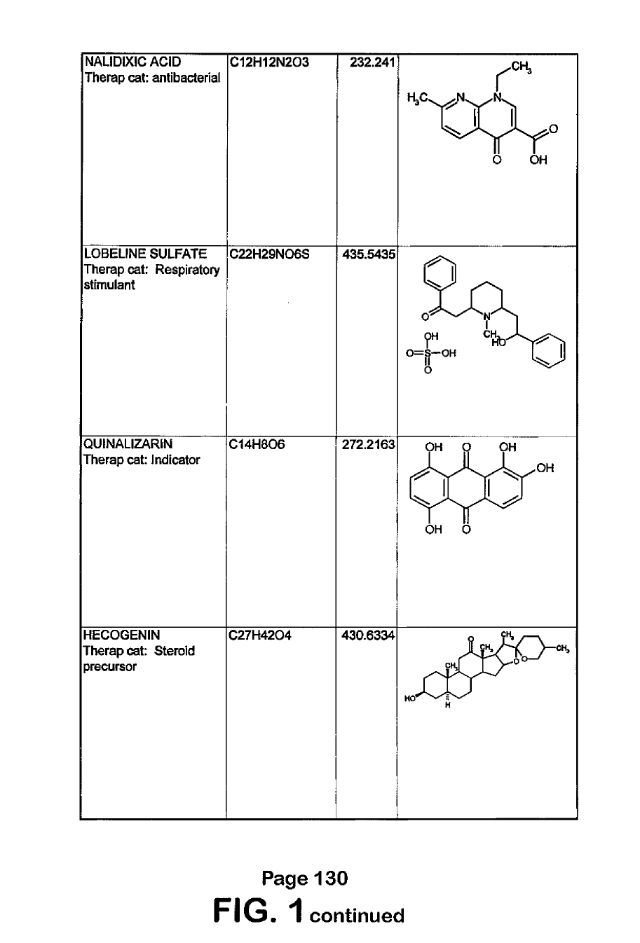

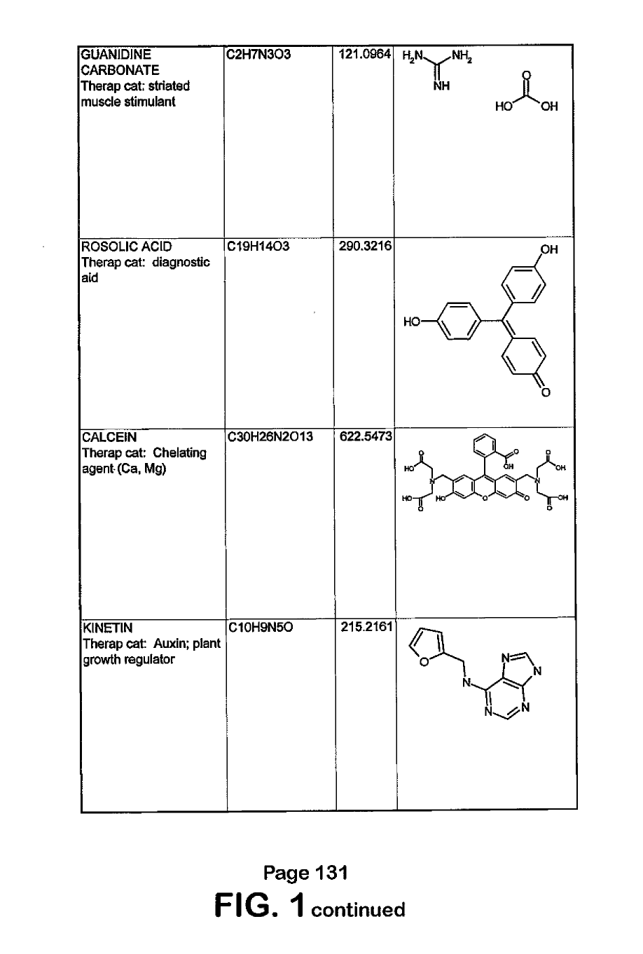

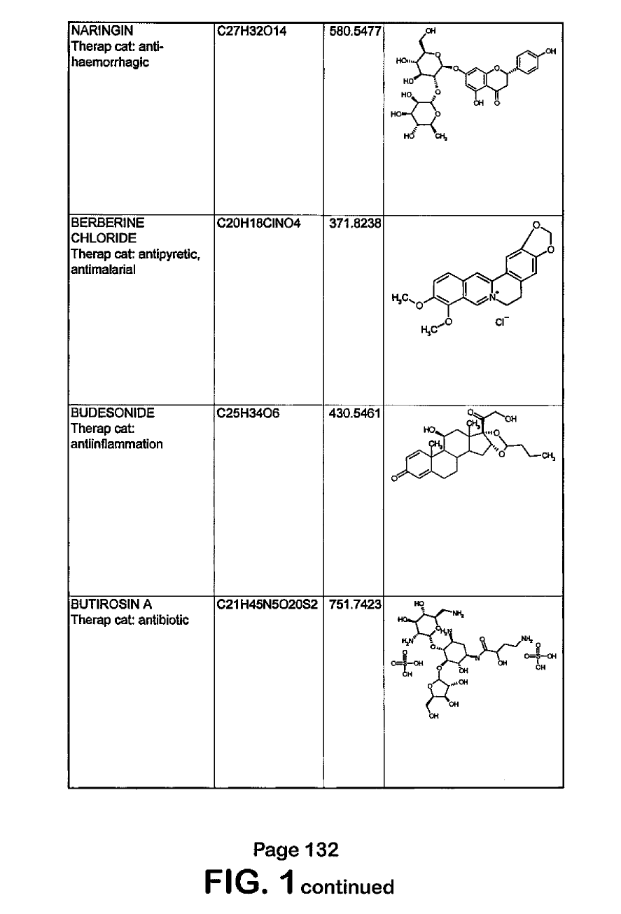

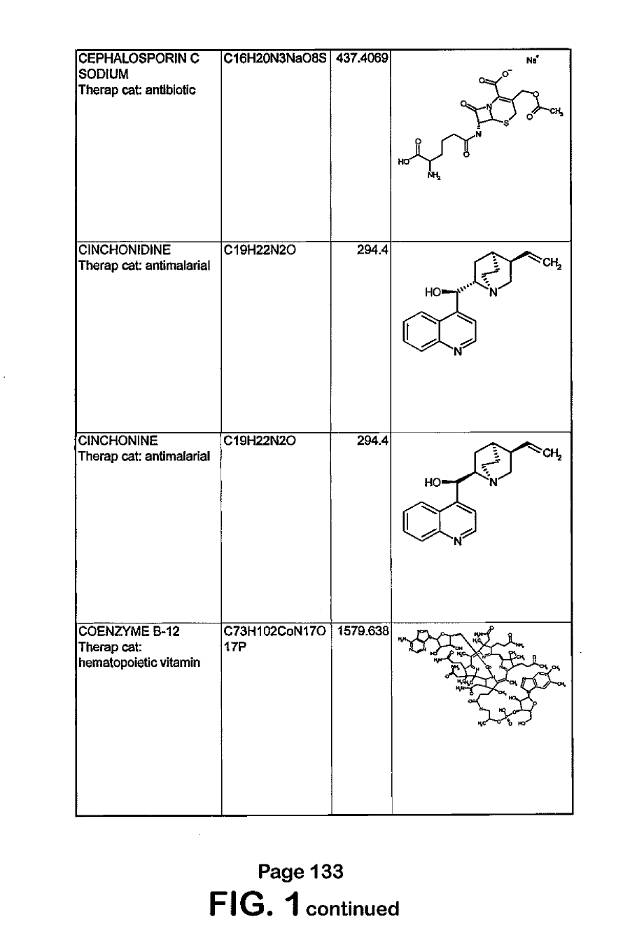

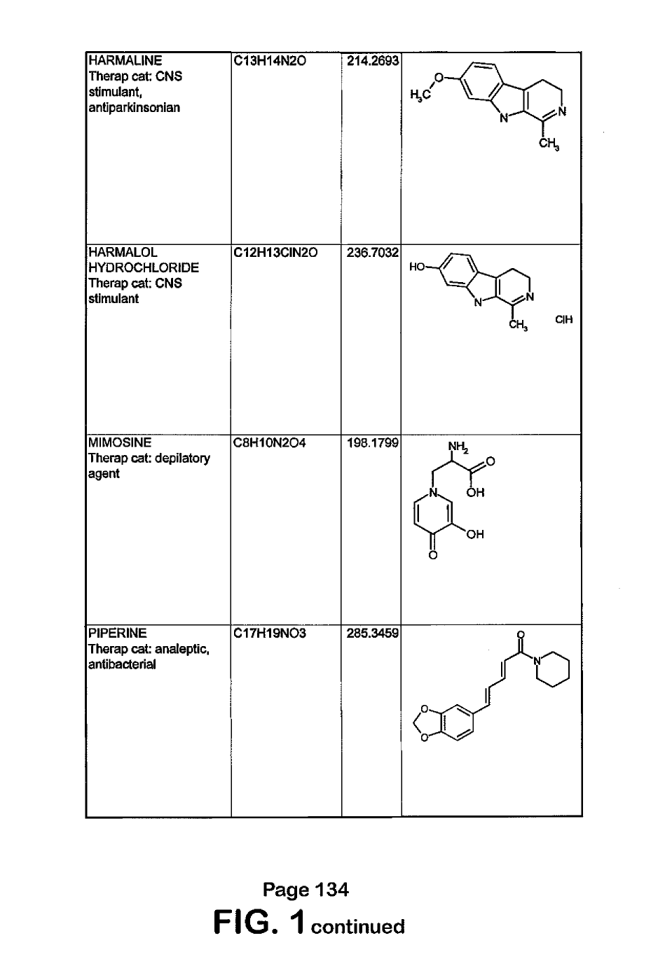

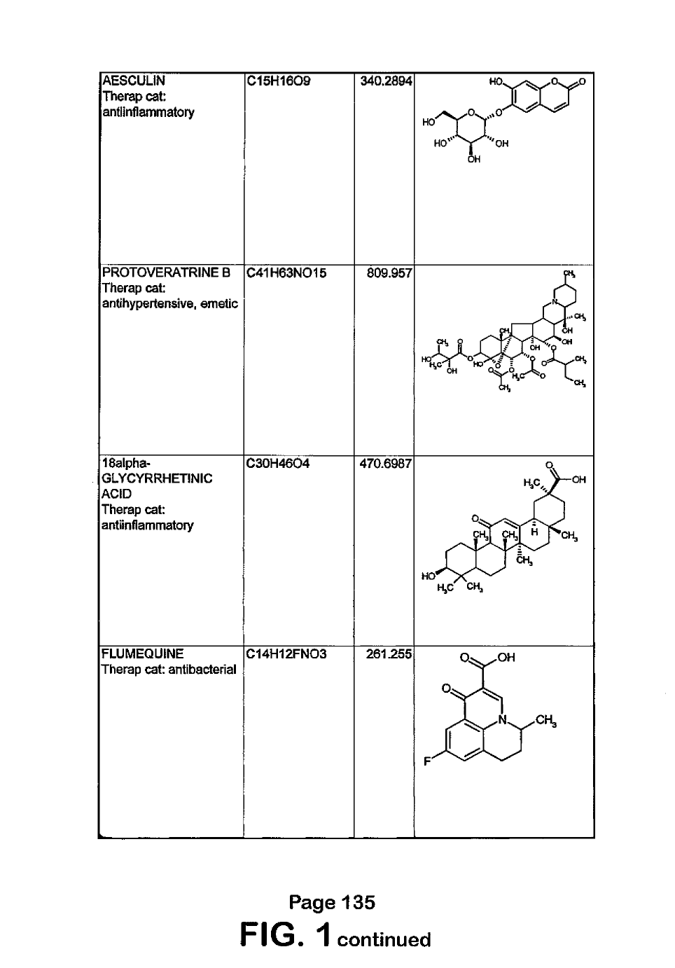

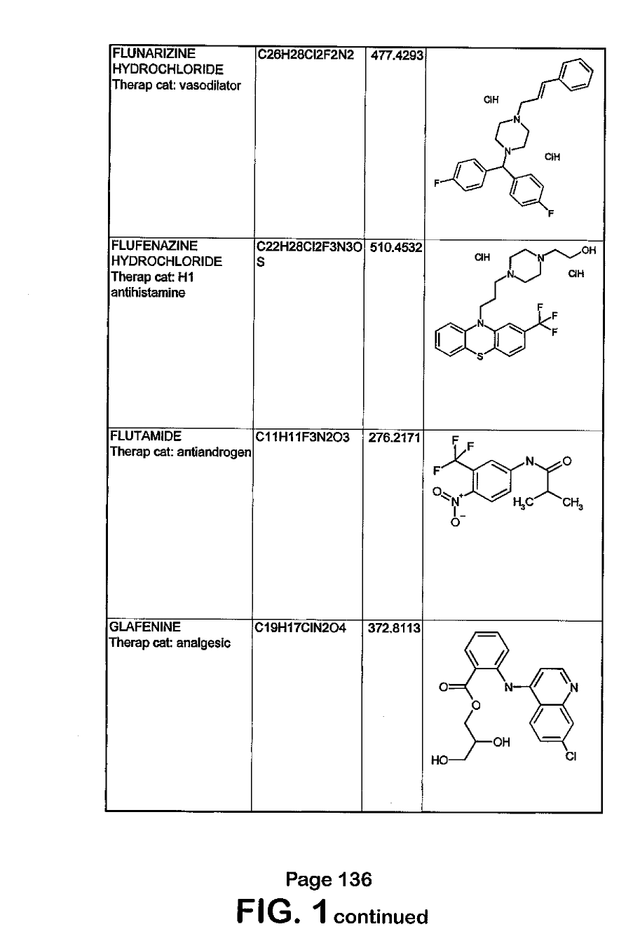

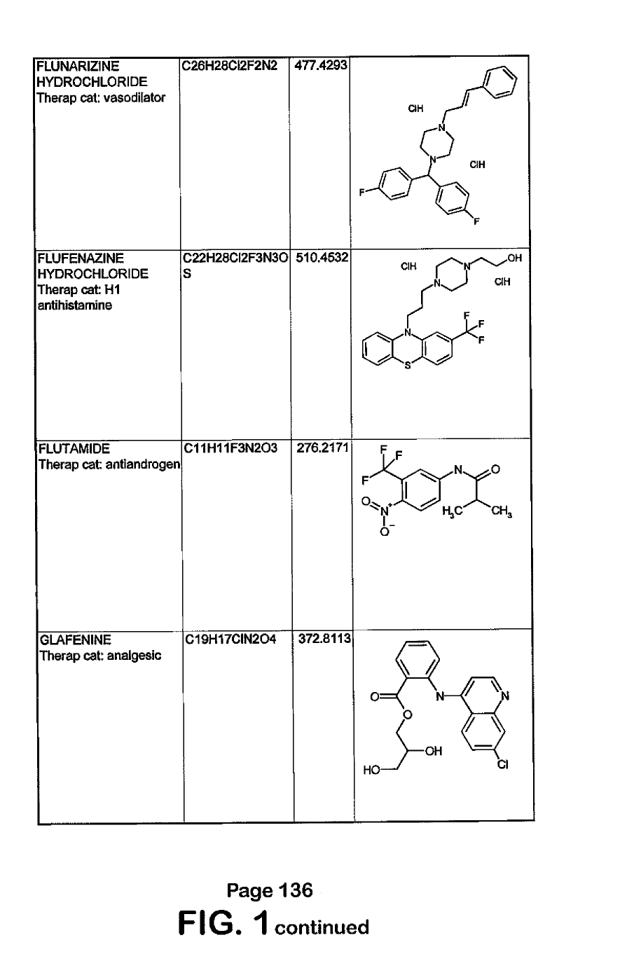

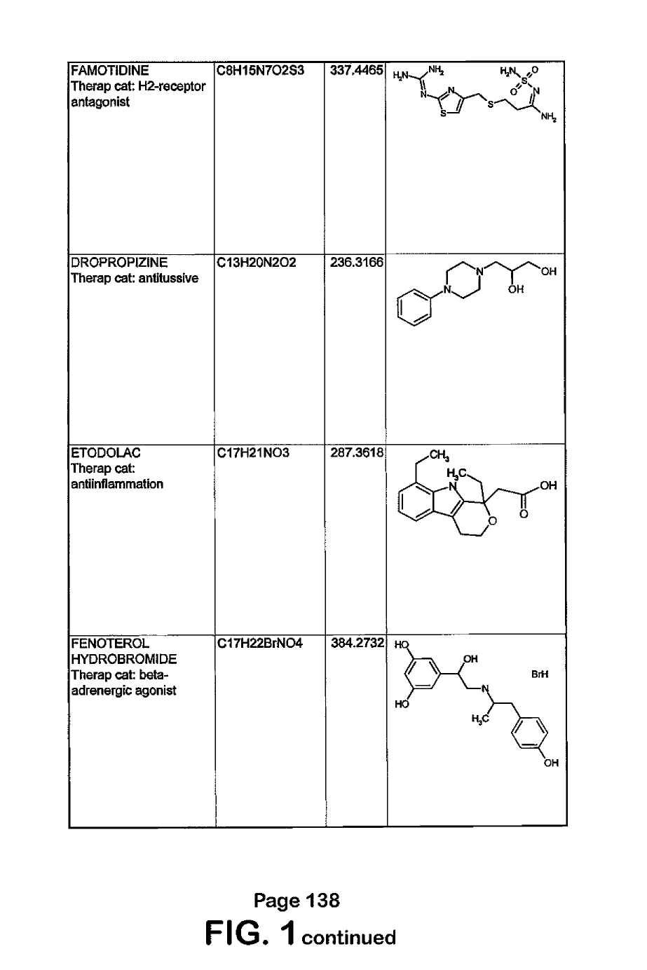

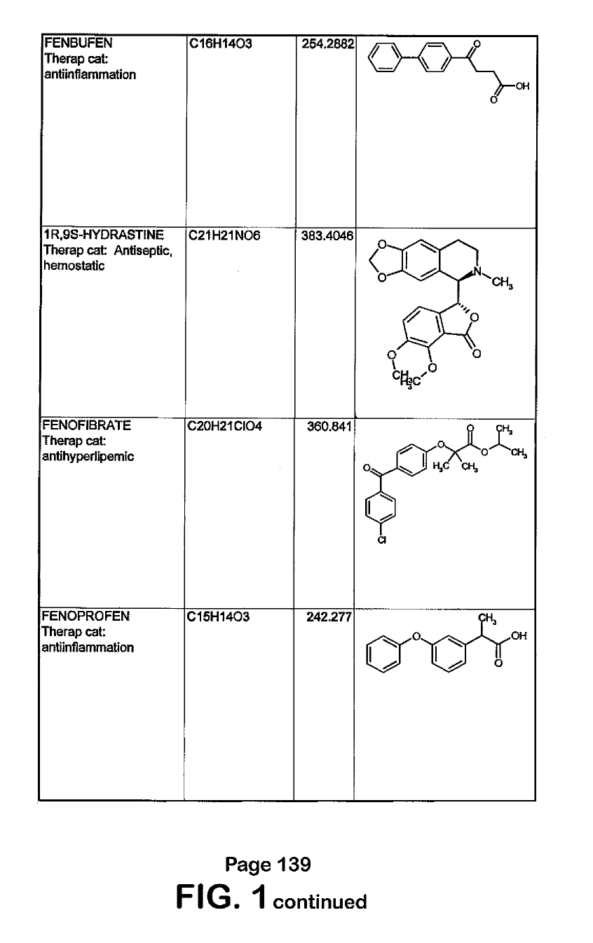

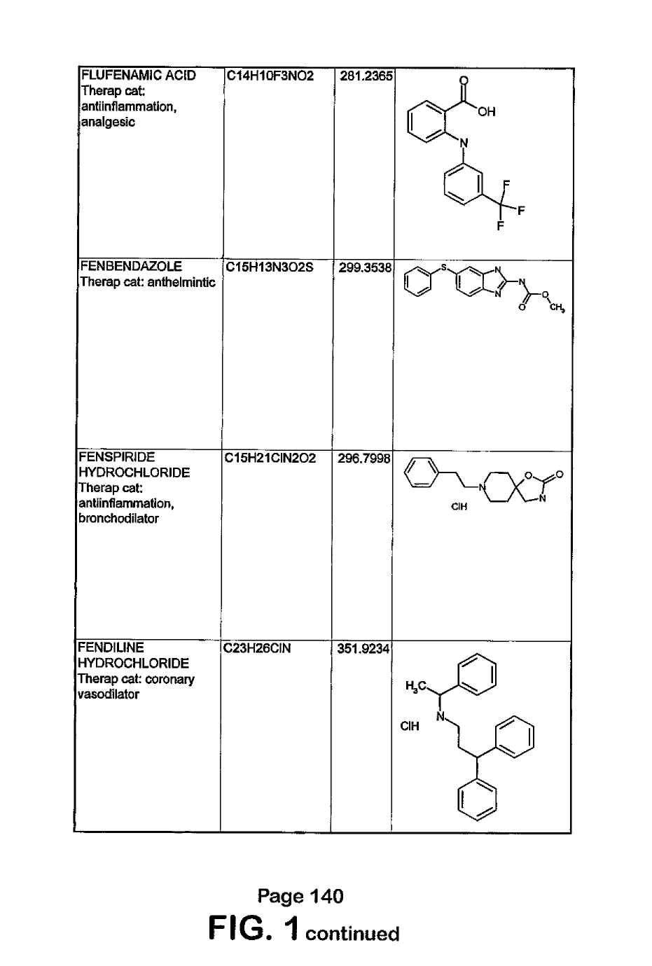

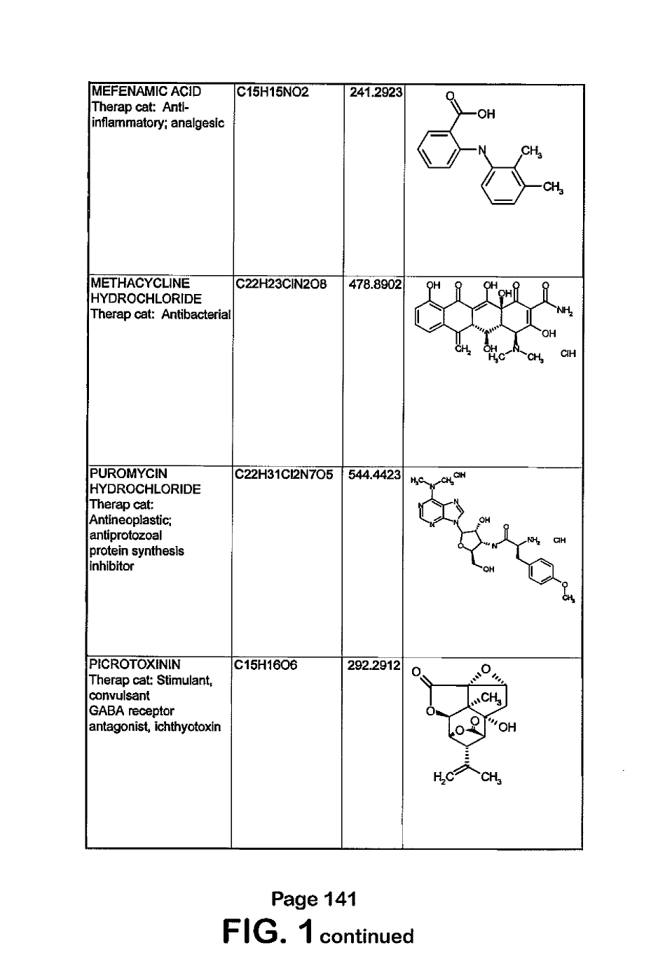

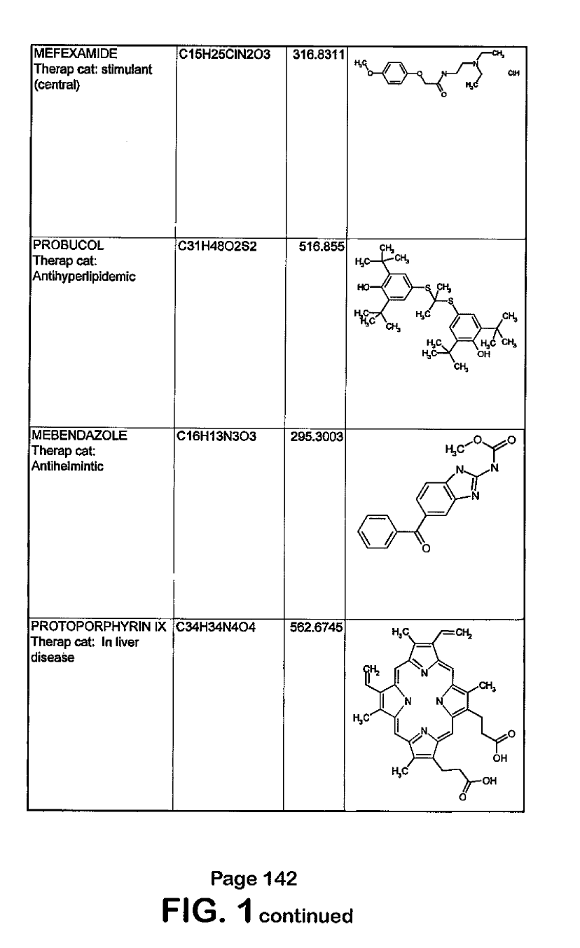

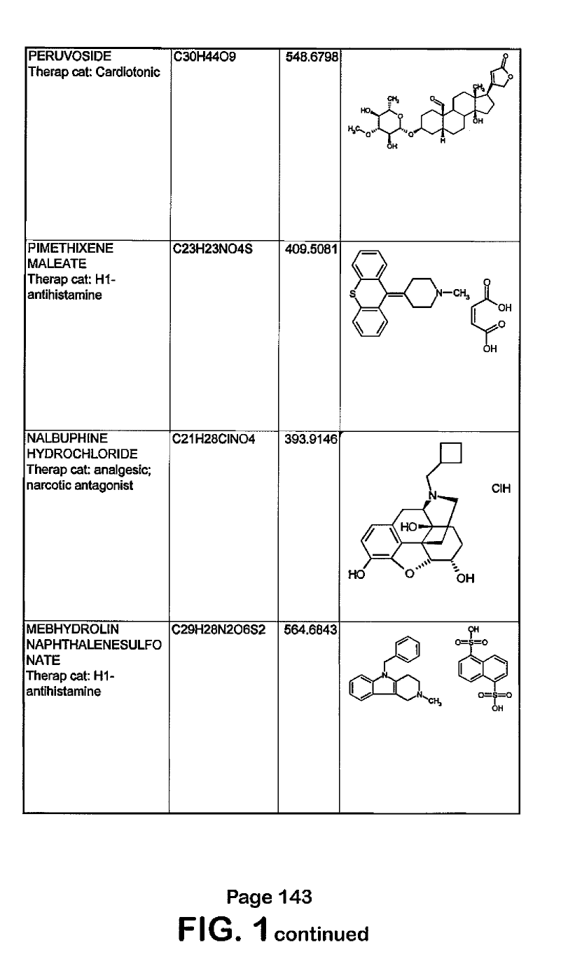

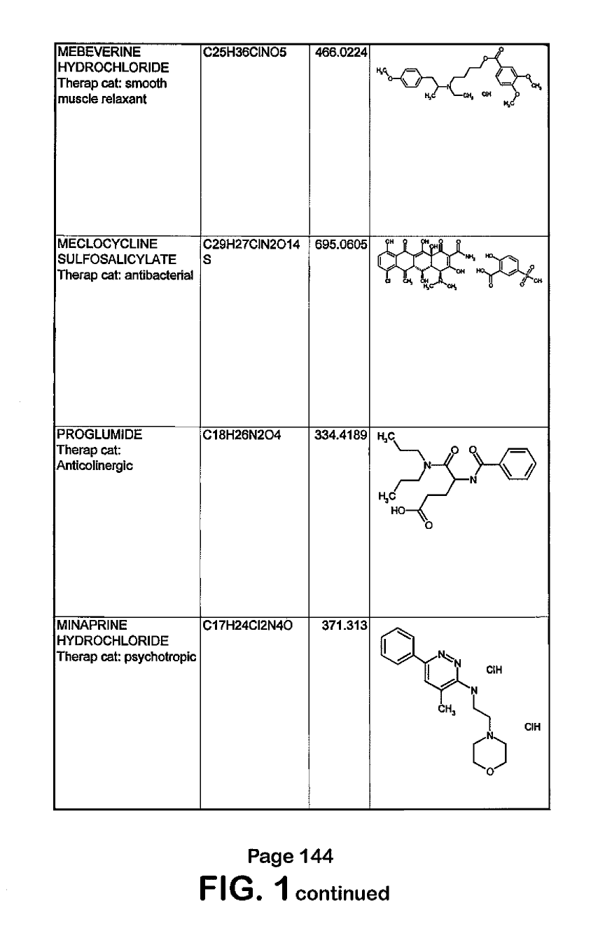

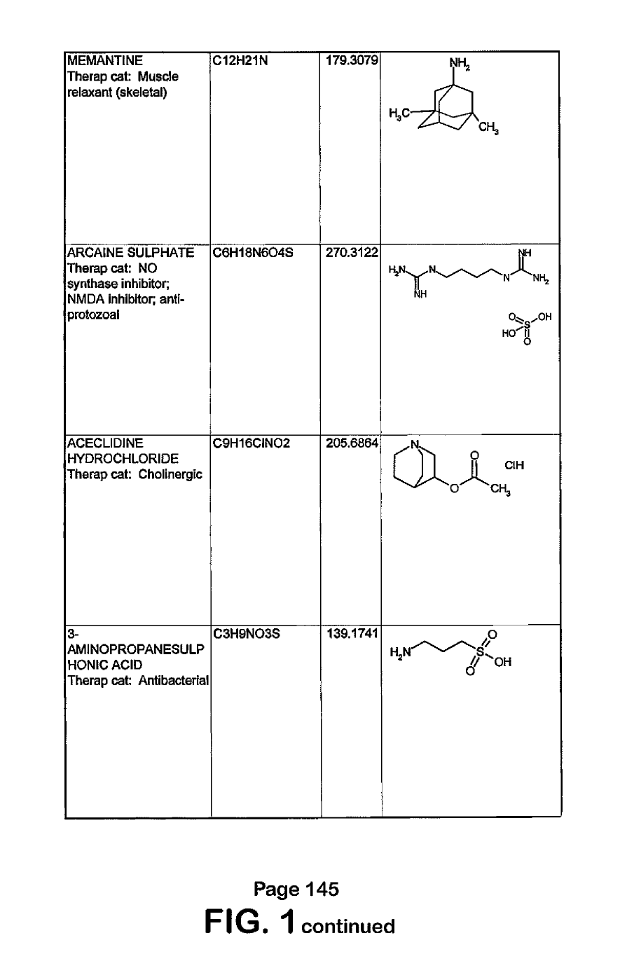

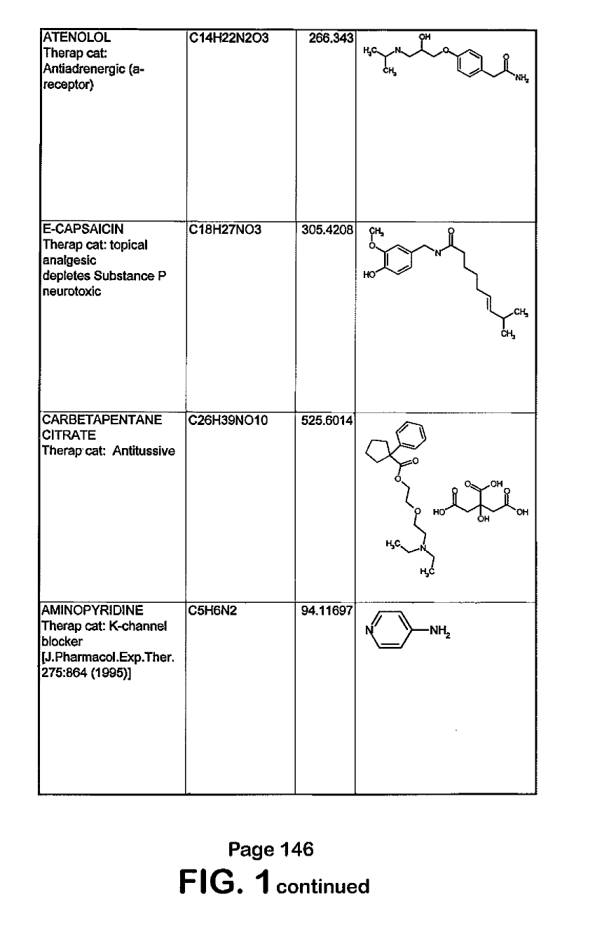

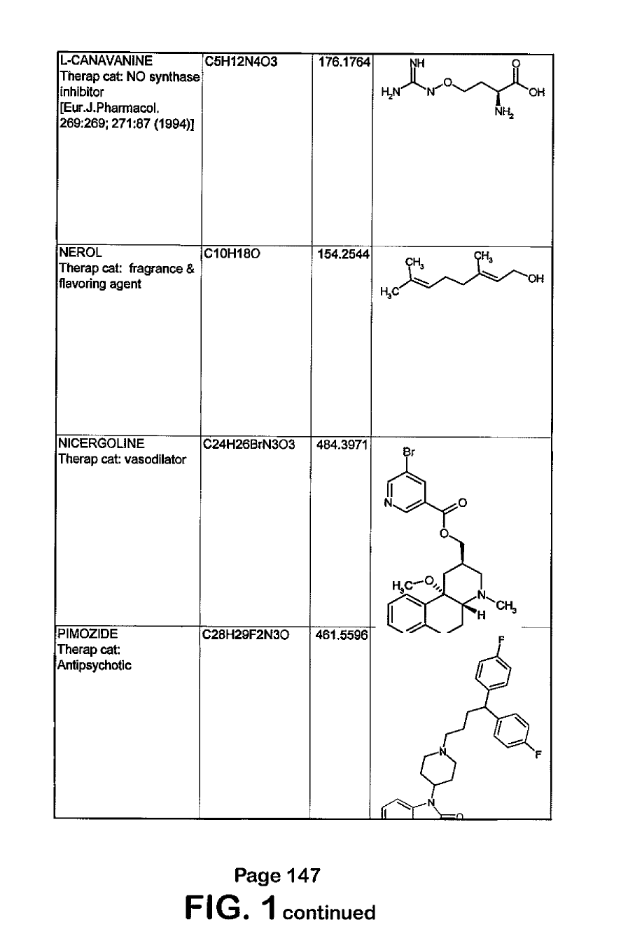

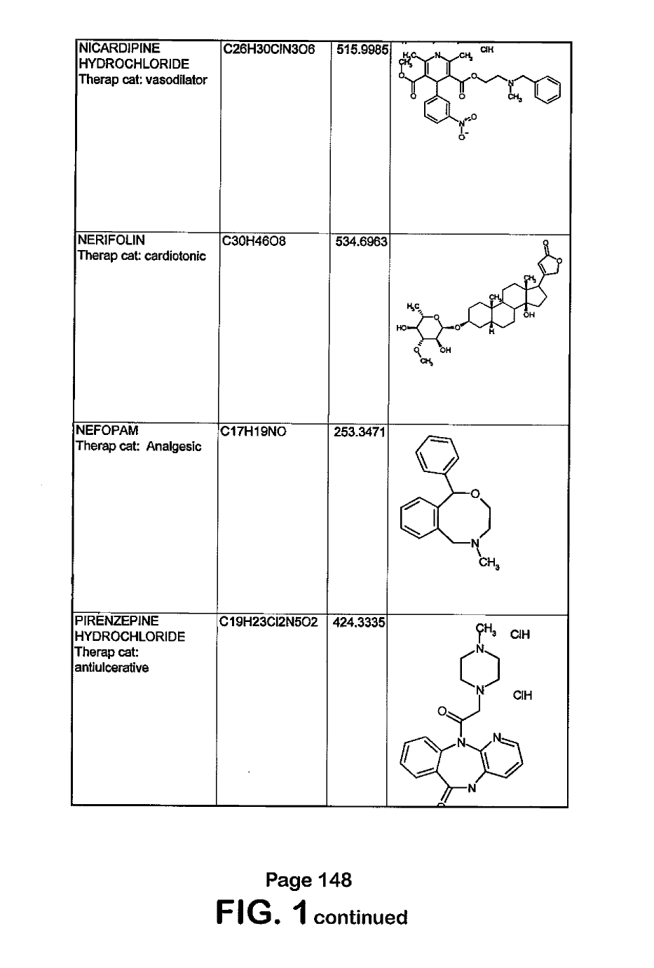

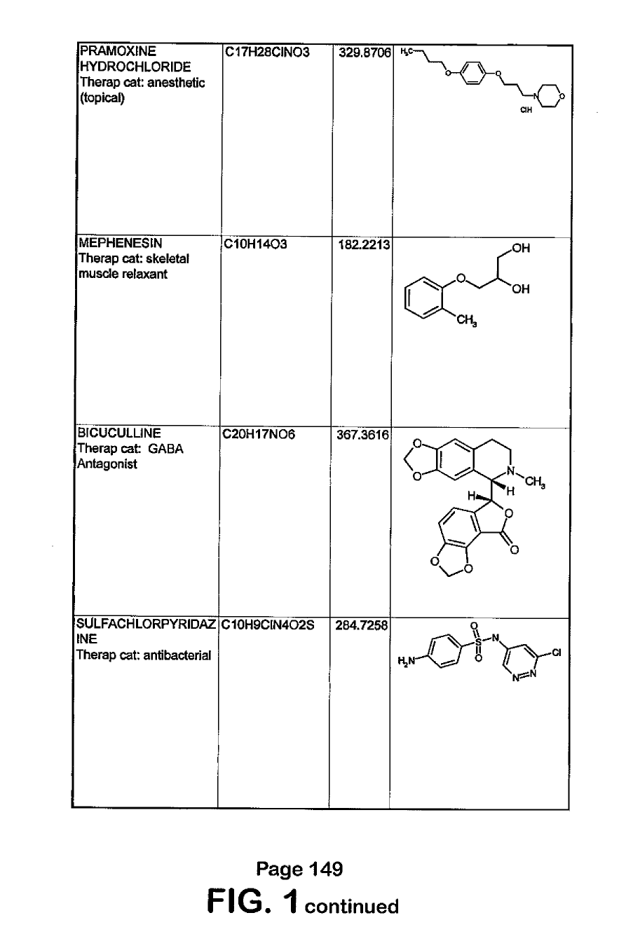

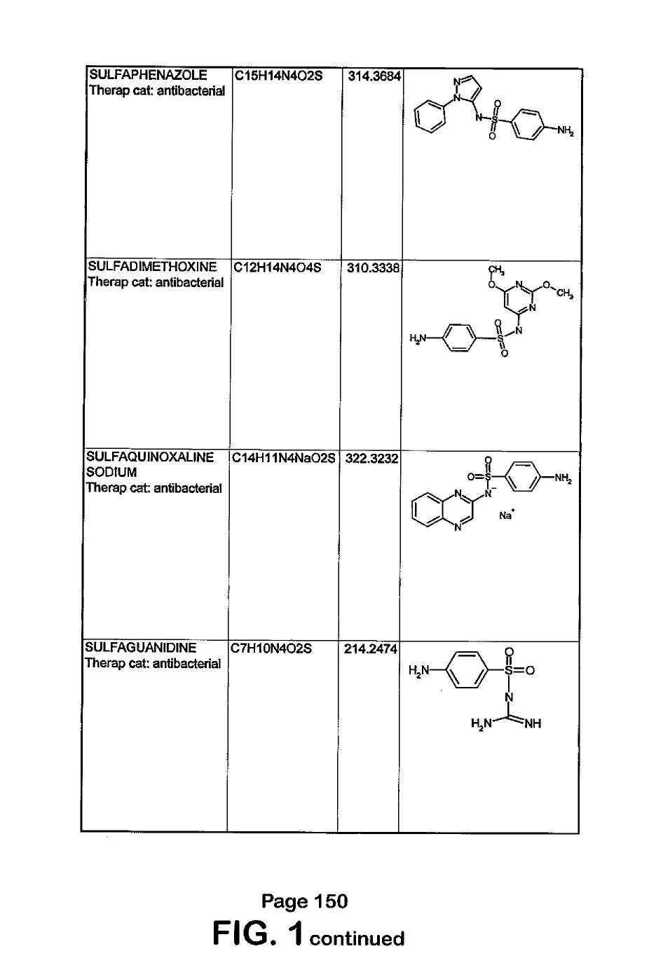

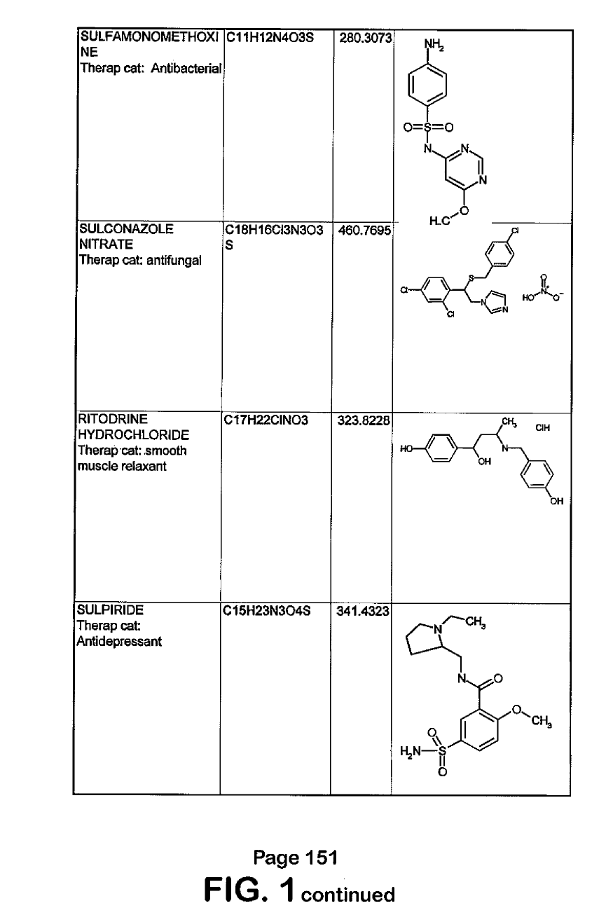

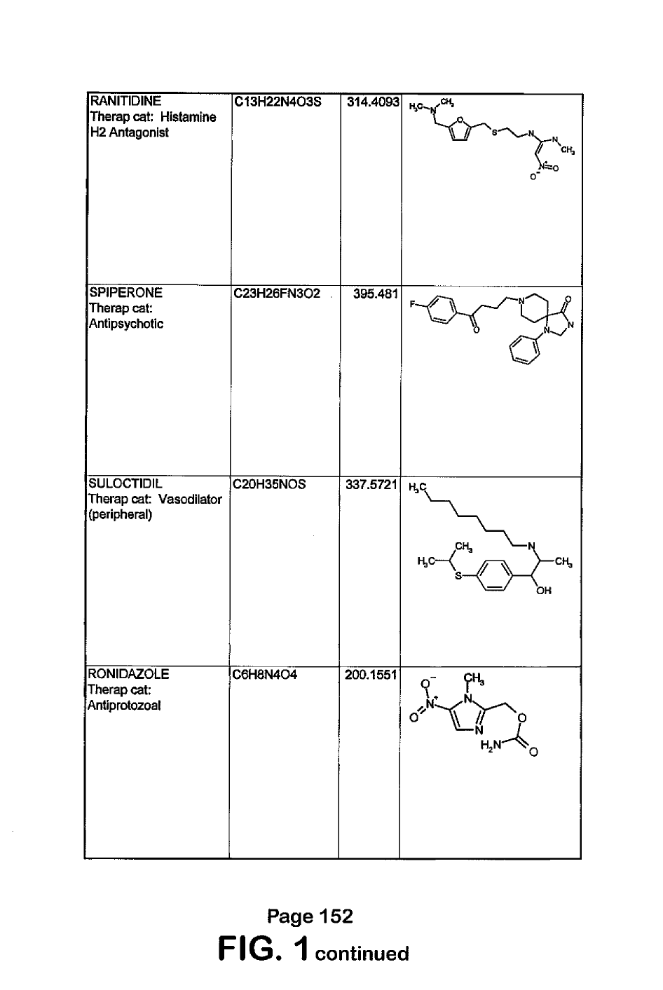

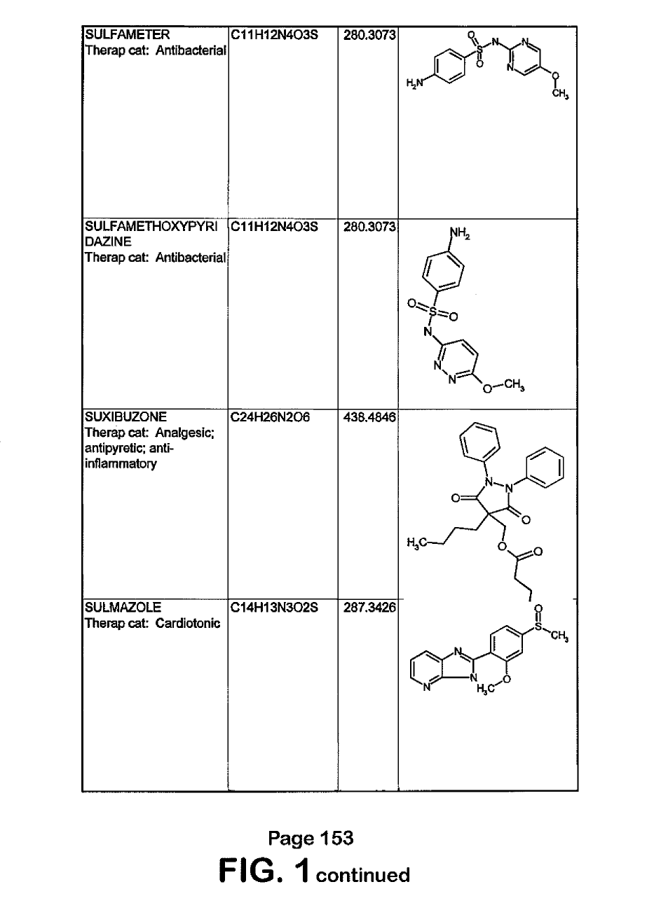

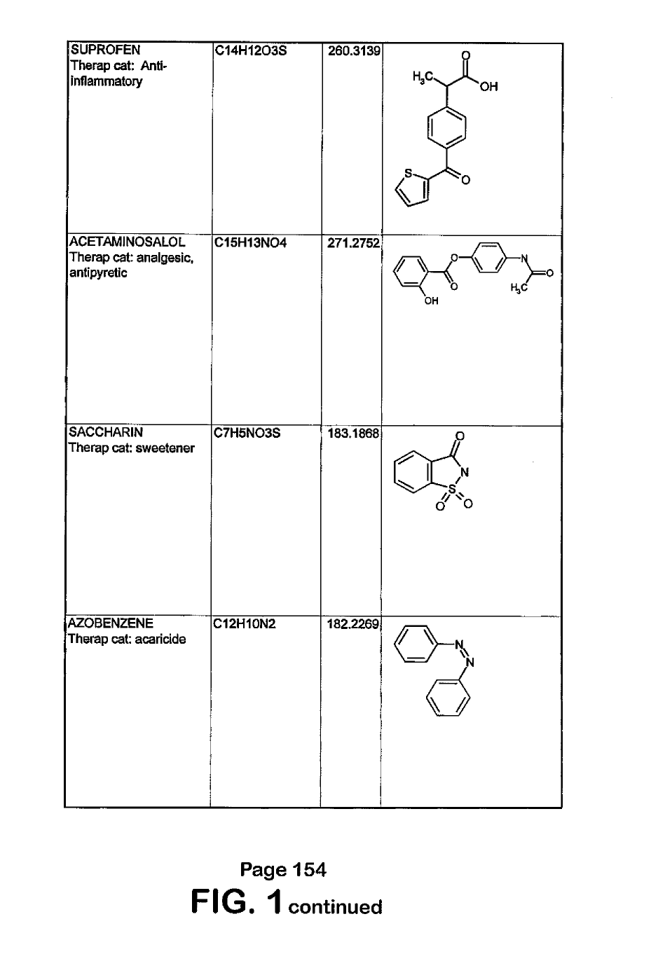

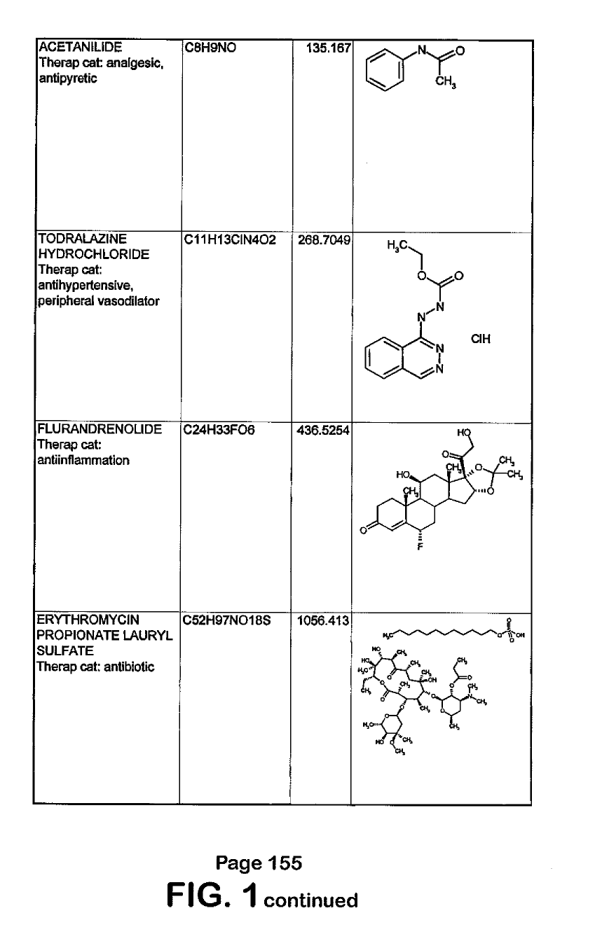

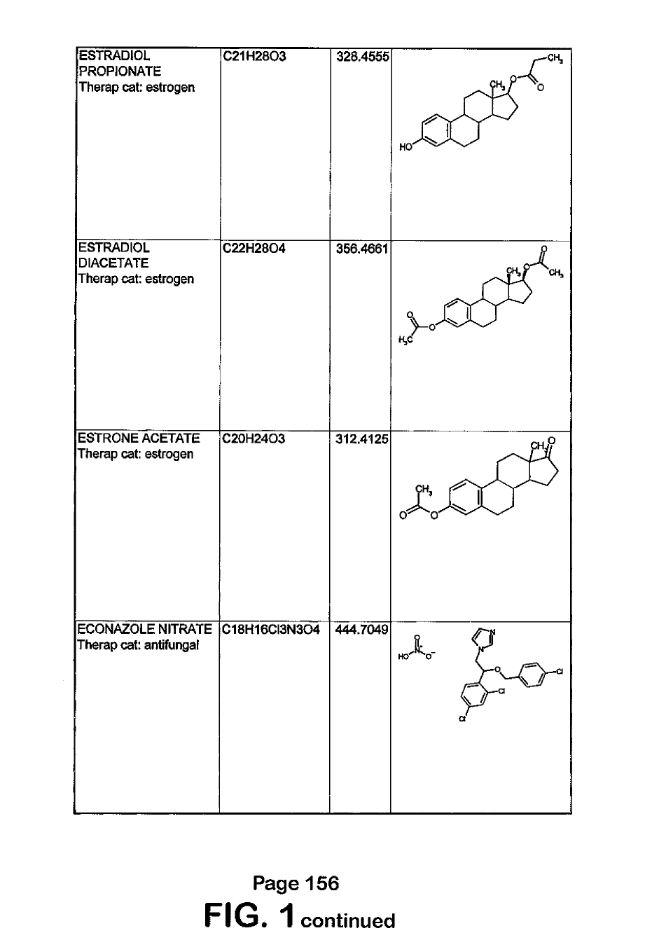

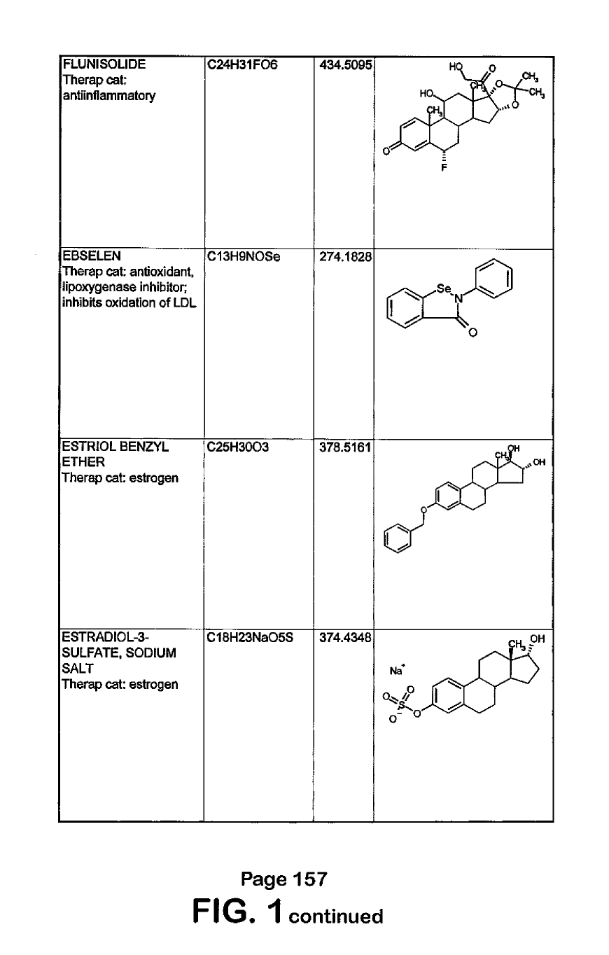

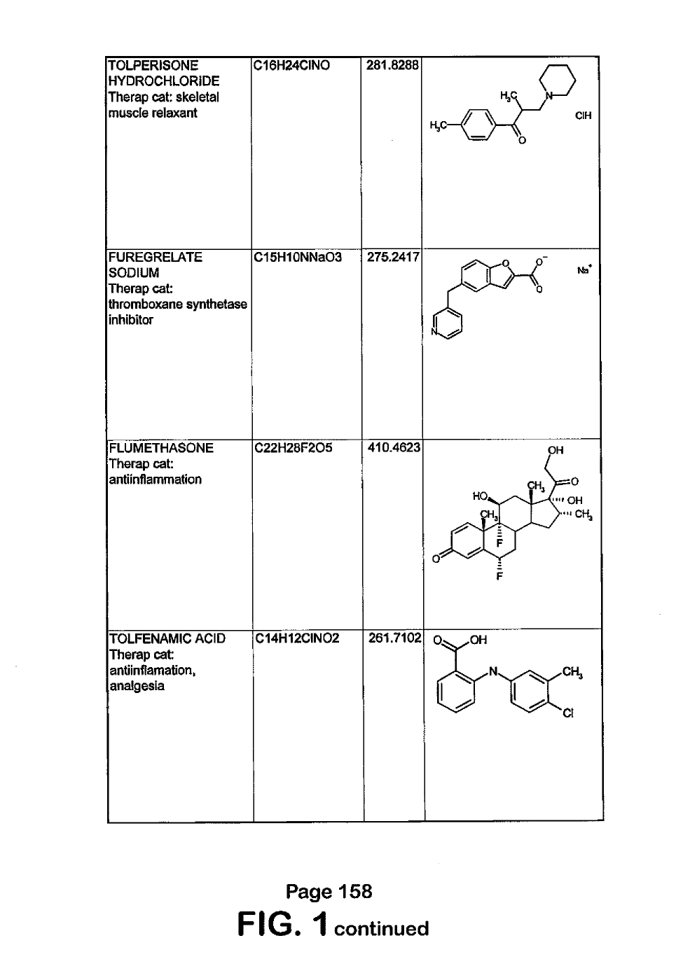

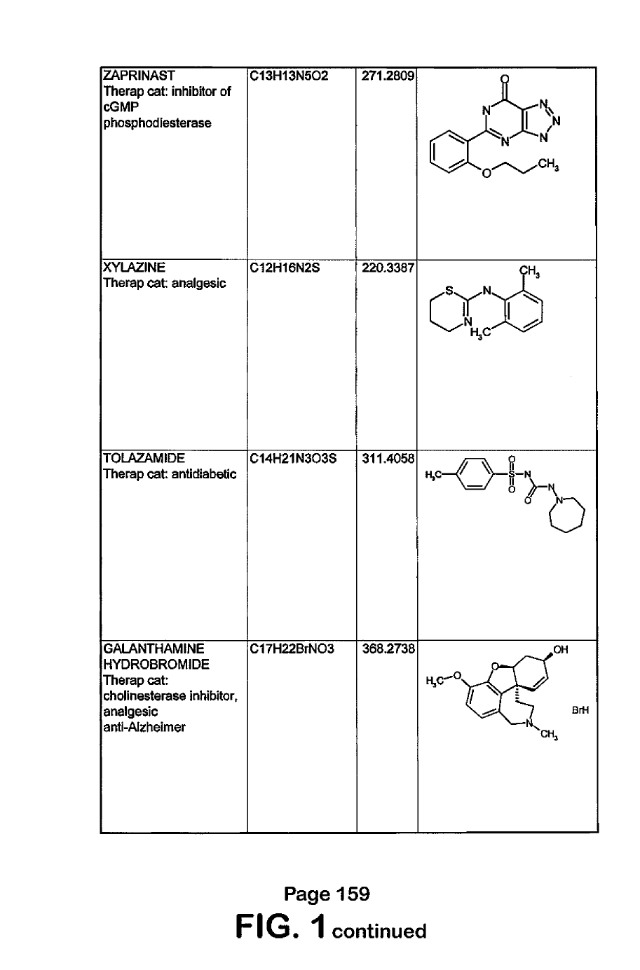

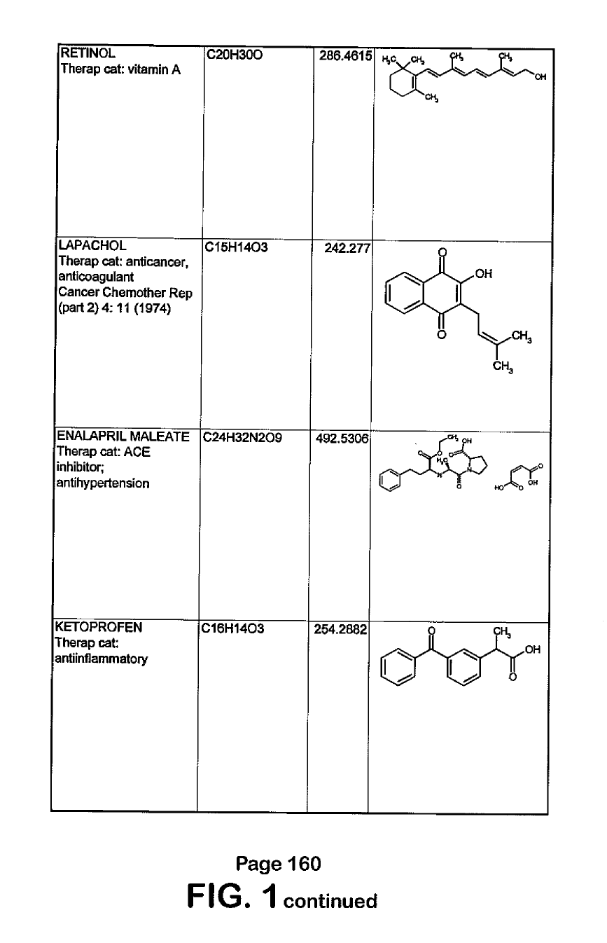

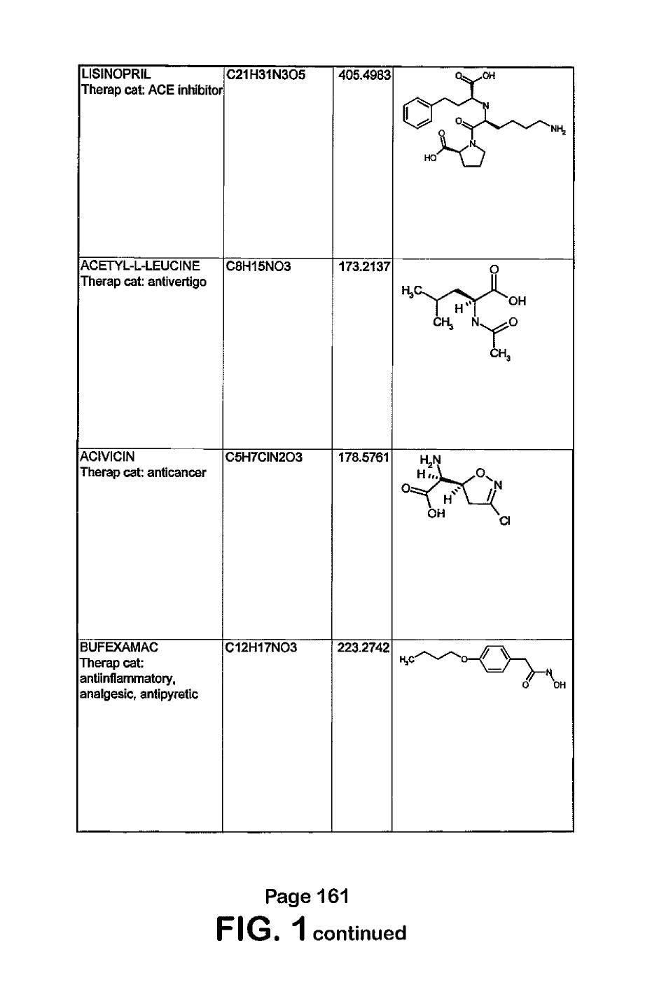

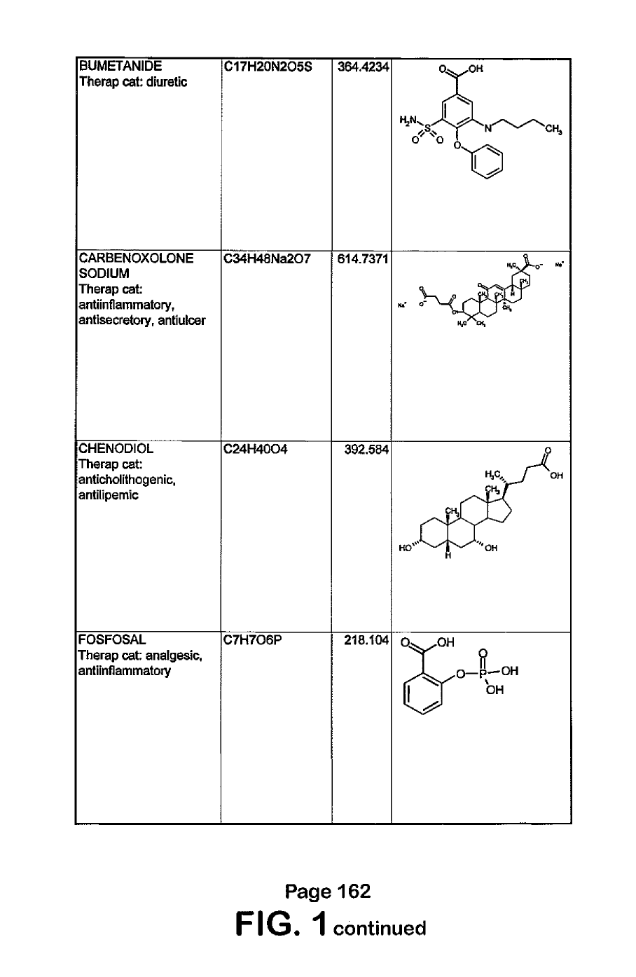

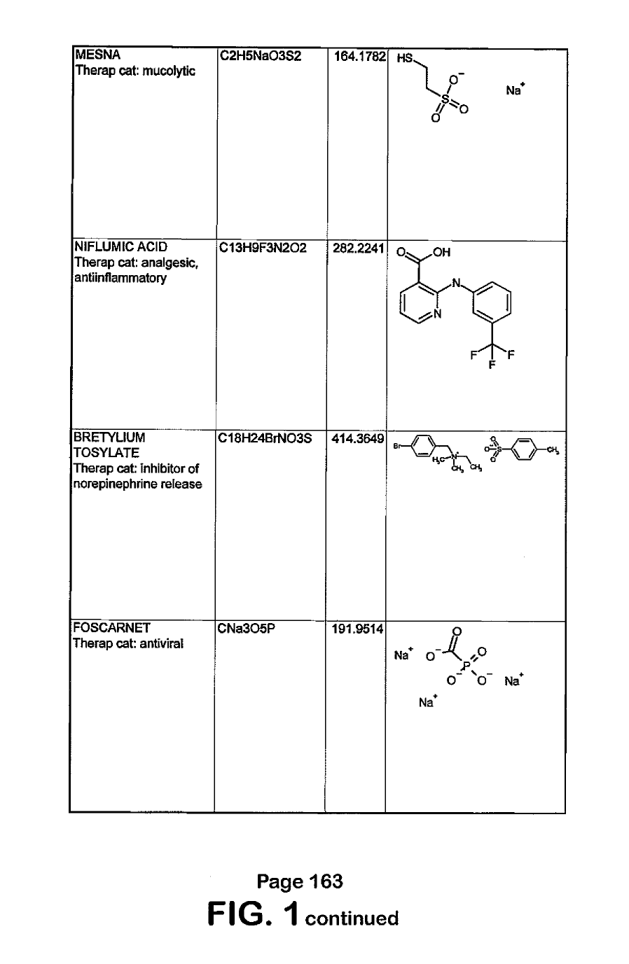

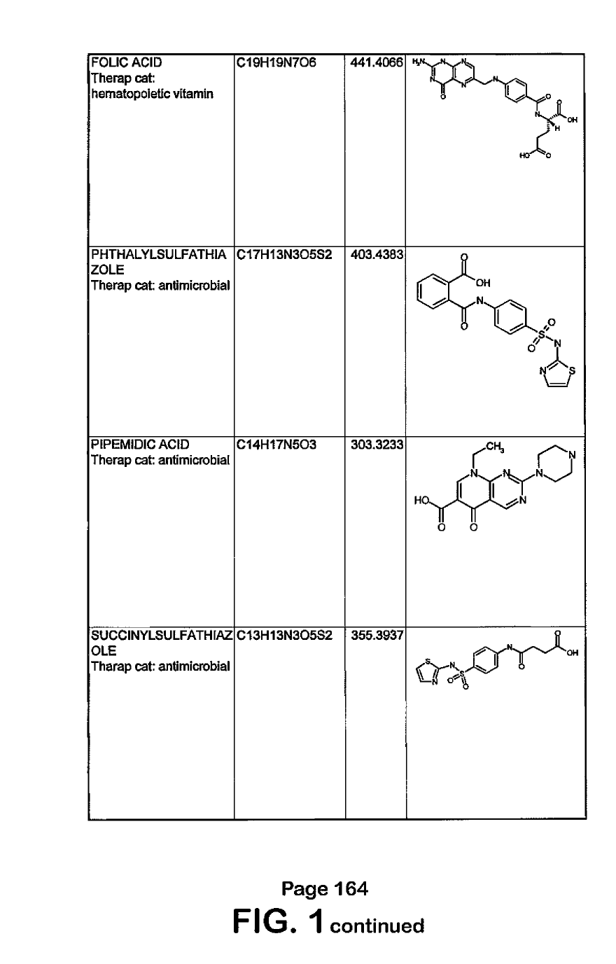

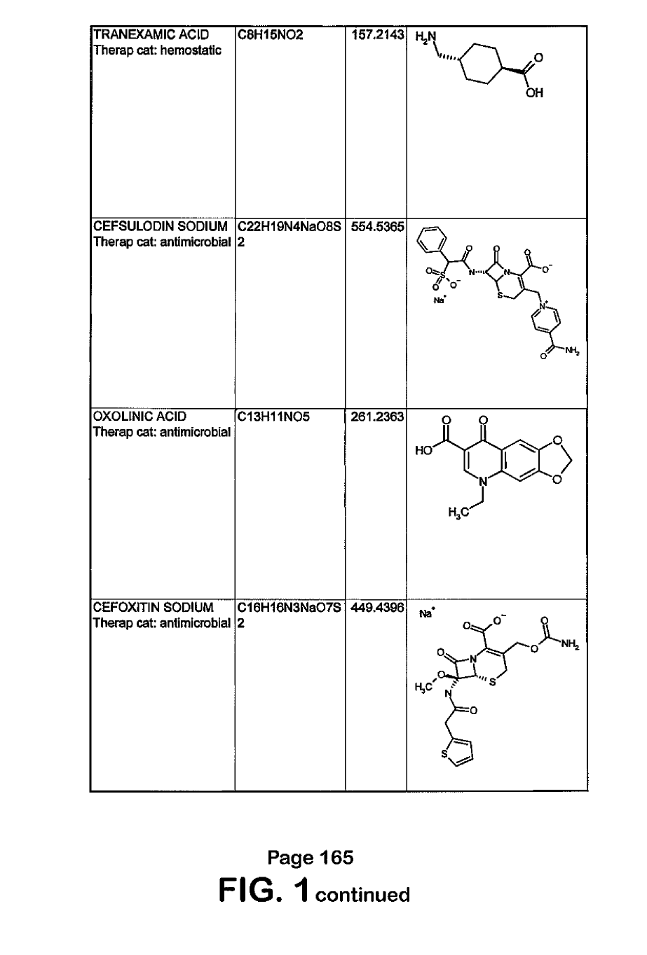

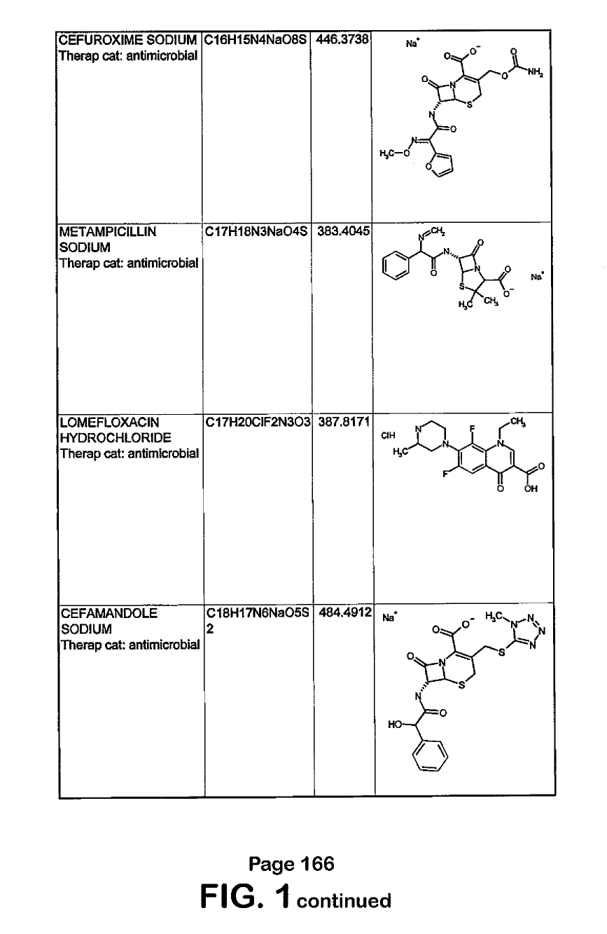

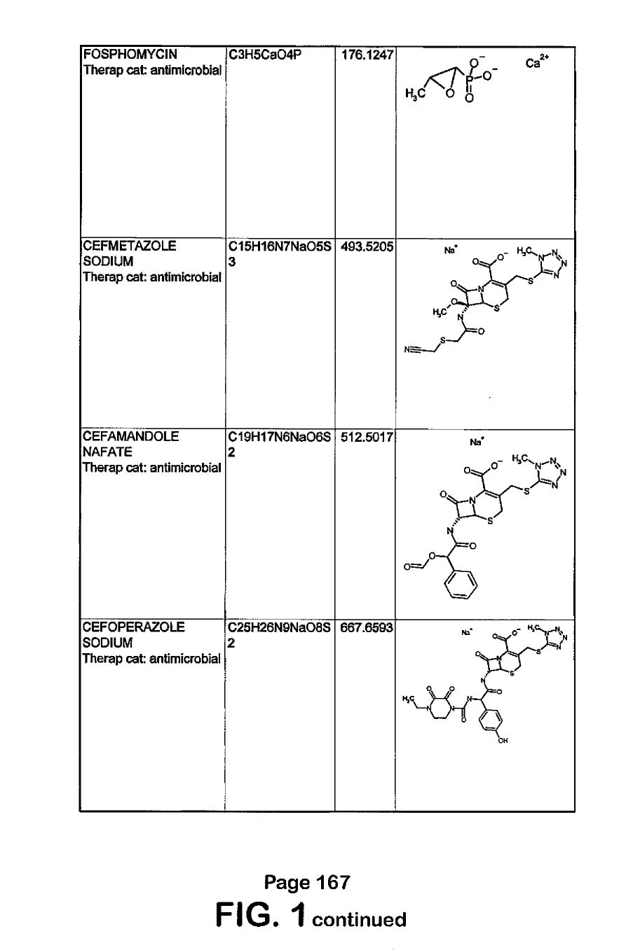

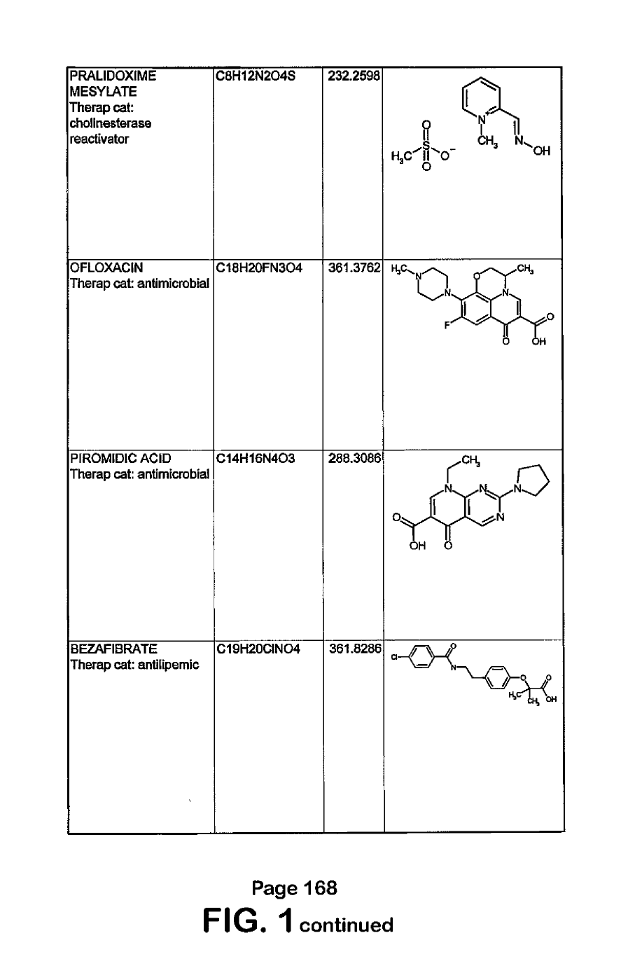

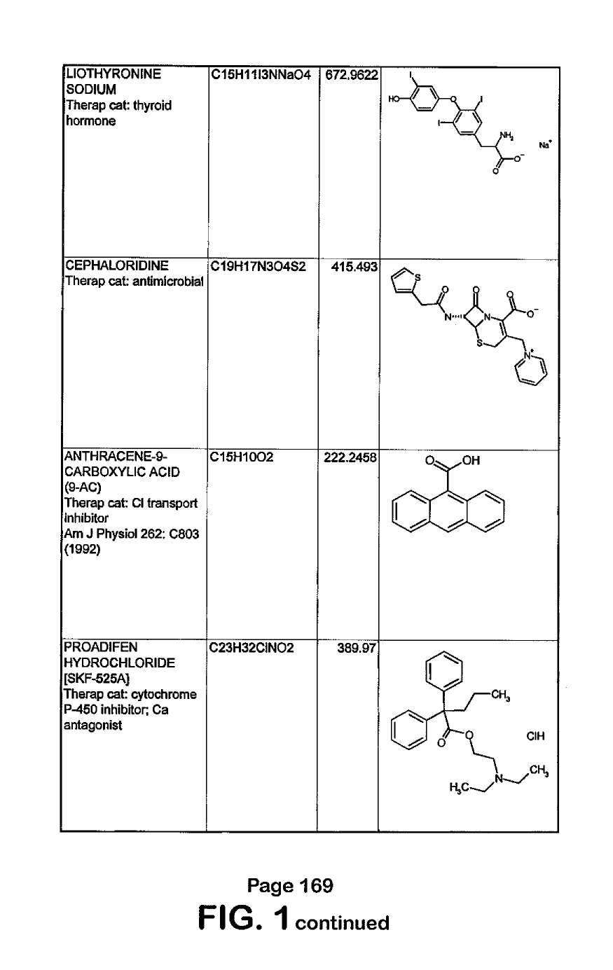

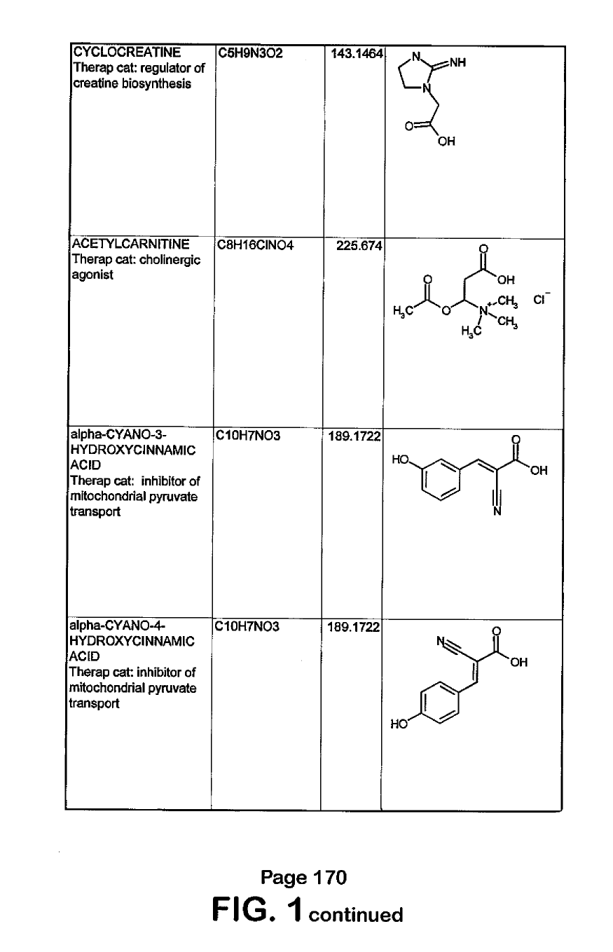

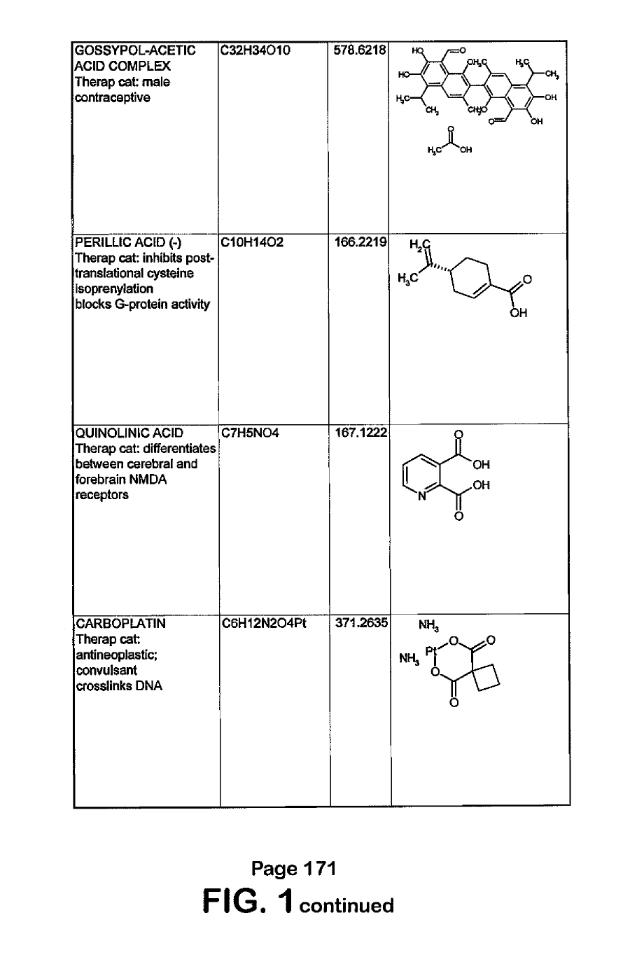

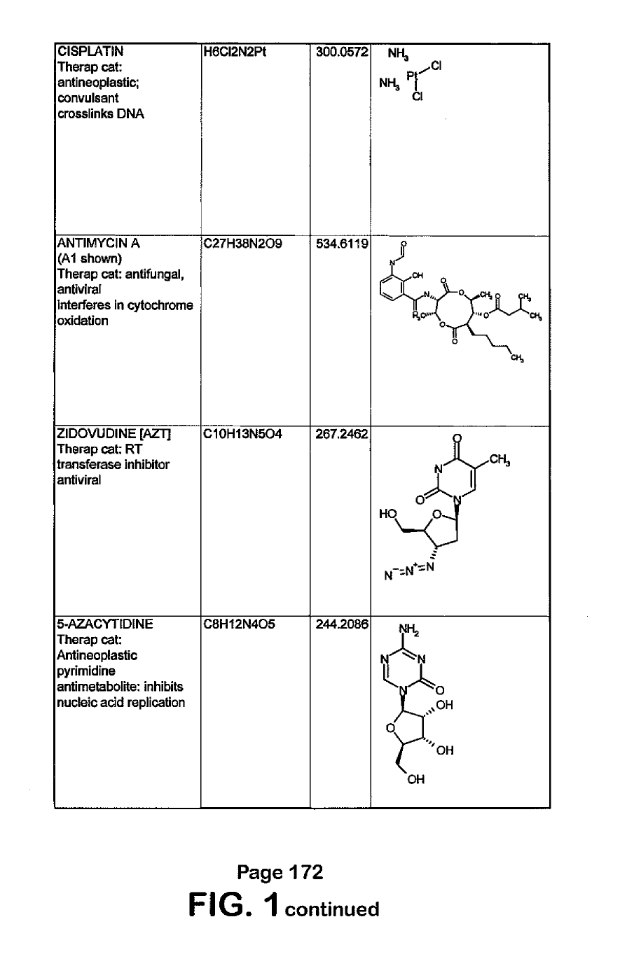

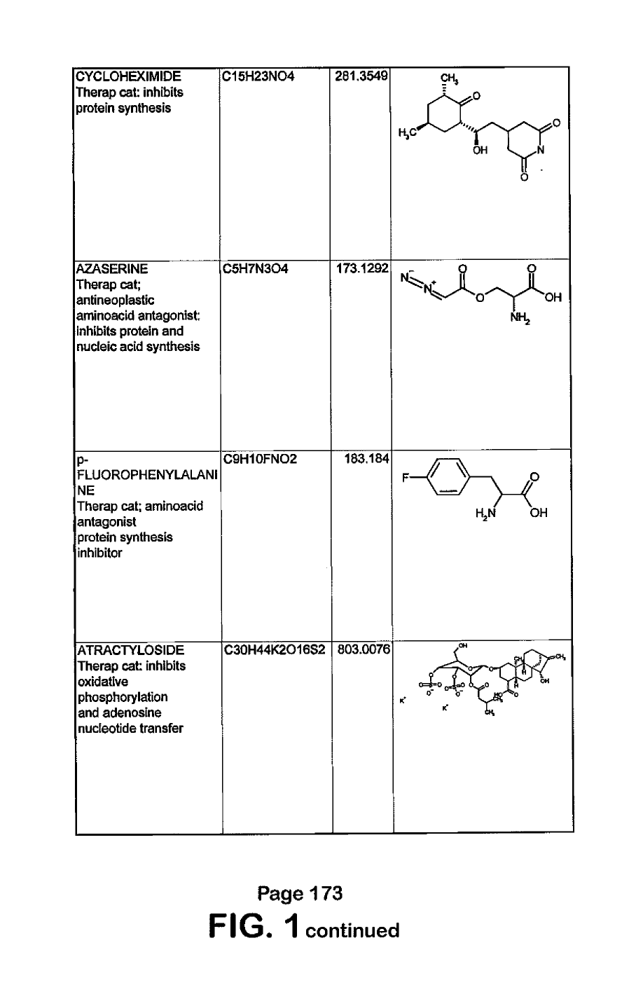

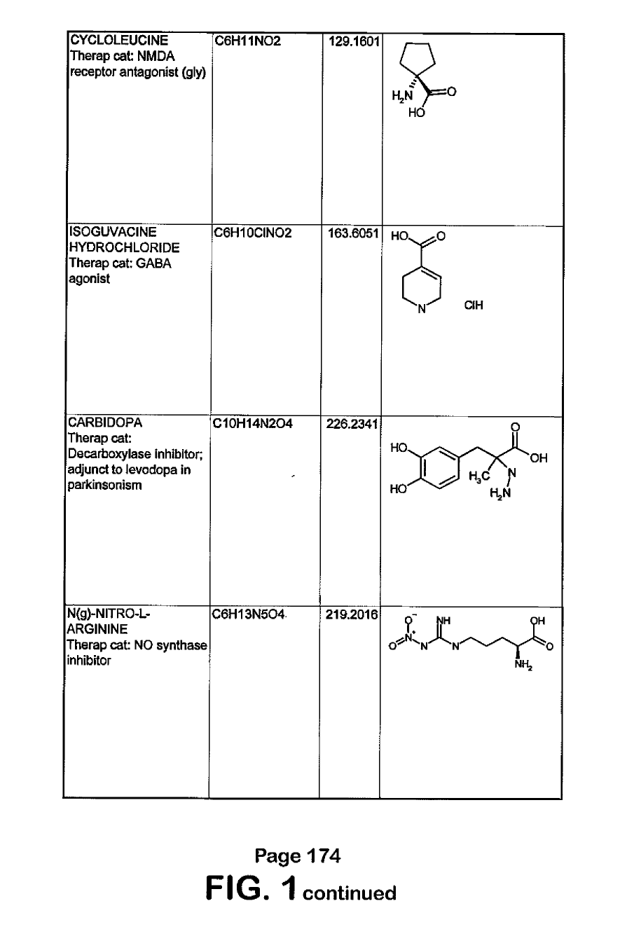

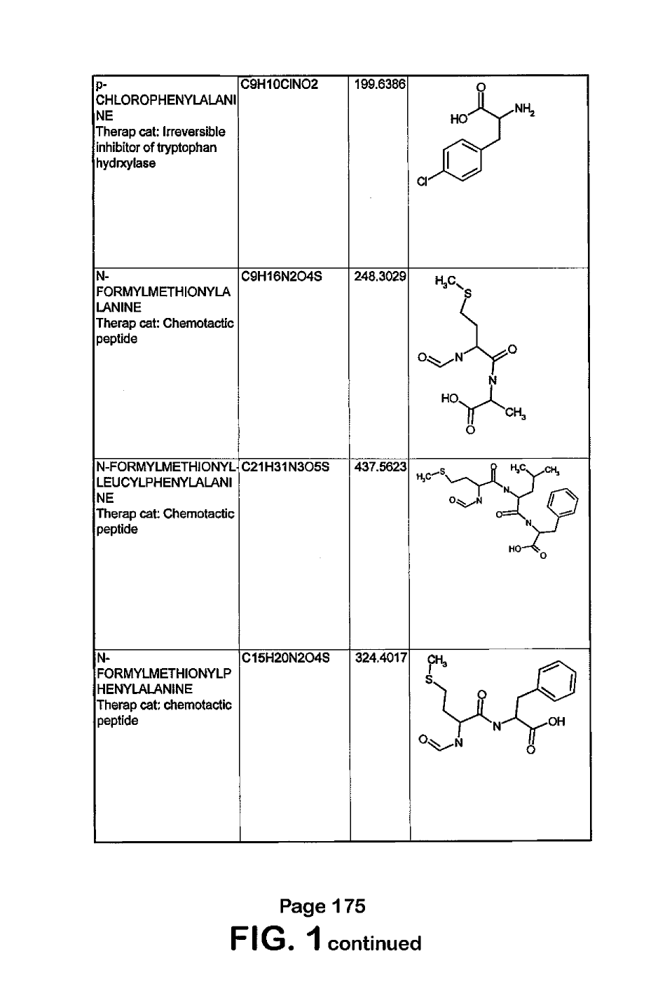

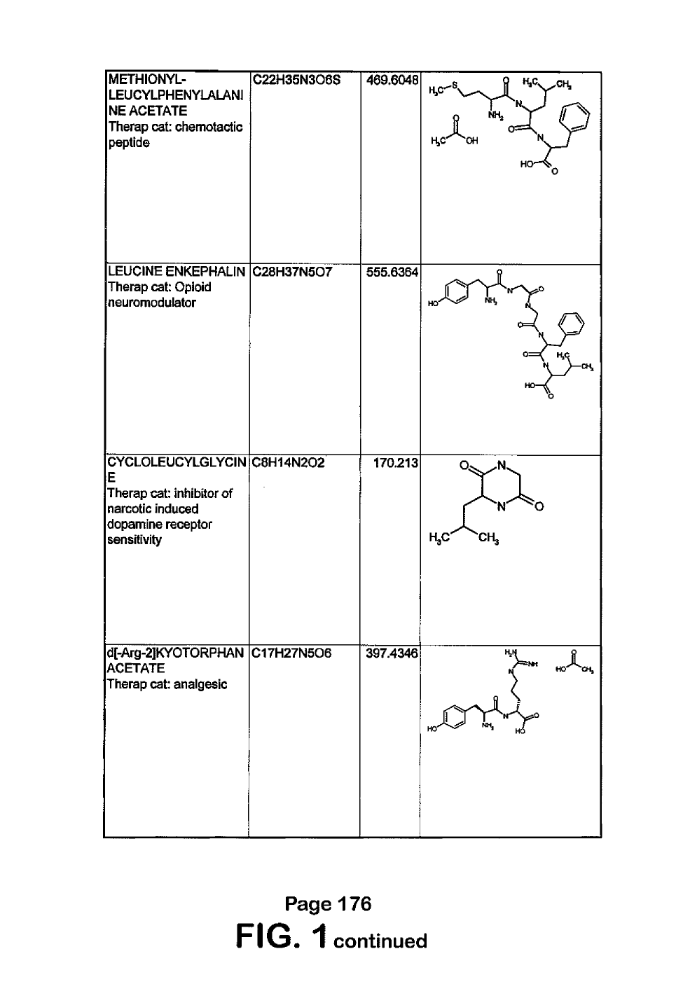

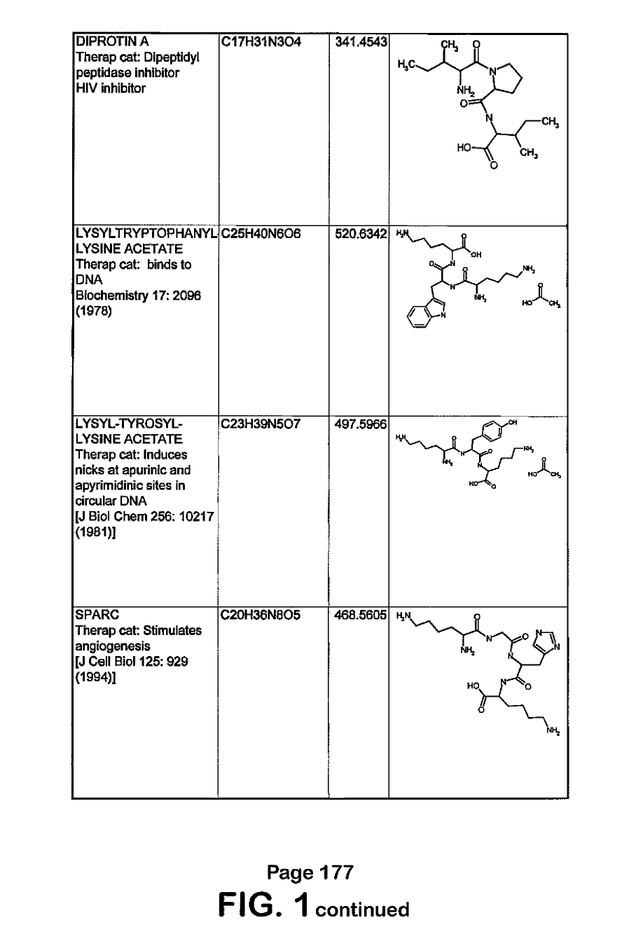

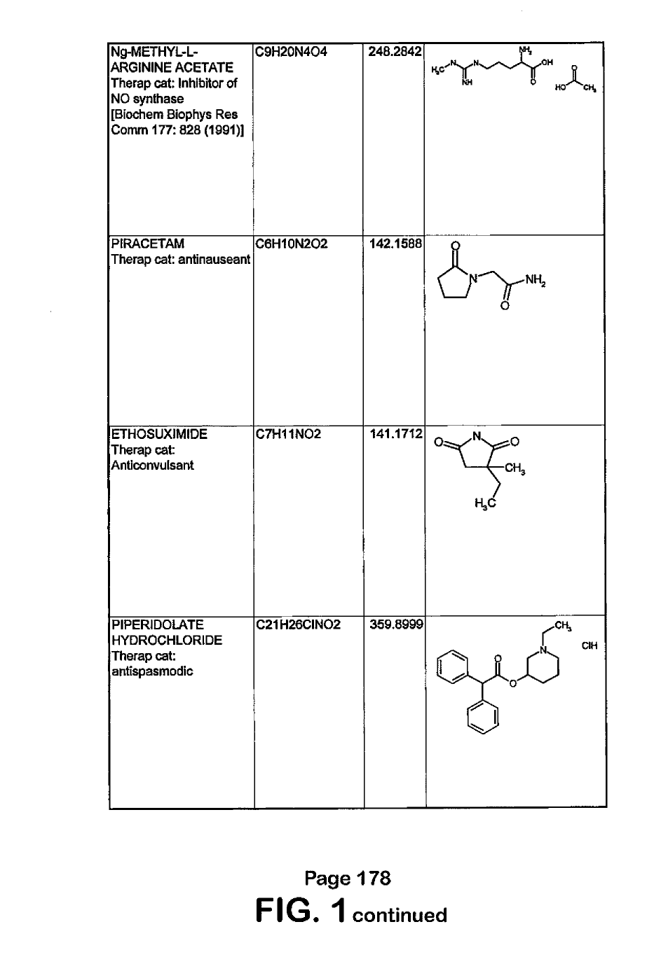

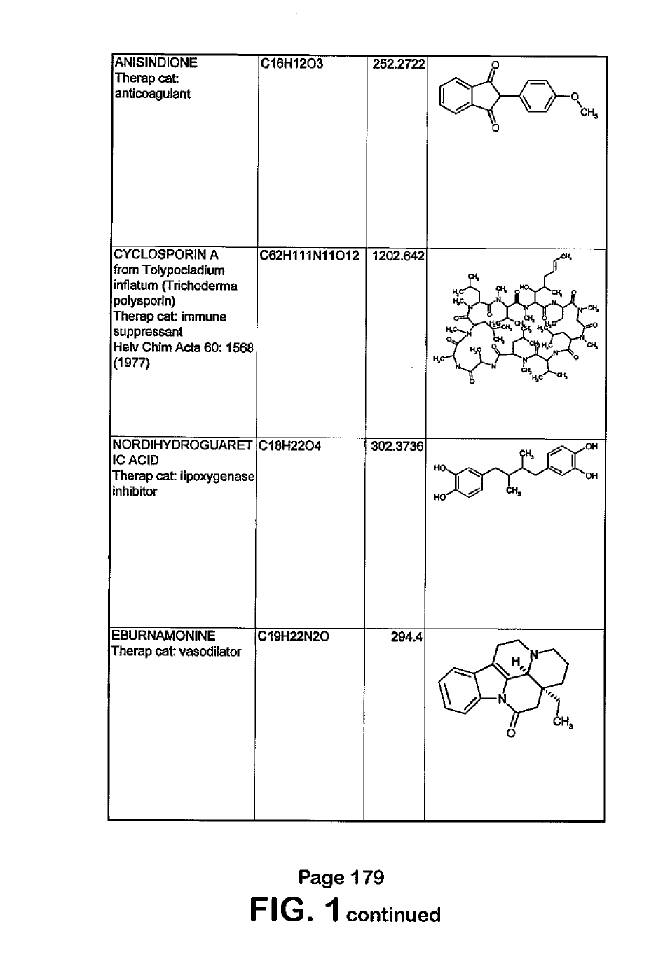

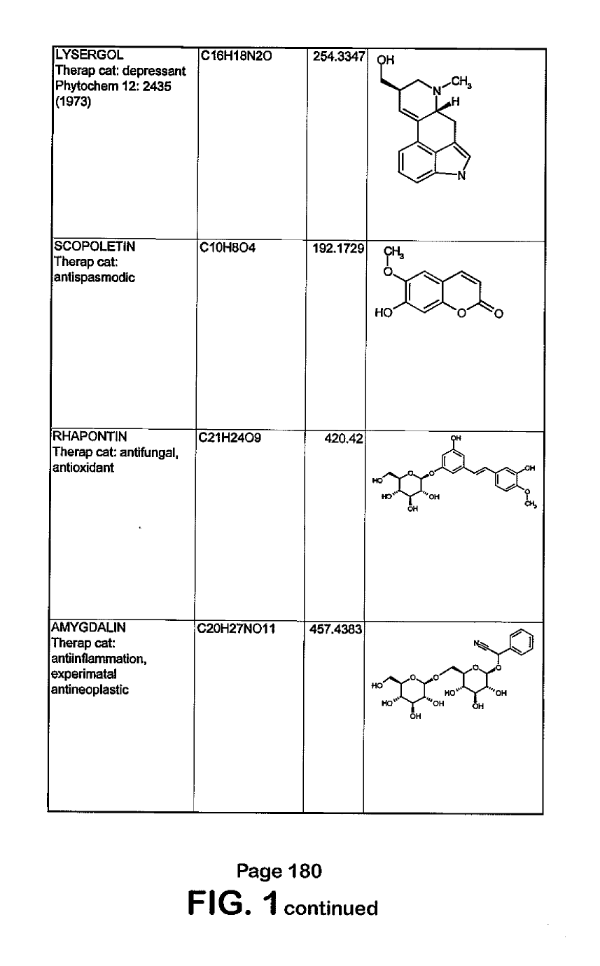

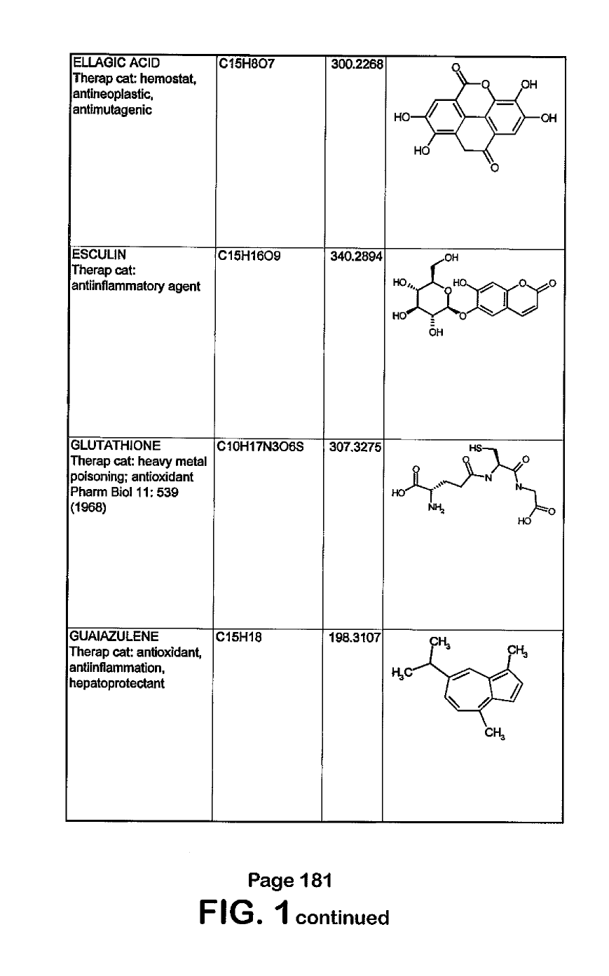

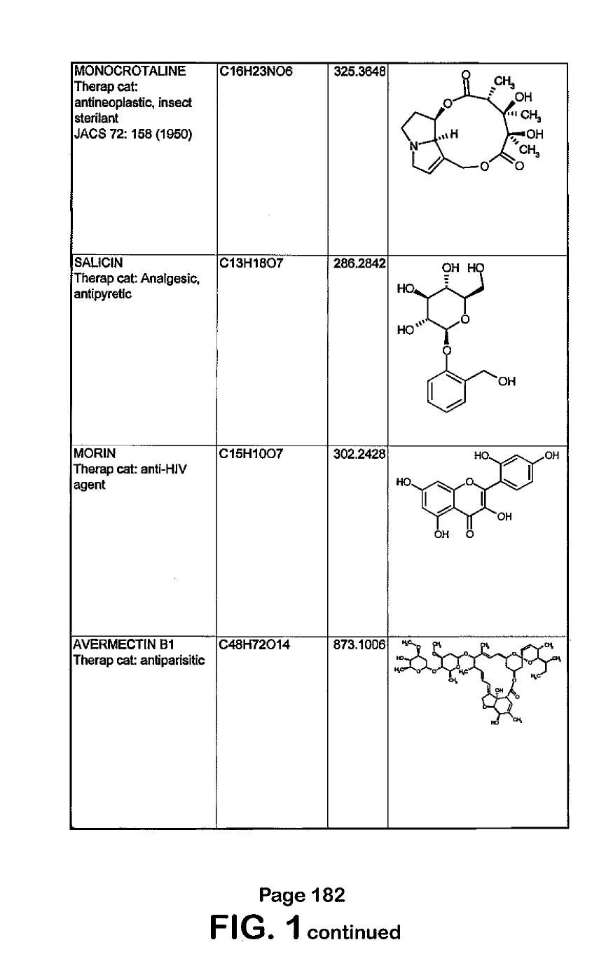

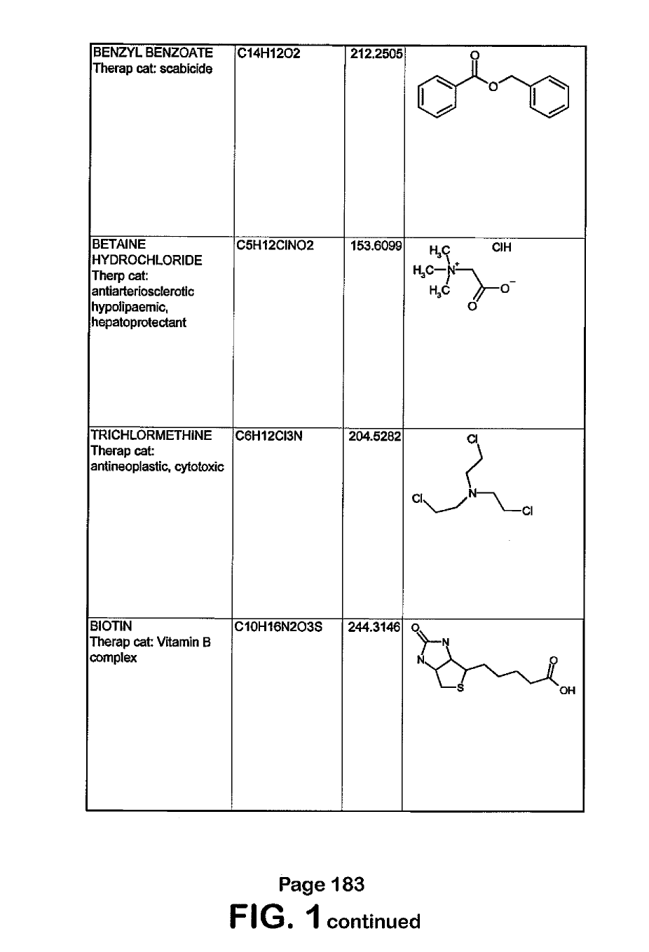

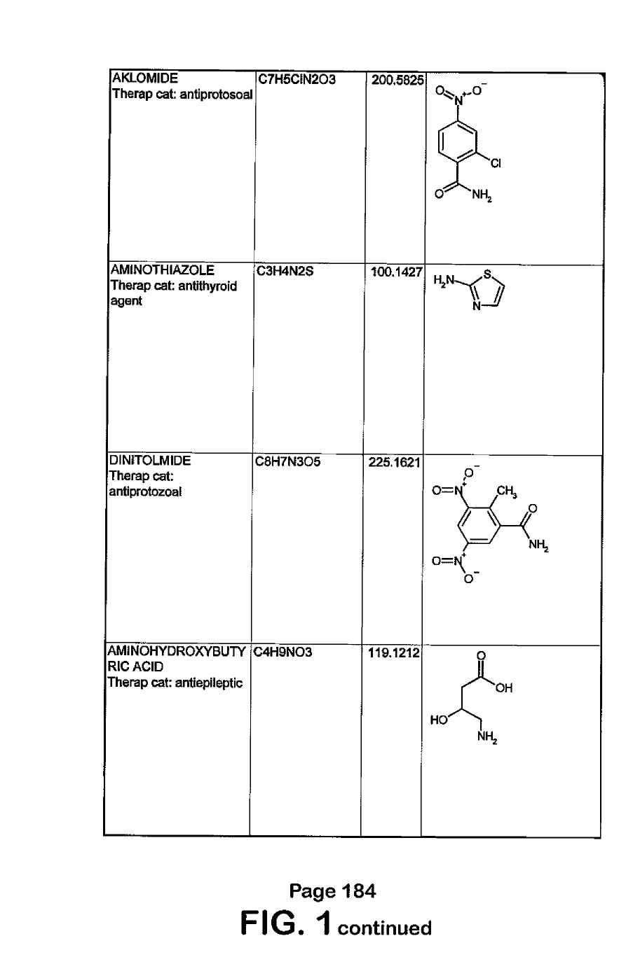

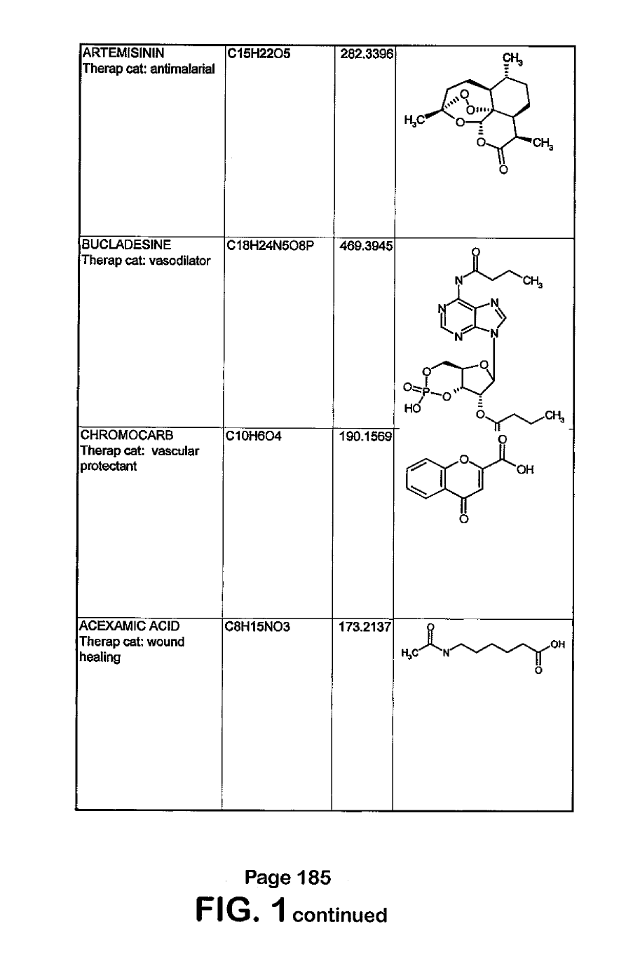

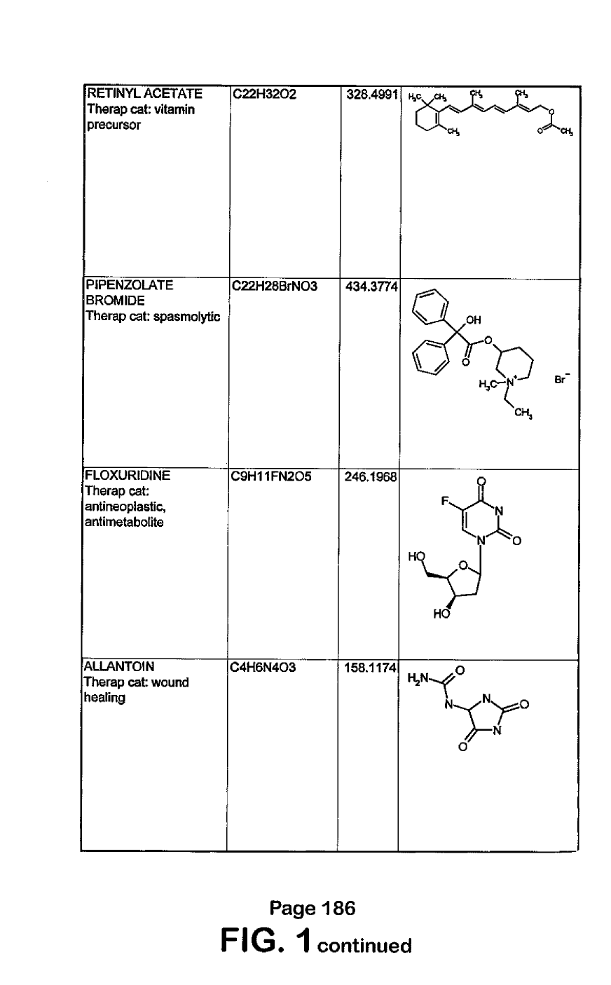

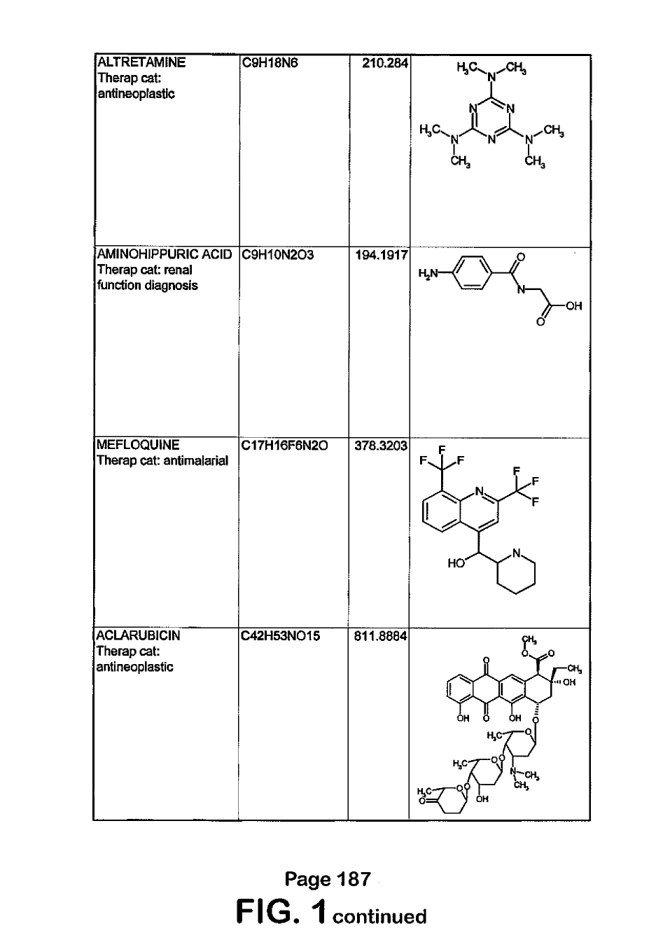

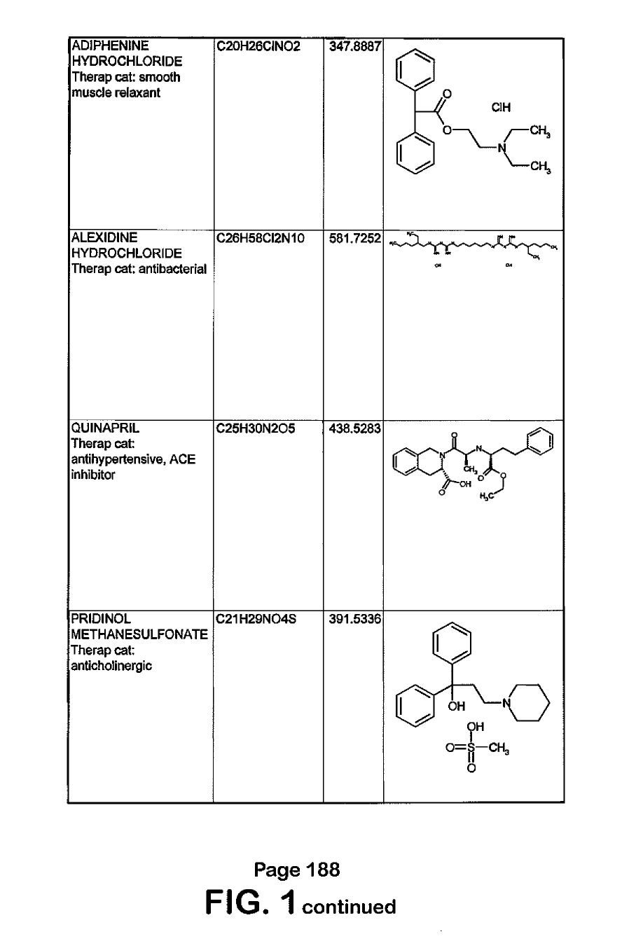

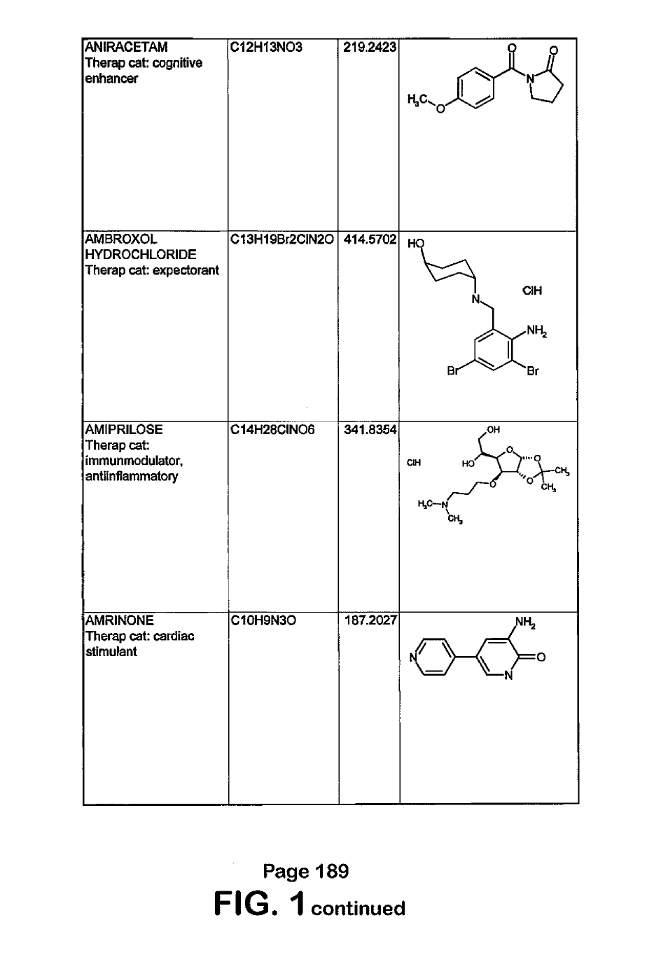

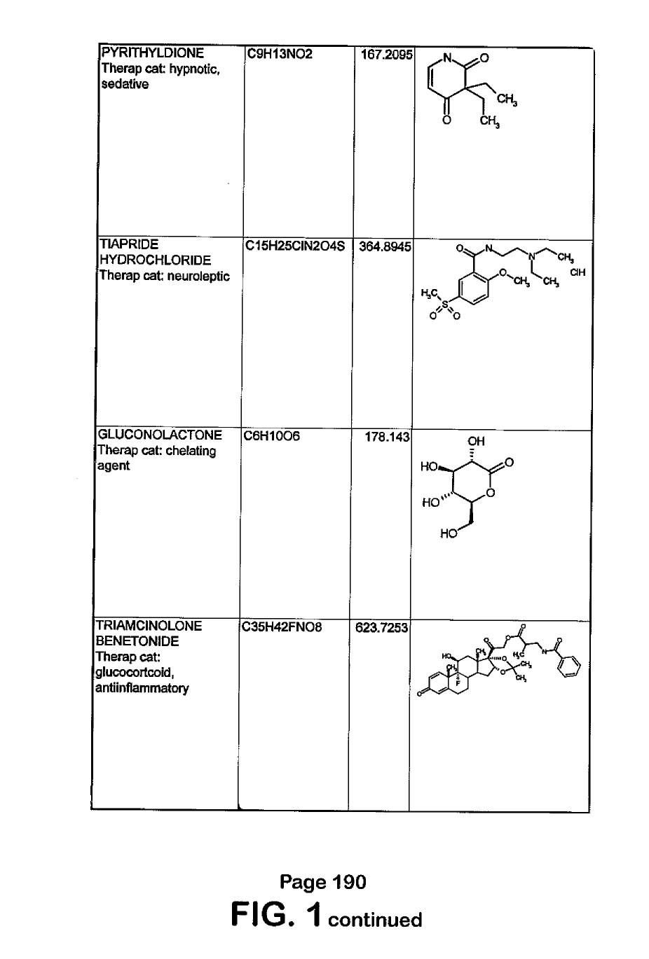

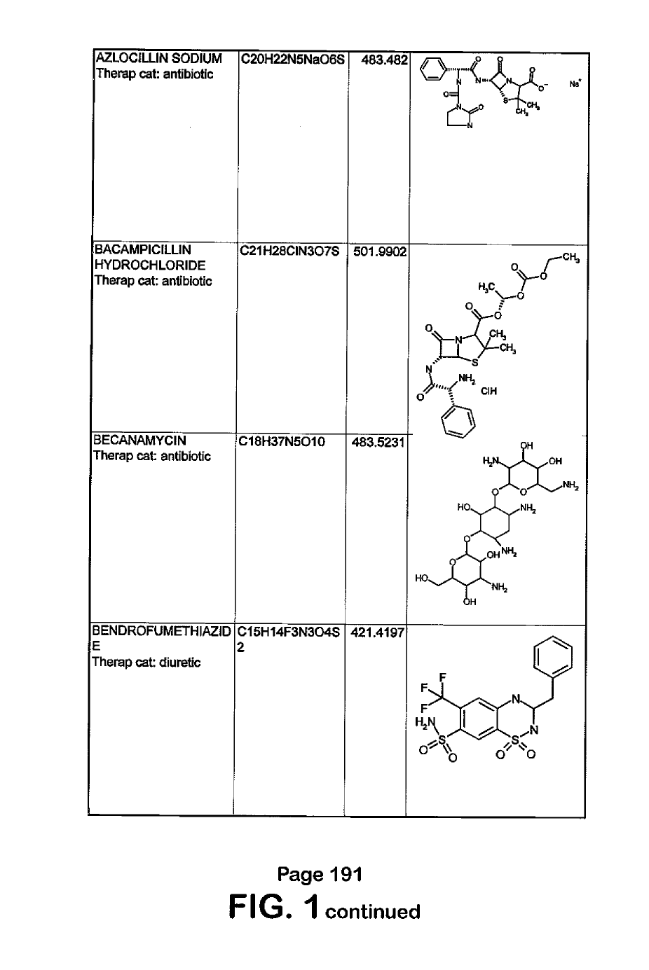

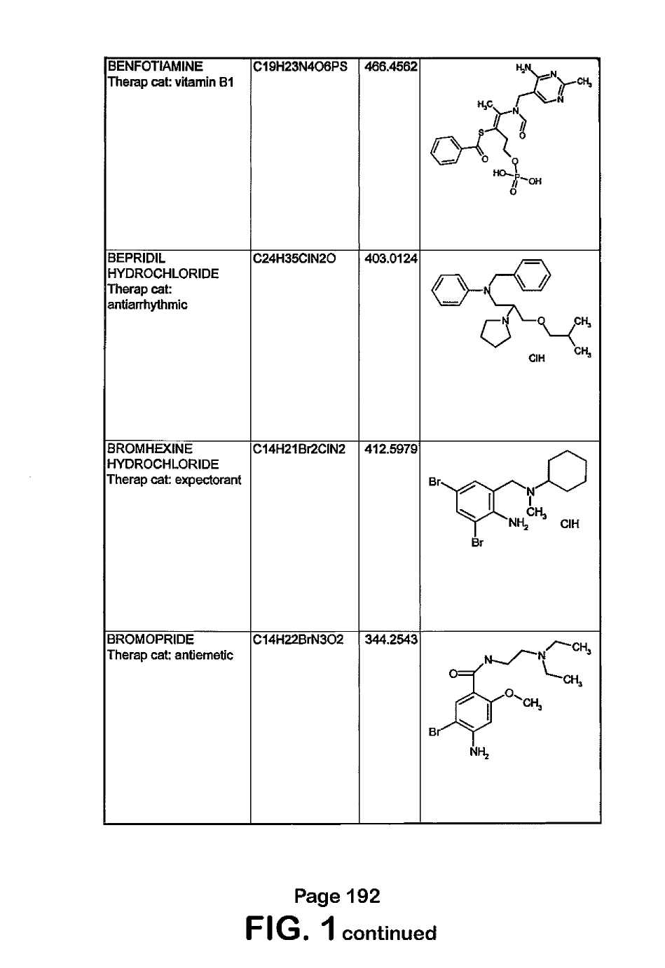

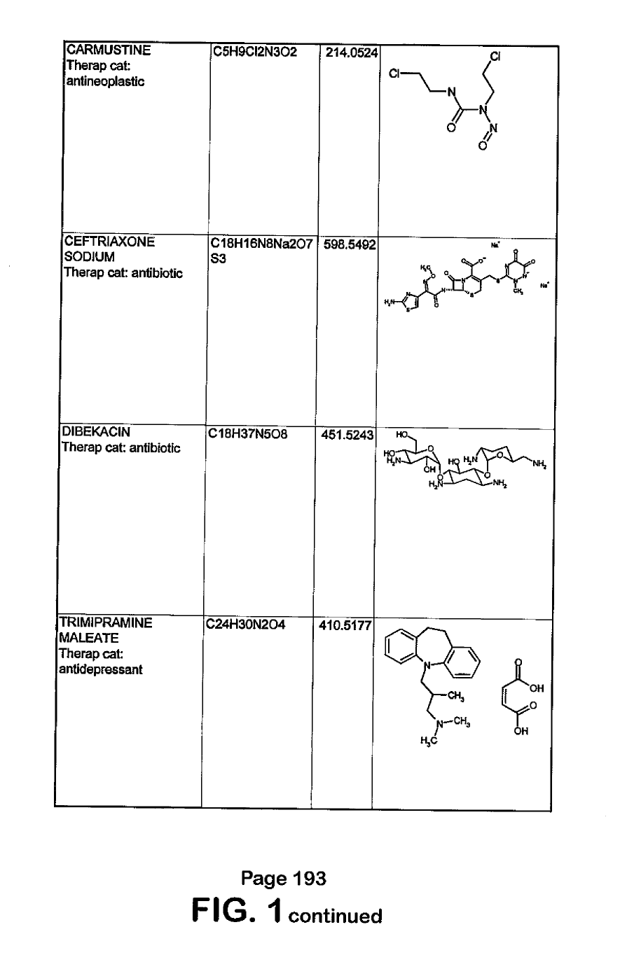

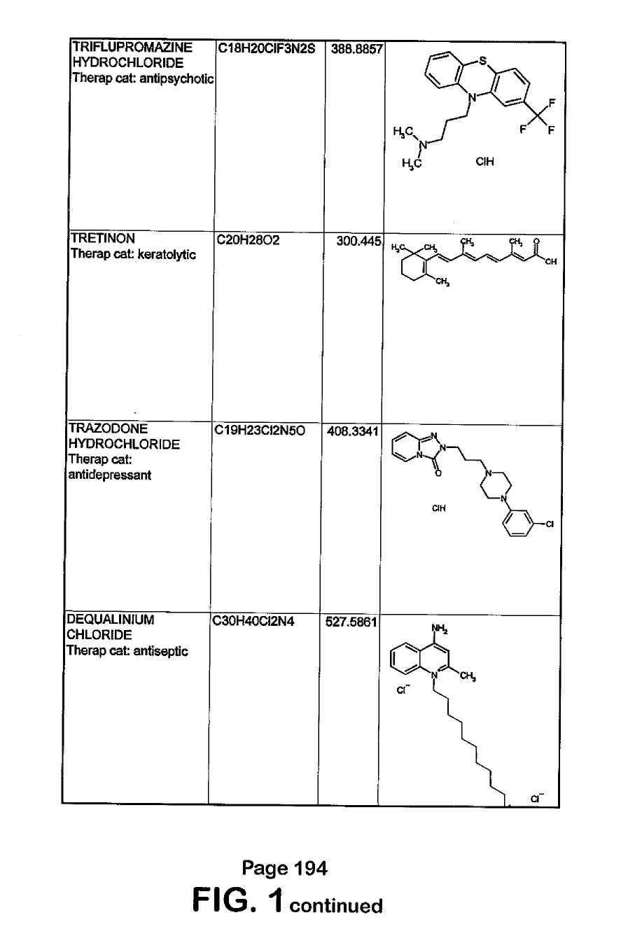

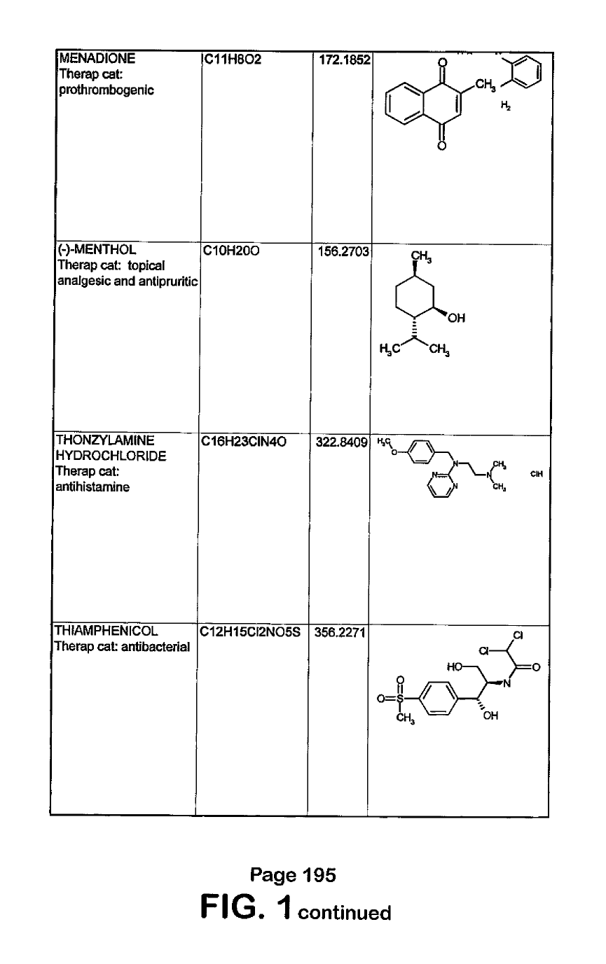

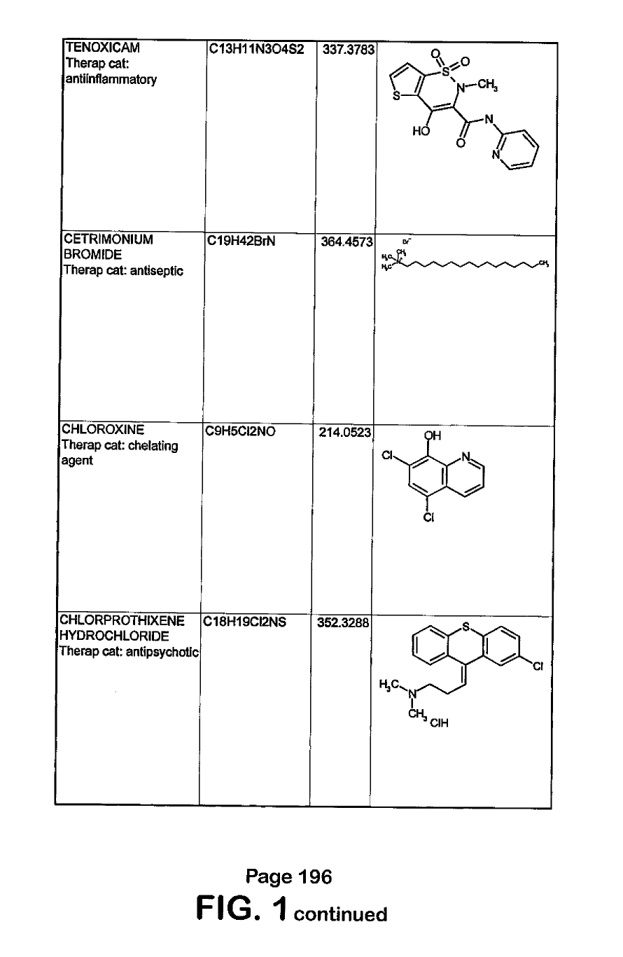

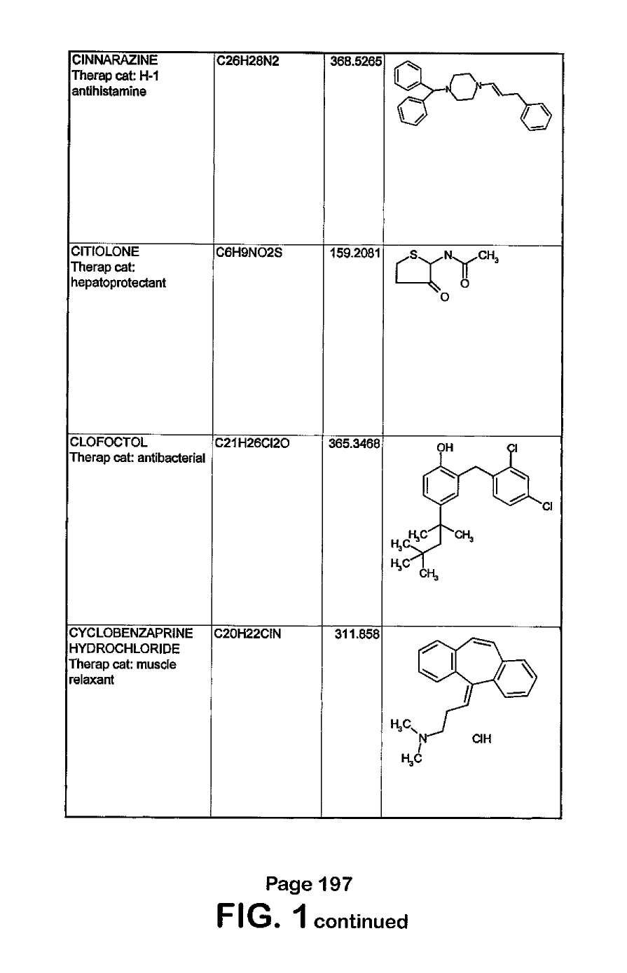

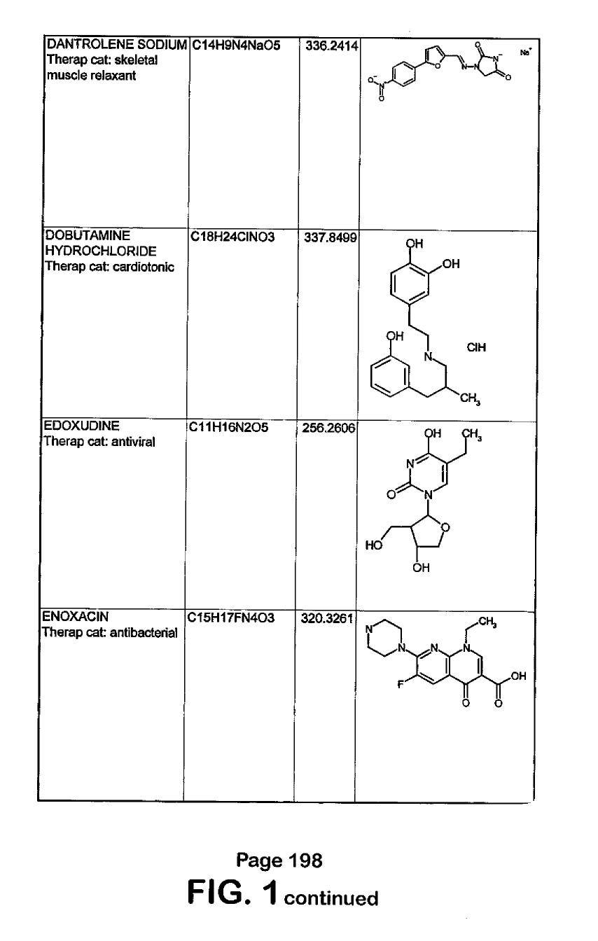

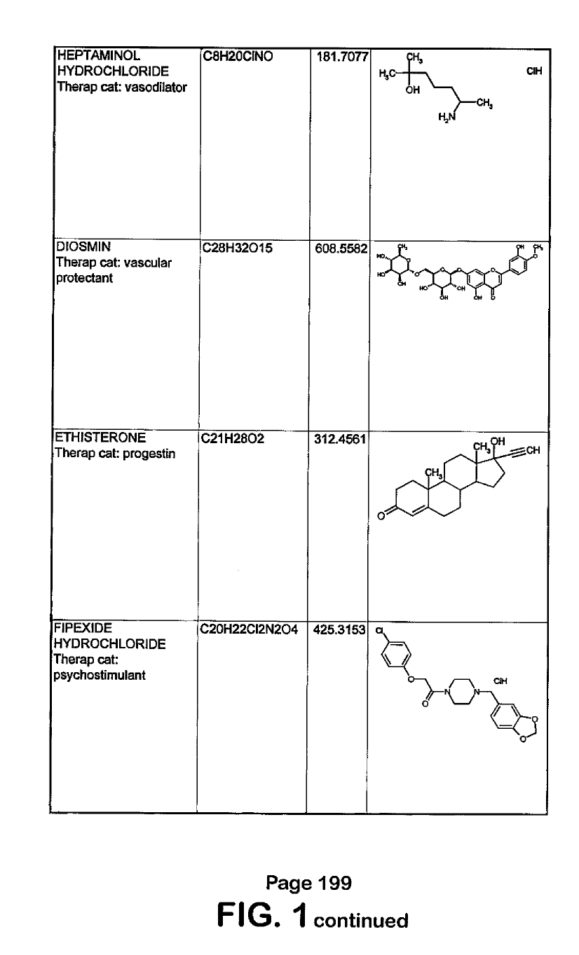

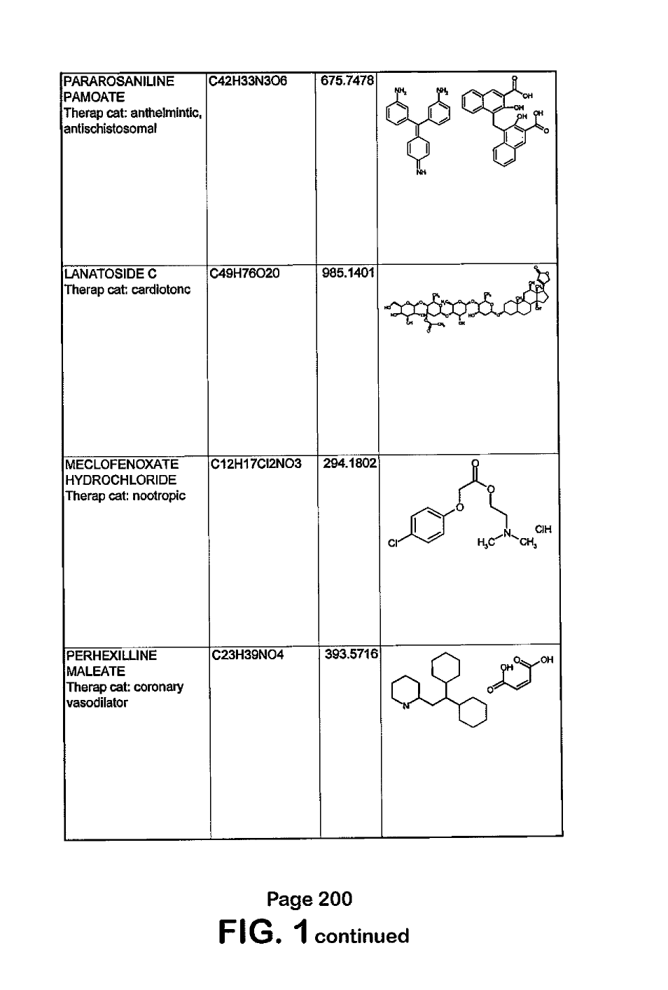

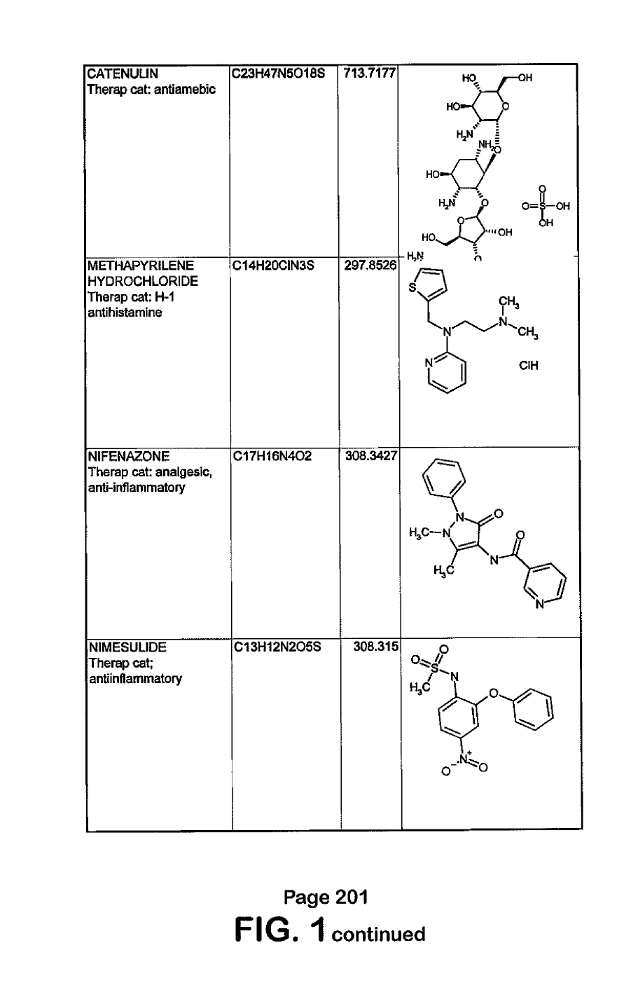

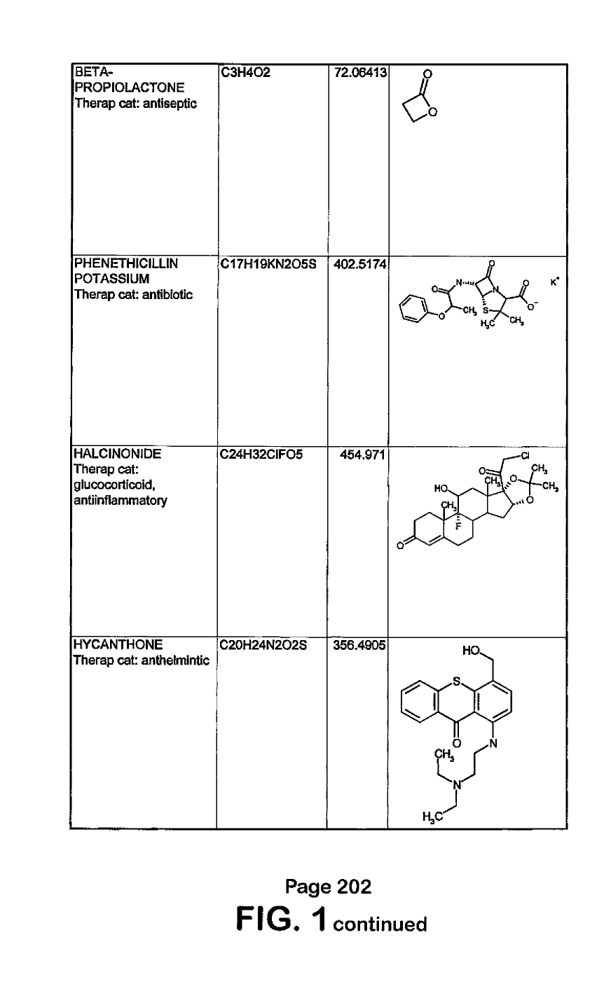

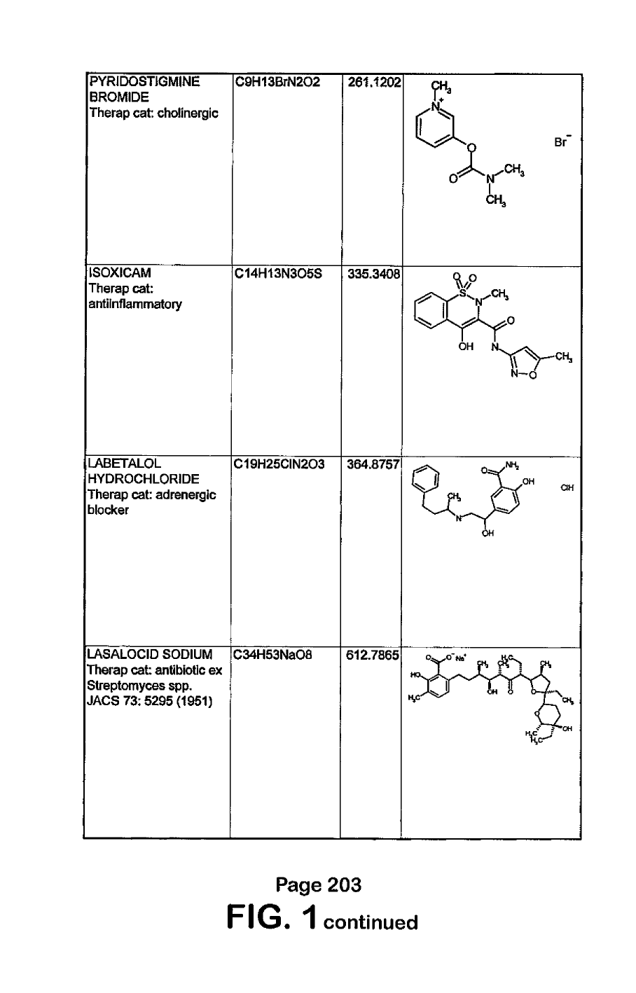

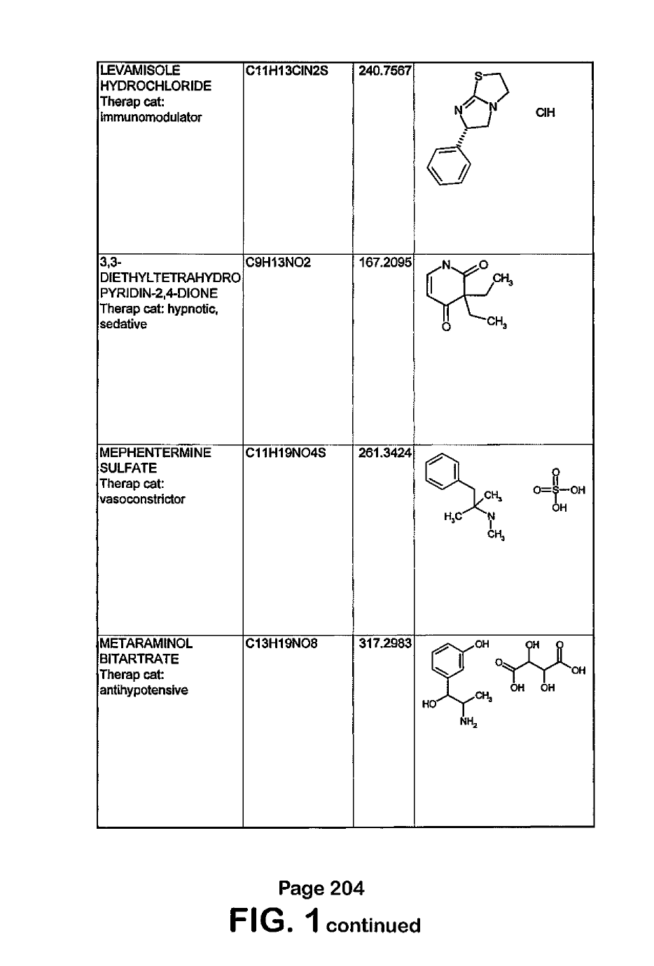

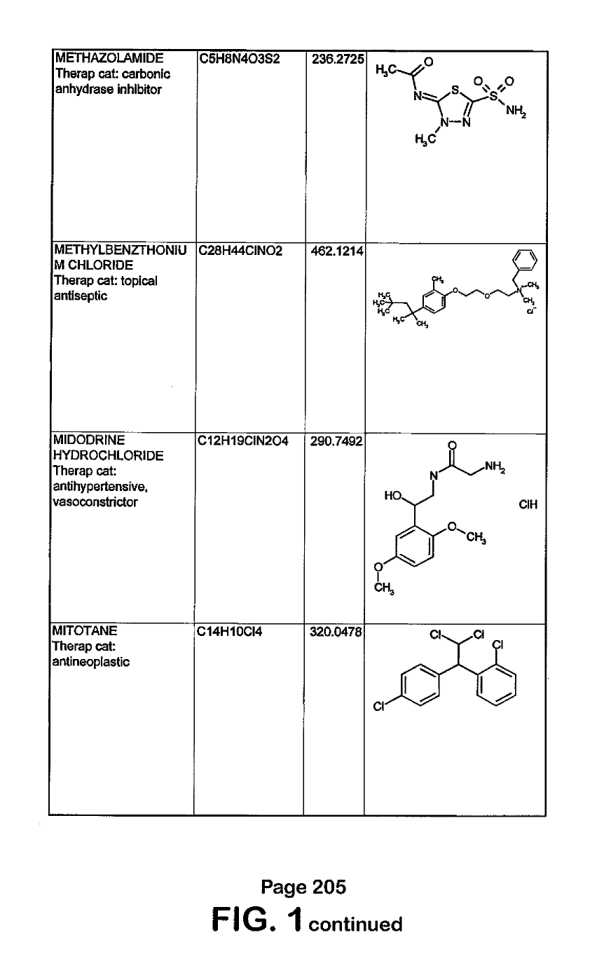

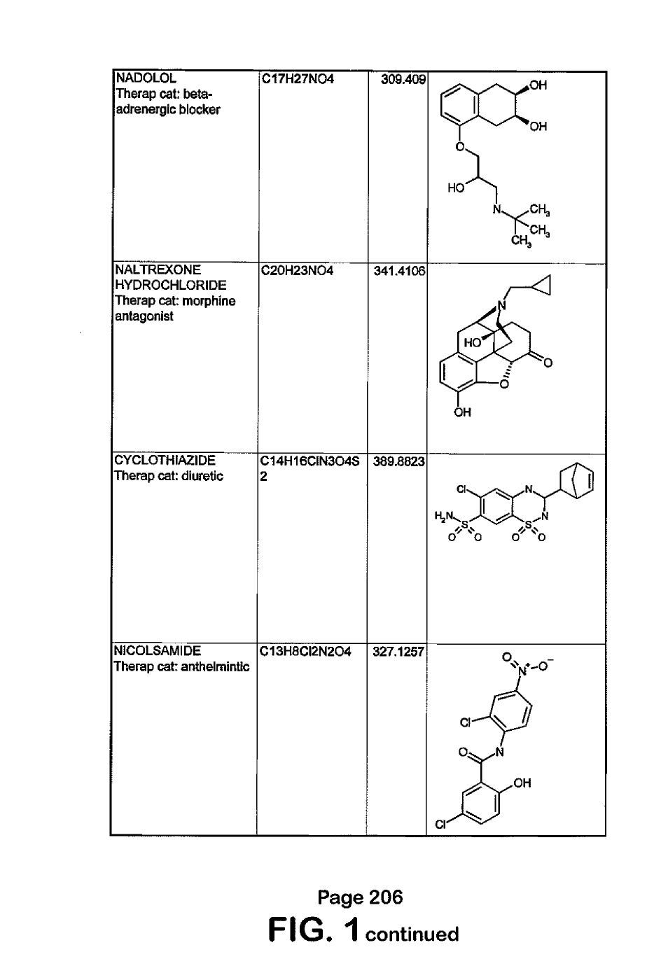

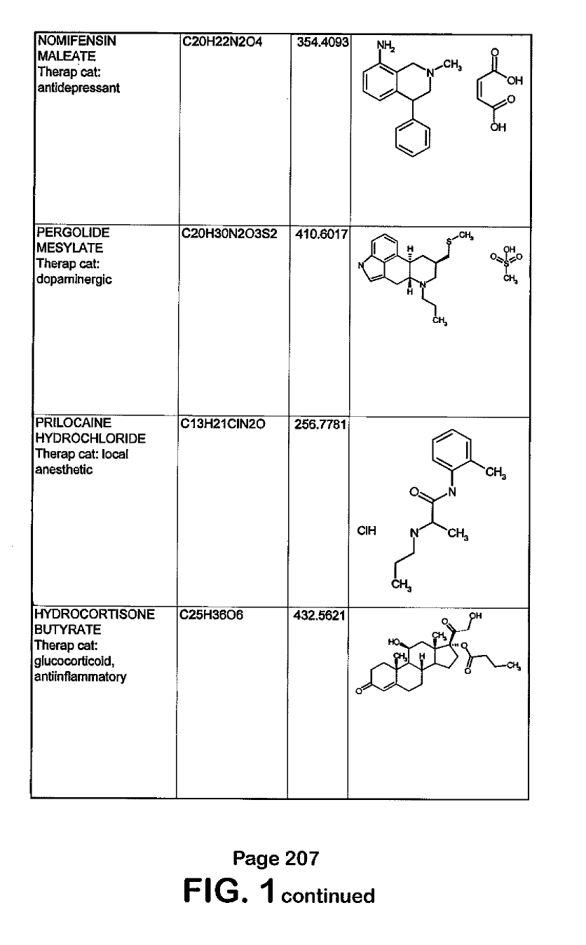

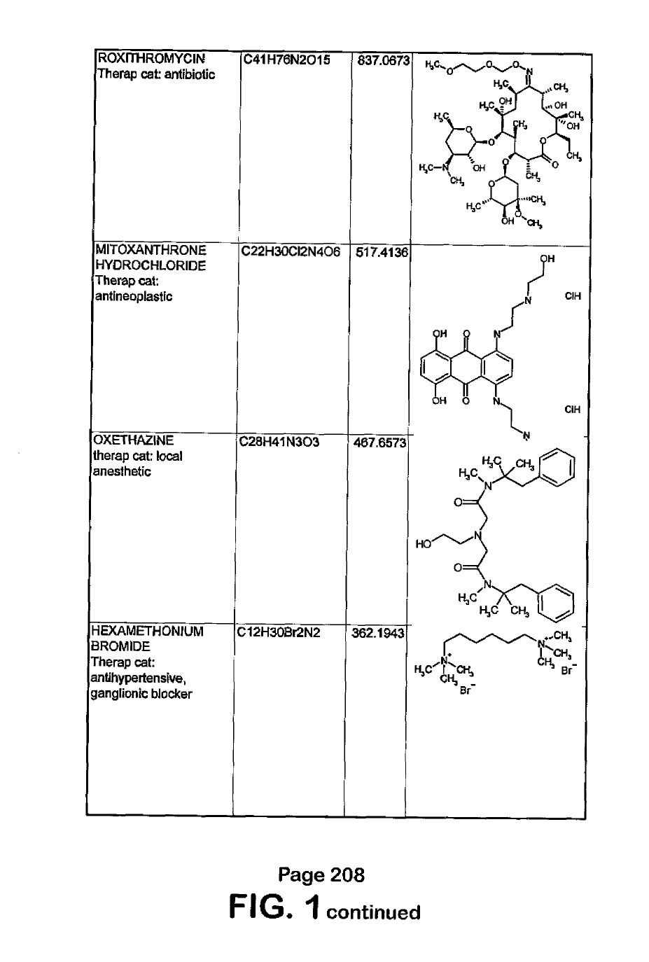

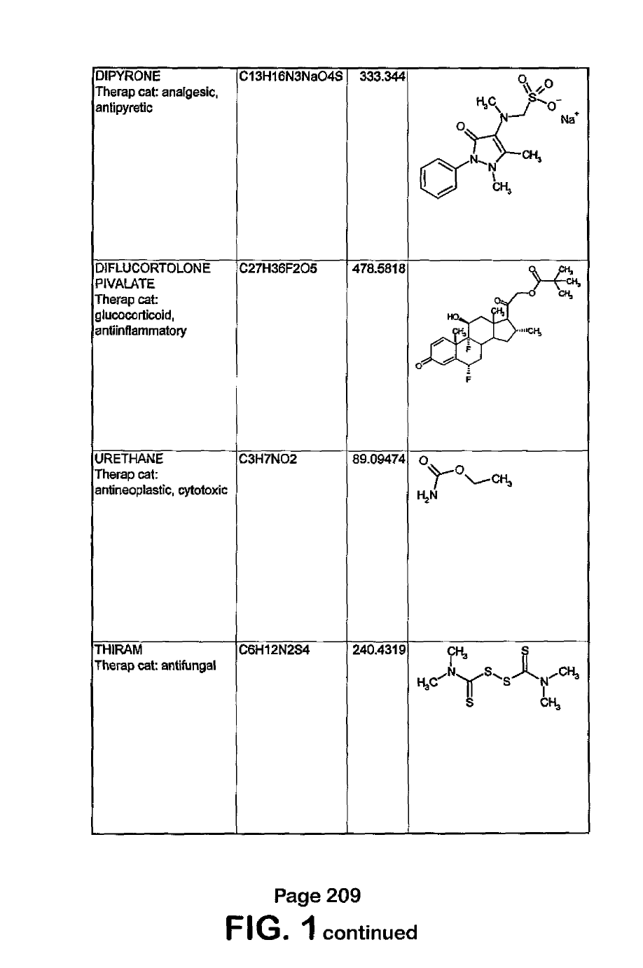

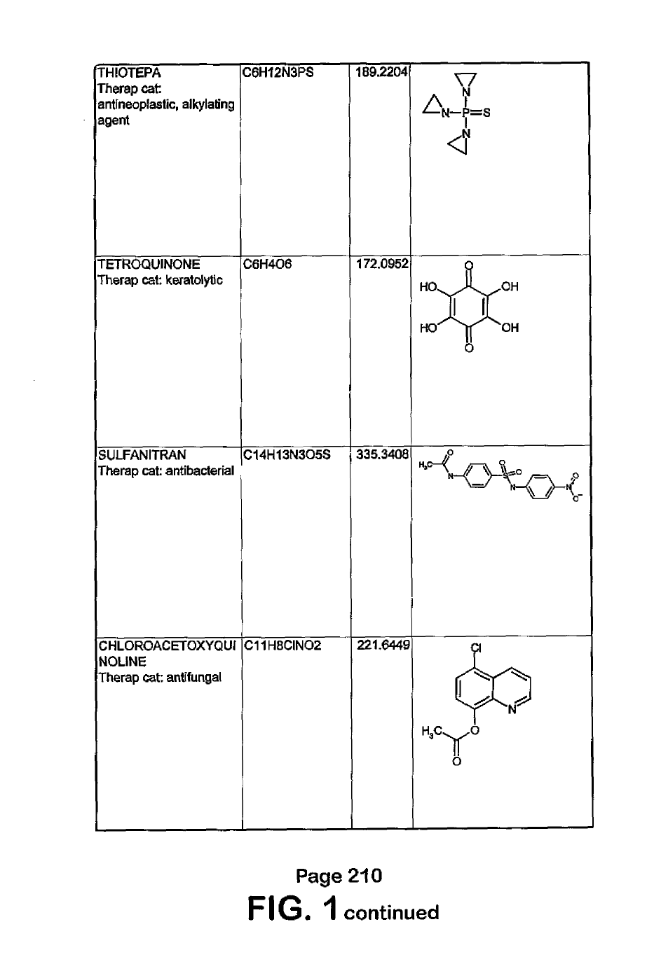

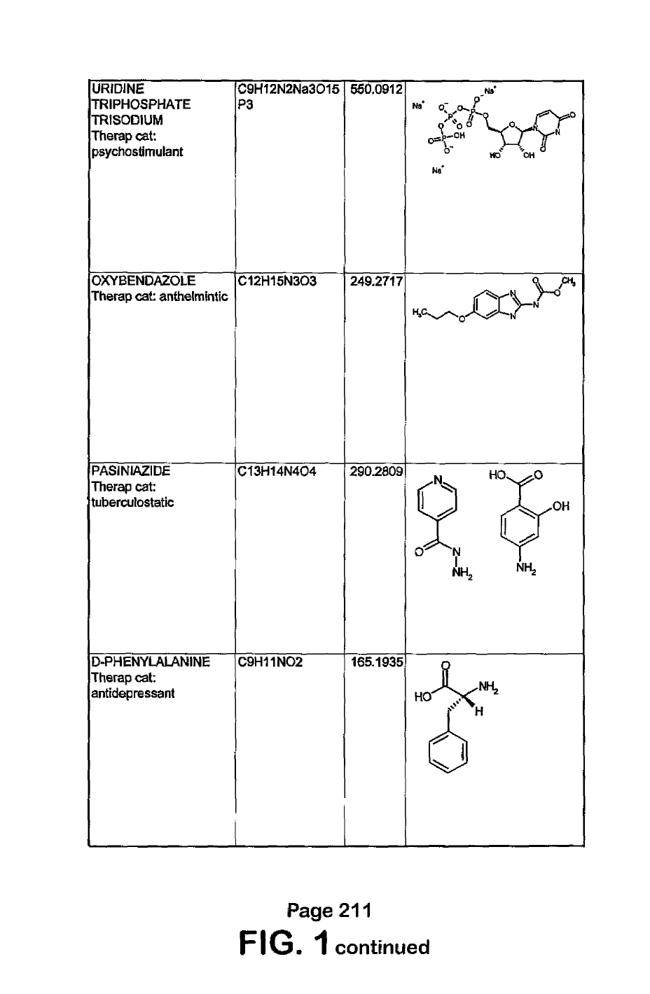

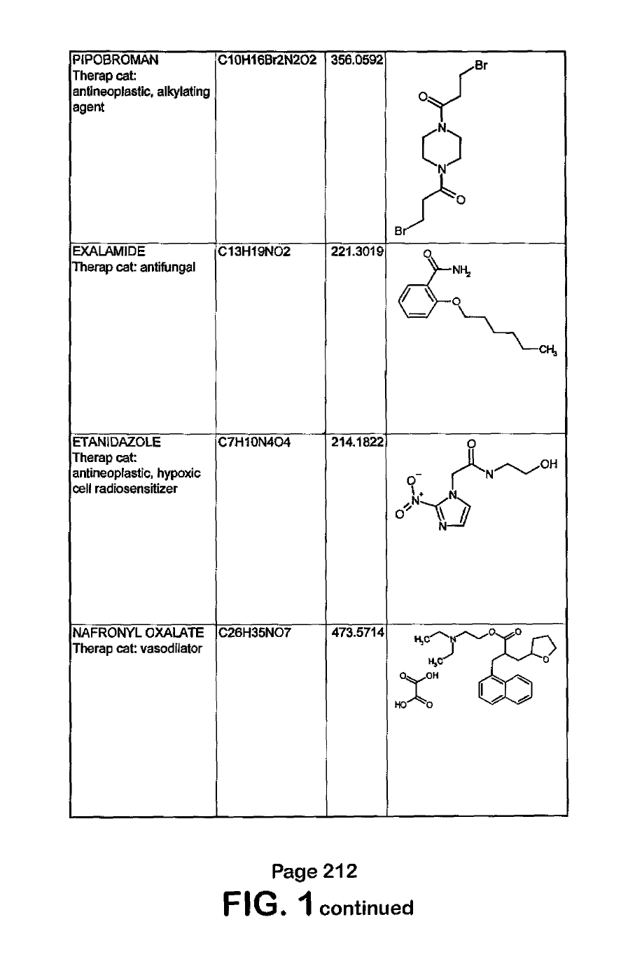

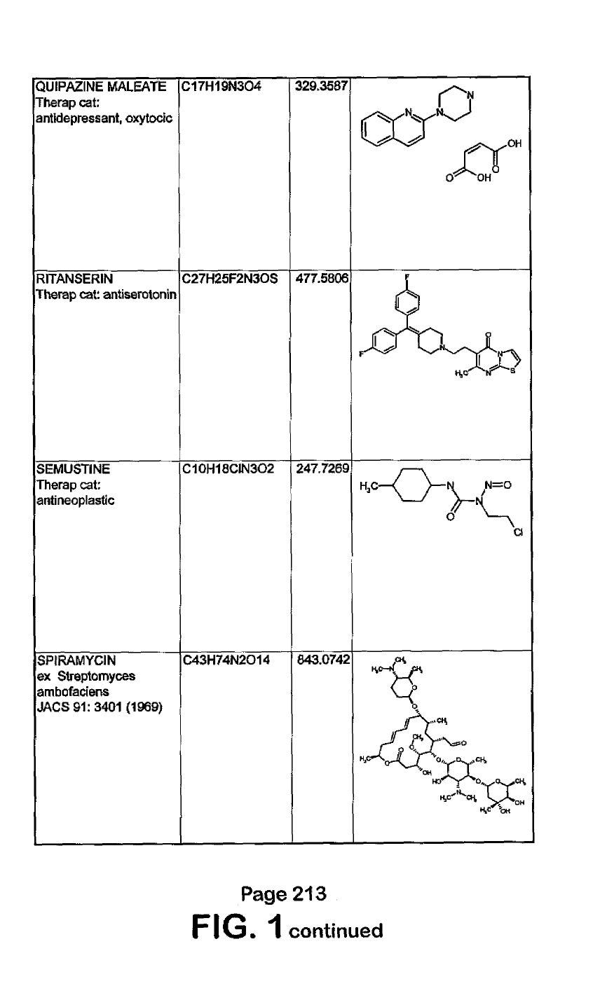

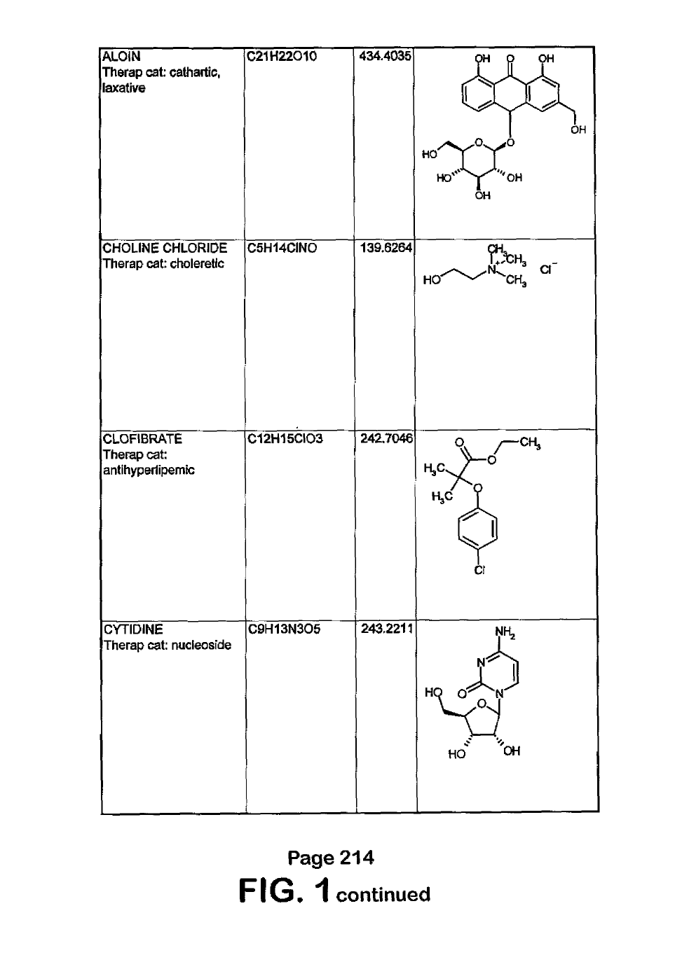

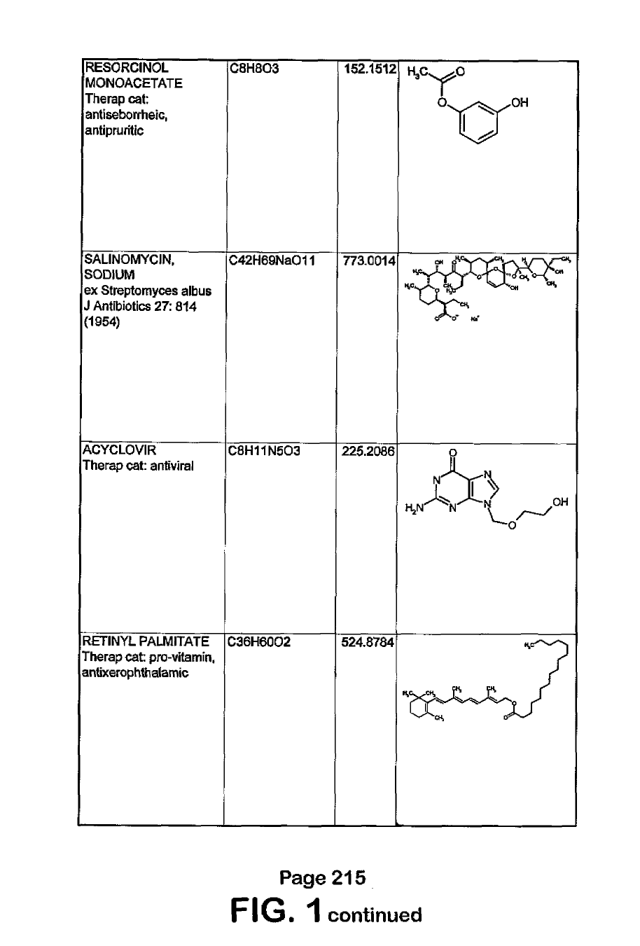

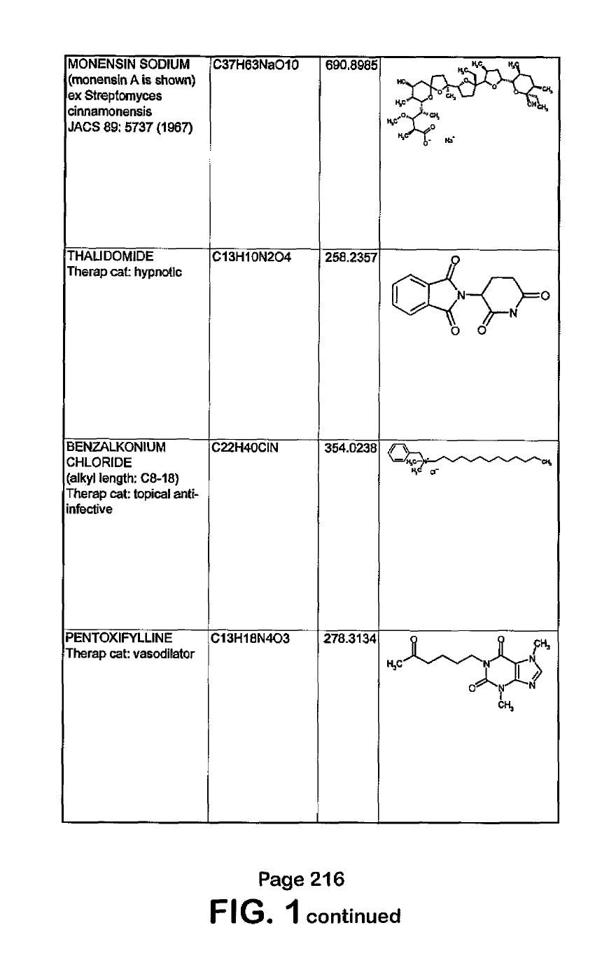

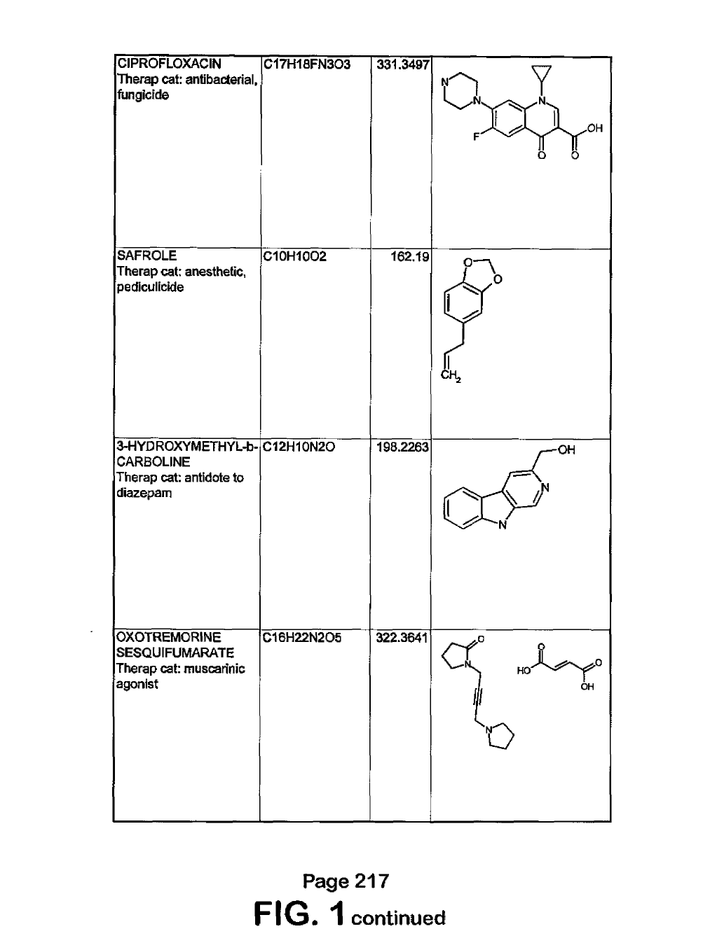

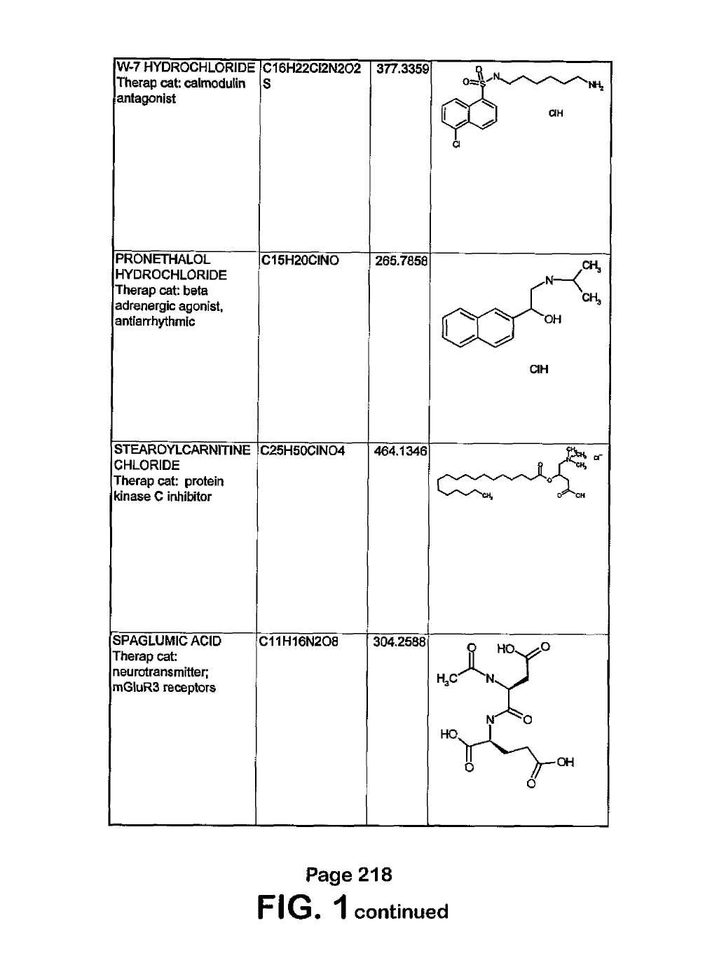

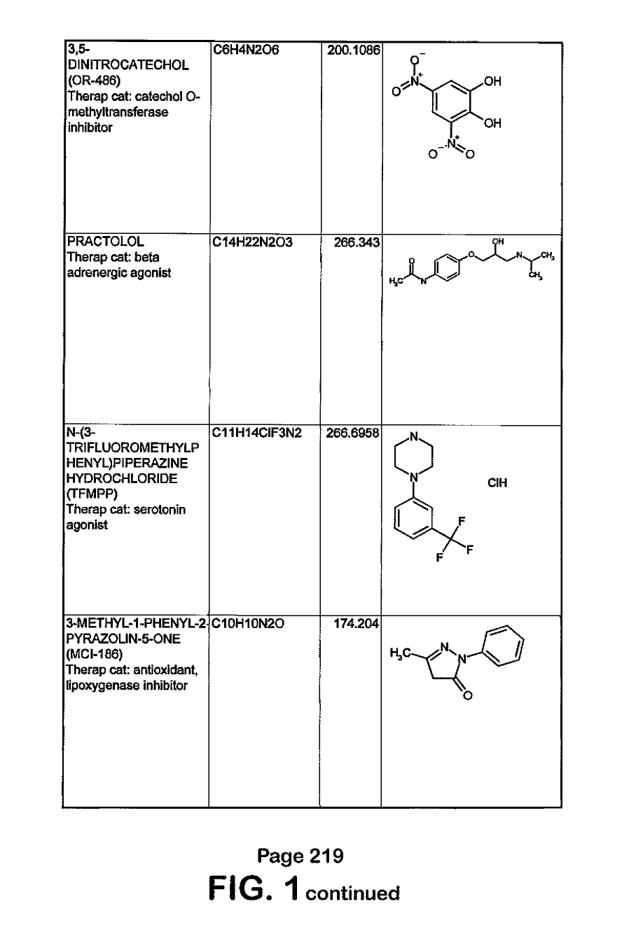

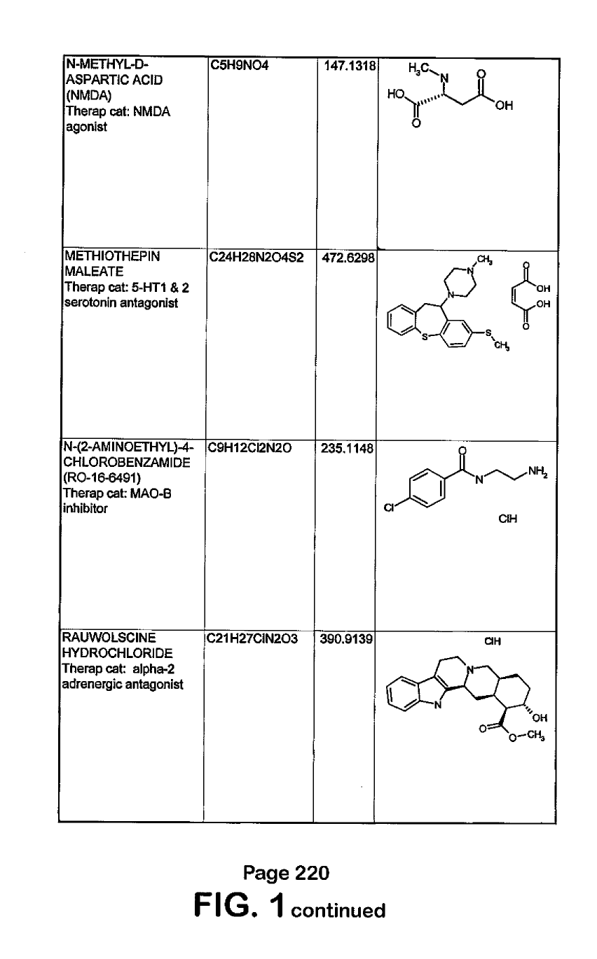

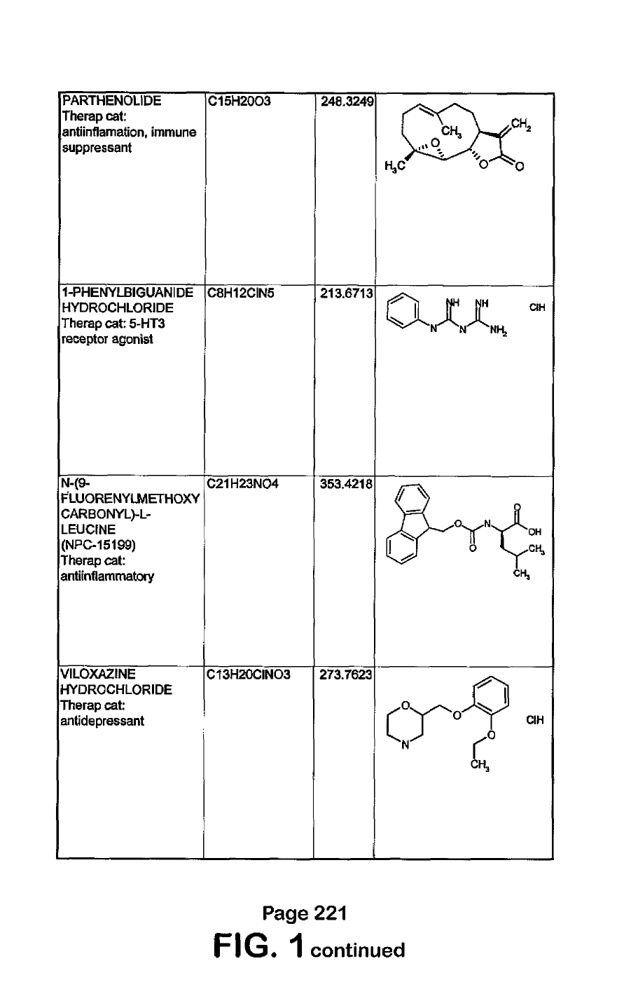

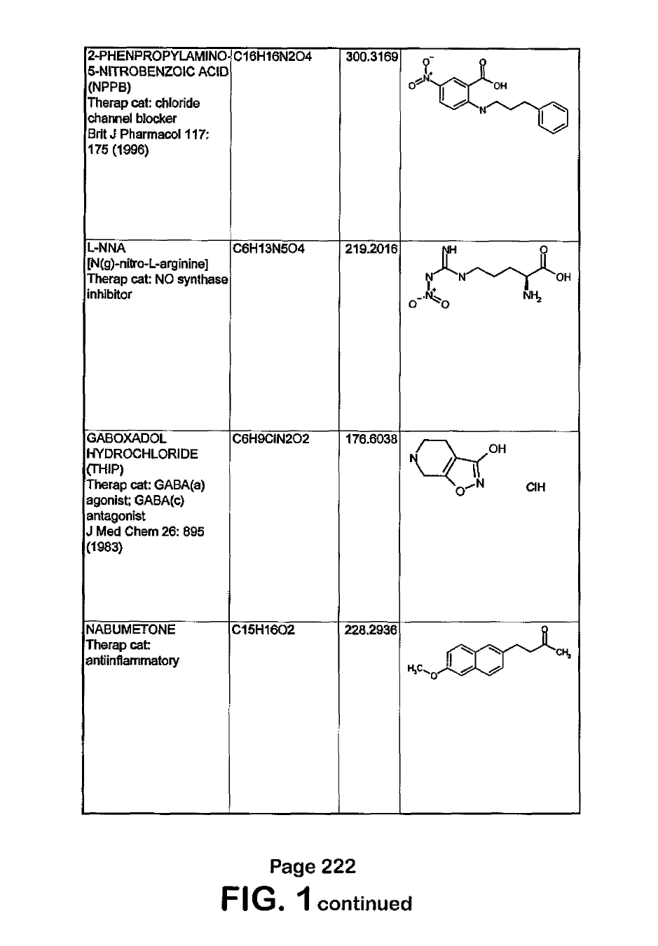

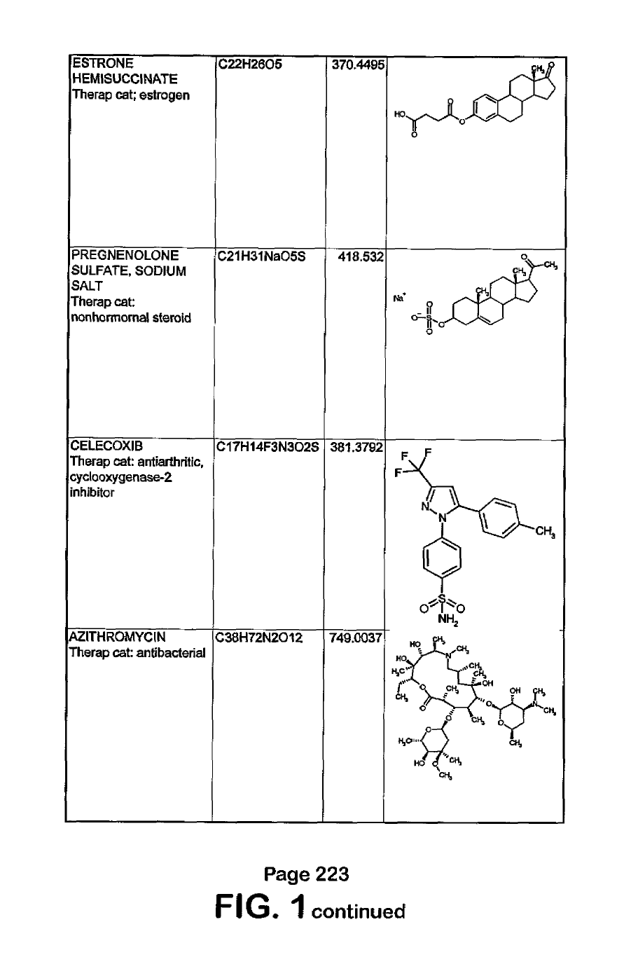

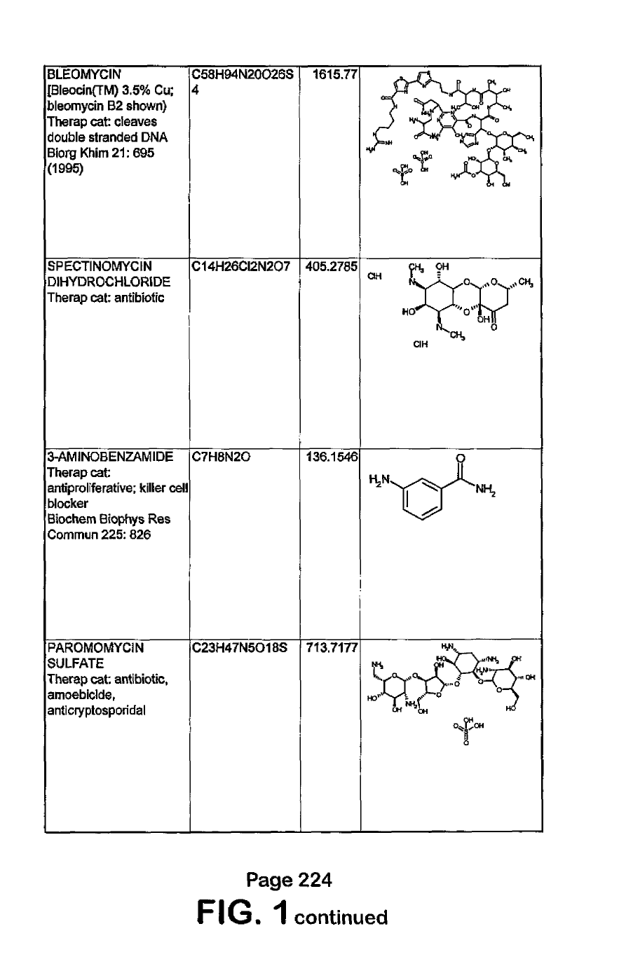

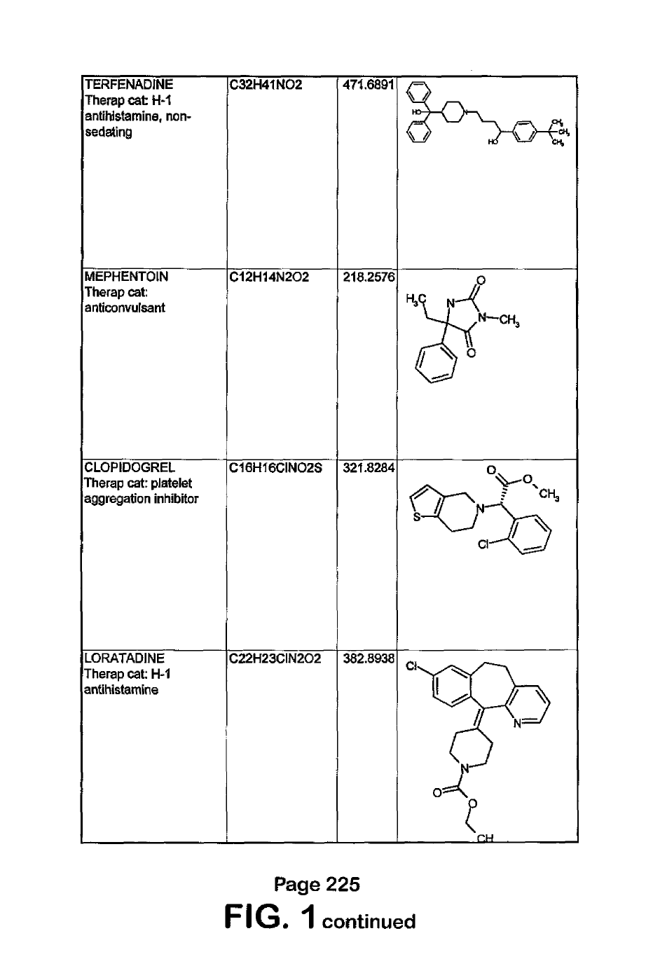

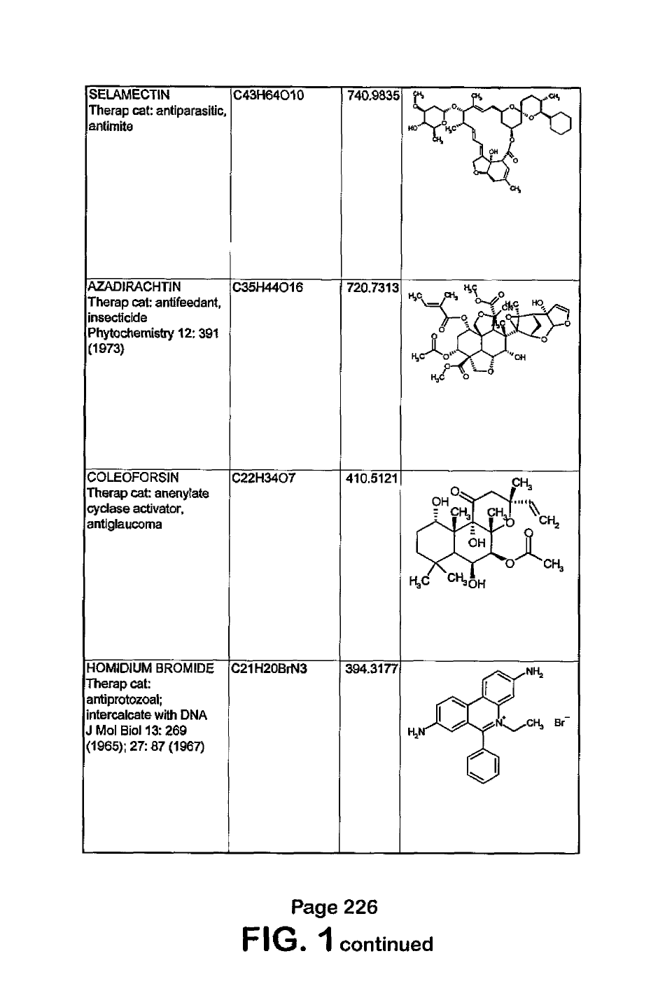

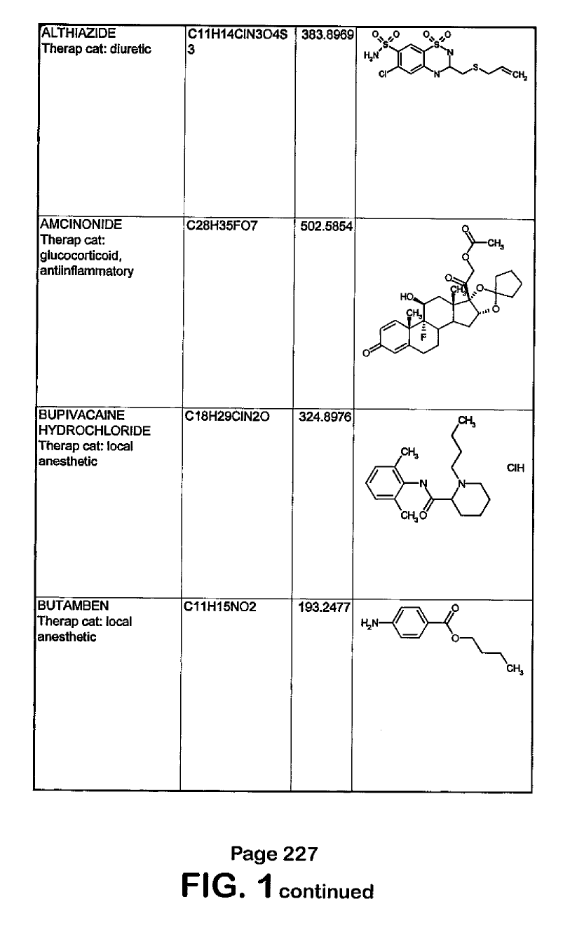

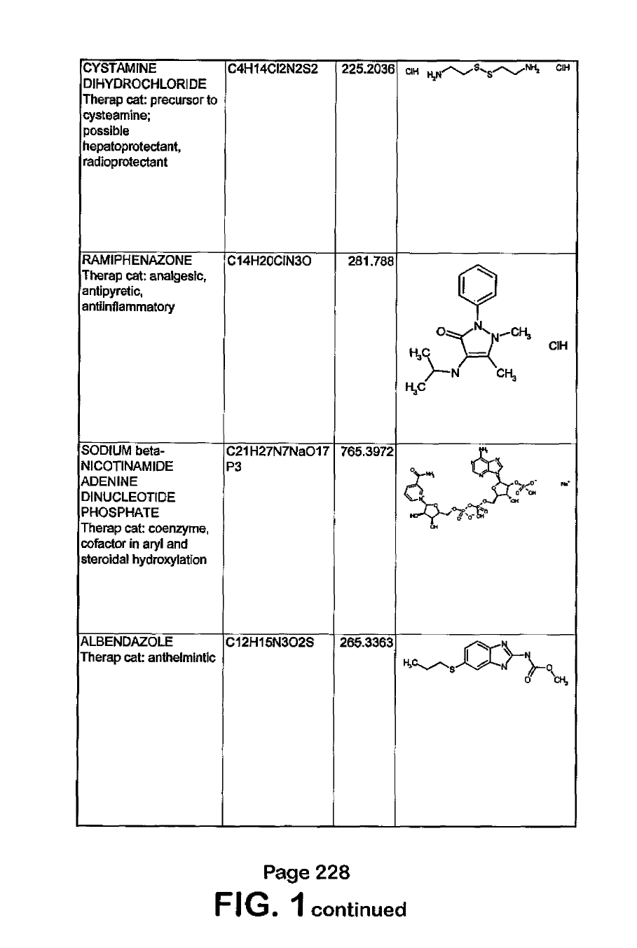

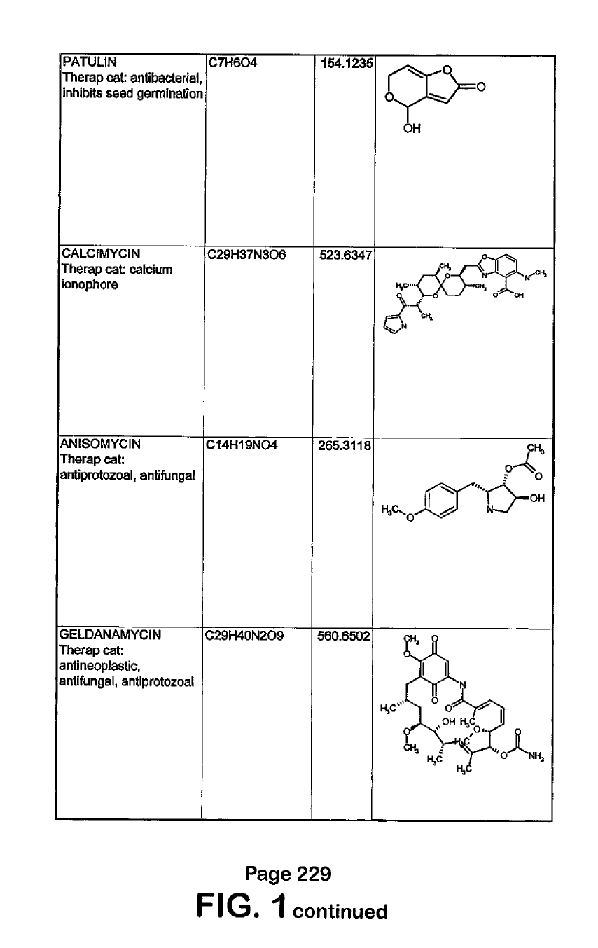

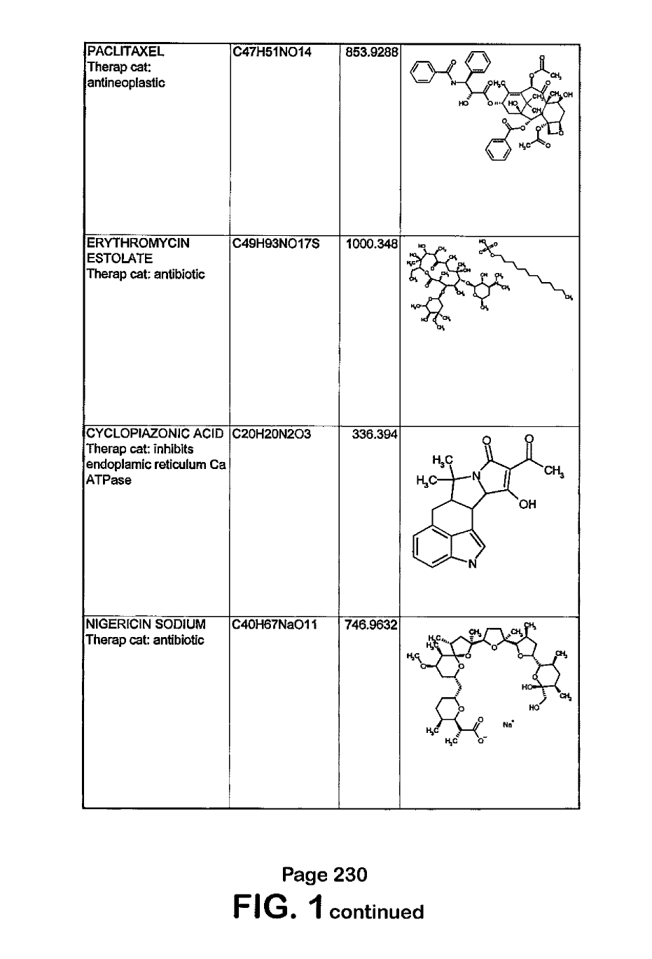

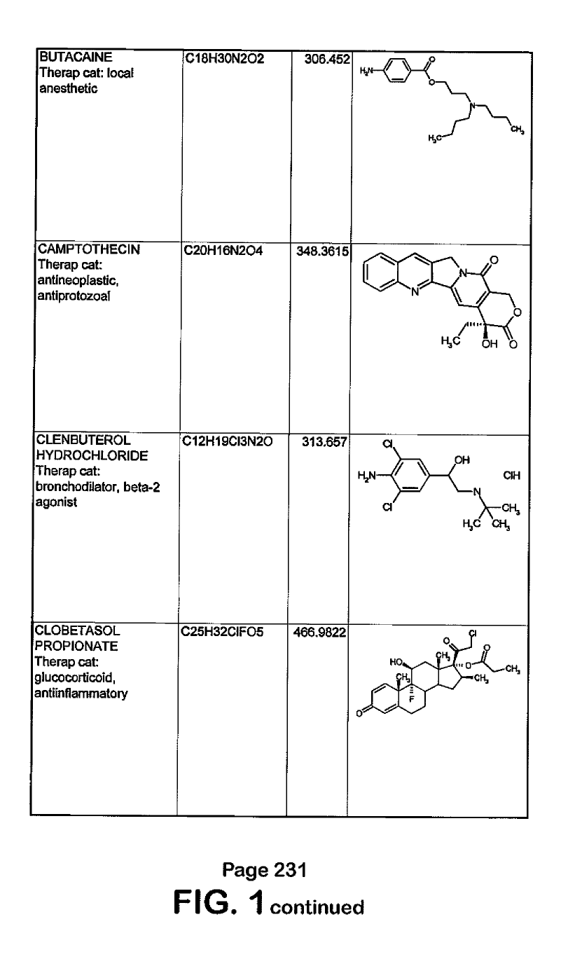

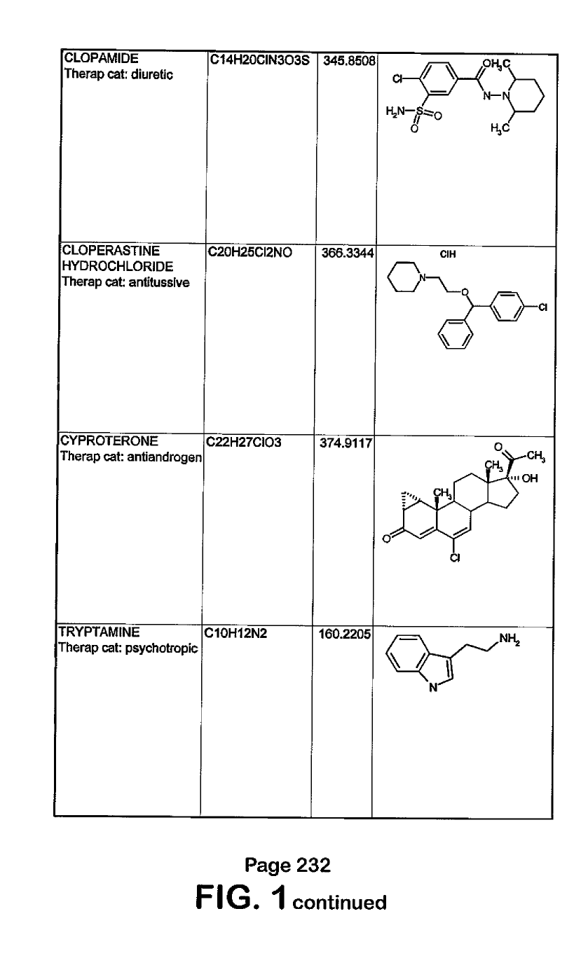

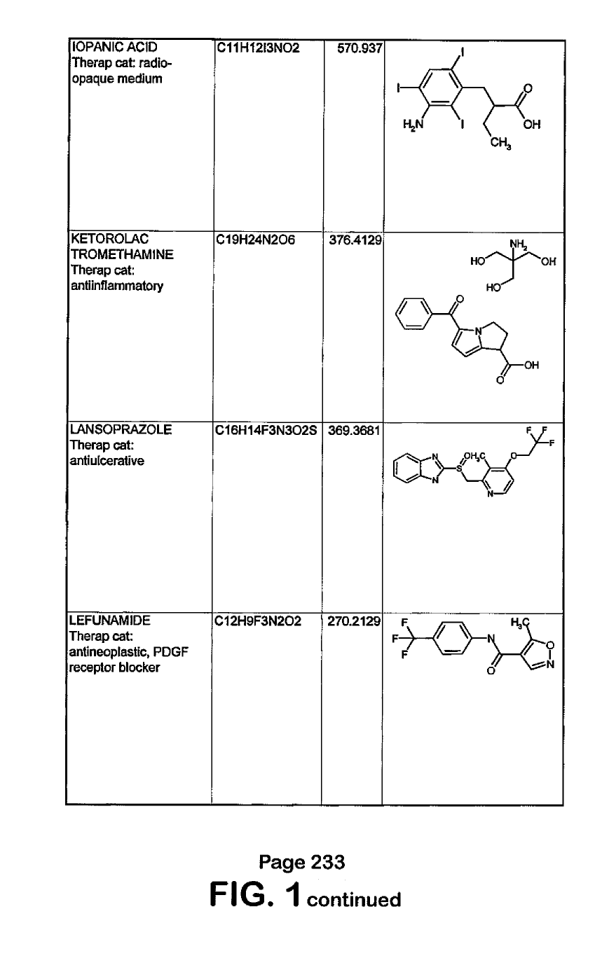

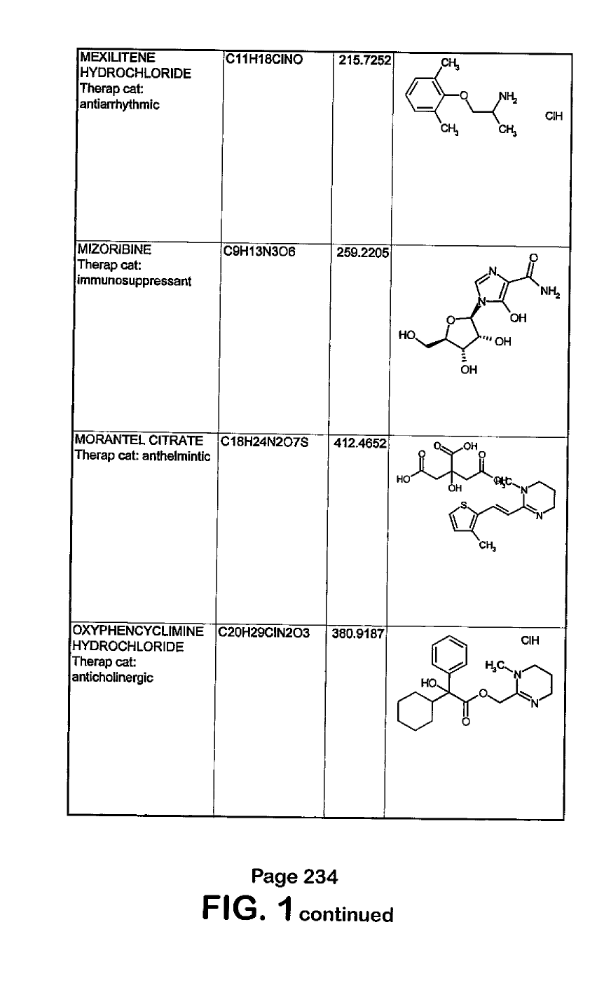

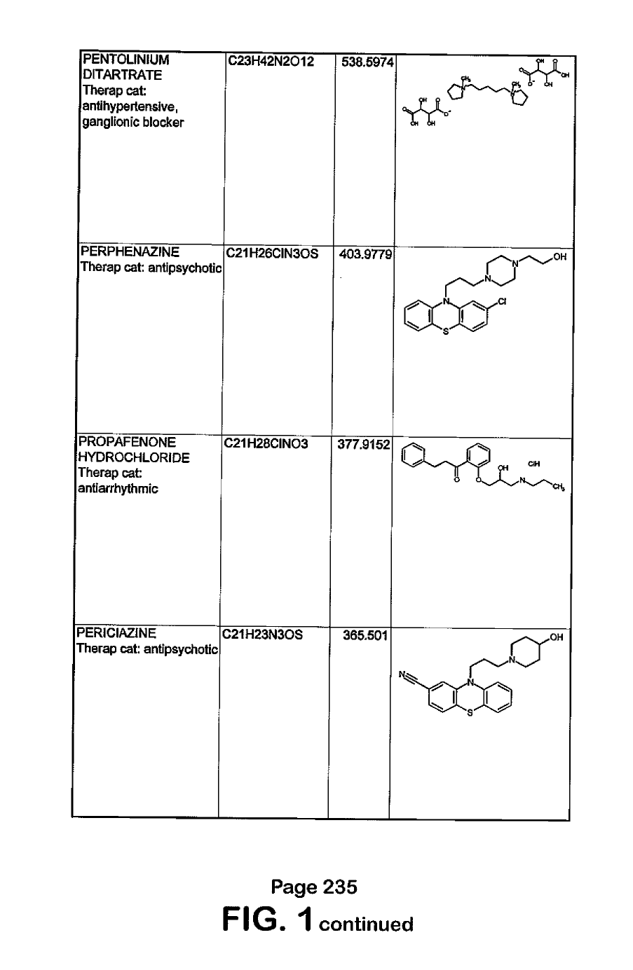

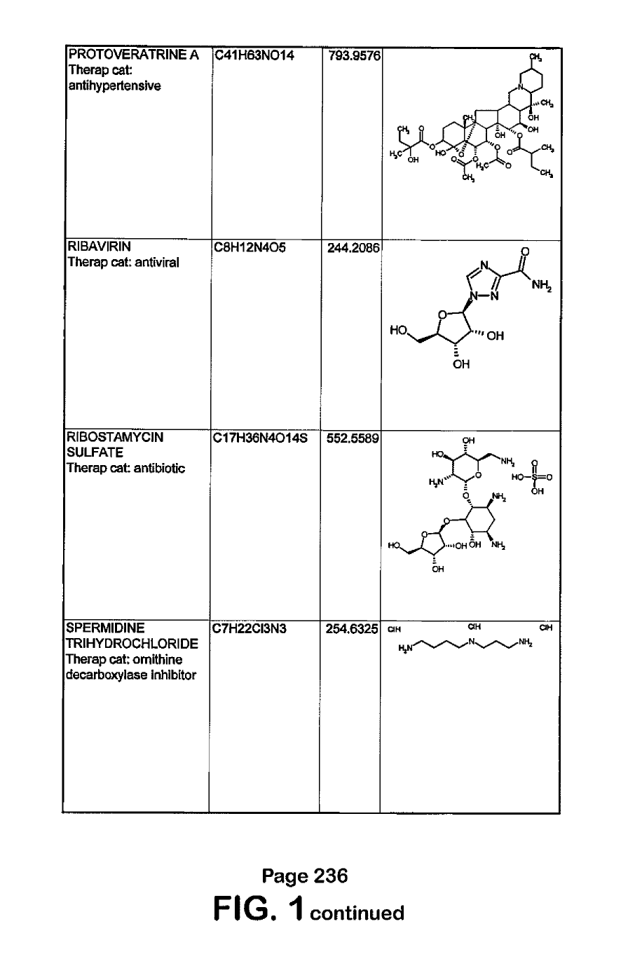

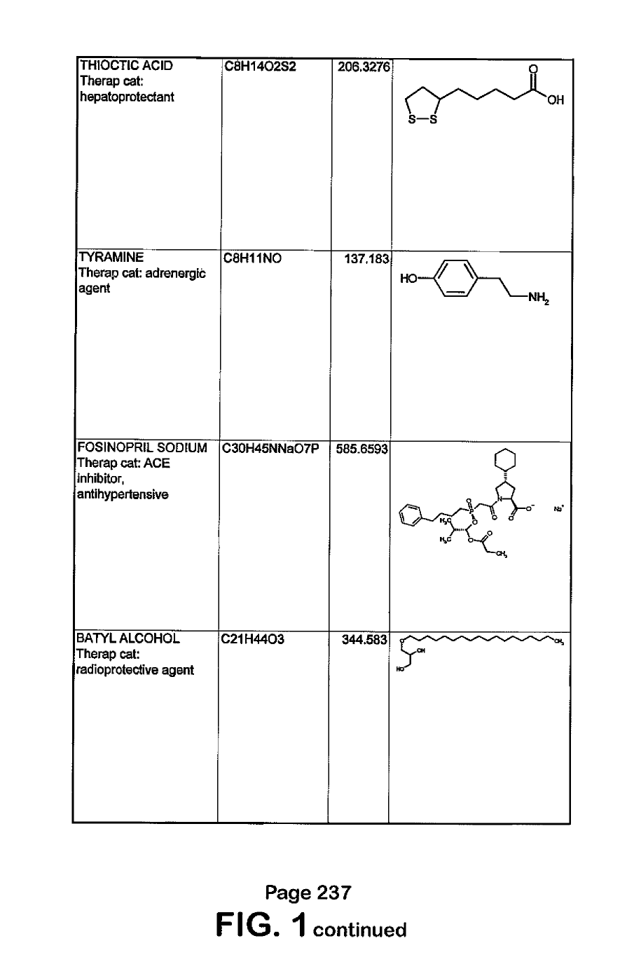

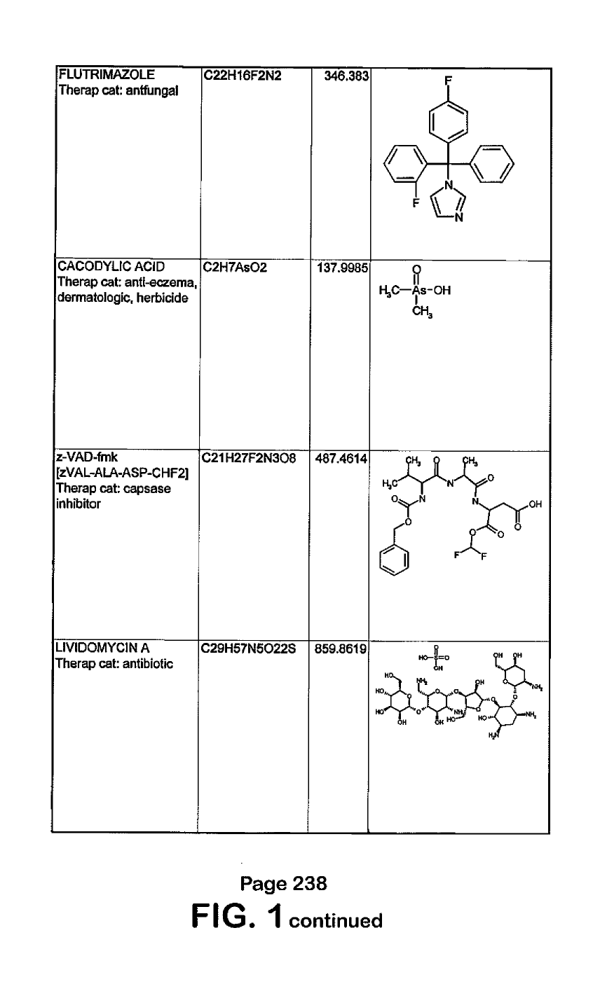

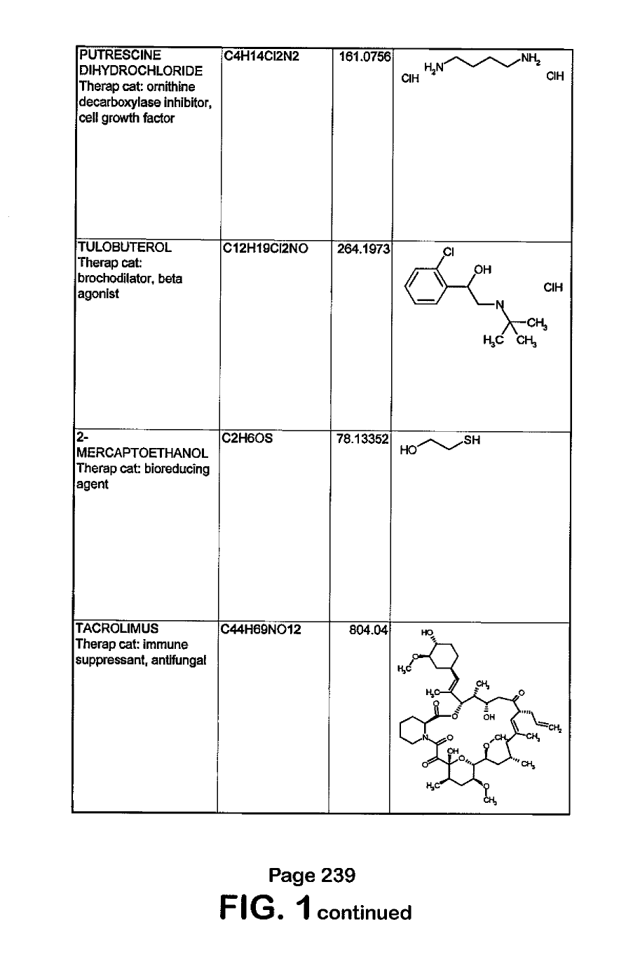

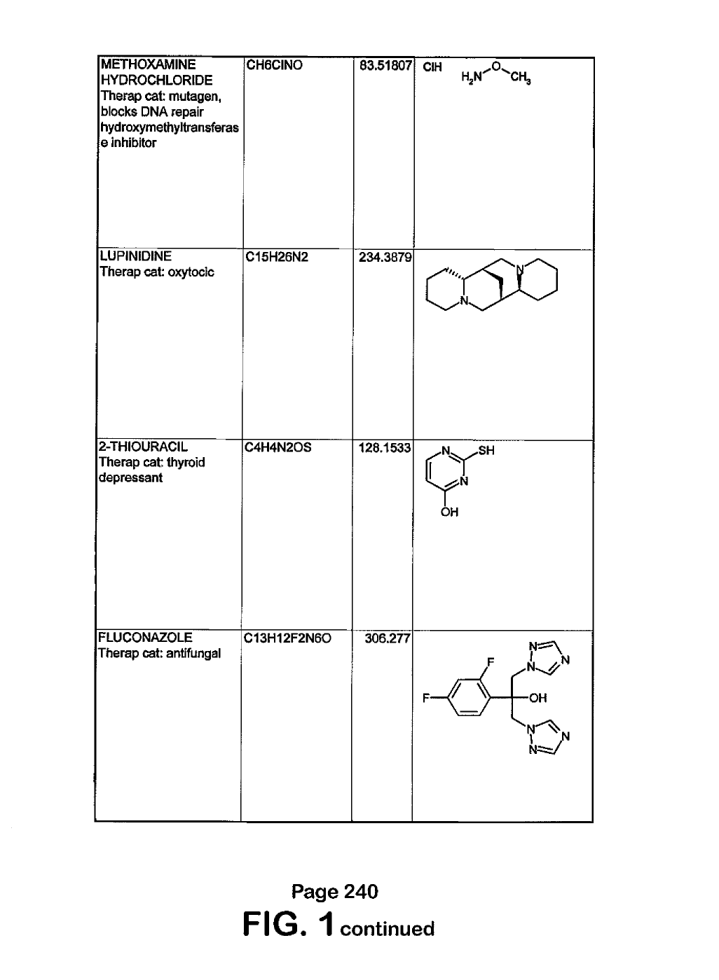

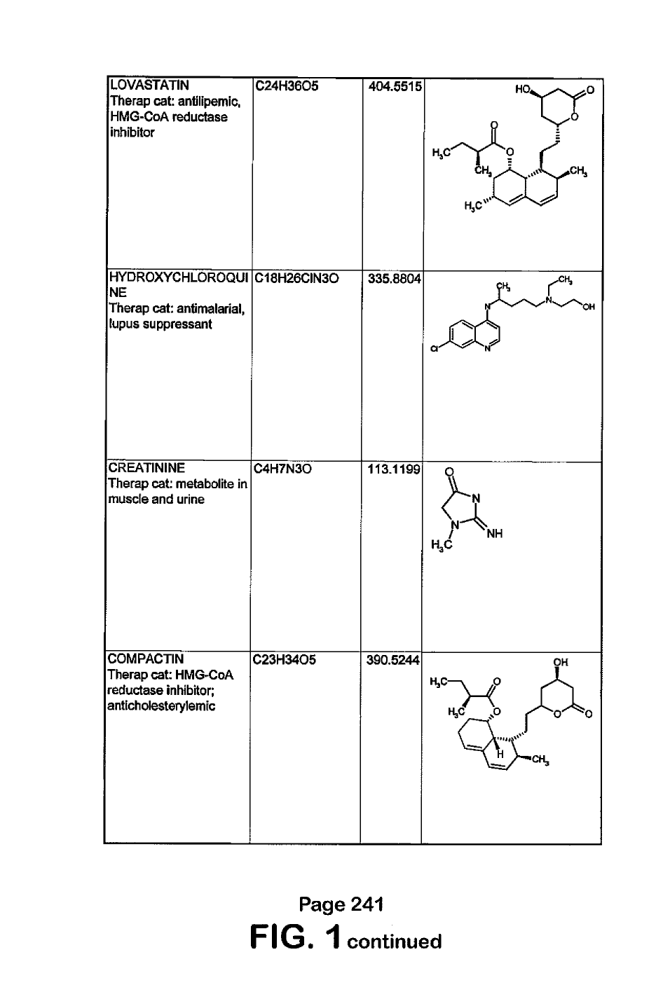

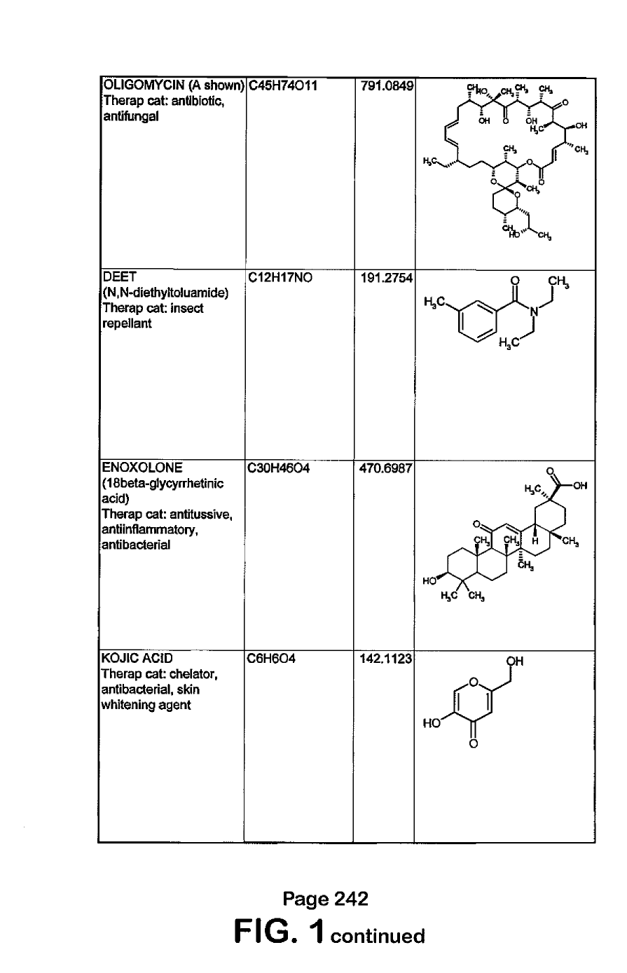

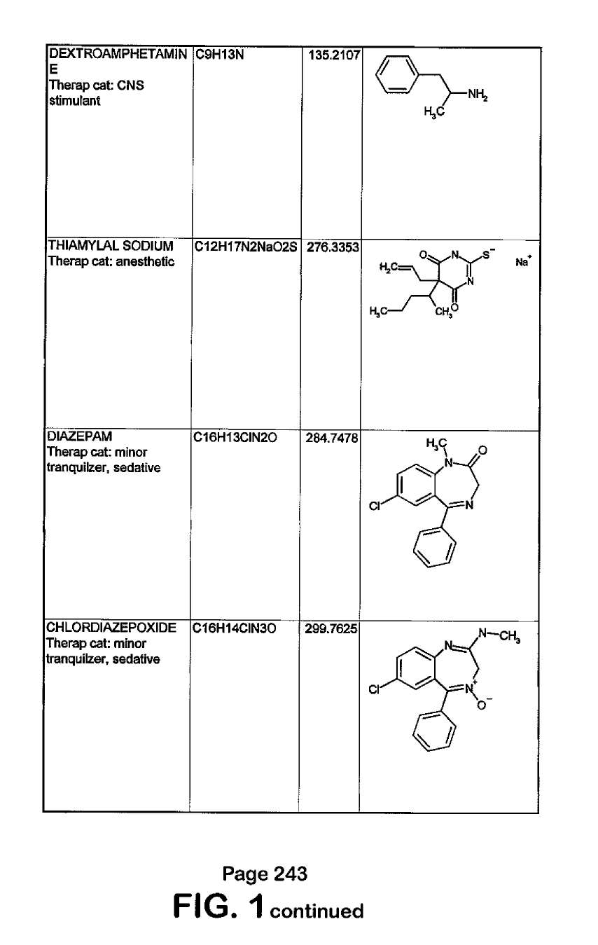

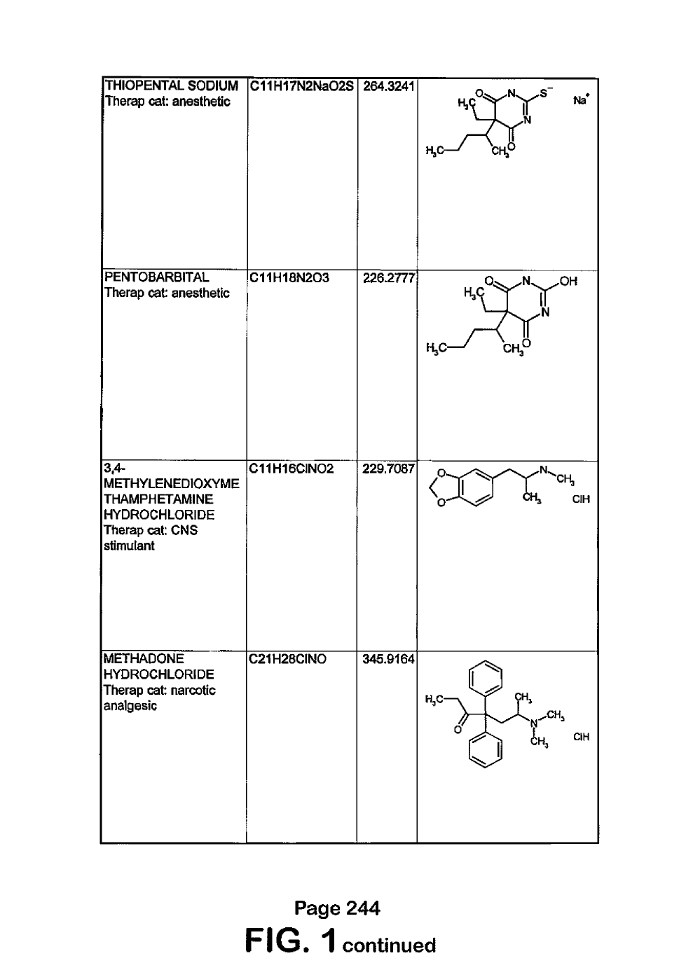

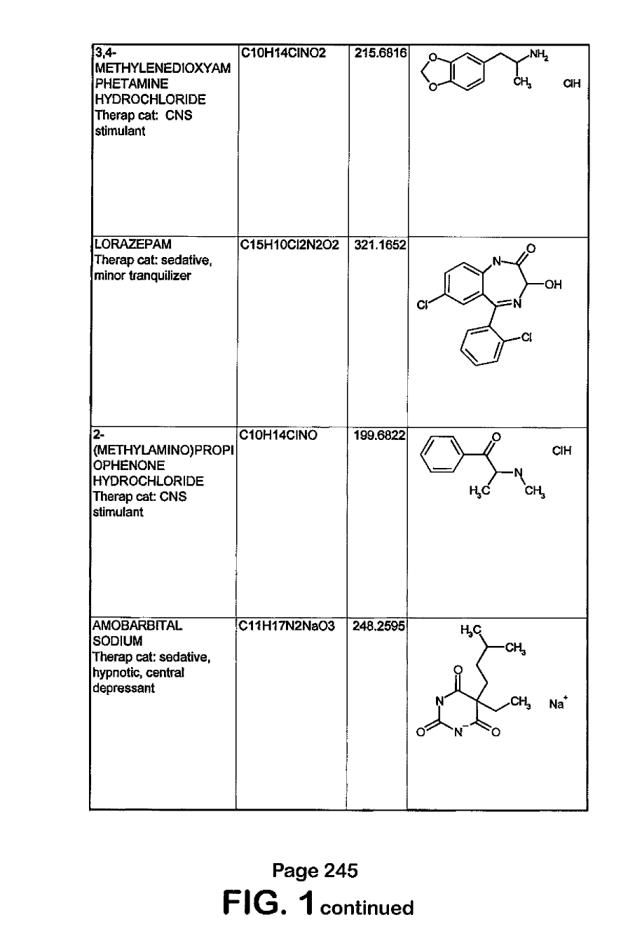

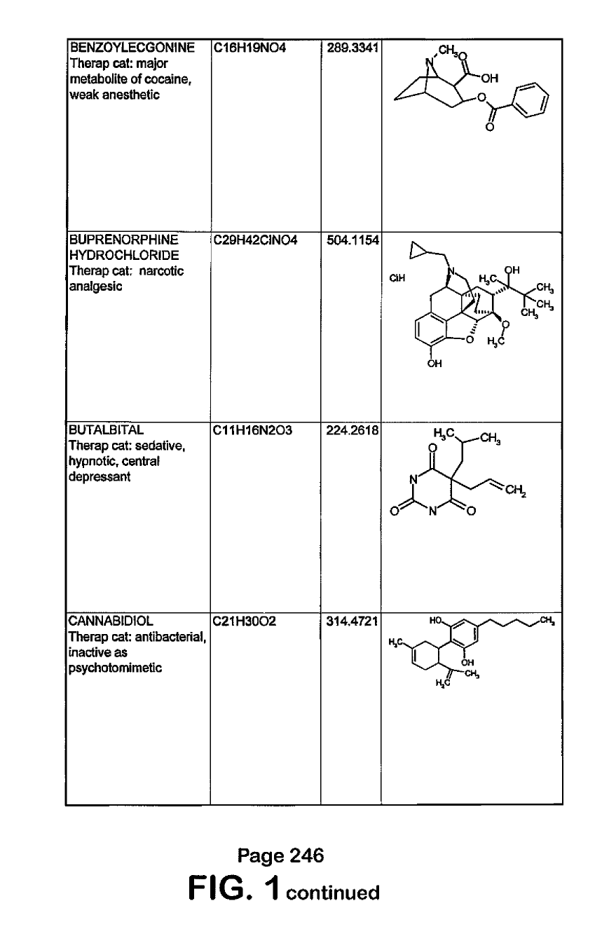

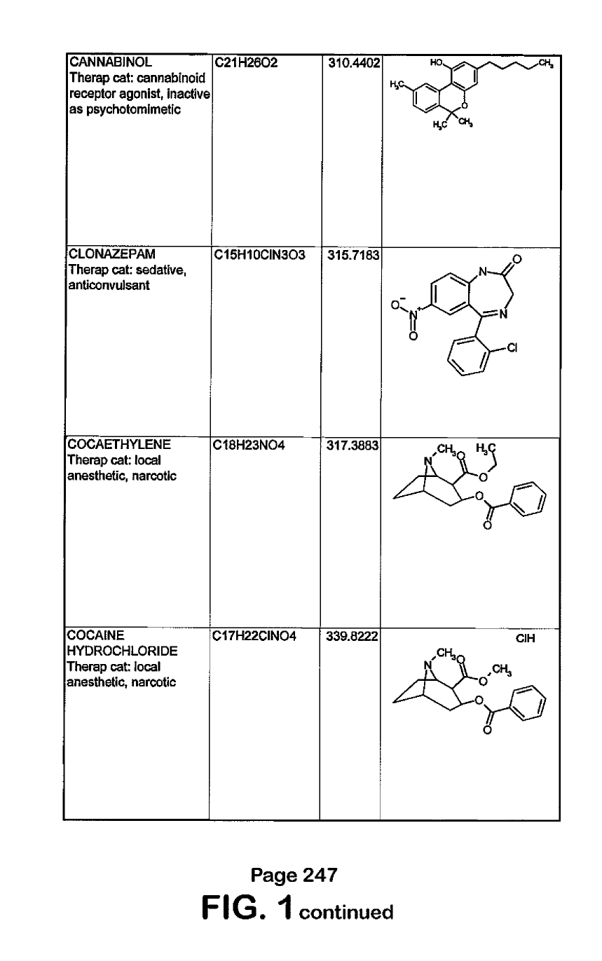

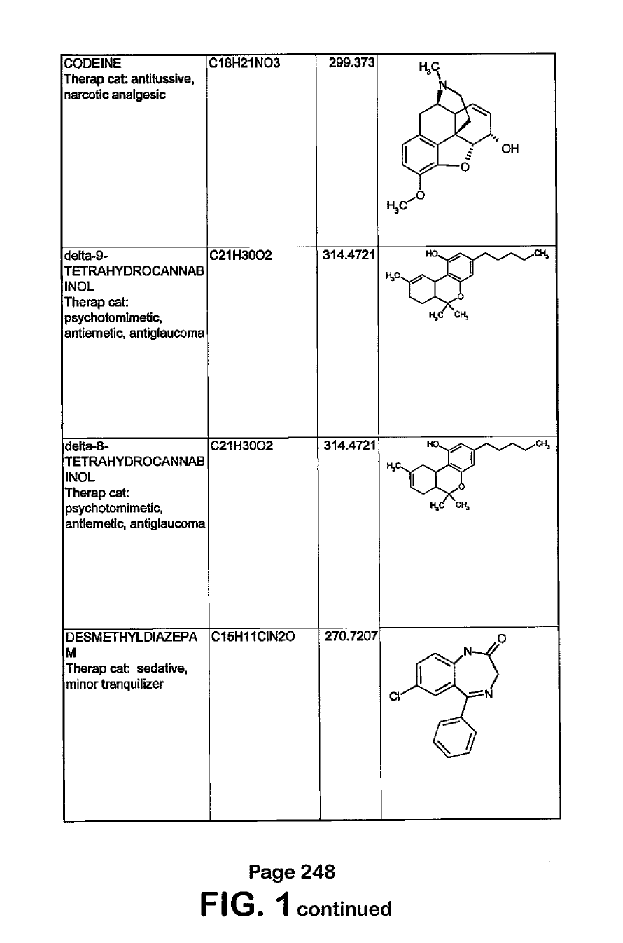

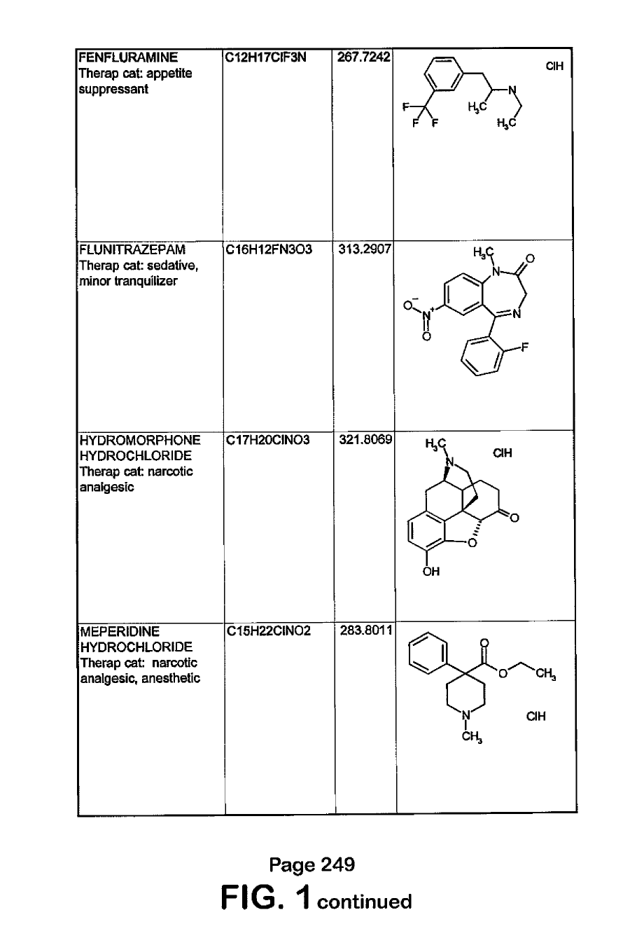

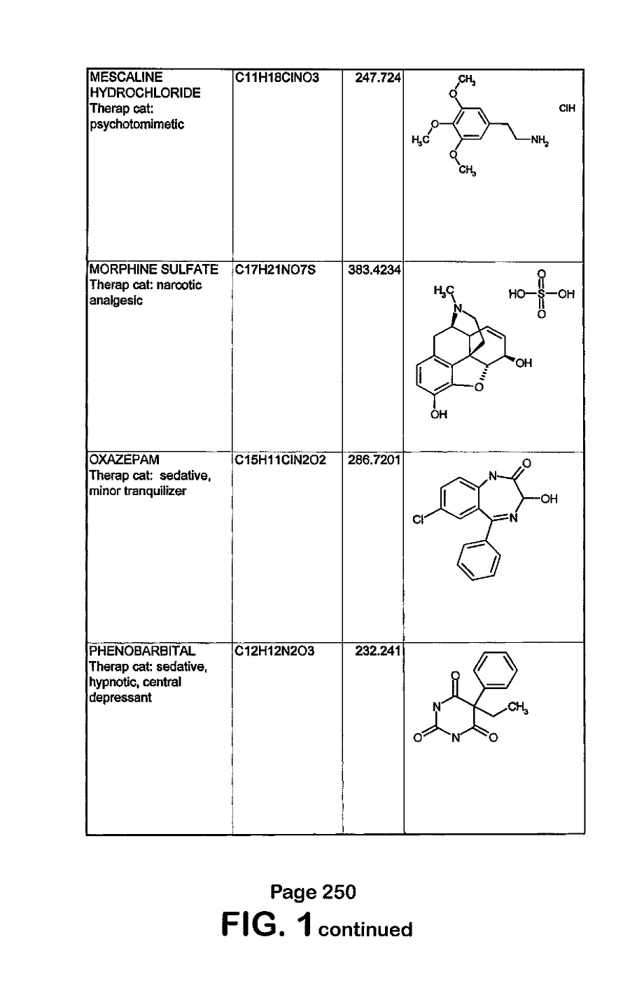

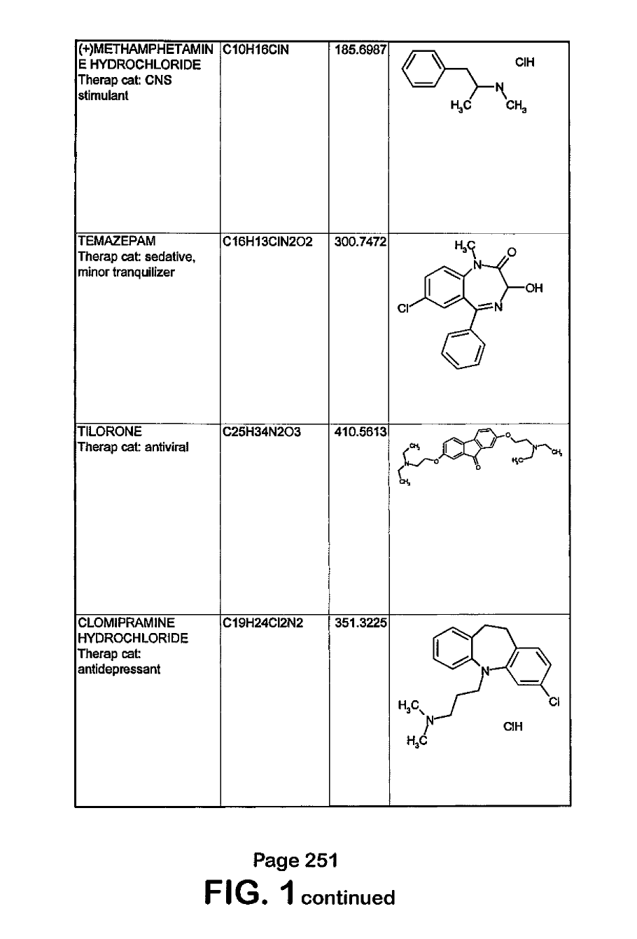

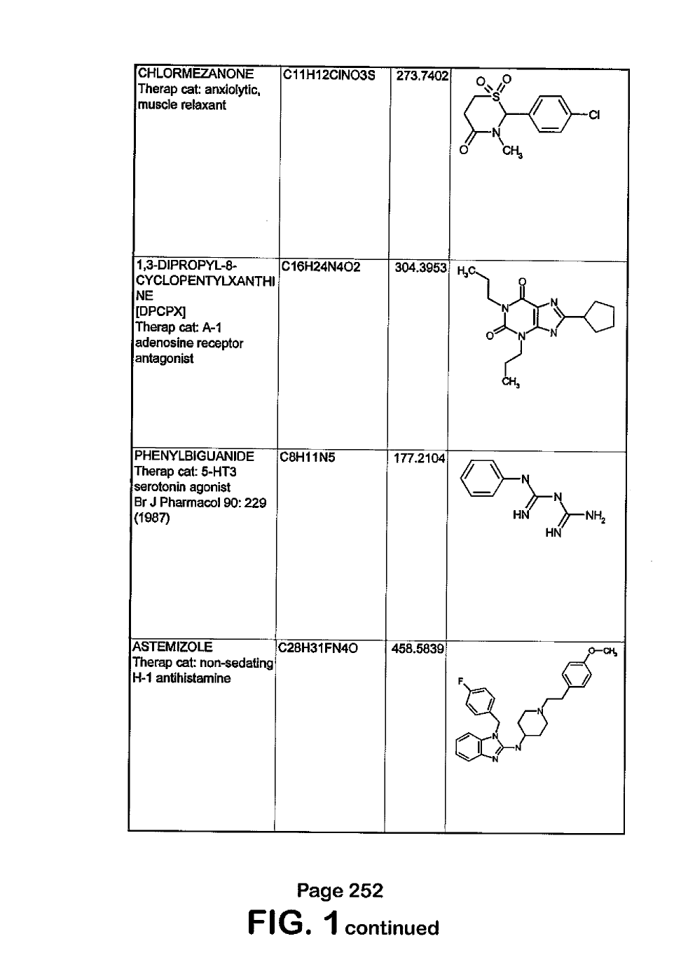

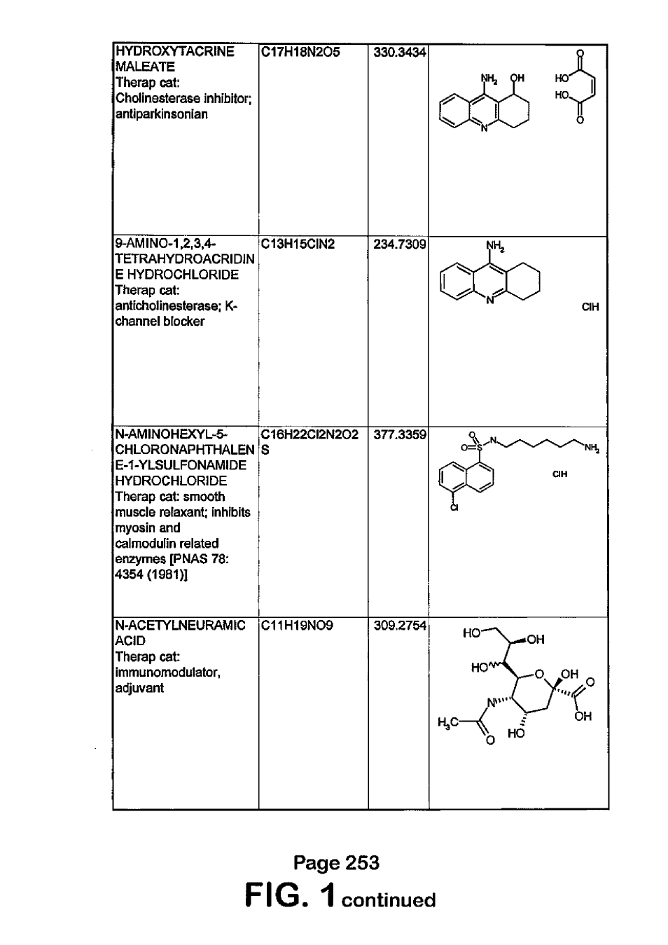

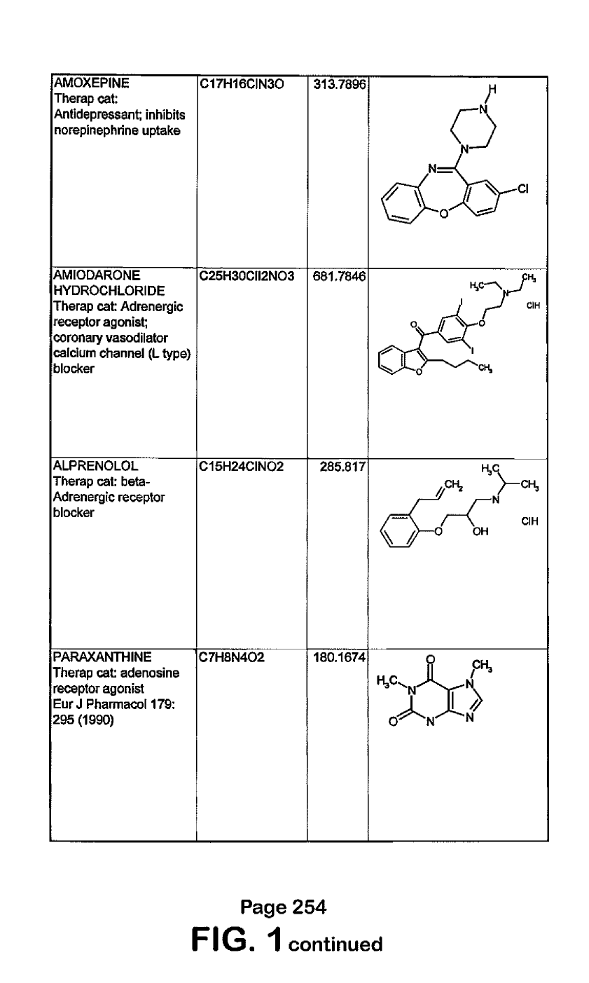

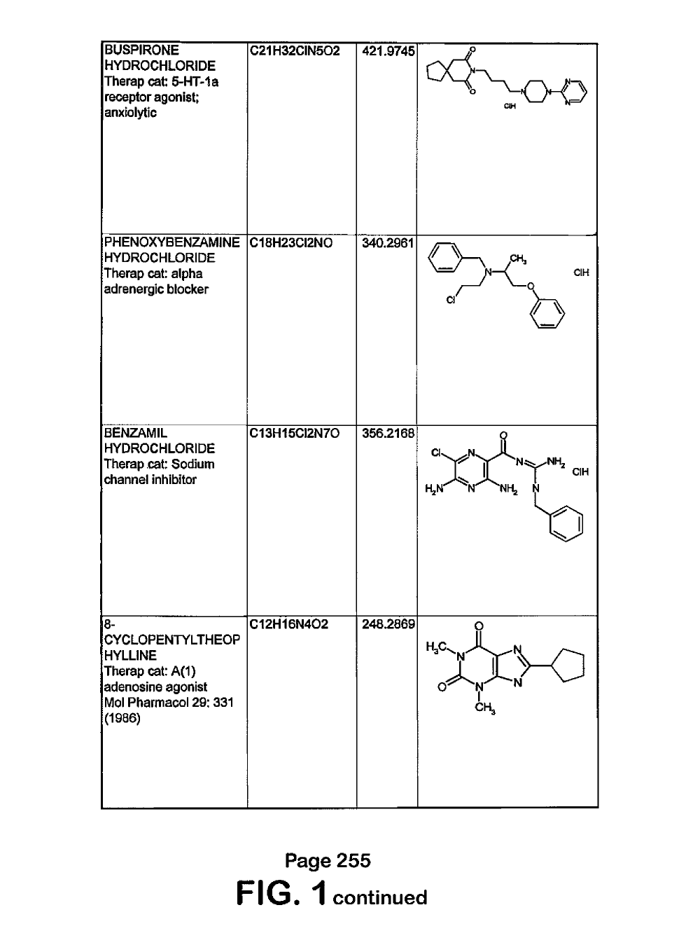

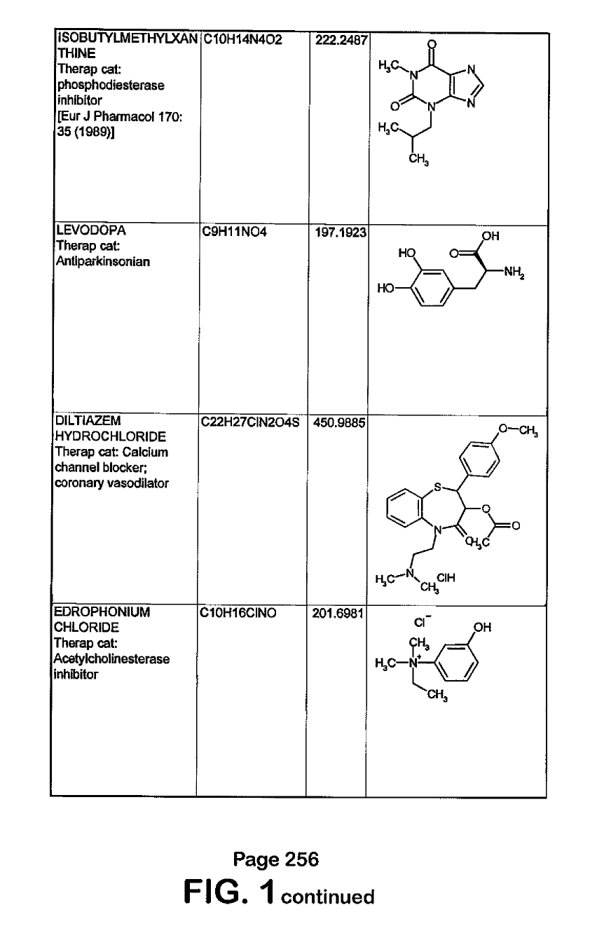

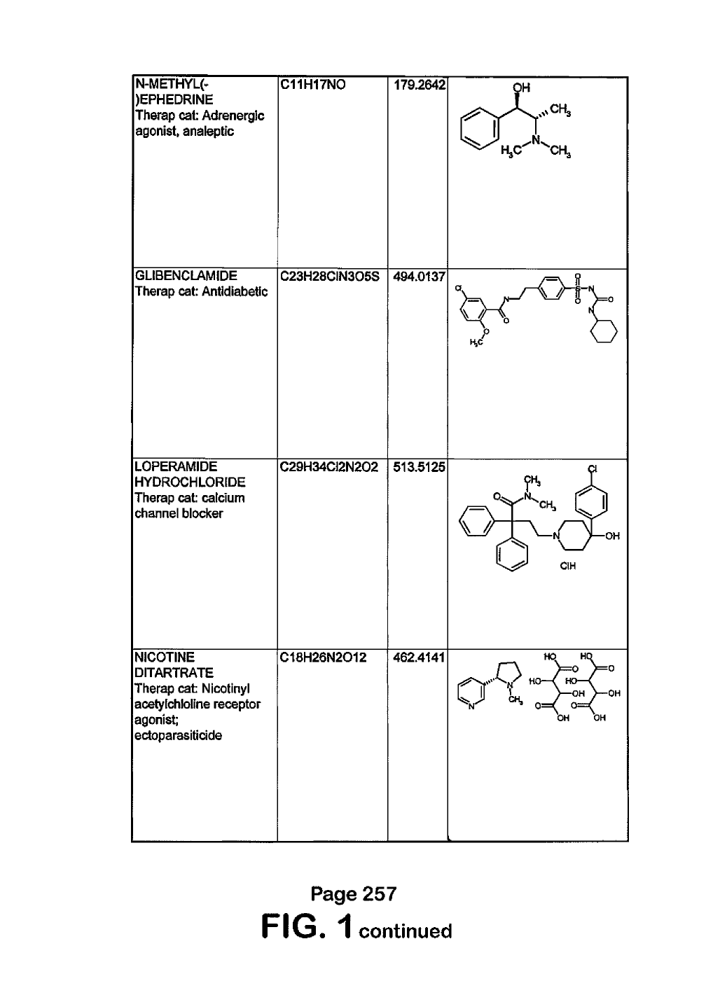

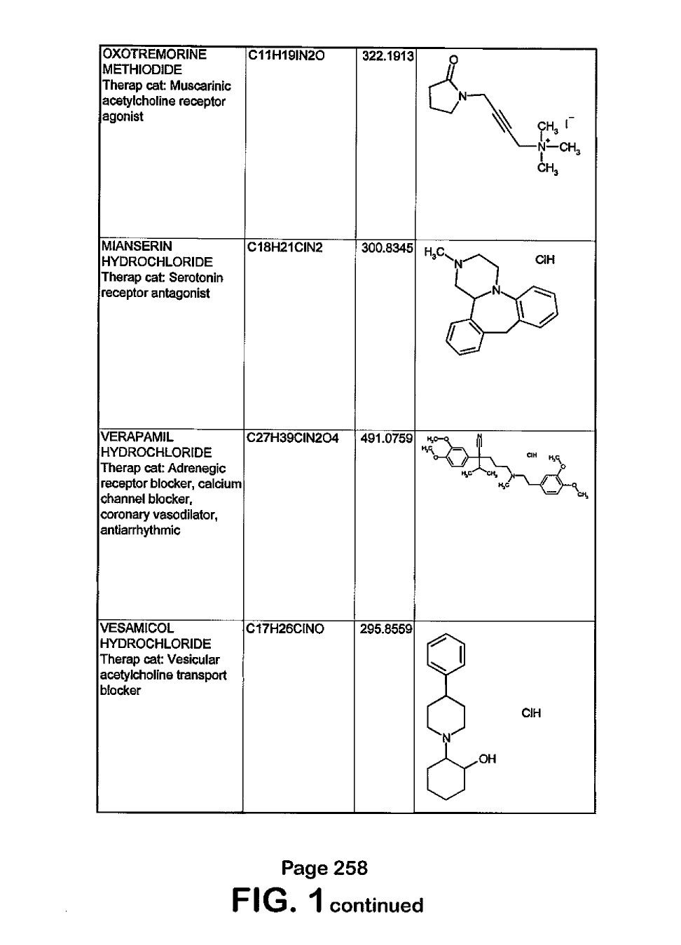

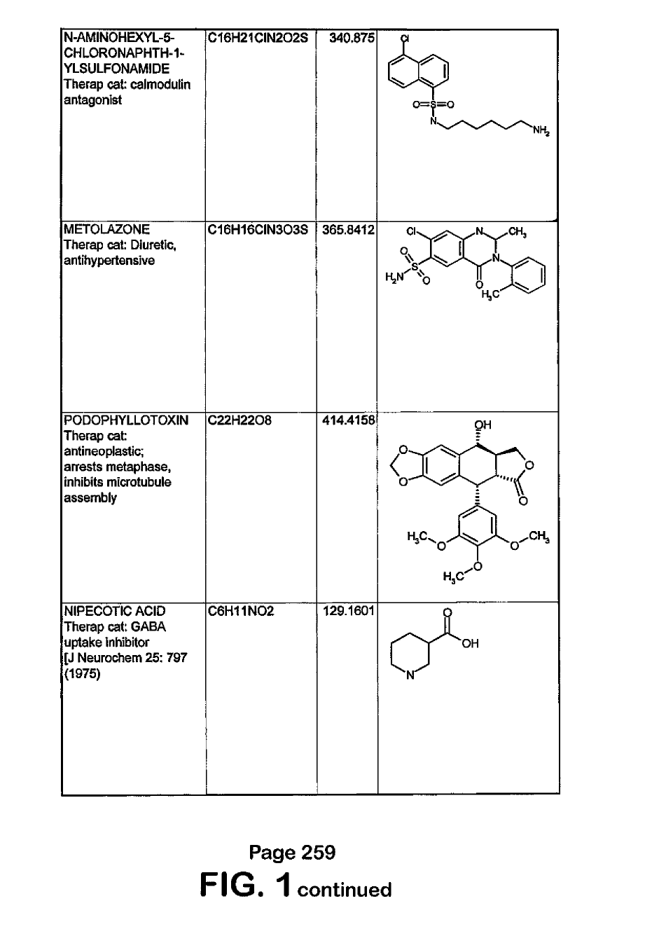

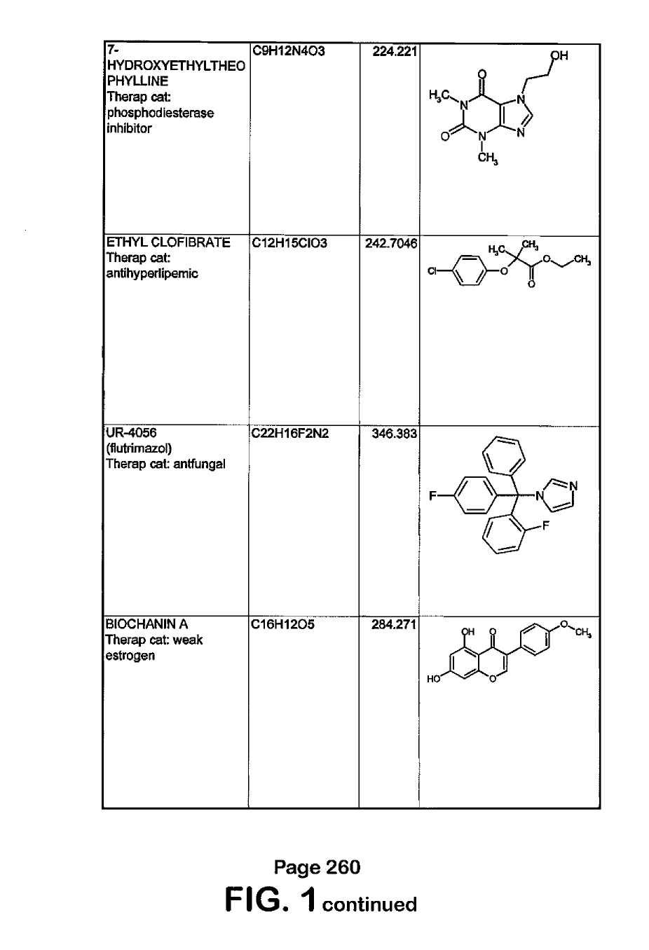

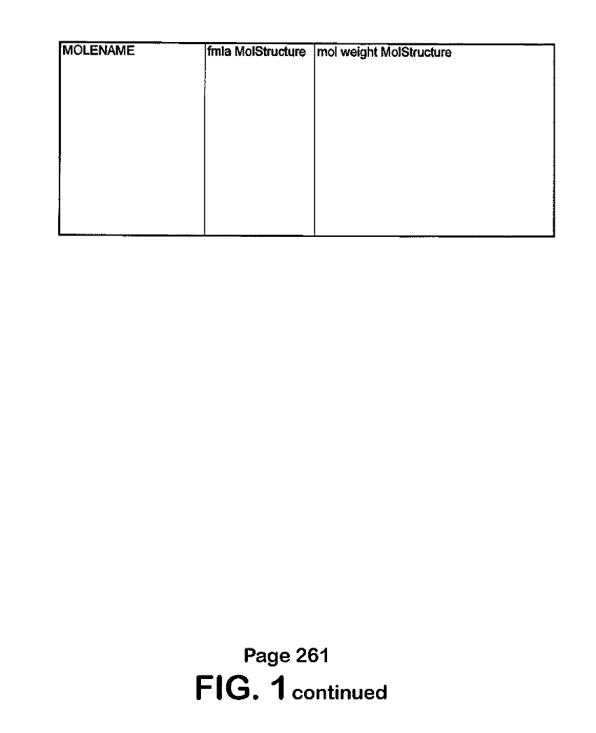

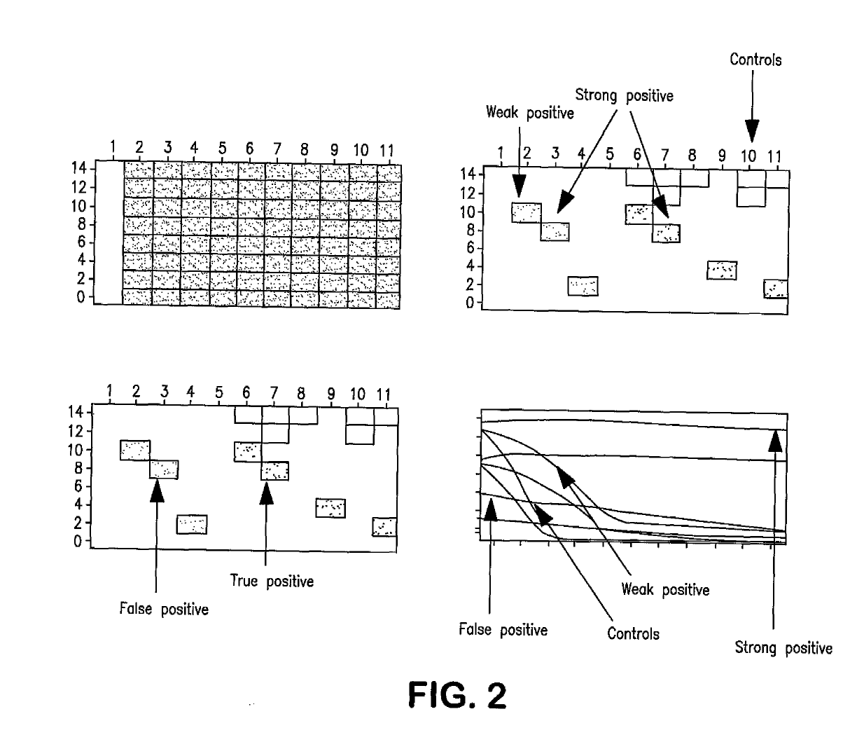

Given the potential clinical impact of drugs that Inhibit PT, a library of 1040 bioactive compounds (see FIG. 1) were screened for their ability to delay PT induction in isolated rat liver mitochondria. This library was primarily composed of clinically-approved drugs that could readily be moved to late stage pre-clinical trials and then into accelerated clinical trials. See Heernskerk, J., et al., Trends Neurosci., 25 (2002) 494-496. The remainder of the library largely consisted of some analogues of these drugs, other known bioactive agents (including natural products, toxins and known inhibitors). Screening of this library was blind and was designed to identify compounds that acted independently of antioxidant activity or ability to chelate Ca²⁺ (FIG. 2). Of the compounds showing “moderate” protection in the initial screen, two were known PT inhibitors, tamoxifen and trifluoperazine (both “positive controls”).

Primary and follow-up screens identified potentially useful compounds that were PT Inhibitors at 10 μM (FIG. 2). Primary screens were done using a previously described high throughput adaptation of a standard spectrophotometric assay based on PT-induced swelling following a Ca²⁺ challenge, see Kristal, B. S., et al., J. Biol. Chem 274:23169-75, 1999. Assays were performed on mitochondria energized with succinate, glutamate/malate, or α-ketoglutarate. In the initial pass, drug effects were scored as strong protection (basically no change in absorbance), moderate protection (visually observable delay in loss of absorbance), or no protection. Basic scoring of the screen is presented in FIG. 2. The top left is a view of a screening plate showing raw data; top right are seven compounds of interest and six controls are highlighted for demonstration purposes. This view shows a change in absorbance from the starting point and has advantages for identifying compounds that are relatively weakly protective at the doses tested; bottom left depicts the change to absolute absorbance and highlights the true vs false positives; bottom right are expanded views of one example of each class (note that all studies were conducted simultaneously at two wavelengths to avoid wavelength specific effects due to compound absorption). Subsequent assays (see, Table 1) repeated this approach using inducers optimized to overcome protection mediated by chelators (high C²⁺ or antioxidants (high tert-butyl hydroperoxide), based on the rationale that it was unlikely to identify novel drug activities in these categories.

TABLE 1

| | | | Additions | | Strong | Moderate |
|---|---|---|---|---|---|---|
| Screen | Description | Purpose | to buffer | Substrates | Inhibitors | Inhibitors |
| 1 | Low Ca²⁺/ | Primary | 10 μM | Succinate | 14/8 | 28/36 |
| | High PO₄ | screen | (succinate) | Glutamate/ | | |
| | | | or 20 μM | Malate | | |
| | | | CaCl₂ | α-keto- | | |
| | | | (glutamate/ | glutarate | | |
| | | | malate) | | | |
| | | | 25 mM | | | |
| | | | KPO₄ | | | |
| 2 | High Ca²⁺/ | Remove | 50 μM | Succinate | 11/6 | 14/26 |
| | Low PO₄ | chelators; | CaCl₂ | Glutamate/ | | |
| | | Remove | 750 mM | Malate | | |
| | | false | KPO₄ | α-keto- | | |
| | | positives | | glutarate | | |
| 3 | Low Ca²⁺/ | Remove | 10 μM | Succinate | 11/4 | 14/23 |
| | High ROS | anti- | CaCl₂ | Glutamate/ | | |
| | | oxidants | 400 mM | Malate | | |
| | | | KPO₄ | α-keto- | | |
| | | | 50 μM tert- | glutarate | | |
| | | | butyl | | | |
| | | | hydro- | | | |
| | | | peroxide | | | |
| 4 | Respiration | Remove | None | Succinate | 11/4 | 13/15 |
| | Test | strong | | Glutamate/ | | |
| | | respiratory | | Malate | | |
| | | inhibitors | | α-keto- | | |
| | | | | glutarate | | |
| 5 | 4- | Remove | ADP, | Glutamate/ | 10/4 | 13/10 |
| | parameter | uncouplers | CaCl₂ | Malate | | |
| | study | | | | | |

These studies reveal that these compounds are robust inhibitors of PT (they inhibit against multiple inducers) and they do not act by chelating Ca²⁺ (or they would not protect equivalently against high Ca²⁺ challenge). The protection against high doses of the oxidant tert-butyl-hydroperoxide indicates that these compounds are unlikely to be acting either as general antioxidants or as specific protectors of the dithiol moiety proposed as a PT regulator (termed the “S-site”, without wishing to be bound by theory, it is postulated to reside on the adenine nucleotide translocase, a postulated component of the transition pore), or the high dose oxidants would readily overwhelm protection.

When the blind was broken (referring to the blind study supra), it was determined that the strong compounds included a plate positive control (cyclosporin A) and compounds with known mitochondrial effects that were apparently not therapeutically useful. Of the compounds that had shown moderate protection, two were known PT inhibitors, tamoxifen and trifluoperazine (our other two “positive controls”).

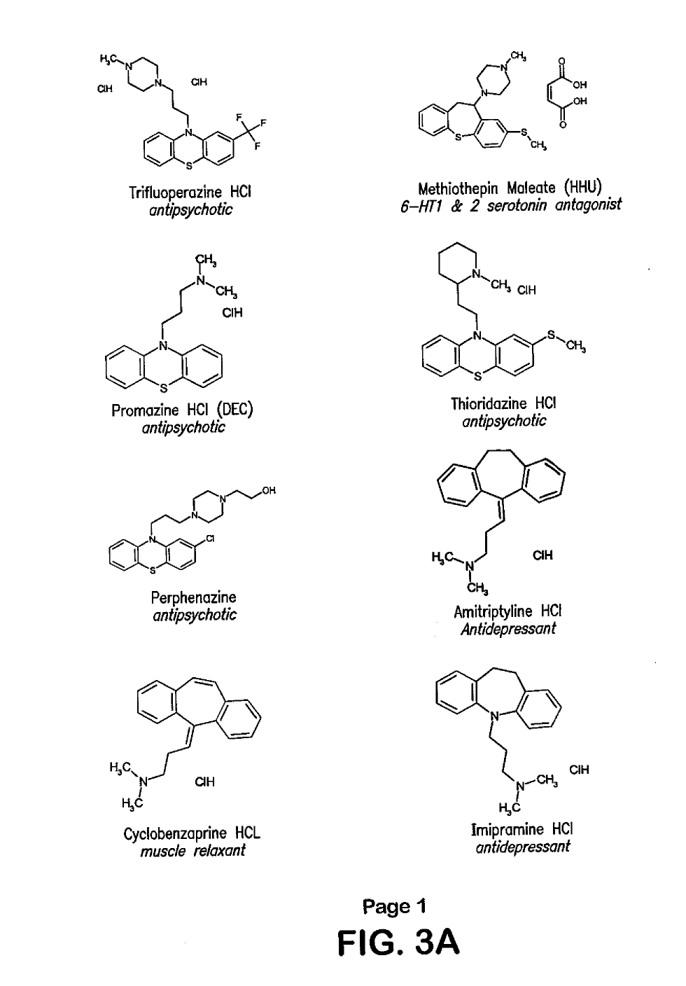

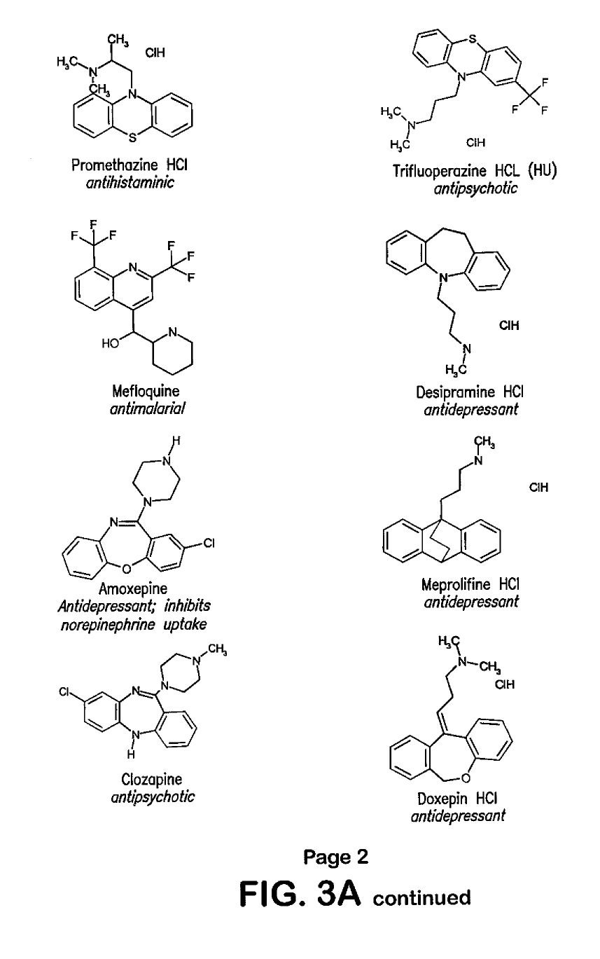

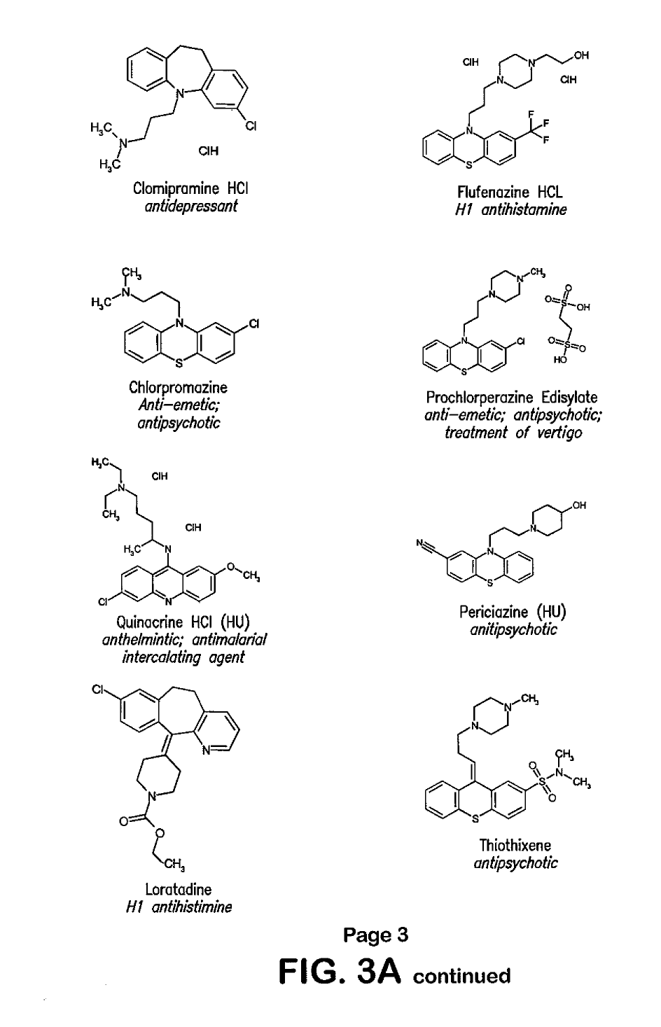

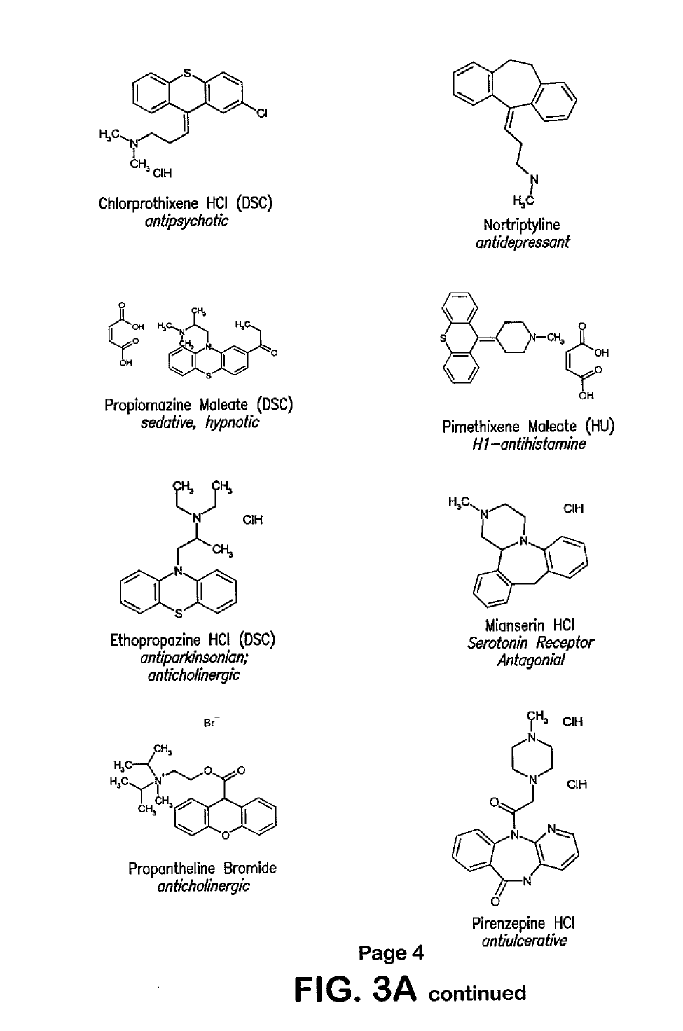

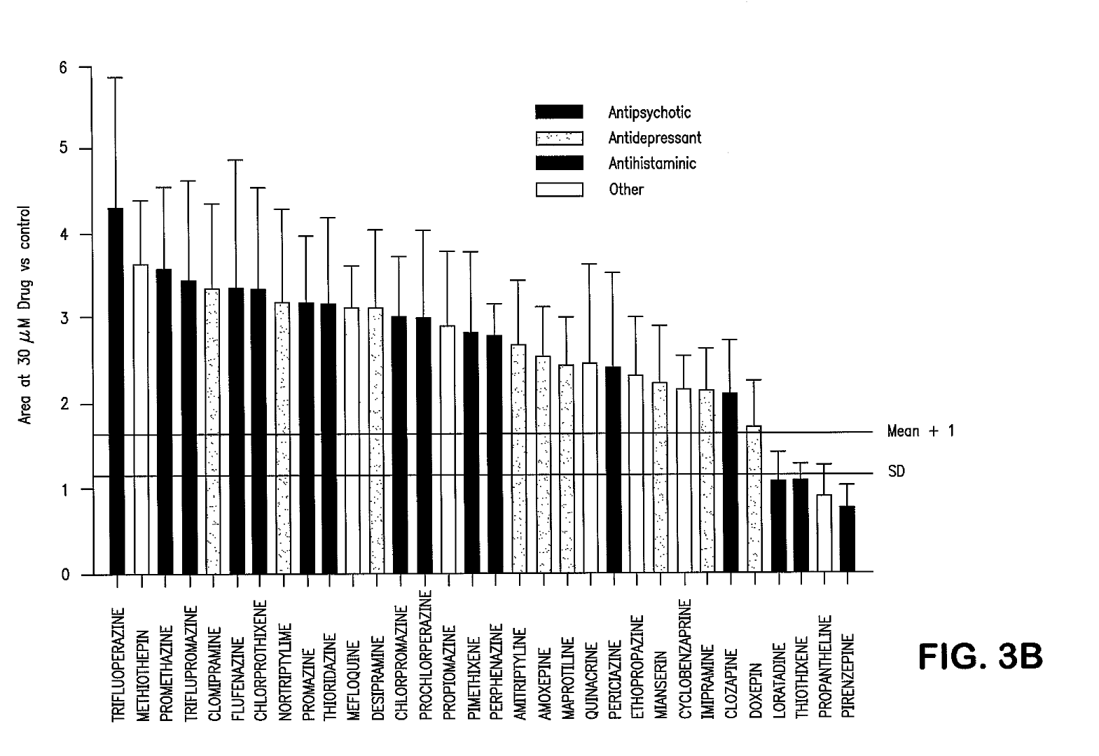

Inspection of the structures of the compounds that showed moderate protection revealed that trifluoperazine and additional compounds were from a specific subclass of heterocyclics and their structural analogues—including tricyclics/phenothiazines—a major class of psychotropic drugs used clinically since the 1950's. Retrospective analysis showed that some compounds in the library had a common chemical motif that includes or approximates the tricyclic/heterocyclic backbone (with either a six or seven member central ring, see, FIG. 3). Each of these compounds was re-assayed against three models of PT induction and ranked according to the resistance it conferred to induction. Of the experimental set, 28 of 32 compounds were protective at p<0.05; p values range down to 10⁻⁷, see Table 2 below. Of these compounds, 21 of 28 (75%) and 12 of 28 (43%) were protective at 10 and 3 μM, respectively. Searches of the scientific literature and the US FDA approved drug list indicate that 23 of these agents are in clinical use, and four others are approved but no longer in active clinical use. Four others were approved, at least for clinical trials, in at least one country. Only one (methiothepin) appears not to have been used in humans.

TABLE 2
A list of compounds that demonstrate
protection against PT induction.

| | | | |
|---|---|---|---|
| Trifluoperazine | Nortriptyline | Propiomazine | Periciazine |
| Methiothepin | Promazine | Pimethixene | Ethopropazine |
| Promethazine | Thioridazine | Perphenazine | Mianserin |
| Triflupromazine | Mefloquine | Amitriptyline | Cyclobenzaprine |
| Clomipramine | Desipramine | Amoxepine | Imipramine |
| Flufenazine | Chlorpromazine | Maprotiline | Clozapine |
| Chlorprothixene | Prochlorperazine | Quinacrine | Doxepin |

FIG. 3a depicts the drug structure, name, and therapeutic class of each heterocyclic-related structure in the compound library. Compounds are ranked best to worst (right to left, top to bottom) based on the data in panel B. As one measure of potential clinical utility, human use approval was examined using a list of US-FDA-approved drugs. Drugs currently in clinical use (23 of 32) have no abbreviations following their name. Where abbreviations follow the drug names they refer to: NHU (not used in humans 1 of 32); HU (used in humans, current status and countries of acceptance uncertain, 4 of 32); DSC (FDA-approved but believed discontinued, 4 of 32).

FIG. 3b depicts data showing the relative areas under the curve for the 32 heterocyclics studied at 30 μM. Bars are coded by each drug's therapeutic class. Minimal protection was observed at concentrations≦1 μM, and these data were pooled with the no drug controls to serve as the control data set. (Total control data set, N=1207, area relative to control: 1.14±0.48, mean ± SD). Mean coefficient of variation (CV) for individual experiments was 32%; mean CV within a specific challenge was 15%. Protection observed was statistically significant at p<0.05 for all agents (p values left to right, 0.0003, 0.00001, 0.00007, 0.0005, 0.001, 0.006, 0.002, 0.002, 0.0001, 0.0001, 0.000005, 0.0007, 0.00007, 0.002, 0.0005, 0.003, 0.0000008, 0.0004, 0.0002, 0.0002, 0.03, 0.02, 0.002, 0.001, 0.00006, 0.002, 0.005, 0.03, 0.9, 0.003, 0.06, 0.02). Compounds were assayed in triplicate at 7 concentrations (30, 10, 3, 1, 0.3, 0.1, and 0.03 μM) against three different challenges (50 μM Ca²⁺/2.5 mM K—PO₄, 25 μM Ca²⁺/5 mM K—PO₄, 25 μM Ca²⁺/2.5 mM K—PO₄/100 μM tert-butyl hydroperoxide). Protection (as area under the curve) at 10 μM (the concentration used in the initial screen) was found to be directly proportional to protection at 30 μM. To address potential inaccuracies introduced by calculation of the area under the curve, protection was also assayed as percent absorbance remaining when absorbance of an unprotected sample had dropped and stabilized. Protection assayed in this manner was proportional to that found by the area method. “N” for each drug is 7 to 9. Horizontal lines show mean and mean plus one standard deviation.

There is a wide range of potency for the compounds of the present invention Overall, the heterocyclics and related compounds described herein represent a series of structurally related inhibitors that can help elucidate PT. Of the group studied, 4 compounds are not true heterocyclics (one non-carbon atom in the “central” ring), suggesting the classical heterocyclic structure itself may not be mandatory. Mefloquine has only two rings, suggesting the three-ring backbone common in this class may be a reflection of the set of compounds tested rather than an obligate structural feature. Preliminary structure activity relationship (SAR) analysis, based on the final evidence that 28 of 32 heterocyclics and their structural analogues are protective vs 10 of 1008 non-heterocyclics indicates that the heterocyclic backbone's activity is highly significant within the intact screening set.

Also, one skilled in the art will appreciate that the compounds of the present invention that are effective against PT induction can serve to generate a pharmacophore.

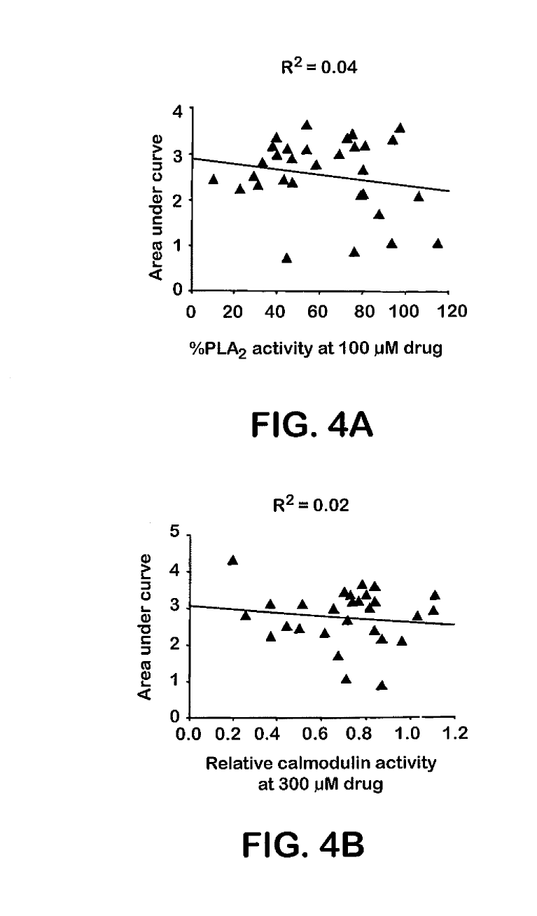

One heterocyclic, trifluoperazine, is identified as a PT inhibitor and has been previously shown to be protective against ischemia-reperfusion injury in multiple models. Trifluoperazine can inhibit both phospholipase A₂ (PLA₂) (see, Broekemeier, K. M., et al., Biochemistry, 34 (1995) 16440-49, the entire teaching of which is incorporated herein by reference) and calmodulin (Mehrotra, S., et al., Mol. Cell. Biochem., 124 (1993) 101-106, the entire teaching of which is incorporated herein by reference). Both of these activities have been hypothesized to be protective against PT. PLA₂ inhibition, in particular, has received substantial attention despite some evidence arguing it is not the mechanism by which trifluoperazine protects against PT. Literature analysis suggested that other tricyclics and phenothiazines might also inhibit phospholipase A₂ or calmodulin activity. Therefore, the capacity of each tricyclic in the library to inhibit PLA₂ or calmodulin was assayed. The ability to inhibit either PLA₂ or calmodulin did not correlate with the ability to inhibit PT in the compound set tested (FIG. 4).

FIG. 4 depicts data showing that the inhibition of phospholipase A₂ and calmodulin is not related to inhibition of PT induction. Specifically, in FIG. 4a, PLA₂ activity in presence of 100 μM compound (presented as percent of activity in absence of drug) of 31 of the heterocyclics was plotted vs protection against PT protection (as area under the curve at 30 μM), had a regression line y=2.9−0.006×, r₂=0.04. N=4−6 for each compound demonstrating the lack of correlation between lipase activity and protection against PT induction. FIG. 4b demonstrates the lack of correlation between calmodulin activity and protection against PT induction. Calmodulin activity in presence of 300 μM compound (presented as ratio of activity to untreated plate controls) of 28 of the heterocyclics was plotted vs protection against PT protection (as area under the curve at 30 μM), regression line y=3.1−0.46×, r₂=0.02. N=3 for each compound. Loratadine and pirenzepine showed apparent calmodulin activation and were not plotted.

The ability of the heterocyclics to inhibit phospholipase A₂ was determined based on a modification of the method of Meshulam, et al., J. Biol. Chem., 267:21465-70, 1992, the entire teaching of which is incorporated herein be reference. Briefly, the phospholipase A₂ substrate Bis-BODIPY-glycerophosphocholine (bis-BODIPY FL C11-PC) is incorporated into mitochondrial membranes and causes fluorescence self-quenching. Separation of the fluorophores on hydrolytic cleavage of one of the acyl chains by phospholipase A₂ results in increased fluorescence. The reaction buffer contained sucrose (310 mM), HEPES (3 mM), CaCl₂ (40 μM, K—PO₄ (2.5 mM), succinate (5-10 mM) or glutamate/malate (5 mM), bis-BODIPY FL C11-PC (0.1 ml), and mitochondria (1 mg/mL). For determining a compound's action on phospholipase activity mitochondria were treated with the compound in concentrations from 0.3-300 μM. The kinetics of the fluorescence was monitored for 120 minutes at an excitation of 488 nm and emission of 530 nm. The data obtained indicated that protection against mitochondrial permeability transition is independent of phospholipase activity.

The ability of the heterocyclics to inhibit calmodulin function was assessed by examining the effect of these agents on calmodulin-dependent calcineurin activity. Calcineurin activity was determined by following the absorbance of the product of calcineurin/PP2B-induced hydrolysis of calcineurin-specific substrate p-nytriphenyl phosphate (pNPP) in the presence of cofactors (e.g., Ca, Ni, calmodulin). The specific enzyme activity represents the inverse of the mass of calmodulin required to give half the maximal reaction rate. Determination of calcineurin/PP2B activity included two steps—enzyme activation and the enzymatic reaction. For the activation step, calcineurin, calmodulin 1 μg, CaCl₂ (0.1 mM), NiCl (1 mM) and 1% bovine serum albumin were incubated for 15-30 minutes at 37° C. The Enzymatic reaction step was started by the addition to the reaction mixture calcineurin substrate (pNPP) and further incubated for 10-30 minutes at 37° C. for color development Absorbance was monitored at 405-410 nm. For determination of a test compound's action on calcineurin/PP2B activity, individual heterocyclic compounds were added to the reaction mixture during the step of enzyme activation in concentration from 0.3 to 300 μM. The data obtained indicated that protection against mitochondrial permeability transition is independent of calmodulin.

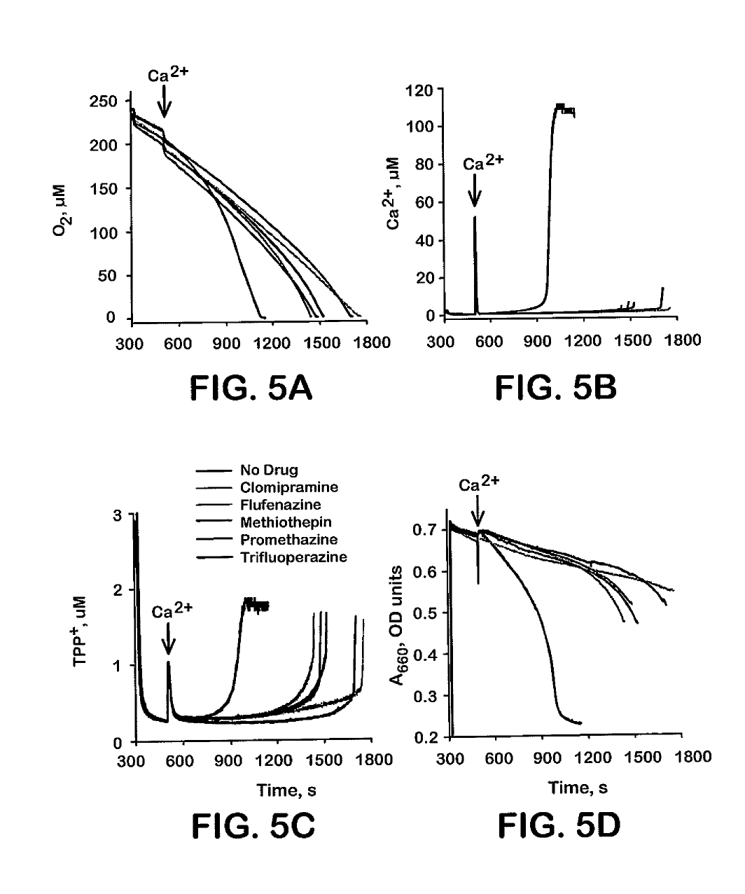

Mitochondrial physiological parameters were assessed to determine whether these heterocyclics and related compounds interfered direly with mitochondrial functions, and whether such interference might underlie their protection (e.g., respiratory inhibitors can score as protective in some assays, as can uncouplers, but neither would be optimal as a therapeutic candidate). Representative data from sties under conditions favoring PT induction are shown in FIG. 5. Compounds shown represent some of the structural and functional diversity present in the heterocyclics (flufenazine and promethazine [antihistaminics]; methiothepin [serotonin modulator]; clomipramine [antidepressant]).

FIG. 5 illustrates that the heterocyclic compounds delay PT induction in isolated liver mitochondria without impairing mitochondrial physiology. FIG. 5a depicts oxygen consumption measured by Clark electrode. Oxygen concentration in buffer decreases as the signal decreases. FIG. 5b depicts Ca²⁺ transport measured by Ca²⁺— selective electrode. The Ca²⁺ in buffer increases as the signal increases. FIG. 5c depicts Δψ measured using a TPP⁺ electrode. The Δψ parameter decreases as the signal increases. TPP⁺ uptake determined by comparison with standard curve after correction for shift in the electrode signal is induced by the compounds examined. Mitochondria appear to take up equal levels of TPP⁺ in the presence and absence of drug and restore TPP⁺ and Ca²⁺ levels after recovering from Ca²⁺ pulse, suggesting that these drugs have minimal or no direct effect on Δψ. Changes in Δψ consistent with the slight increases in respiration (equivalent to approximately 5% of maximally uncoupled mitochondria) observed with some drags is likely below the limit of detection. FIG. 5d depicts mitochondrial swelling (absorbance) monitored by a light emitting diode BET) at A₆₆₀. Mitochondrial are more swollen when signal decreases. Assay in 300 mM sucrose, 2.5 mM K—PO₄, 3 mM HERPES, pH 7.2 and 5 mM succinate. Experiments were stopped shortly after oxygen was consumed.

At 10 μM, these compounds protected against PT induction (bottom right of FIG. 5) without affecting initial oxygen consumption prior to PT induction (top left), Ca²⁺ transport (top right), or resting or recovered Δψ (bottom left). The addition of the high Ca²⁺-dose rapidly induces PT in the control sample, but the drug treated samples are largely protected. These compounds (i.e., flufenazine, methiothepin, and clomipramine) have many of the different structural elements present in the heterocyclics. Preliminary SAR analysis has revealed that the heterocyclics backbone's activity has a p value of <10⁻²⁰ (based on Chem-Tree analysis) within the intact NINDS dataset. This suggests that a useful group of inhibitors have been identified and that these compounds can be used as probes to better understand the relationships between in vivo and in vitro activity.

Liver mitochondrial respiration was measured in 250 mM sucrose, 3 mM MgCl2, 0.5 mM EDTA, 10 mM KH₂PO₄ (pH 7.2). The buffer was equilibrated at 28° C. for not less than an hour before taking measurements. Respiration was initiated by adding mitochondria isolate to the oxygraph cell (0.9±0.2 mg/mL protein). State 3 respiration was initiated with ADP (˜200 μM). The substrates were added at either 5 or 10 mM for α-ketoglutarate and glutamate/malate and 10-20 mM for succinate. Respiration rates were determined using a PC-controlled Strathkelvin Instruments Limited 928 6-Channel Dissolved Oxygen System with two standard Clark-type electrodes fitted to two standard Gilson 1.7-1.8 mL oxygraph cells. Traces were analyzed using the Strathkelvin data acquisition and analysis program included with the instrument. The lines of best fit were calculated and plotted to polarographic traces using the built-in software function based on least squares regression analysis. State 3 respiration rates were calculated when stabilized, i.e., 1.0-1.5 minutes after initiating the state. Respiration before and after ADP consumption was used to calculated RCR. All experiments were carried out in triplicate. All substrates used in mitochondrial respiration experiments were dissolved in respiration buffer (see below) and brought to neutral pH.

The experimental system employed comprises a respiration chamber that can be sealed and placed in a light tight outer chamber. Electrodes and sensors are positioned in a single plane around the outside of the chamber—this system enables simultaneous measurement of oxygen uptake, membrane potential, calcium, and light scattering (absorbance). Oxygen uptake, membrane potential, and Cab are measured using Clark, TPP⁺, and Ca²⁺-sensitive electrodes, respectively see, Zhu, S., et al., Nature 417:74-78, 2002. A₆₆₀ is measured using a diode. The chamber is water-jacketed to allow thermostabilization (15-to-1 water volume to sample volume −1 mL, closed chamber). Samples were constantly stirred using a top-mounted magnetic stir bar. Signals measured from electrodes and sensors are recorded on a computer through a commercially available L-154 card. Software has been written to enable data collection and analysis. All experiments were carried out in triplicate. Plots shown in the paper are representative.

Heterocyclic-mediated protection was next examined in two models of stroke: (i) oxygen-glucose deprivation (OGD) of cultured primary cerebrocortical neurons, and; (ii) in the mouse, middle cerebral artery occlusion (MCAO)/reperfusion.

Middle cerebral artery occlusion/reperfusion studies were conducted as previously described. See, Friedlander, R. M., et al., J. Exp. Med., 185 (1997) 933-40, and Plesnila, N., et al., PNAS USA, 98 (2001) 15318-23, the entire teachings of which are incorporated herein by reference. All animal experiments were conducted in accordance with National Institutes of Health and institutional guidelines. Male C57/B6 (Charles River Laboratories) mice weighing 18-29 g were housed in standard temperature (22±1° C.) and light-controlled (light on 07:00-21:00) environment with ad libitum access to food and water. Weight of animals undergoing stroke were restricted to less than 22 g. Animals undergoing stroke were randomized to two groups: untreated OP injection with 0.9% saline) and treated (IP injection with promethazine solution). Weight of animals undergoing sham operations was liberalized to 18-29 g.

Surgery and Occlusion: Immediately before surgery, each mouse was anesthetized with 2% isoflurane (70% N2O/30% O2). During surgery, isoflurane was lowered to 1%. A midline incision was made in the neck and the right common carotid artery was dissected. The right MCA was occluded with a 7-0 nylon filament after permanent ligation of the external carotid artery and temporary ligation of the common carotid artery. See, Hara, H., et al., PNAS USA, 94 (1997) 2007-12, and Friedlander, R. M., et al., J. Exp. Med., 185 (1997) 933-40, the entire teachings of which are incorporated herein by reference. Right MCA occlusion was defined as greater than or equal to a 90% drop of peak velocity measured by the laser Doppler flowmetry drop. See, Huang, Z, et al., Science, 265 (1994) 1883-85, the entire teaching of which is incorporated herein by reference. The occluding filament was removed after 120 minutes. Sham operations consisted of the same duration and concentration of anesthesia, midline incision, doppler probe insertion over right temporal bone, and dissection of common carotid artery but no manipulation of the common carotid artery or external carotid artery. The surgeon was bided to treatment group. All mice were sacrificed at 24 hrs post perfusion. Experiments were in accordance with protocols approved by the Harvard Medical School Animal Care Committee.

Drug Treatment: Both treated and control groups received two IP injections: the first one delivered 1 hr prior to ischemia and the second delivered 12 hrs later. Each i.p. dose was 10 mg/kg (in 0.5 ml) of promethazine dissolved in 0.9% NaCl or an equal volume of buffer alone. See, Galeotti, N., et al., Neuroscience, 109 (2202) 811-818, the entire teaching of which is incorporated herein by reference. Determination of Infarct Volume: After 24 hrs of reperfusion, the mice were sacrificed. The brains were quickly removed and chilled for 2 minutes. The cerebellum was removed, Coronal sections (2 mm thick; n=6) were cut in a craniotome. Each slice was immersed in a saline solution containing 20 mg/cc 2,3,5-triphenyltetrazolium chloride (Sigma) at 37° C. for 30 min. After staining, each slice was scanned by an HP scanjet 4200 C, The unstained areas in each hemisphere were quantified and the ischemic area per slice=left (normal side)-right (ischemic side). See, Hara, H., et al., PNAS USA, 94 (1997) 2007-12; Yrjanheikki, J., et al., PNAS USA, 96 (1999) 13496-500; and Swanson, R. A., et al., J. Cereb. Blood Flow Metab., 10 (1990) 290-93, the entire teachings of which are incorporated herein by reference. The infarct volume was calculated by summing up the infracted areas in the 6 slices and calculating the volume for a cylinder (area×height).

Neurological Examination scores: Scores follow Bederson et al., Stroke, 17 (1986) 472-76, the entire teachings of which are incorporated herein by reference, 0: no neurological deficits; 1: failure to extend the left forepaw; 2: circling to contralateral side; 3: loss of ability to walk or righting reflex. The examiner was blinded to treatment group.

Neurotoxicity studies were conducted as previously described. See, Plesnila, N., et al., PNAS USA, 98 (2001) 15318-323, the entire teaching of which is incorporated herein by reference. Cerebral cortex of mouse embryos at day 15 (E₁₅) were freed from meninges and separated from olfactory bulb and hippocampus. Trypsinized cells were suspended in medium (neurobasal medium (NBM) with 2% (vol/vol) B27 supplement/2 mM glutamine/100 units/ml penicillin, streptomycin (Gibco) and seeded at a density of 2×10₄/cm₂ poly-lysin-coated dishes. Cells were used for extents on day seven of culture. Cell death was evaluated by the LDH release assay.

Promethazine was chosen as the proof of principle compound for three reasons: (i) higher tolerated dose: Trifluoperazine, the strongest PT inhibitor, is only used in humans at ˜10 mg/day, whereas promethazine is used at doses as high as 3 mg/kg (>15-fold higher for almost all individuals). Furthermore, trifluoperazine has limited utility for long-term use in humans because of its toxicity. Although not relevant for the short-term treatment required by stroke, this limitation would present complications for the treatment of chronic neurodegenerative diseases. Methiothepin, which is approximately equivalent to promethazine in potency, has not been used in humans; (ii) promethazine is a well-tolerated drug with few adverse side effects, and, as opposed to the heterocyclics that are primarily neurotropic, promethazine has comparatively minor neurological side effects; (iii) promethazine does not appear to be a strong PLA₂ or calmodulin inhibitor, and thus was also chosen in order to clarify the relationships between these two activities and the actions of our compounds of interest as potential PT inhibitors.

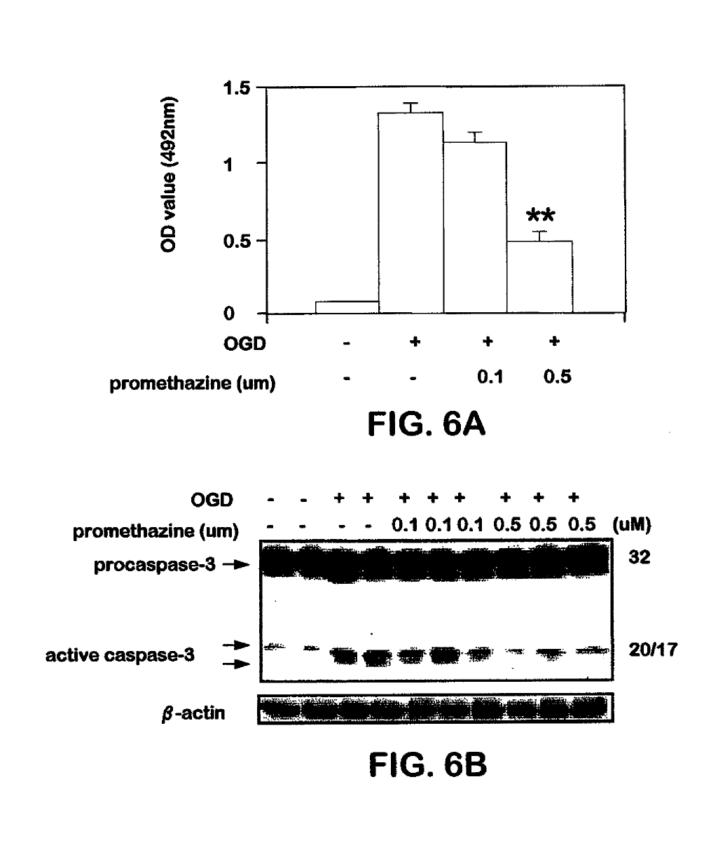

OGD of primary cerebrocortical neurons is a widely used cell culture model of stroke-related pathology. Promethazine inhibited OGD-mediated neuronal death, as determined by reduction of lactate dehydrogenase (LDH) release into the media. Inhibition of neuronal death was observed at su-micromolar concentrations of promethazine (0.1 μM, p=0.05; p=0.5 μM, p<0.001). See FIG. 6. Caspase-3 activation plays a key role in hypoxia/ischemia-mediated injury. To gam insight into the mechanism of promethazine-mediated neuroprotection, it was evaluated whether this compound could inhibit OGD-mediated caspase-3 activation. As expected from a PT inhibitor, promethazine-treated neurons exposed to OGD showed reduced caspase-3 activation, supporting the hypothesis that the drug interferes with caspase-mediated cell death.

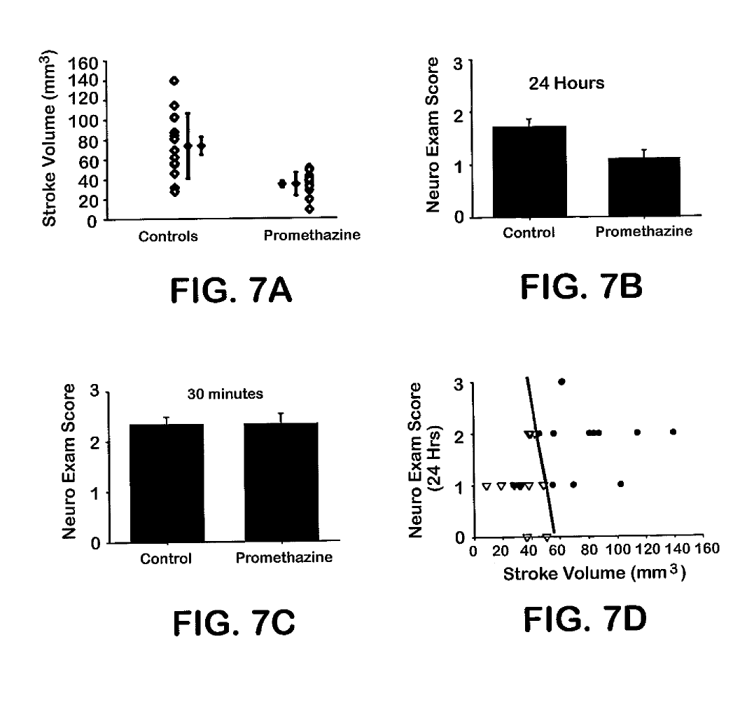

Since promethazine reduced OGD-mediated neuronal death, it was evaluated whether it could also ameliorate ischemic damage in vivo, in which activation of the PT and caspase-3 may play roles. Following MCAO, promethazine treated nice showed a 53% reduction in lesion size compared with saline-treated mice (n=13, p<0.005) (FIG. 7a). Both maximal ifarct size and variability of the injury were reduced 65%. Consistent with a reduction in ischemic damage, the neurologic impairment score of promethazine treated nice was significantly improved 24 hours following MCAO (p<0.05) (FIG. 7b). Cerebral blood flow (measured by a laser-doppler flowmeter) and systemic blood pressure did not differ between die treated and control groups. As expected from studies of other neuroprotectants, the neurologic score was not different at the 30 minute post-ischemia evaluation (FIG. 7c). (See, Hara, H., et al., PNAS USA, 94 (997) 2007-12; and Friedlander, R. M., et al., J. Exp. Med., 185 (1997) 933-940, the entire teachings of which are incorporated herein in their entirely by reference.) Protection of both neural function and structure was consistent across all individual mice in the study ((see, FIG. 7d), diagonal line added for emphasis).

These data are in accord with and significantly extend findings of other groups who have provided evidence linking the PT to excitotoxic and OGD injury in cultured neurons (Schinder, et al., J. Neurosci., 16 (1996) 6125-33; and Khaspekov, L., et al., Eur. J. Neurosci., 11(1999) 3194-98, the entire teachings of which are incorporated herein in their entirety) and to damage subsequent to ischemia/reperfusion in intact animals (Matsumoto, S., et al., J. Cereb. Blood Flow Metab., 19 (1999) 736-41, the entire teaching of which is incorporated herein by reference). The Matsumoto et al. and Khaspekov et al. studies are of particular note, as they used the N-Met-Val analog of cyclosporin A, which has been considered not to react with calcineurin. Retrospective literature analysis indicates that several Heterocyclics and related analogues had shown some protection against ischemia (Zivin, J. A., et al., Brain Res., 482 (1989) 189-93; Nakata, N., et al., Brain Res., 590 (1992) 48-52; and Karasawa, Y., et al., Psychopharmacology (Berl), 109 (1992) 264-70, the entire teachings of are incorporated herein in their entirety), PT (Rodrigues, T., et al., Br. J. Pharmacol., 136 (2002) 136-142; and Broekemeier, K. M., et al., Biochem. Biophys. Res. Commun., 163 (1989) 561-566, the entire teachings of which are herein incorporated in their entirety), or PT-like phenomena (Mehrotra, S., et al., Mol. Cell Biochem., 124 (1993) 101-06, the entire teaching of which is incorporated herein by reference), but that the linkages and the existence of a structural class with a common ability was not previously recognized and many of the compounds tested in these studies have significant side effects, cannot be used clinically at the appropriate doses, and/or cannot be used for long periods of time.

A retrospective literate search reveals the existence of several previously unconnected studies on these compounds that can now been brought together to help prepare these drugs for clinical deployment as neuroprotectants. The phenothiazines, chlorpromazine (thorazine) and trifluoperazine reduced functional impairment in rabbit models of cerebral and spinal ischemia (Nieminen, A. L., et al., Neurosci., 75 (1996) 993-97, the entire teaching of which is incorporated herein by reference). Clomipramine (20 mg/kg) has been shown to protect against hippocampal CA1 neuronal damage in a gerbil ischemia-reperfusion model. Protection was linked to inhibition of extracellular increases in excitatory amino acids (Matsumoto, S., et al., J. Cereb. Blood Flow Metab., 19 (1999) 736-41). Mianserin (5-20 mg/kg) also protects in the gerbil ischemia model, and protection was shown to be independent of effects on the serotonergic and cholinergic systems (Scheff, S. W., et al., J. Neurotrauma, 16 (1999) 783-792, the entire teaching of which is incorporated herein by reference). Quinacrine (mepacrine), a typical because of its aromatic central ring, has been noted as a protective agent in the heart during ischemia, but effects on PLA₂ were considered the critical mechanism. Histamine has been shown to cause swelling of liver mitochondria, and antihistamines to retard it (Zhu, S., et al., Nature, 417 (2002) 74-78, the entire teaching of which is incorporated herein by reference). Thioridazine has been suggested to inhibit apoptosis and delay PT induction by acting to reduce mitochondrial oxidative stress (Friberg, H., et al., J. Neurosci., 18 (998) 5151-59, the entire teaching of which is incorporated herein by reference).

While the mechanism of action against the PT remains enigmatic, it does not appear that known clinical (e.g., neurotransmitter modulation) or accompanying biochemical properties (e.g., PLA₂ or calmodulin inhibition) mediate the protection observed. Thus, the data is most consistent with the active pharmacophore being the basic class structure, rather than a specific subclass as defined either clinically (e.g., antidepressant), biochemically (e.g., PLA₂ inhibition) or structurally (heterocyclic).

These results appear to expand the potential clinical uses of the heterocyclics, tricyclics, phenothiazine and their structural analogs. These agents offer potential as long-term prophylactics for individuals at risk for stokes and heart attacks. They are also appropriate for long term use in individuals with neurodegenerative diseases, such as Amyotrophic Lateral Sclerosis (ALS), Parkinson's, Alzheimer's, and Huntington's diseases. The heterocyclics, tricyclics, phenothiazine are also appropriate for acute management of stroke and heart attack. Additionally, these compounds can also be used in spinal cord injury cases, traumatic injury to the brain or any other neurological insult, chemical toxicity, liver, muscle, kidney and alike reperfusion. The list of disease has not been exhausted here, however, one skilled in the art will appreciate the significance that these compounds have in treating diseases. As clinical mitochondrial protective agents, these drugs offer at least six major advantages: (i) most if not all of the compounds studied here are FDA approved, meaning that they offer the potential for rapid movement into pre-clinical and clinical trials as adjunct therapies for the disorders noted above; (ii) most if not all are known to cross the blood brain barrier, (iii) some of these drugs have been used for five decades, and most are safe for long term use (a notable exception is phenothiazine and trifluoperazine); (iv) they appear active close to the clinical range (the maximal therapeutic dose of promethazine is 3 mg/kg, and several have LD50s in animals 5-10 times higher than promethazine; (v) the multiple available series and clinical classes of these agents mean that there are potentially multiple agents that can be combined to minimize unwanted side effects or maximize desired ones (e.g., antidepressants are often prescribed for some neurological disorders); and (vi) the mechanism of mitochondrial protection by the three neuroprotectants creatine, minocycline and the heterocyclics all differ, suggesting potential for synergistic protection by these tree well tolerated agents.

In another embodiment, methods for protecting a subject against a mitochondrial component-mediated disease are disclosed. In one aspect of is embodiment, a subject is administered a therapeutically effective amount of a heterocyclic, tricyclics and phenothiazine or related compound of the present invention, i.e., a compound that protects against mitochondrial permeability transition.

In yet another embodiment, methods for treating a subject having a disease or undergoing an event in which mitochondrial triggers of cell death are activated or at risk of being activated are disclosed. In one aspect of this embodiment, a subject is administered a therapeutically effective amount of a compound of the present invention, i.e., a compound that protects against mitochondrial permeability transition and/or mitochondrial release of sequestered factors that contribute to cell death cascades.

Any of the identified PT protective compounds of the present invention can be administered to a subject, including a human, by itself, or in pharmaceutical compositions where it is mixed with suitable carriers or excipients at doses therapeutically effective to prevent, treat or ameliorate a variety of disorders, including those characterized by that outlined herein. A therapeutically effective amount or dose further refers to that amount of the compound sufficient result in the prevention or amelioration of symptoms associated with such disorders. Techniques for formulation and administration of the compounds of the instant invention may be found in Goodman and Gilman's The Pharmacological Basis of Therapeutics, Pergamon Press, latest edition.

The compounds of the present invention can be targeted to specific sites by direct injection into those sites. Compounds designed for use in the central nervous system should be able to cross the blood-brain barrier or be suitable for administration by localized injection.

Pharmaceutical compositions suitable for use in the present invention include compositions wherein the active ingredients are contained in an effective amount to achieve its intended purpose. Moreover, combinations of the agents described herein can also be employed. More specifically, a therapeutically effective amount means an amount effective to prevent development of or alleviate the existing symptoms and underlying pathology of the subject being treating. Determination of the effective amounts is well within the capability of those skilled in the art.

For any compound used in the methods of the present invention, the therapeutically effective dose can be estimated initially from cell culture assays. For example, a dose can be formulated in animal models to achieve a circulating concentration range that includes the IC₅₀ (the dose where 50% of the cells show the desired effects) as determined in cell culture. Such information can be used to more accurately determine useful doses in humans.

A therapeutically effective dose refers to that amount of the compound that results in the attenuation of symptoms or a prolongation of survival in a subject. Toxicity and therapeutic efficacy of such compounds can be determined by standard pharmaceutical procedures in cell cultures or experimental animals, e.g., for determining the IC₅₀ (the dose lethal to 50% of a given population) and the IC₅₀ (the dose therapeutically effective in 50% of a given population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage can vary within this range depending upon the dosage form employed and the route of administration utilized. The exact formulation, route of administration and dosage can be chosen by the individual physician in view of a patients condition. Dosage amount and interval can be adjusted individually to provide plasma levels of the active moiety which are sufficient to maintain the desired effects.

In one aspect, a therapeutically effective dose (effective dose or amount) ranges from about 0.1 mg/kg/body weight to about 100 mg/kg/body weight. In a particular aspect, an effective dose ranges from about 5 mg/kg/body weight to about 50 mg/kg/body weight.

In case of local administration or selective uptake, the effective local concentration of the drug may not be related to plasma concentration.

The amount of composition administered will, of course, be dependent on the subject being treated, on the subjects weight, the severity of the affliction, the manner of administration and the judgment of the prescribing physician.

The pharmaceutical compositions of the present invention can be manufactured in a manner that is itself known, e.g., by means of conventional mixing, dissolving, granulating, levigating, emulsifying, encapsulating, entrapping or lyophilizing processes.

Pharmaceutical compositions for use in accordance with the present invention thus can be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention can be formulated in aqueous solutions, preferably in physiologically compatible buffers such as Hank's solution, Ringer's solution, or physiological saline buffer. For transmucosal administration, penetrants appropriate to the barriers to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinyl-pyrrolidone (PVP). If desired, disintegrating agents can be added, such as the cross-linked polyvinyl pyrrolidone, agar, or alginic acid or a salt thereof such as sodium alginate.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodi-fluoromethane, trichlorofluoromethane, dichlorotetrafluoromethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The compounds can be formulated for parenteral administration by injection, e.g., by bolus injection or continuous infusion Formulations for injection can be presented in unit dosage for, e.g., in ampoules or in multidose containers, with an added preservatives. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspension. Suitable lipohilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions.

Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations previously described, the compounds can also be formulated as a depot preparation. Such long acting formulations can be administer by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

A pharmaceutical carrier for the hydrophobic compounds of the invention is a co-solvent system comprising benzyl alcohol, a non-polar surfactant, a water-miscible organic polymer, and an aqueous phase. Naturally, the proportions of a co-solvent system can be varied considerably without destroying its solubility and toxicity characteristics. Furthermore, the identity of the cosolvent components can be varied.

Alternatively, other delivery systems for hydrophobic pharmaceutical compounds can be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds can be delivered using a sustained-release system, such as semipermeable matrices of solid hydrophobic polymers containing the therapeutic agent. Various of sustained-release materials have been established and are well known to those skilled in the art. Sustained-release capsules can, depending on their chemical nature, release the compounds for a few weeks up to over 100 days. Depending on the chemical nature and the biological stability of the therapeutic reagent, additional strategies for protein stabilization can be employed.

The pharmaceutical compositions also can comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Many of the compounds of the invention can be provided as salts with pharmaceutically compatible counterions. Pharmaceutically compatible salts can be formed with many acids, including but not limited to hydrochloric, sulfuric, acetic, lactic, tartaric, malic, succinic, etc. Salts tend to be more soluble in aqueous or other protonic solvents that are the corresponding free base forms.

Suitable routes of administration can, e.g., include oral, rectal, transmucosal, transdermal, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one can administer the compound in a local rather than systemic manner, e.g., via injection of the compound directly into an affected area, often in a depot or sustained release formulation.

Furthermore, one can administer the compound in a targeted drug delivery system e.g., in a liposome coated with an antibody specific for affected cells. The liposomes will be targeted to and taken up selectively by the cells.

The compositions can, if desired, be presented in a pack or dispenser device which can contain one or more unit dosage forms containing the active ingredient. The pack can, e.g., comprise metal or plastic foil, such as a blister pack. The pack or dispenser device can be accompanied by instruction for administration. Compositions comprising a compound of the invention formulated in a compatible pharmaceutical carrier can also be prepared, placed in an appropriate container, and labeled for treatment of an indicated condition. Suitable conditions indicated on the label can include treatment of a disease such as described herein.

### EXAMPLES

### Example 1

### Promethazine as a Neuroprotectant

Male Lewis 344 rats were administered 3-nitroproprionic acid by subcutaneous Alzet pumps. 3-Nitropropionic acid is a mitochondrial toxin which causes selective degeneration in the basal ganglia, mice, rats and primates. The lesions that it produces highly mimic the neuropathologic features associated with Huntington's Disease. The male Lewis rats weighed from about 340 to abut 370 grams and were obtained from Charles River Laboratories. At twelve weeks of age, the subcutaneous pumps were implanted. The concentration for each individual rat was calculated so that the animals received a dose of 38 mg/kg/d. Control animals were implanted with empty osmotic pumps. The 3-nitropropionic acid was administered for one week. Animals were treated with either phosphate buffered saline (PBS) promethazine in PBS at doses of 5 to 40 mg/kg/d. One week after the administration of the compound the rats were deeply anesthetized and transcardially perfused with 4% paraformaldehyde in PBS. Subsequently, 50 μm sections were obtained and stained for nissel substance. The lesion volumes were then calculated using stereologic procedures.

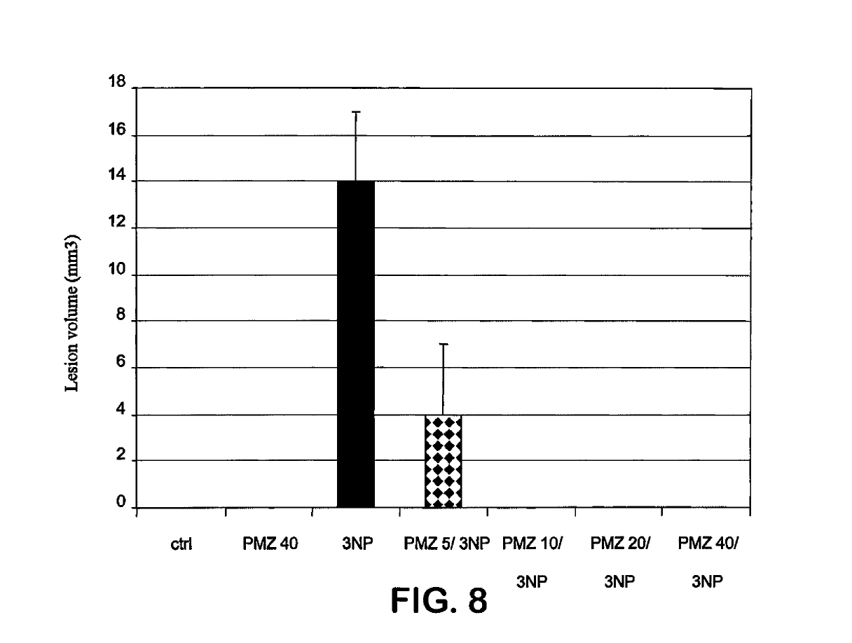

As demonstrated by FIG. 8, increasing dosages of promethazine blocked 3-nitropropionic induced lesions. Striatal lesion volume was determined in Lewis rats treated with 3-nitropropionic acid with or without promethazine. This bar graph demonstrates that without promethazine, the 3-nitropropionic acid produced significant striatal lesion volume (˜14 mm³). However, when the rats were treated with promethazine, a significant diminishment in lesion volume is observed (˜4 mm³).

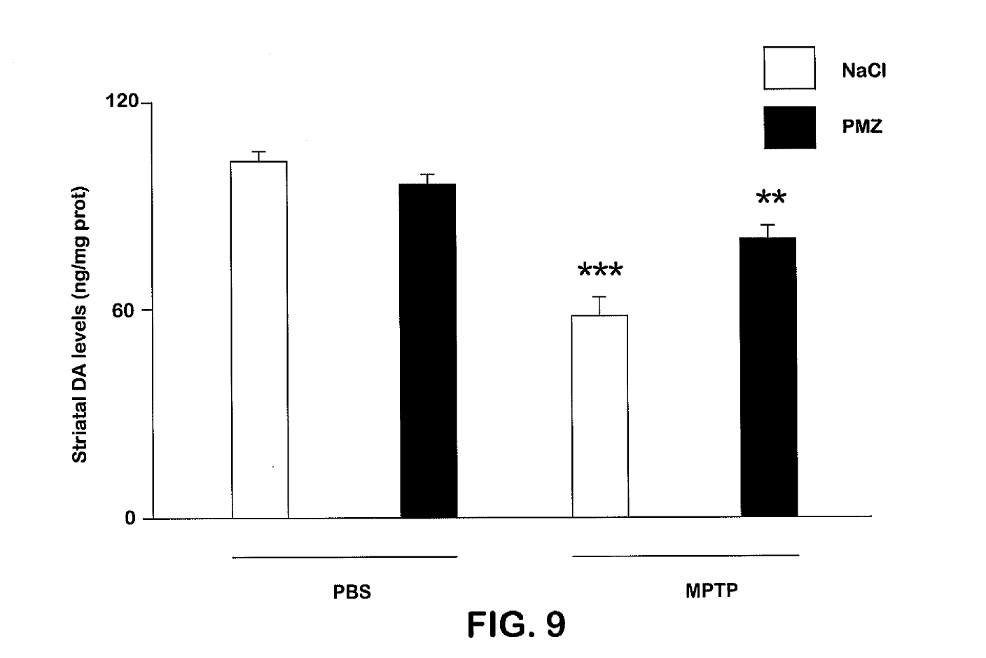

1-Methyl-4-phenyl-1,2,3,6-tetrahydropyridine (MPTP) has been employed as an experimental animal model for Parkinson's Disease. As evidenced by FIG. 9, promethazine appeared to have no effect on striatal dopamine levels at base line. Following the administration of MPTP there was a significant reduction in striatal dopamine levels which was significantly attenuated by promethazine administration at a dose of 20 mg/kg.

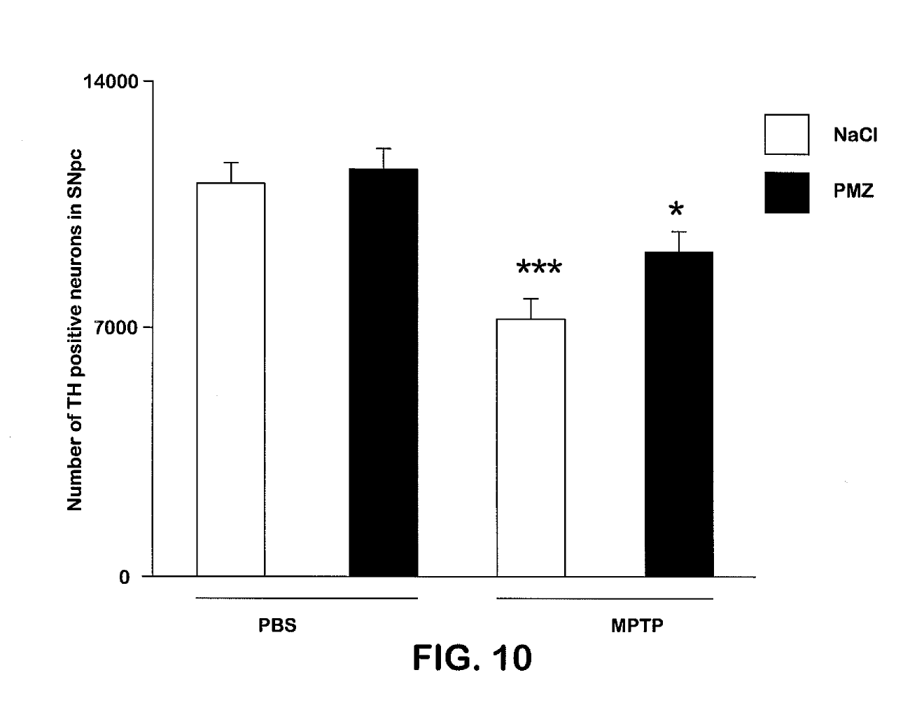

FIG. 10 provides evidence demonstrating that there was significant attenuation of loss of dopaminergic neurons in the substantia nigra pars compacta. It is important to note that a loss of neurons in this area is a characteristic feature of Parkinson's Disease. As shown in FIG. 10, there was a 50% cell loss following administration of MPTP and this was significantly attenuated by the promethazine administration.

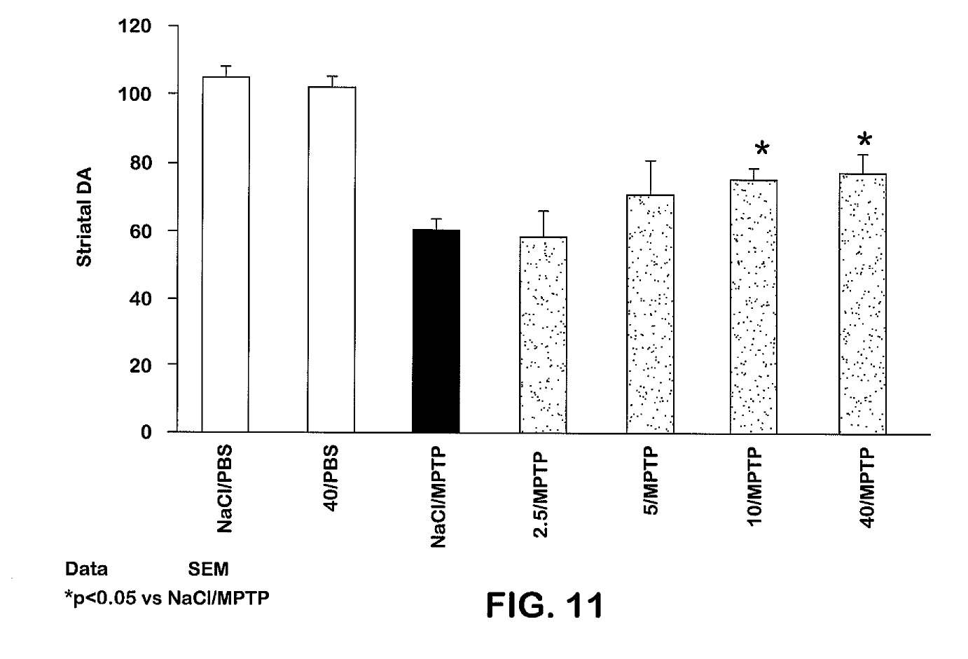

FIG. 11 represents a dose response curve. The effects of promethazine at 2.5, 5, and 40 mg/kg were examined. This figure demonstrates that there is dose-dependent neuroprotection with optimal effects at 10 and 40 mg/kg, which are significantly different from each other.

### Example 2

### Nortriptyline as a Neuroprotectant

Using the mouse middle cerebral artery occlusion (MCAO) protocol as published in Zhang et al., PNAS 2003 Dec. 23; 100(26):16012-7. Epub 2003 Dec. 8, we demonstrated that nortriptyline mediates neuroprotection following MCAO.

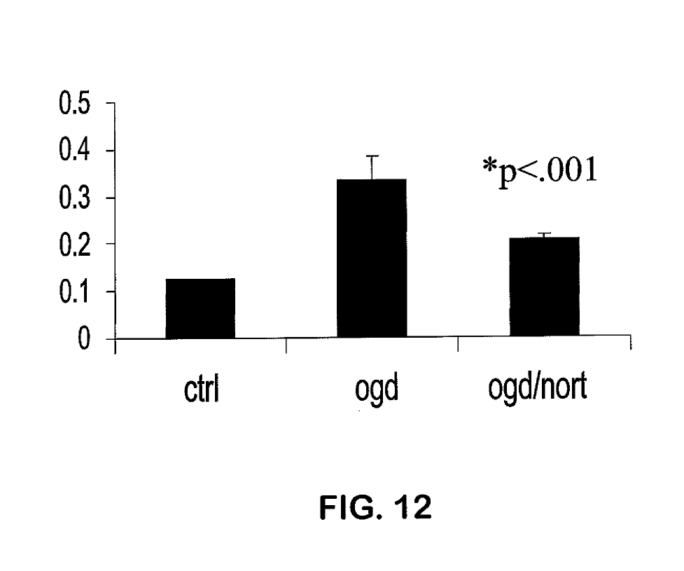

FIG. 12 shows the efficacy of nortriptyline in mitigating oxygen-glucose deprivation (OGD). Specifically, FIG. 12 demonstrate the use of nortriptyline in preventing cell death, as measured by LDH release (y-axis).

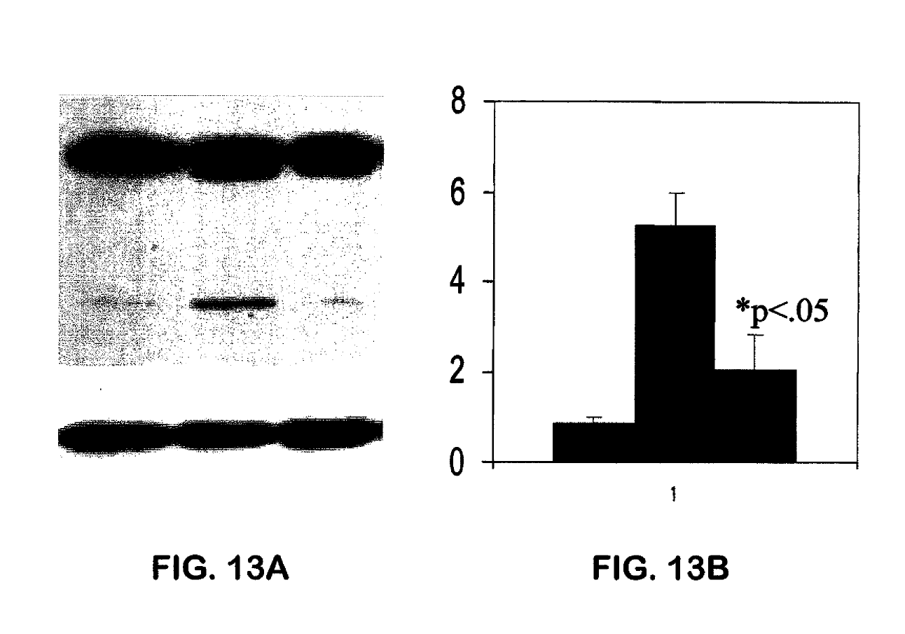

Nortriptyline has also been shown to inhibit OGD-mediated caspase-3 activation. See, FIG. 13. Following OGD, we detected caspase-3 activation by Western blot (FIG. 13a, second lane). Addition of nortriptyline inhibits caspase-3 activation (FIG. 13a third lane). FIG. 13b represents the densitometry for FIG. 13a.

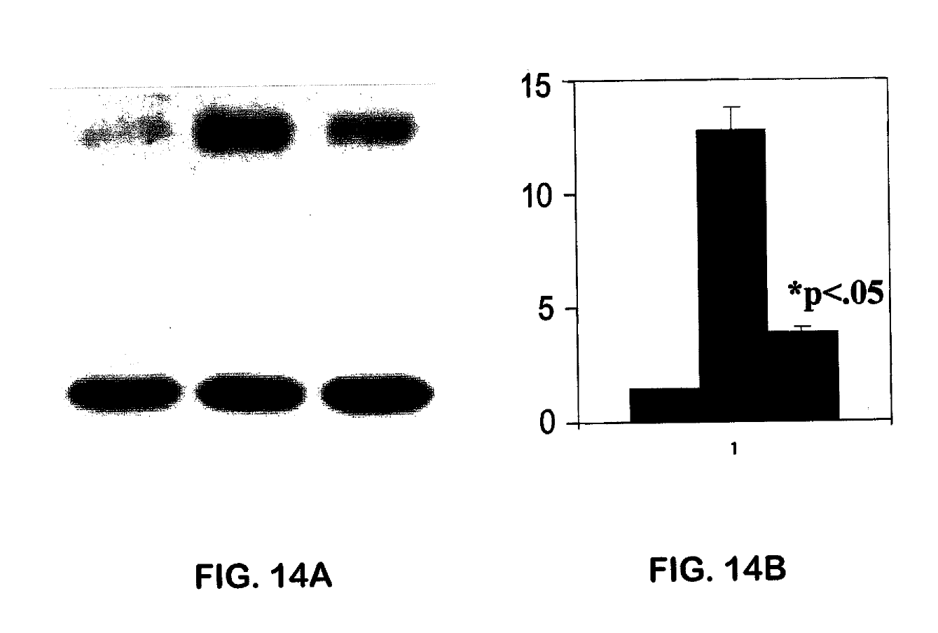

FIG. 14 illustrates the utility of nortriptyline in Inhibiting OGD mediated cytochrome C release. Inhibition of mitochondrial cytochrome C release is likely responsible for the inhibition of caspase-3 activation and neuroprotection. It is important to note however, that cytochrome C can serve as a marker for other propagating factors that are not mediated by caspase-3.

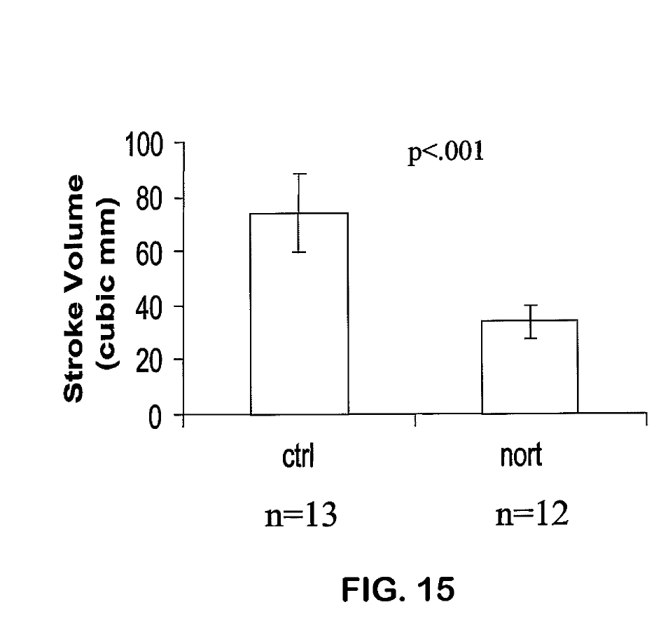

Nortriptyline was also found to reduce infarct volume. FIG. 15 shows the effect of nortriptyline on stroke volume. With the administration of nortriptyline a significant reduction in stroke volume was achieved. The significance of which is that inhibition of cytochrome C release by nortiptilyne, leading to inhibition of caspase-3 activation, likely leads to inhibition of neuronal cell death and neuroprotection.

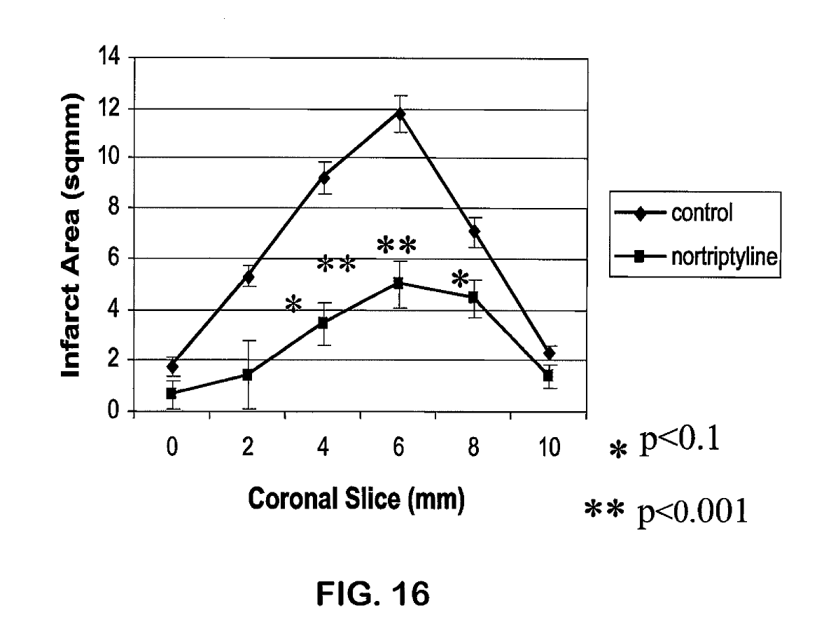

FIG. 16 shows the effect of nortriptyline on infarct volume in coronal slices. Clearly, nortriptyline decreases the infarct area as compared to the controls.

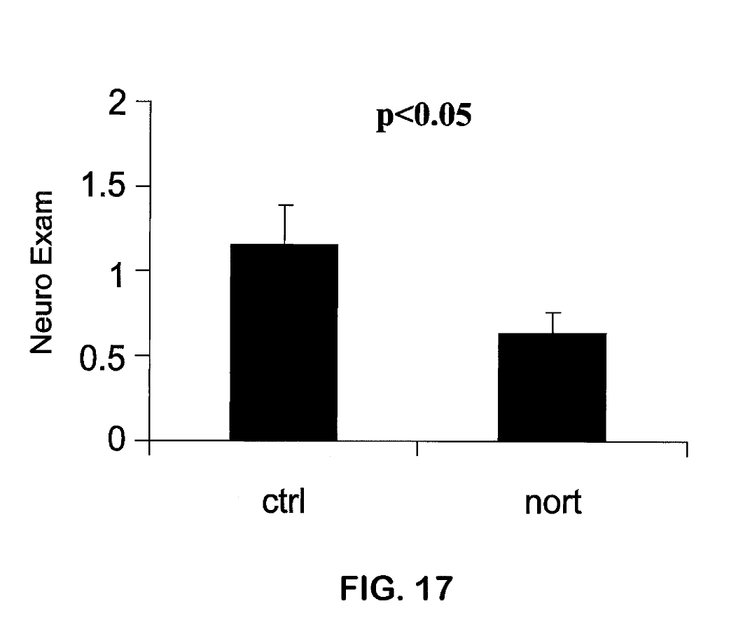

FIG. 17 shows that administration of nortriptyline improves a neurological examination following cerebral ischemia.

While this invention has been particularly shown and described with references to specific embodiments, it will be understood by those skilled in the art that various changes in form and details may be made therein without departing from the spirit and scope of the invention.

1. A composition used to prevent or treat mitochondrial-based pathological states, comprising a heterocyclic, tricyclic or phenothiazine compound, wherein said compound has at least a two member central ring.

2. The composition of claim 1 selected from the group consisting of methiothepin, promethazine, triflupromazine, clomipramine, flufenazine, chlorprothixene, nortriptyline, promazine, thioridazine, mefloquine, desipramine, chlorpromazine, prochlorperazine, propiomazine, pimethixene, perphenazine, amitriptyline, amoxepine, maprotiline, quinacrine, periciazine, ethopropazine, mianserin, cyclobenzaprine, imipramine, clozapine, and doxepin.

3. The composition of claim 2, wherein said composition is methiothepin.

4. The composition of claim 2, wherein said composition is promethazine.

5. The composition of claim 2, wherein said composition is triflupromazine.

6. The composition of claim 2, wherein said composition is clomipramine.

7. The composition of claim 2, wherein said composition is flufenazine.

8. The composition of claim 2, wherein said composition is chlorprothixene.

9. The composition of claim 2, wherein said composition is nortriptyline.

10. The composition of claim 2, wherein said composition is promazine.

11. The composition of claim 2, wherein said composition is thioridazine.

12. The composition of claim 2, wherein said composition is mefloquine.

13. The composition of claim 2, wherein said composition is desipramine.

14. The composition of claim 2, wherein said composition is chlorpromazine.

15. The composition of claim 2, wherein said composition is prochlorperazine.

16. The composition of claim 2, wherein said composition is propiomazine.

17. The composition of claim 2, wherein said composition is pimethixene.

18. The composition of claim 2, wherein said composition is perphenazine.

19. The composition of claim 2, wherein said composition is amitriptyline.

20. The composition of claim 2, wherein said composition is amoxepine.

21. The composition of claim 2, wherein said composition is maprotiline.

22. The composition of claim 2, wherein said composition is quinacrine.

23. The composition of claim 2, wherein said composition is periciazine.

24. The composition of claim 2, wherein said composition is ethopropazine.

25. The composition of claim 2, wherein said composition is mianserin.

26. The composition of claim 2, wherein said composition is cyclobenzaprine.

27. The composition of claim 2, wherein said composition is imipramine.

28. The composition of claim 2, wherein said composition is clozapine.

29. The composition of claim 2, wherein said composition is doxepin.

30. A method for protecting a subject against a mitochondrial component-mediated disease, comprising administering to said subject an effective amount of a compound effective in inhibiting mitochondrial permeability transition.

31. The method of claim 30, wherein said compound is mefloquine.

32. The method of claim 30, wherein said effective amount ranges from about 0.1 mg/kg to about 100 mg/kg.

33. The method of claim 32, wherein said effective amount ranges from about 5 mg/kg to about 50 mg/kg.

34. The method of claim 32, wherein said disease is stroke, heart attack, neurological insult, brain trauma, spinal cord injury, chemical toxicity, liver, muscle, kidney reperfusion or a combination thereof.

35. A method for treating a subject affected with a mitochondrial component-mediated disease by administering to said subject a therapeutic dose of a compound effective in inhibiting mitochondrial permeability transition.

36. The method of claim 35, wherein said compound is mefloquine.

37. The method of claim 35, wherein said effective amount ranges from about 0.1 mg/kg to about 100 mg/kg.

38. The method of claim 35, wherein said effective amount ranges from about 5 mg/kg to about 50 mg/kg.

39. The method of claim 35, wherein said disease is stroke, heart attack, neurological insult, brain trauma, spinal cord injury, chemical toxicity, liver, muscle, kidney reperfusion or a combination thereof.

40. The method of claim 39, wherein said neurological insult is a neurodegenerative disease.

41. The method of claim 40, wherein said neurodegenerative disease is Amyotrophic Lateral Sclerosis, Parkinson's disease, Huntington's disease, Alzheimer's disease, heart attack, stroke, or a combination thereof.

42. A pharmaceutical preparation comprising a drug and a suitable drug carrier, wherein said drug is selected from the group consisting of methiothepin, promethazine, triflupromazine, clomipramine, flufenazine, chlorprothixene, nortriptyline, promazine, thioridazine, mefloquine, desipramine, chlorpromazine, prochlorperazine, propiomazine, pimethixene, perphenazine, amitriptyline, amoxepine, maprotiline, quinacrine, periciazine, ethopropazine, mianserin, cyclobenzaprine, imipramine, clozapine, and doxepin.

43. The pharmaceutical preparation of claim 42, wherein said suitable carrier is selected from the group consisting of a tablet, pill, dragee, capsule, liquid, gel, syrup, slurry, suspension and the like.

44. The pharmaceutical preparation of claim 42 further comprising a suitable excipient.

45. The pharmaceutical preparation of claim 44, wherein said excipient is selected from the group consisting of sugars, including lactose, sucrose, mannitol, sorbitol, cellulose, including maize starch, wheat starch, rice starch, potato starch, gelatin, gum tragacanth, methyl cellulose, hydroxypropylmethylcellulose, sodium carboxymethyl cellulose, polyvinyl-pyrrolidone (PVP) and a combination thereof.
